(19) United States
(12) Patent Application Publication
(10) Pub. No.: US 2025/0283164 A1
(43) Pub. Date: Sep. 11, 2025

(54) RNA-BINDING BY TRANSCRIPTION FACTORS

(71) Applicant: Whitehead Institute for Biomedical Research, Cambridge, MA (US)

(72) Inventors: Richard A. Young, Cambridge, MA (US); Jonathan Henninger, Cambridge, MA (US); Ozgur Oksuz, Cambridge, MA (US)

(21) Appl. No.: 18/859,711

(22) PCT Filed: Apr. 25, 2023
(86) PCT No.: PCT/US2023/066220

§ 371 Date: Oct. 24, 2024

Related U.S. Application Data
(60) Provisional application No. 63/334,651, filed on Apr. 25, 2022.

(51) Int. Cl. C12Q 1/6869; A61K 48/00; C12Q 1/6806; C12Q 1/6809; C12Q 1/6844; C12Q 1/6876

(52) U.S. Cl. CPC C12Q 1/6869; A61K 48/005; C12Q 1/6806; C12Q 1/6809; C12Q 1/6844; C12Q 1/6876

(57) ABSTRACT

Expression of a target gene is modulated by an agent that modulates binding between a selected ribonucleic acid (RNA) transcribed from at least one regulatory element of a target gene and a transcription factor that binds to both the RNA and the at least one regulatory element. The agent is selected to bind to an RNA having binding affinity for a region of the transcription factor that is at least nine contiguous amino acids, at least one amino acid of the region is arginine, and a majority of amino acids of the region are arginine or lysine. Modulating binding between the RNA and the transcription factor modulates expression of the target gene.

### RELATED APPLICATION

This application is a U.S.C. § 371 national phase application of PCT International Application No. PCT/US2023/066220, filed on Apr. 25, 2023, which claims the benefit of U.S. Provisional Application No. 63/334,651, filed on Apr. 25, 2022. The entire teachings of the above application are incorporated herein by reference.

### GOVERNMENT SUPPORT

This invention was made with government support under GM123511 awarded by the National Institutes of Health (NIH). This invention was made with government support under CA155258 awarded by the National Institutes of Health (NIH). This invention was made with government support under F32CA254216-01 awarded by the National Institutes of Health (NIH). The government has certain rights in the invention.

### BACKGROUND

Transcription factors (TFs) bind specific sequences in promoter-proximal and distal DNA elements in order to regulate gene transcription. Active promoters and enhancer elements are transcribed bi-directionally (see e.g., Core et al., 2008; Seila et al., 2008; and Sigova et al., 2013). Although various models have been proposed for the roles of RNA species produced from these regulatory elements, their functions are not fully understood (Kim et al., 2010; Wang et al., 2011; Melo et al., Mol Cell 49, 524-535 (2013); Lai et al., 2013; Lam et al., 2013; Li et al., 2013; Kaikkonen et al., 2013; Mousavi et al., 2013; Di Ruscio et al., 2013; and Schaukowitch et al., 2014).

### SUMMARY

Transcription factors (TFs) orchestrate the gene expression programs that define each cell's identity. The canonical TF accomplishes this with two domains, one that binds specific DNA sequences and the other that binds protein coactivators or corepressors. We find that at least half of TFs also bind RNA, doing so through a previously unrecognized domain with sequence and functional features analogous to the arginine-rich motif of the HIV transcriptional activator Tat. RNA binding contributes to TF function by promoting the dynamic association between DNA, RNA and TF on chromatin. TF-RNA interactions are a conserved feature essential for vertebrate development and disrupted in disease. We propose that the ability to bind DNA, RNA and protein is a general property of many TFs and is fundamental to their gene regulatory function.

In some aspects, described herein is a method of modulating expression of a target gene in a subject. The method involves administering to the subject an oligonucleotide that is antisense to a ribonucleic acid (RNA) that binds a region of a transcription factor for the target gene, whereby binding between the oligonucleotide and the RNA inhibits binding between the RNA and the transcription factor, thereby modulating expression of the target gene. The region of the transcription factor is at least nine contiguous amino acids, at least one amino acid of the region is arginine, and a majority of amino acids of the region are arginine or lysine.

In some aspects, described herein is a method of modulating expression of a target gene. The method involves providing an agent that modulates binding between a selected ribonucleic acid (RNA) transcribed from at least one regulatory element of a target gene and a transcription factor which binds to both the RNA and the at least one regulatory element, wherein the agent is selected to bind to an RNA having binding affinity for a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and contacting the agent with a cell that exhibits aberrantly increased or decreased expression of the target gene or aberrantly increased or decreased activity of a gene product of the target gene.

In some aspects, the methods described herein further include identifying the RNA that binds the region of the transcription factor for the target gene. Identifying the RNA that binds to the region of the transcription factor for the target gene can include: a) crosslinking the RNA to the transcription factor for the target gene by: i) contacting the transcription factor with 4-thiouridine (4SU); and ii) exposing the transcription factor to ultraviolet radiation, thereby generating an RNA-transcription factor complex; b) immunoprecipitating the RNA-transcription factor complex; c) lysing the RNA from the RNA-transcription factor complex; and d) sequencing the RNA.

Identifying the RNA that binds to the region of the transcription factor for the target gene can include computational analysis of an overlap of genomic binding sites for the transcription factor and sequencing of RNA transcribed from the genomic binding site.

The RNA can be transcribed from a genomic locus within 1 kilobase of a genomic locus bound by the transcription factor. The RNA can be transcribed from a genomic locus more than 1 kilobase of a genomic locus bound by the transcription factor.

A first or last amino acid of the region of the transcription factor is within 10 amino acids of a DNA-binding domain of the transcription factor. Binding between the oligonucleotide and the RNA causes a change in secondary structure of the RNA.

The RNA can bind to the transcription factor with a Kd from 40 nM to 1200 nM. The RNA can be seven to fifteen nucleotides. The RNA can be eleven nucleotides. The RNA can be at least seven nucleotides. The RNA can be no more than fifteen nucleotides.

At least 75% of amino acids of the region of the transcription factor can be arginine or lysine. At least 80% of amino acids of the region of the transcription factor are arginine or lysine. At least 85% of amino acids of the region of the transcription factor are arginine or lysine. At least 90% of amino acids of the region of the transcription factor are arginine or lysine. The transcription factor can include a DNA binding domain selected from the group consisting of a zinc finger, leucine zipper, helix-turn-helix, winged helix-turn-helix, helix-loop-helix, high mobility group (HMG) box, and GB-fold. The transcription factor can be a human transcription factor.

A method of identifying transcription factors that bind to RNA includes: a) crosslinking an RNA to the transcription factor by: i) contacting the transcription factor with 4-thiouridine (4SU); and ii) exposing the transcription factor to ultraviolet radiation, thereby generating an RNA-transcription factor complex; and b) performing liquid chromatography with tandem mass spectrometry (LC-MS/MS) to identify transcription factors that bind to the RNA.

A method of modulating expression of a target gene in a subject includes: administering to the subject an oligonucleotide that is antisense to a ribonucleic acid (RNA) that binds a region of a transcription factor for the target gene, whereby binding between the oligonucleotide and the RNA inhibits binding between the RNA and the transcription factor, thereby modulating expression of the target gene, wherein the region of the transcription factor is at least nine contiguous amino acids, at least one amino acid of the region is arginine, and a majority of amino acids of the region are arginine or lysine.

A method of modulating expression of a target gene includes: a) providing an agent that modulates binding between a selected ribonucleic acid (RNA) transcribed from at least one regulatory element of a target gene and a transcription factor which binds to both the RNA and the at least one regulatory element, wherein the RNA is selected based on its ability to bind to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and b) contacting the agent with a cell that exhibits aberrantly increased or decreased expression of the target gene or aberrantly increased or decreased activity of a gene product of the target gene.

A method of modulating expression of a target gene includes modulating binding between a ribonucleic acid (RNA) transcribed from at least one regulatory element of a target gene and a transcription factor which binds to both the RNA and the at least one regulatory element, wherein the RNA binds to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene.

A method of modulating expression of a target gene includes: a) providing an agent that modulates binding between a selected ribonucleic acid (RNA) transcribed from at least one regulatory element of a target gene and a transcription factor which binds to both the RNA and the at least one regulatory element, wherein the selected RNA has been demonstrated to bind to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and; and b) contacting the agent with a cell that exhibits aberrantly increased or decreased expression of the target gene or aberrantly increased or decreased activity of a gene product of the target gene.

In some aspects, described herein is the insight that the activation or repression activity of any transcription factor may involve its interaction with regulatory RNAs at the locus where they are transcribed. The use of a RNA-binding moiety such as an anti-sense oligonucleotide (ASO) directed to any one gene's regulatory RNA(s) can be predicted to cause an increase or decrease in transcription of that gene, allowing for upregulation or downregulation of a specific gene. This might be because an activating TF is stabilized at the locus by binding both DNA and RNA, and similarly, a repressing TF might be stabilized at the locus by binding both DNA and RNA. ASOs or other RNA-binding moieties would bind the regulatory RNA and interfere with one or the other type of regulatory TF. For example, transcription of a gene may be increased by administration of a RNA-binding moiety (e.g., an ASO) that binds to a regulatory RNA that would otherwise stabilize a repressing TF at the locus. Transcription of a gene may be decreased by administration of a RNA-binding moiety (e.g., an ASO) that binds to a regulatory RNA that would otherwise stabilize an activating TF at the locus. Such RNA-binding moieties may be useful as therapeutic agents in any of a wide variety of disorders in which aberrantly increased or decreased transcription plays a role or in which increasing or decreasing the transcription of a gene could provide a therapeutic benefit.

In some aspects, an assay may be used to identify agents that, when added to a system comprising an RNA (e.g., a labeled RNA such as a fluorescently labeled RNA) and a transcription factor, increase or decrease binding of the transcription factor to RNA (e.g., regulatory RNA). For example, a test agent may be added to such a system and the effect of the test agent on binding of the RNA to the transcription factor may be measured.

In some aspects, an assay such may be used to identify a mutation in a transcription factor (e.g., in a basic patch of a TF) that alters binding of a transcription factor to a regulatory RNA.

In some aspects, an assay may be used to identify a subject harboring a mutation that alters binding of a TF to a regulatory RNA. Such a subject may be a candidate for therapy with an agent that addresses such altered binding.

In one aspect, the presently disclosed subject matter provides a method of modulating expression of a target gene, the method comprising modulating binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene. In some embodiments, the RNA is a non-coding RNA selected from the group consisting of enhancer RNA, promoter RNA, super-enhancer constituent RNA, and combinations thereof. In some embodiments, at least one regulatory element is selected from the group consisting of an enhancer, a promoter, a super-enhancer constituent, and combinations thereof.

In some embodiments, modulating binding comprises promoting binding between the RNA and the transcription factor. In some embodiments, promoting binding between the RNA and the transcription factor stabilizes occupancy of the transcription factor at the at least one regulatory element, thereby increasing expression of the target gene. In some embodiments, promoting binding between the RNA and the transcription factor comprises tethering an RNA that binds to the transcription factor to a DNA sequence in proximity to the at least one regulatory element.

In some embodiments, modulating binding comprises interfering with binding between the RNA and the transcription factor. In some embodiments, interfering with binding between the RNA and the transcription factor destabilizes occupancy of the transcription factor at the at least one regulatory element, thereby decreasing expression of the target gene.

In some embodiments, modulating expression of the target gene occurs in vitro or ex vivo. In some embodiments, modulating expression of the target gene comprises contacting a cell with an effective amount of an agent which interferes with binding between the RNA and the transcription factor.

In some embodiments, modulating expression of the target gene occurs in vivo. In some embodiments, modulating expression of the target gene comprises administering to a subject an effective amount of a composition which interferes with binding between the RNA and the transcription factor. In some embodiments, the composition comprises an agent which binds to the transcription factor in a manner that prevents the transcription factor from binding to the RNA. In some embodiments, the agent does not compete with a DNA sequence in the at least one regulatory element for binding to the transcription factor. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the agent comprises a decoy RNA. In some embodiments, the decoy RNA comprises a synthetic RNA selected from the group consisting of: (i) a synthetic RNA having a nucleotide sequence that is homologous to the RNA transcribed from the at least one regulatory element; (ii) a synthetic RNA having a nucleotide sequence that is homologous to an RNA binding site for the transcription factor; (iii) a synthetic RNA that binds to the transcription factor at a site other than the DNA binding domain of the transcription factor; (iv) a synthetic RNA having a nucleotide sequence that is at least partially complementary to the RNA transcribed from the at least one regulatory element; and (v) a synthetic RNA having a nucleotide sequence that is at least partially complementary to a binding site for the transcription factor in the RNA transcribed from the at least one regulatory element. In some embodiments, the synthetic RNA comprises a nucleotide sequence that comprises an RNA binding site for the transcription factor. In some embodiments, the synthetic RNA comprises a length of between 10 nucleotides and 300 nucleotides. In some embodiments, the synthetic RNA comprises a length of between 30 and 60 nucleotides.

In some embodiments, the synthetic RNA contains at least one modification.

In some embodiments, the composition comprises an agent which binds to the RNA in a manner that prevents the transcription factor from binding to the RNA. In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. In some embodiments, the agent is an RNA interfering agent selected from the group consisting of a ribozyme, guide RNA, small interfering RNA (siRNA), short hairpin RNA or small hairpin RNA (shRNA), microRNA (miRNA), post-transcriptional gene silencing RNA (ptgsRNA), short interfering oligonucleotide, antisense oligonucleotide, aptamer, and CRISPR RNA.

In some embodiments, the composition modifies at least one nucleotide of a DNA sequence of the at least one regulatory element in a manner that prevents RNA transcribed from the at least one regulatory element from binding to the transcription factor. In some embodiments, the composition comprises a genomic editing system selected from the group consisting of a CRISPR\Cas system, zinc finger nucleases (ZFNs), Transcription Activator-Like Effector Nucleases (TALENs), and engineered meganuclease re-engineered homing endonucleases.

In some embodiments, the composition comprises an agent which prevents exosomal degradation of untethered RNA in proximity to the at least one regulatory element or the transcriptional machinery. In some embodiments, the agent inhibits a component of the exosome. In some embodiments, the agent inhibits a component of the exosome via RNA interference.

In some embodiments, the target gene comprises a gene for which increased or aberrant transcription is associated with a disease, condition, or disorder. In some embodiments, the disease, condition, or disorder is selected from the group consisting of a cancer, a genetic disorder, a liver disorder, a neurodegenerative disorder, and an autoimmune disease. In some embodiments, the target gene comprises an oncogene. In some embodiments, the target gene comprises at least one mutation in the at least one regulatory element, wherein the at least one mutation results in the transcription factor binding to RNA transcribed from the at least one regulatory element in a manner that stabilizes occupancy of the transcription factor to the at least one regulatory element, thereby increasing expression of the target gene. In some embodiments, the at least one mutation comprises a single nucleotide polymorphism.

In some aspects, the presently disclosed subject matter provides a method of identifying a candidate agent that interferes with binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein decreased binding of the transcription factor to the RNA transcribed from the at least one regulatory element in the presence of the test agent as compared to the absence of the test agent indicates that the test agent is a candidate agent that interferes with binding between the RNA and the transcription factor.

In some embodiments, the methods further comprise identifying a transcription factor that binds to RNA transcribed from at least one regulatory element and to the at least one regulatory element. In some embodiments, the methods further comprise identifying an RNA binding domain of the transcription factor. In some embodiments, the methods further comprise identifying a consensus motif in the RNA transcribed from the at least one regulatory sequence for the RNA binding domain of the transcription factor.

In some embodiments, assessing binding comprises contacting a complex or mixture comprising the transcription factor, the at least one regulatory element, and the RNA transcribed from the at least one regulatory element with the test agent. In some embodiments, the methods further comprise assessing whether the test agent is capable of binding to the transcription factor at a site other than a DNA binding domain of the transcription factor. In some embodiments, the test agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the test agent comprises a decoy RNA. In some embodiments, the decoy RNA comprises a synthetic RNA selected from the group consisting of: (i) a synthetic RNA having a nucleotide sequence that is homologous to the RNA transcribed from the at least one regulatory element; (ii) a synthetic RNA having a nucleotide sequence that is homologous to an RNA binding site for the transcription factor; (iii) a synthetic RNA that binds to the transcription factor at a site other than the DNA binding domain of the transcription factor; (iv) a synthetic RNA having a nucleotide sequence that is at least partially complementary to the RNA transcribed from the at least one regulatory element; and (v) a synthetic RNA having a nucleotide sequence that is at least partially complementary to a binding site for the transcription factor in the RNA transcribed from the at least one regulatory element. In some embodiments, the synthetic RNA comprises a nucleotide sequence that comprises an RNA binding site for the transcription factor. In some embodiments, the synthetic RNA comprises a length of between 10 nucleotides and 300 nucleotides. In some embodiments, the synthetic RNA comprises a length of between 30 and 60 nucleotides. In some embodiments, binding is performed in a cell. In some embodiments, the methods comprise performing cross-linking immunoprecipitation (CLIP) with the RNA and the transcription factor.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning. A Laboratory Manual, 3^{rd} ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange 10^{th} ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, available on the World Wide Web: ncbi.nlm.nih.gov/omim, and in Online Mendelian Inheritance in Animals (OMIA), a database of genes, inherited disorders and traits in animal species (other than human and mouse), available on the World Wide Web: omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-F. Transcription factor binding to RNA in cells. (FIG. 1A) Schematic of DNA-binding and effector domains in transcription factors from different families (PDB accession numbers in Methods). (FIG. 1B) Experimental scheme for RBR-ID in human K562 cells. 4SU-labeled RNAs are crosslinked to proteins with UV light. RNA-binding peptides are identified by comparing the levels of crosslinked and unbound peptides by mass spectrometry. (FIG. 1C) Volcano plot of TF peptides in RBR-ID for human K562 cells with select highlighted TFs (dotted line at p=0.05). Each marker represents the peptide with maximum RBR-ID score for each protein. (FIG. 1D) Volcano plot of all detected peptides in RBR-ID for human K562 cells with select highlighted RBPs (dotted line at p=0.05). Each marker represents the peptide with maximum RBR-ID score for each protein. (FIG. 1E) ChIP-seq and CLIP signal for GATA2 at the HINT1 locus in K562 cells. (FIG. 1F) Meta-gene analysis of input-subtracted CLIP signal centered on GATA2 or RUNX1 ChIPseq peaks in K562 cells.

FIGS. 2A-C. Transcription factor binding to RNA in vitro. (FIG. 2A) Experimental scheme for measuring the equilibrium dissociation constant (Kd) for protein-RNA binding. Cy5-labeled RNA and increasing concentrations of purified proteins are incubated and protein-RNA interactions is measured by fluorescence polarization assay. (FIG. 2B) Fraction bound RNA with increasing protein concentration for established RNA-binding proteins, GFP, and the restriction enzyme BamHI (error bars depict s.d.). (FIG. 2C) Fraction bound RNA with increasing protein concentration for select transcription factors (error bars depict s.d.). A summary of Kd values for established RNA-binding proteins and TFs are indicated.

FIGS. 3A-H. An arginine-rich domain in transcription factors. (FIG. 3A) Plot depicting the probability of a basic patch as a function of the distance from either DNA-binding domains (dotted line) or all other annotated structured domains (black). (FIG. 3B) Sequence logo (SEQ ID NO: 5) derived from a position-weight matrix generated from the basic patches of TFs. (FIG. 3C) Cumulative distribution plot of maximum cross-correlation scores between proteins and the Tat ARM (*p<0.0001, Mann Whitney U test) for the whole proteome excluding TFs (black line) or TFs alone (dotted line). (FIG. 3D) Diagram of select TFs and their cross-correlation to the Tat ARM across a sliding window (*maximum scoring ARM-like region). Evolutionary conservation as calculated by ConSurf (Methods) is provided as a heatmap below the protein diagram. (FIG. 3E) Fraction bound RNA with increasing protein concentration for wildtype (WT) or deletion (ΔARM) TFs (KLF4 WT vs ΔARM: p=0.017; SOX2 WT vs ΔARM: p=0.0012; GATA2 WT vs ΔARM: p=0.018). (FIG. 3F) Gel shift assay for 7SK RNA with synthesized peptides encoding wildtype or R/K>A mutations of TF-ARMs. HIV Tat ARM (SEQ ID NO: 9); WT KLF4 ARM (SEQ ID NO: 10); R/K>A KLF4-ARM (SEQ ID NO: 11); WT SOX2-ARM (SEQ ID NO: 12): R/K>A SOX2-ARM (SEQ ID NO: 13); WT GATA2-ARM (SEQ ID NO: 14); R/K>A GATA2-ARM (SEQ ID NO: 15). (FIG. 3G) Experimental scheme for Tat transactivation assay. RNA Pol II transcribes the luciferase gene in the presence of Tat protein and bulge-containing TAR RNA. Indicated TF-ARMs are tested for their ability to replace Tat ARM. (FIG. 3H) Bar plots depicting the normalized luminescence values for the Tat transactivation assay with or without the TAR RNA bulge with the indicated TF-ARM replacements. Values are normalized to the control condition (p_{adj}<0.0001 for Tat RK>A compared to No Tat, WT Tat, KLF4, SOX2, and all conditions with TAR deletion; p_{adj}=0.0086 for Tat RK>A compared to GATA2, Sidak multiple comparison test).

FIGS. 4A-F. TF-ARMs enhance chromatin occupancy and gene expression. (FIG. 4A) Meta-gene analysis of CUT&Tag for WT or ΔARM HA-tagged KLF4 or SOX2, centered on called WT peaks in mESCs. (FIG. 4B) Example tracks of CUT&Tag (spike-in normalized) at specific genomic loci. (FIG. 4C) Diagram of KLF4 and its cross-correlation to the Tat ARM (dotted), predicted disorder (black line), DNA-binding domain (large cross-hatched boxes) and predicted disordered domain (small cross-hatching). (FIG. 4D) Side and top views of the crystal structure of KLF4 with DNA (PDB: 6VTX) or AlphaFold predicted structure (ID: O43474) and ARM-like domain (SEQ ID NO: 16) (FIG. 4E) Experimental scheme for TF gene activation assays. KLF4 ZFs are replaced either by GAL4 or TetR DBD. The effect of KLF4-ARM mutation or replacement of KLF4-ARM with Tat-ARM on gene activation is tested by UAS or TetO containing reporter system. (FIG. 4F) Normalized luminescence of gene activation assays, normalized to the “No TF” condition (error bars depict s.d., GAL4: p<0.0001 for all pairwise comparisons except WT vs. Tat-ARM, p=0.3363; TetR: NoTF vs. WT, p<0.0001, NoTF vs. R/K>A, p=0.5668, NoTF vs. Tat-ARM, p=0.0002, WT vs. R/K>A, p=0.0003, WT vs. Tat-ARM, p=0.7126, Tat-ARM vs. R/K>A, p=0.0008, one-way ANOVA)

FIGS. 5A-C. A role for TF RNA-binding regions in TF nuclear dynamics. (FIG. 5A) Cartoon depicting a 3-state model of TF diffusion. (FIG. 5B) Example of single nuclei single-molecule tracking traces for KLF4-WT and KLF4-ARM deletion. The traces are separated by their associated diffusion coefficient (Dimm: <0.04 μm2s-1; Dsub: 0.04-0.2 μm2s-1; Dfree: >0.2 μm2s-1). For each nucleus, 500 randomly sampled traces are shown. (FIG. 5C) Dot plot depicting the fraction of traces in the immobile, subdiffusive, or freely diffusing states. Each marker represents an independent imaging field (comparing WT and ARM deletion, p<0.0001 for KLF4free, SOX2free, CTCFfree, GATA2free, RUNXlfree, KLF4sub, GATA2sub, RUNX1sub, KLF4imm, SOX2imm, RUNX1imm; p=0.0094 for SOX2sub; p=0.0101 for CTCFsub, p=0.0034 for CTCFimm, p=0.38 for GATA2imm, two-tailed Student's t-test; error bars depict 95% C.I.).

FIGS. 6A-I. TF-ARMs are essential for normal development and disrupted in disease. (FIG. 6A) Experimental scheme for injection of zebrafish embryos with morpholinos and rescue by co-injection with the indicated mRNAs (hpf=hours post-fertilization). (FIG. 6B) Representative images of injected zebrafish embryos at 48 hpf. (FIG. 6C) Scoring of zebrafish anterior-posterior axis growth. (FIG. 6D) The landscape of mutations in TF-ARMs associated with human disease. (FIG. 6E) Examples of disease-associated mutations in TF-ARMs. (FIG. 6F) Line plot of the observed frequency or expected frequency of mutations for amino acids in TF-ARMs (SEQ ID NO: 17) (p=2.7×10-74 for enrichment of mutations in arginine, one-side binomial test with Benjamini-Hochberg correction). (FIG. 6G) Representation of the ESR1 protein and its correlation to the Tat ARM (*Maximum scoring ARM-like region). The selected mutation is provided in blue. (FIG. 6H) Gel shift assay with 7SK RNA and synthesized peptides for Tat-ARM-WT, Tat-ARM-R52A, ESR1-ARM-WT, and ESR1-ARM-R269C. (FIG. 6I) Tat transactivation reporter assay with wildtype or mutant versions of Tat and ESR1 ARMs and a version of the reporter without the Tat-binding TAR bulge. Values are normalized to the Tat-ARM-WT condition.

FIGS. 7A-C. Transcription factors harbor functional RNA-binding domains. (FIG. 7A) A model depiction of a previously unrecognized RNA-binding domain in a large fraction of transcription factors and its role in TF function. (FIG. 7B) Various ways by which RNA interactions could impact TF function at the molecular scale. (FIG. 7C) Various ways by which RNA interactions could impact TF function at the mesoscale.

FIGS. 8A-G. RNA-binding TFs in mammalian cells (Related to FIGS. 1A-F). (FIG. 8A) Scatter plot of 4SU-mediated fold change vs. protein abundance (raw peptide counts of—4SU condition) for the K562 RBR-ID (transcription factors in open circles). (FIG. 8B) Venn diagram depicting overlap of RBR+ protein hits and TFs for K562 cells (p=9.3e-9, Fisher's exact test). (FIG. 8C) Venn diagram depicting overlap of RBR+ protein hits and TFs for mES cells (p=0.02, Fisher's exact test). (FIG. 8D) Volcano plot of TF peptides in RBR-ID for murine embryonic stem cells with select highlighted TFs (dotted line at p=0.10). Each marker represents the peptide with maximum RBR-ID score for each protein. (FIG. 8E) Volcano plot of all detected peptides in RBR-ID for murine embryonic stem cells with select highlighted RBPs (dotted line at p=0.10). Each marker represents the peptide with maximum RBR-ID score for each protein. (FIG. 8F) List of RBRID+ TFs (p<0.05, log 2FC>0) for K562 RBR-ID categorized by DBD family (FIG. 8G) List of RBRID+ TFs (p<0.10, log 2FC>0) for mESC RBR-ID categorized by DBD family.

FIGS. 9A-E. Transcription factor binding to various RNAs (Related to FIGS. 1A-F and 2A-C). (FIG. 9A) Gel electrophoresis of UV-crosslinked HA-FLAG-GATA2 with visualization of RNA via IR800 adapter (top) and Western blot (bottom). (FIG. 9B) ChIP-seq and CLIP signal for YY1 and CTCF at the Trim28 and TP53 genomic loci (FIG. 9C) Meta-gene analysis of CLIP signal centered on YY1 or CTCF ChIP-seq peaks (FIG. 9D) Fraction bound RNA with increasing protein concentration for 6 TFs and 4 RNA species per TF. (FIG. 9E) Table of apparent Kd values for the binding assays in (B) (p-values comparing random RNA to pRNA, eRNA, and 7SK RNA respectively—KLF4: 0.06, 6.24e-6, 1.88e-4; SOX2: 0.09, 0.81, 0.013; GATA2: 0.47, 1.05e-5, 0.10; MYC: 0.84, 0.15, 0.11; RARA: 0.53, 0.17, 0.17; STAT3: 0.26, 0.99, 0.33).

FIGS. 10A-D. Sequence analysis of RNA-binding regions in transcription factors (Related to FIGS. 3A-H). (FIG. 10A) Scheme to search for structured RNA-binding domain motifs in transcription factors. (FIG. 10B) Scatter plot depicting the HMMER log 2-odds ratio score for the 4 most abundant RNAbinding domains (RRM, KH, ZnF-CCCH, DEAD) for select RBPs and all human TFs. (FIG. 10C) Evolutionary conservation analysis using Shannon entropy for TF-ARMs or TFs excluding the ARMs. (FIG. 10D) Diagram of KLF4, SOX2, and GATA2 and their cross-correlation to the Tat ARM (black), predicted disorder (black line), DNA-binding domain (large cross-hatched boxes) and predicted disordered domain (small cross-hatching).

FIGS. 11A-D. Transcription factor binding to DNA in vitro (Related to FIGS. 3A-H). (FIG. 11A) Gel shift assay of the synthesized SOX2-ARM peptide with DNA or RNA. (FIG. 111B) Gel shift assay of the synthesized KLF4-ARM peptide with DNA or RNA. (FIG. 11C) Fraction bound motif-containing DNA with increasing protein concentration for SOX2 (SOX2 495 WT vs ΔARM: p=0.11, error bars depict s.d.). (FIG. 11D) Fraction bound motif-containing DNA with increasing protein concentration for KLF4 (KLF4 WT vs ΔARM: p=8.75e-6; error bars depict s.d.)

FIGS. 12A-B. Crosslinking of TF-ARMs to RNA in cells (Related to FIGS. 3A-H). (FIG. 12A) Global analysis of RBR-ID+ peptide enrichment near known RNA-binding domains, TF-ARMs, or randomized peptides near ARMs. (FIG. 12B) Examples of RBR-ID+ peptides for select TFs.

FIGS. 13A-D. Transcription factor enrichment in sub-nuclear fractions (Related to FIGS. 4A-F). (FIG. 13A) Western blot of histone H3 and HA-tagged wildtype or ARM-mutant KLF4 and SOX2 in nucleoplasmic (N) or chromatin (C) fractions. (FIG. 13B) Quantification of the relative intensity in N and C fractions of the samples in (A). (FIG. 13C) Western blot of Sox2 or Klf4 and histone H3 in nucleoplasmic (N) or chromatin (C) fractions with or without RNase treatment. (FIG. 13D) Quantification of the relative intensity in N and C fractions of the samples in (C).

FIGS. 14A-E. Controls for in vivo experiments (Related to FIGS. 5A-C and 6A-I). (FIG. 14A) Example of single nuclei single-molecule tracking traces for wildtype and ARM-mutant SOX2 and CTCF in mESCs, and GATA2 and RUNX1 in K562 cells. The traces are separated by their associated diffusion coefficient (Dimm: <0.04 μm2s-1; Dsub: 0.04-0.2 μm2s-1; Dfree: >0.2 μm²s⁻¹). For each nucleus, up to 500 randomly sampled traces are shown. (FIG. 14B) Distribution of diffusion constants (D) for WT and ARM-mutant TFs. (FIG. 14C) Stable dwell times for KLF4, SOX2, and CTCF (error bars depict s.e.m.). Fraction of traces in 3-state model across different expression levels of KLF4. (FIG. 14D) Table providing trajectory metrics across the different KLF4 expression levels. (FIG. 14E) Western blot of lysates from zebrafish embryos injected with mRNA.

### DETAILED DESCRIPTION

A description of example embodiments follows.

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The presently disclosed subject matter provides methods, compositions, and kits for modulating expression of a target gene, and related methods of treating diseases, conditions, and disorders in which aberrant transcription (e.g., increased or decreased) of a target gene is implicated. The presently disclosed subject matter relies on work described herein that demonstrates that RNA transcribed from regulatory elements of a target gene binds to and stabilizes transcription factors occupying those regulatory elements. Without wishing to be bound by theory, it is believed that binding between the RNA transcribed from the regulatory elements of the target gene creates a positive feedback loop, for example, where the transcription factors stimulate local transcription, and newly transcribed nascent RNA reinforces local transcription factor occupancy thereby further stimulating local transcription. Accordingly, in some aspects, the presently disclosed subject matter provides a method of modulating expression of a target gene comprising modulating binding between an RNA transcribed from at least one regulatory element of a target gene and a transcription factor which binds to both the RNA and the regulatory element. In other words, the methods of the presently disclosed subject matter involve modulating transcription of target genes (and expression products of genes) by targeting the RNA transcribed from regulatory elements of target genes whose expression is regulated by transcription factors which are bound by such RNA while the transcription factor occupies the regulatory elements from which the RNA was transcribed. The methods of modulating gene expression disclosed herein may in some embodiments be used for therapeutic purposes, for example, to decrease expression of a target gene whose aberrant or increased transcription is implicated in a disease, condition, or disorder (e.g., a cancer, genetic disorder, etc.) or to increase expression of a target gene whose aberrant or decreased transcription is implicated in a disease, condition, or disorder (e.g., a cancer, genetic disorder, etc.).

### Methods for Modulating Expression of a Target Gene

As used herein, the term “transcription factor” refers to a protein that binds to a regulatory element of a target gene to modulate, e.g., increase or decrease, expression of the target gene. The presently disclosed subject matter contemplates the use of any transcription factor that is capable of simultaneously binding to both DNA sequences of regulatory elements and RNA sequences transcribed from those regulatory elements. As used herein, “simultaneously binding” of a transcription factor to both DNA sequences of regulatory elements and RNA sequences transcribed from those regulatory elements means that the transcription factor is capable of binding both the DNA sequence and the RNA sequence at the same time for at least a portion of a related activity (e.g., transcription of the target gene to produce an mRNA encoding a protein) even though the transcription factor might not be bound to both the DNA sequence and the RNA sequence at the same time throughout the related activity. For the avoidance of doubt, simultaneous binding contemplates situations in which the DNA sequence is occupied by the transcription factor before the transcribed RNA sequence is bound, as well as those in which the transcribed RNA sequence is bound even though the transcription factor is not occupying the DNA sequence.

In some embodiments, the transcription factor is not Yin-Yang 1 (YY1).

In some embodiments, the transcription factor is not Yin-Yang 1 (YY1). In some embodiments, the transcription factor is not Krueppel-like factor 4 (KLF4). In some embodiments, the transcription factor is not Ronin (Thap11). In some embodiments, the transcription factor is not RE1-silencing transcription factor (REST). In some embodiments, the transcription factor is not PR domain zinc finger protein 14 (PRDM14). In some embodiments, the transcription factor is not CCCTC-binding factor (CTCF). In some embodiments, the transcription factor is not p53. In some embodiments, the transcription factor is not Signal transducer and activator of transcription 1 (STAT1). In some embodiments, the transcription factor is not TLS/FUS. In some embodiments, the transcription factor is not BRCA1. In some embodiments, the transcription factor is not DLX2. In some embodiments, the transcription factor is not ESR1. In some embodiments, the transcription factor is not FUS. In some embodiments, the transcription factor is not KIN. In some embodiments, the transcription factor is not KU. In some embodiments, the transcription factor is not NACA. In some embodiments, the transcription factor is not NCL. In some embodiments, the transcription factor is not NFKB1. In some embodiments, the transcription factor is not NFYA. In some embodiments, the transcription factor is not NR3C1. In some embodiments, the transcription factor is not RARA. In some embodiments, the transcription factor is not RUNX1. In some embodiments, the transcription factor is not SOX2. In some embodiments, the transcription factor is not TCF7. In some embodiments, the transcription factor is not or TP53.

In some embodiments, the transcription factor is not BRCA1. In some embodiments, the transcription factor is not CTCF. In some embodiments, the transcription factor is not DLX2. In some embodiments, the transcription factor is not ESR1 (Estrogen receptor). In some embodiments, the transcription factor is not FUS (TLS). In some embodiments, the transcription factor is not KIN (KIN17). In some embodiments, the transcription factor is not KLF4. In some embodiments, the transcription factor is not KU (Saccharomyces). In some embodiments, the transcription factor is not NACA (α-NAC). In some embodiments, the transcription factor is not NCL (Nucleolin). In some embodiments, the transcription factor is not NFKB1 (and RELA). In some embodiments, the transcription factor is not NFYA (NF-YA). In some embodiments, the transcription factor is not NR3C1 (Glucocorticoid receptor). In some embodiments, the transcription factor is not PRDM14. In some embodiments, the transcription factor is not RARA (RARα). In some embodiments, the transcription factor is not RE1-silencing transcription factor (REST). In some embodiments, the transcription factor is not Ronin (Thap11). In some embodiments, the transcription factor is not RUNX1 (AML1). In some embodiments, the transcription factor is not SOX2. In some embodiments, the transcription factor is not STAT1. In some embodiments, the transcription factor is not TCF7 (TCF-1). In some embodiments, the transcription factor is not TP53 (p53). In some embodiments, the transcription factor is not YY1.

Other transcription factors that bind both DNA and RNA can be identified using methods known to a person with ordinary skill in the art, such as cross-linking immunoprecipitation (CLIP) and chromatin immunoprecipitation (ChIP).

In some embodiments, any region of the transcription factor can bind to the RNA or at least one regulatory element as long as the RNA and the regulatory element are not binding in the same region and therefore competing for binding to the transcription factor. DNA binding motifs can occur throughout a transcription factor and are not limited to one specific region. In some embodiments, the transcription factor comprises an N-terminal region and a C-terminal region, wherein the N-terminal region binds to either the RNA or the at least one regulatory element, and the C-terminal region binds to the RNA or the at least one regulatory element which is not bound to the N-terminal region. In some embodiments, a region (e.g., one or more domains) of the transcription factor between the C-terminal region and the N-terminal region (i.e., central region) binds to the RNA and/or at least one regulatory element.

In some embodiments, either the N-terminal region or the C-terminal region comprises a DNA binding domain selected from the group consisting of a zinc finger, leucine zipper, helix-turn-helix, winged helix-turn-helix, helix-loop-helix, HMG-box, and GB-fold. In some embodiments, either the N-terminal region or the C-terminal region comprises an RNA binding domain. Non-limiting examples of RNA binding domains contemplated herein, such as the RNA Recognition Motif (RRM), the K homology (KH) domain, the CCCH zinc finger domain, the Like Sm domain, the Cold-shock domain, the PUA domain, the Ribosomal protein Si-like domain, the Surp module/SWAP domain, the Lupus La RNA-binding domain, the PWI domain, the YTH domain, the THUMP domain, the Pumilio-like domain, the Sterile alpha motif, the C2H2 zinc finger domain, the RNP-1 motif, and the RNP-2 motif can be found in the database of RNA-binding protein specificities (RBPDB;<rbpdb.ccbr.utoronto.ca>). In some embodiments, at least one of the N-terminal region, the central region, or the C-terminal region of the transcription factor comprises a DNA binding domain, and at least one of the N-terminal region, the central region, or the C-terminal region lacking the DNA binding domain contains an RNA binding domain.

In some embodiments, modulating binding comprises promoting binding between the RNA and the transcription factor. As used herein, “binding” between the RNA and the transcription factor includes binding via non-covalent interactions, such as van der Waals interactions, electrostatic interactions (salt bridges), dipolar interactions (hydrogen bonding), and entropic effects (hydrophobic interactions). It is believed that promoting binding between the RNA and the transcription factor stabilizes occupancy of the transcription factor at the at least one regulatory element, thereby increasing expression of the target gene (e.g., increasing transcription).

Accordingly, in some embodiments, the disclosure provides a method of increasing expression of a target gene, the method comprising promoting binding between a ribonucleic acid (RNA) and a transcription factor which binds to both the RNA and the at least one regulatory element, wherein promoting binding between the RNA and the transcription factor stabilizes occupancy of the transcription factor at the at least one regulatory element, thereby increasing expression of the target gene.

The term “stabilizes occupancy” means that the transcribed RNA keeps the transcription factor sufficiently bound to, or close enough to, the at least one regulatory element for the transcription of the target gene to occur, for example, by increasing the binding affinity or apparent binding affinity of the transcription factor to one of its consensus motifs in the at least one regulatory element. Without wishing to be bound by theory, it is believed that the RNA transcribed from the at least one regulatory element captures the transcription factor via relatively weak interactions as it is dissociating from the at least one regulatory element, which allows the transcription factor to rebind to nearby DNA sequences, thus creating a kinetic sink that increases transcription factor occupancy on the at least one regulatory element. In some embodiments, stabilizing occupancy of the transcription factor at the at least one regulatory element increases the level of transcription of the target gene by at least about 1-fold, 1.1-fold, 1.2-fold, 1.3-fold, 1.4-fold 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold or more, e.g., within a cell, tissue, or subject. In some embodiments, stabilizing occupancy of the transcription factor at the at least one regulatory element increases the level of transcription of the target gene by between 1-fold and 5-fold. In some embodiments, stabilizing occupancy of the transcription factor at the at least one regulatory element increases the level of transcription of the target gene by between 1-fold and 2-fold. In some embodiments, the binding affinity or the apparent binding affinity of the transcription factor for at least one regulatory element is increased by about 1-fold, 1.1-fold, 1.2-fold, 1.3-fold, 1.4-fold 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold or more, e.g., within a cell, tissue, or subject. In some embodiments, the binding affinity or the apparent binding affinity of the transcription factor for at least one regulatory element is increased by between 1-fold and 5-fold. In some embodiments, the binding affinity or the apparent binding affinity of the transcription factor for at least one regulatory element is increased by between 1-fold and 2-fold.

In some embodiments, determining whether promoting binding between an RNA and a transcription factor stabilizes occupancy of the transcription factor at the at least one regulatory element and/or increases transcription of the target gene comprising the at least one regulatory element can be achieved by detecting levels of mRNA encoded by the target gene. In some embodiments, determining whether promoting binding between an RNA and a transcription factor stabilizes occupancy of the transcription factor at the at least one regulatory element and/or increases transcription of the target gene comprising the at least one regulatory element can be achieved by detecting levels and/or activity of protein encoded by the target gene.

A variety of methods for detecting levels of mRNA and/or levels and/or activity of protein expressed by a target gene are well known in the art. The presently disclosed subject matter contemplates the use of any such method. Examples of such suitable methods include RNA-Seq, RT-PCR, real-time PCR, Northern blotting, Western blotting, in situ hybridization, oligonucleotide arrays (e.g., microarray) or chips, to name more than a few. In some embodiments determining whether promoting binding between an RNA and a transcription factor stabilizes occupancy of the transcription factor at the at least one regulatory element and/or increases transcription of the target gene comprising the at least one regulatory element may be performed using a reporter construct comprising a nucleic acid sequence encoding a reporter protein operably linked to the regulatory element of interest. One could detect the reporter protein as an indicator of transcription driven by the regulatory element (e.g., in the presence of a test agent being tested for its ability to interfere with or promote binding between the RNA and the transcription factor). It should be appreciated that such reporter construct could also be used to determine whether inhibiting binding between an RNA and a transcription factor destabilizes occupancy of the transcription factor at the at least one regulatory element and/or decreases transcription of the target gene comprising the at least one regulatory element. In some embodiments, a fluorescent reporter RNA can be used as an indicator of transcription driven by the regulatory element (e.g., in the presence of a test agent being tested for its ability to interfere with or promote binding between the RNA and the transcription factor). Examples of suitable fluorescent reporter RNAs include RNA mimics of green fluorescent protein (see, e.g., Paige et al., “RNA Mimics of Green Fluorescent Protein,” Science. 2011 (333): 642-646, which is incorporated herein by reference). It should be appreciated that transcription of the target gene can be modulated by promoting binding between the RNA transcribed from the at least one regulatory element, as well as by promoting binding between RNA that is not transcribed from the at least one regulatory element but nevertheless is capable of binding to the transcription factor either at the same RNA binding domain at which the transcription factor binds the RNA transcribed from the at least one regulatory element, or at another site of the transcription factor that is distinct from the DNA binding domain (and/or does not interfere with binding between the transcription factor and the at least one regulatory element). That is, the presently disclosed subject matter contemplates the use of any RNA that is capable of binding to the transcription factor in a way that stabilizes occupancy of the transcription factor at the at least one regulatory element.

In some embodiments, promoting binding between the RNA and the transcription factor comprises tethering an RNA that binds to the transcription factor to a DNA sequence proximal to the at least one regulatory element. In some embodiments, the RNA is tethered to a DNA sequence proximal to at least one regulatory element. In some embodiments, the RNA is tethered within at least one regulatory element. In these embodiments, the RNA that is tethered is not the RNA transcribed from a regulatory element or an RNA that is released by RNA polymerase. Rather, the RNA that is tethered is a synthetic RNA that binds to the transcription factor in a way that stabilizes the transcription factor. In some embodiments, the tethered RNA is homologous to the RNA transcribed from a regulatory element.

The term “homologous” means that a polynucleotide, such as an RNA, comprises a sequence that has a desired identity, for example, at least 60% identity, preferably at least 70% sequence identity, more preferably at least 80%, still more preferably at least 90% and even more preferably at least 95%, compared to a reference sequence. In some embodiments, the synthetic RNA is at least 81% identical to RNA transcribed from the at least one regulatory element. In some embodiments, the synthetic RNA is at least 82% identical to RNA transcribed from the at least one regulatory element. In some embodiments, the synthetic RNA is at least 83% identical to RNA transcribed from the at least one regulatory element. In some embodiments, the synthetic RNA is at least 84% identical to RNA transcribed from the at least one regulatory element. In some embodiments, the synthetic RNA is at least 85% identical to RNA transcribed from the at least one regulatory element. In some embodiments, the synthetic RNA is at least 86% identical to RNA transcribed from the at least one regulatory element. In some embodiments, the synthetic RNA is at least 87% identical to RNA transcribed from the at least one regulatory element. In some embodiments, the synthetic RNA is at least 88% identical to RNA transcribed from the at least one regulatory element. In some embodiments, the synthetic RNA is at least 89% identical to RNA transcribed from the at least one regulatory element. In some embodiments, the synthetic RNA is at least 90% identical to RNA transcribed from the at least one regulatory element. In some embodiments, the synthetic RNA is at least 91% identical to RNA transcribed from the at least one regulatory element. In some embodiments, the synthetic RNA is at least 92% identical to RNA transcribed from the at least one regulatory element. In some embodiments, the synthetic RNA is at least 93% identical to RNA transcribed from the at least one regulatory element. In some embodiments, the synthetic RNA is at least 94% identical to RNA transcribed from the at least one regulatory element. In some embodiments, the synthetic RNA is at least 95% identical to RNA transcribed from the at least one regulatory element. In some embodiments, the synthetic RNA is at least 96% identical to RNA transcribed from the at least one regulatory element. In some embodiments, the synthetic RNA is at least 96% identical to RNA transcribed from the at least one regulatory element. In some embodiments, the synthetic RNA is at least 97% identical to RNA transcribed from the at least one regulatory element. In some embodiments, the synthetic RNA is at least 98% identical to RNA transcribed from the at least one regulatory element. In some embodiments, the synthetic RNA is at least 99% identical to RNA transcribed from the at least one regulatory element. In some embodiments, the synthetic RNA comprises at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element. Determining optimal alignment is within the purview of one of skill in the art. For example, there are publically and commercially available alignment algorithms and programs such as, but not limited to, ClustalW, Smith-Waterman in matlab, Bowtie, Geneious, Biopython and SeqMan.

In some embodiments, modulating binding comprises interfering with binding between the RNA and the transcription factor. In some embodiments, the disclosure provides a method of decreasing expression of a target gene, the method comprising interfering with binding between a ribonucleic acid (RNA) transcribed from at least one regulatory element of a target gene and a transcription factor which binds to both the RNA and the at least one regulatory element, wherein interfering with binding between the RNA and the transcription factor destabilizes occupancy of the transcription factor at the at least one regulatory element, thereby decreasing expression of the target gene.

The term “destabilizes occupancy” means that the transcribed RNA weakens the attraction or interaction between the transcription factor and the at least one regulatory element (e.g., by decreasing the binding affinity or apparent binding affinity of the transcription factor and the at least one regulatory element) and/or reduces the local concentration of the transcription factor in proximity to the at least one regulatory element, such that the transcription factor does not remain sufficiently bound to, or present at a sufficient concentration in proximity to, the at least one regulatory element for transcription of the target gene to occur. In some embodiments, destabilizing occupancy of the transcription factor at the at least one regulatory element decreases the level of transcription of the target gene by at least about 5%, 10%, 15%, 20%, 25%, 30%, 33%, 35%, 40%, 45%, 50%, 55%, 60%, 66%, 70%, 75%, 80%, 85%, 90%, or 95% or more, e.g., within a cell, tissue, or subject. In some embodiments, the level of transcription of the target gene is decreased within the cell by 100% (i.e., complete inhibition of transcription of the target gene). In some embodiments, the binding affinity or the apparent binding affinity of the transcription factor for at least one regulatory element is reduced by at least about 5%, 10%, 15%, 20%, 25%, 30%, 33%, 35%, 40%, 45%, 50%, 55%, 60%, 66%, 70%, 75%, 80%, 85%, 90%, or 95% or more, e.g., within a cell, tissue, or subject.

In some embodiments, determining whether interfering with binding between an RNA and a transcription factor destabilizes occupancy of the transcription factor at the at least one regulatory element and/or decreases transcription of the target gene comprising the at least one regulatory element can be achieved by detecting levels of mRNA encoded by the target gene. In some embodiments, determining whether interfering with binding between an RNA and a transcription factor destabilizes occupancy of the transcription factor at the at least one regulatory element and/or decreases transcription of the target gene comprising the at least one regulatory element can be achieved by detecting levels and/or activity of protein encoded by the target gene.

In some embodiments, modulating expression of the target gene occurs in vitro or ex vivo. In some embodiments, modulating expression of the target gene comprises contacting a cell with an effective amount of a composition and/or agent which promotes binding between the RNA and the transcription factor. In some embodiments, modulating expression of the target gene comprises contacting a cell with an effective amount of a composition and/or agent which interferes with binding between the RNA and the transcription factor. As used herein “contacting the cell” and the like, refers to any means of introducing an agent into a target cell in vitro or in vivo, including by chemical and physical means, whether directly or indirectly or whether the agent physically contacts the cell directly or is introduced into an environment (e.g., culture medium) in which the cell is present or to which the cell is added. Contacting also is intended to encompass methods of exposing a cell, delivering to a cell, or ‘loading’ a cell with an agent by viral or non-viral vectors, and wherein such agent is bioactive upon delivery. The method of delivery will be chosen for the particular agent and use. Parameters that affect delivery, as is known in the art, can include, inter alia, the cell type affected and cellular location. In some embodiments, “contacting” includes administering the agent to an individual. In some embodiments, “contacting” refers to exposing a cell or an environment in which the cell is located to one or more presently disclosed agents.

The present disclosure contemplates the use of any composition and/or agent that is capable of interfering with binding between the RNA transcribed from at least one regulatory element and the transcription factor itself. In some embodiments, modulating expression of the target gene occurs in vivo. In some embodiments, modulating expression of the target gene comprises administering to a subject an effective amount of a composition which interferes with binding between RNA transcribed from at least one regulatory element and the transcription factor.

The presently disclosed subject matter contemplates modulating expression (e.g., increasing and/or decreasing transcription) in cells, tissues, and subjects. In some embodiments, the cell or tissue includes one of the following: mammalian cell, e.g., human cell; fetal cell; embryonic stem cell or embryonic stem cell-like cell, e.g., cell from the umbilical vein, e.g., endothelial cell from the umbilical vein; muscle, e.g., myotube, fetal muscle; blood cell, e.g., cancerous blood cell, fetal blood cell, monocyte; B cell, e.g., Pro-B cell; brain, e.g., astrocyte cell, angular gyrus of the brain, anterior caudate of the brain, cingulate gyrus of the brain, hippocampus of the brain, inferior temporal lobe of the brain, middle frontal lobe of the brain, brain cancer cell; T cell, e.g., naive T cell, memory T cell; CD4 positive cell; CD25 positive cell; CD45RA positive cell; CD45RO positive cell; IL-17 positive cell; a cell that is stimulated with PMA; Th cell; Th17 cell; CD255 positive cell; CD127 positive cell; CD8 positive cell; CD34 positive cell; duodenum, e.g., smooth muscle tissue of the duodenum; skeletal muscle tissue; myoblast; stomach, e.g., smooth muscle tissue of the stomach, e.g., gastric cell; CD3 positive cell; CD14 positive cell; CD19 positive cell; CD20 positive cell; CD34 positive cell; CD56 positive cell; prostate, e.g., prostate cancer; colon, e.g., colorectal cancer cell; crypt cell, e.g., colon crypt cell; intestine, e.g., large intestine; e.g., fetal intestine; bone, e.g., osteoblast; pancreas, e.g., pancreatic cancer; adipose tissue; adrenal gland; bladder; esophagus; heart, e.g., left ventricle, right ventricle, left atrium, right atrium, aorta; lung, e.g., lung cancer cell; skin, e.g., fibroblast cell; ovary; psoas muscle; sigmoid colon; small intestine; spleen; thymus, e.g., fetal thymus; breast, e.g., breast cancer; cervix, e.g., cervical cancer; mammary epithelium; liver, e.g., liver cancer; DND41 cell; GM12878 cell; H1 cell; H2171 cell; HCC1954 cell; HCT-116 cell; HeLa cell; HepG2 cell; HMEC cell; HSMM tube cell; HUVEC cell; IMR90 cell; Jurkat cell; K562 cell; LNCaP cell; MCF-7 cell; MMIS cell; NHLF cell; NHDF-Ad cell; RPMI-8402 cell; U87 cell; VACO 9M cell; VACO 400 cell; or VACO 503 cell. In some embodiments, the cell is selected from the group consisting of adipocytes (e.g., white fat cell or brown fat cell), cardiac myocytes, chondrocytes, endothelial cells, exocrine gland cells, fibroblasts, glial cells, hepatocytes, keratinocytes, macrophages, monocytes, melanocytes, neurons, neutrophils, osteoblasts, osteoclasts, pancreatic islet cells (e.g., a beta cell), skeletal myocytes, smooth muscle cells, B cells, plasma cells, T cells (e.g., regulatory, cytotoxic, helper), and dendritic cells.

In some embodiments, the methods, compositions and/or agents disclosed herein can be used to modulate levels of expression of cell type specific genes and/or cell state specific genes. Modulating levels of expression of cell type specific genes and/or cell state specific genes may be useful, for example, to change a cell type from a cell of a first type to a cell of a second type (e.g., directed differentiation of a pluripotent cell to a desired cell type, reprogramming of a somatic cell, e.g., to a pluripotent state, or transdifferentiation of a somatic cell, e.g., to a different somatic cell) or to change a cell from one state to another state (e.g., shifting a cell from an “abnormal” state towards a more “normal” state, shifting a cell from a “disease-associated” state towards a more “healthy” state, shifting the cells from an “activated” state to a “resting” or “non-activated” state, etc.).

A cell type specific gene is typically expressed selectively in one or a small number of cells types relative to expression in many or most other cell types. One of skill in the art will be aware of numerous genes that are considered cell type specific. A cell type specific gene need not be expressed only in a single cell type but may be expressed in one or several, e.g., up to about 5, or about 10 different cell types out of the approximately 200 commonly recognized (e.g., in standard histology textbooks) and/or most abundant cell types in an adult vertebrate, e.g., mammal, e.g., human. In some embodiments, a cell type specific gene is one whose expression level can be used to distinguish a cell, e.g., a cell as disclosed herein, such as a cell of one of the following types from cells of the other cell types: adipocyte (e.g., white fat cell or brown fat cell), cardiac myocyte, chondrocyte, endothelial cell, exocrine gland cell, fibroblast, glial cell, hepatocyte, keratinocyte, macrophage, monocyte, melanocyte, neuron, neutrophil, osteoblast, osteoclast, pancreatic islet cell (e.g., a beta cell), skeletal myocyte, smooth muscle cell, B cell, plasma cell, T cell (e.g., regulatory, cytotoxic, helper), or dendritic cell. In some embodiments a cell type specific gene is lineage specific, e.g., it is specific to a particular lineage (e.g., hematopoietic, neural, muscle, etc.) In some embodiments, a cell-type specific gene is a gene that is more highly expressed in a given cell type than in most (e.g., at least 80%, at least 90%) or all other cell types. Thus specificity may relate to level of expression, e.g., a gene that is widely expressed at low levels but is highly expressed in certain cell types could be considered cell type specific to those cell types in which it is highly expressed. It will be understood that expression can be normalized based on total mRNA expression (optionally including miRNA transcripts, long non-coding RNA transcripts, and/or other RNA transcripts) and/or based on expression of a housekeeping gene in a cell. In some embodiments, a gene is considered cell type specific for a particular cell type if it is expressed at levels at least 2, 5, or at least 10-fold greater in that cell than it is, on average, in at least 25%, at least 50%, at least 75%, at least 90% or more of the cell types of an adult of that species, or in a representative set of cell types. One of skill in the art will be aware of databases containing expression data for various cell types, which may be used to select cell type specific genes. In some embodiments a cell type specific gene is a transcription factor.

In some aspects, modulating binding between an RNA transcribed from at least one regulatory element of a target gene and a transcription factor which binds to both the RNA and the regulatory element shifts a cell from an “abnormal” state towards a more “normal” state.

In some embodiments, modulating binding between an RNA transcribed from at least one regulatory element of a target gene and a transcription factor which binds to both the RNA and the regulatory element shifts a cell from a “disease-associated” state towards a state that is not associated with disease. A “disease-associated state” is a state that is typically found in subjects suffering from a disease (and usually not found in subjects not suffering from the disease) and/or a state in which the cell is abnormal, unhealthy, or contributing to a disease.

In some aspects, modulating binding between an RNA transcribed from at least one regulatory element of a target gene and a transcription factor which binds to both the RNA and the regulatory element reprograms a somatic cell, e.g., to a pluripotent state. In some aspects, modulating binding between an RNA transcribed from at least one regulatory element of a target gene and a transcription factor which binds to both the RNA and the regulatory element can be used to direct differentiation of a cell, e.g., from a pluripotent state to a cell of a desired cell type. In some embodiments, the methods, compositions and agents herein are of use to reprogram a somatic cell, e.g., to a pluripotent state. In some embodiments the methods, compositions and agents are of use to reprogram a somatic cell of a first cell type into a different cell type. In some embodiments, the methods, compositions and agents herein are of use to differentiate a pluripotent cell to a desired cell type.

In some aspects, modulating binding between an RNA transcribed from at least one regulatory element of a target gene and a transcription factor which binds to both the RNA and the regulatory element shifts a cell from an activated state to a resting or non-activated state. In some aspects, modulating binding between an RNA transcribed from at least one regulatory element of a target gene and a transcription factor which binds to both the RNA and the regulatory element shifts a cell from a non-activated state or resting state to an activated state. Another example of cell state is “activated” state as compared with “resting” or “non-activated” state. Many cell types in the body have the capacity to respond to a stimulus by modifying their state to an activated state. The particular alterations in state may differ depending on the cell type and/or the particular stimulus. A stimulus could be any biological, chemical, or physical agent to which a cell may be exposed. A stimulus could originate outside an organism (e.g., a pathogen such as virus, bacteria, or fungi (or a component or product thereof such as a protein, carbohydrate, or nucleic acid, cell wall constituent such as bacterial lipopolysaccharide, and the like) or may be internally generated (e.g., a cytokine, chemokine, growth factor, or hormone produced by other cells in the body or by the cell itself). For example, stimuli can include interleukins, interferons, or TNF alpha. Immune system cells, for example, can become activated upon encountering foreign (or in some instances host cell) molecules. Cells of the adaptive immune system can become activated upon encountering a cognate antigen (e.g., containing an epitope specifically recognized by the cell's T cell or B cell receptor) and, optionally, appropriate co-stimulating signals. Activation can result in changes in gene expression, production and/or secretion of molecules (e.g., cytokines, inflammatory mediators), and a variety of other changes that, for example, aid in defense against pathogens but can, e.g., if excessive, prolonged, or directed against host cells or host cell molecules, contribute to diseases. Fibroblasts are another cell type that can become activated in response to a variety of stimuli (e.g., injury (e.g., trauma, surgery), exposure to certain compounds including a variety of pharmacological agents, radiation, etc.) leading them, for example, to secrete extracellular matrix components. In the case of response to injury, such ECM components can contribute to wound healing. However, fibroblast activation, e.g., if prolonged, inappropriate, or excessive, can lead to a range of fibrotic conditions affecting diverse tissues and organs (e.g., heart, kidney, liver, intestine, blood vessels, skin) and/or contribute to cancer. The presence of abnormally large amounts of ECM components can result in decreased tissue and organ function, e.g., by increasing stiffness and/or disrupting normal structure and connectivity.

In some embodiments, the composition comprises an agent which binds to the transcription factor in a manner that prevents the transcription factor from binding to the RNA transcribed from the at least one regulatory element. In some embodiments, the agent binds to the transcription factor at the same site that the RNA transcribed from at least one regulatory element would bind to the transcription factor. In some embodiments, the agent binds to at least a portion of the same site that the RNA transcribed from at least one regulatory element would bind to the transcription factor (i.e., the agent binds to one or more amino acids of the transcription factor binding site for the RNA transcribed from the at least one regulatory element, but does not bind to all of the amino acids of such site). In some embodiments, the agent binds to the transcription factor in proximity to where RNA transcribed from at least one regulatory element binds to the transcription factor, but the agent masks the RNA binding site so the RNA can no longer bind to the transcription factor. In some embodiments, the agent binds to the transcription factor away from where the RNA transcribed from at least one regulatory element binds to the transcription factor, but the agent causes the transcription factor to change its conformation such that the RNA transcribed from at least one regulatory element can no longer bind to the transcription factor. In some embodiments, binding of the agent to the transcription factor affects another protein or cofactor that interacts with the transcription factor and the other protein or cofactor inhibits the RNA transcribed from at least one regulatory element from binding to the transcription factor.

In some embodiments, the agent which interferes with binding between the RNA and the transcription factor is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof. As used herein, small molecules refers to compounds having a molecular weight of less than about 2 kilodaltons. In some embodiments, the small molecule has a molecular weight of less than about 1000 daltons. In some embodiments, the small molecule has a molecular weight of less than about 500 daltons.

The presently disclosed subject matter contemplates the use of synthetic, chemically modified nucleic acid molecules. The synthetic, chemically modified nucleic acid molecules are useful in the treatment of any disease or condition that responds to modulation of gene expression or activity in a cell, tissue, or organism, and in particular are useful for modulating binding between RNA transcribed from regulatory elements occupied by transcription factors that bind to the transcribed RNA, as well as the regulatory elements. The synthetic, chemically modified nucleic acid molecules can be used to increase or decrease transcription of target genes.

Exemplary nucleic acids include ribonucleic acids (RNAs), deoxyribonucleic acids (DNAs), threose nucleic acids (TNAs), glycol nucleic acids (GNAs), peptide nucleic acids (PNAs), locked nucleic acids (LNAs) or a hybrid thereof (e.g., In some embodiments, the nucleic acids comprise short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against target nucleic acid sequences. In some embodiments, the nucleic acid comprises messenger RNA (mRNA). In some embodiments, the nucleic acids of the invention do not substantially induce an innate immune response of a cell into which the nucleic acid is introduced.

Various modifications to the structures of the nucleic acid can be made to enhance the utility of these molecules. Such modifications will enhance shelf-life, half-life in vitro, stability, and ease of introduction of such oligonucleotides to the target site, e.g., to enhance penetration of cellular membranes, and confer the ability to recognize and bind to targeted cells.

As used herein, “non-nucleotide” means any group or compound which can be incorporated into a nucleic acid chain in the place of one or more nucleotide units, including either sugar and/or phosphate substitutions, and allows the remaining bases to exhibit their enzymatic activity. The group or compound is abasic in that it does not contain a commonly recognized nucleotide base, such as adenosine, guanine, cytosine, uracil or thymine and therefore lacks a base at the 1′-position.

As used herein “nucleotide” as is as recognized in the art to include natural bases (standard), and modified bases well known in the art. Such bases are generally located at the 1′ position of a nucleotide sugar moiety. Nucleotides generally comprise a base, sugar and a phosphate group. The nucleotides can be unmodified or modified at the sugar, phosphate and/or base moiety, (also referred to interchangeably as nucleotide analogs, modified nucleotides, non-natural nucleotides, non-standard nucleotides and other; see, for example, Usman and McSwiggen, supra; Eckstein et al., International PCT Publication No. WO 92/07065; Usman et al., International PCT Publication No. WO 93/15187; Uhlman & Peyman, supra, all are hereby incorporated by reference herein). There are several examples of modified nucleic acid bases known in the art as summarized by Limbach et al., 1994, Nucleic Acids Res. 22, 2183. Some of the non-limiting examples of base modifications that can be introduced into nucleic acid molecules include, inosine, purine, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), propyne, and others (Burgin et al., 1996, Biochemistry, 35, 14090; Uhlman & Peyman, supra). By “modified bases” in this aspect is meant nucleotide bases other than adenine, guanine, cytosine and uracil at 1′ position or their equivalents.

As used herein “abasic” means sugar moieties lacking a base or having other chemical groups in place of a base at the 1′ position, see for example Adamic et al., U.S. Pat. No. 5,998,203.

As used herein “unmodified nucleoside” means one of the bases adenine, cytosine, guanine, thymine, or uracil joined to the 1′ carbon of .beta.-D-ribo-furanose.

As used herein, “modified nucleoside” means any nucleotide base which contains a modification in the chemical structure of an unmodified nucleotide base, sugar and/or phosphate.

In some embodiments, the nucleic acids of the presently disclosed subject matter include phosphate backbone modifications comprising one or more phosphorothioate, phosphonoacetate, and/or thiophosphonoacetate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions. For a review of oligonucleotide backbone modifications, see Hunziker and Leumann, 1995, Nucleic Acid Analogues: Synthesis and Properties, in Modern Synthetic Methods, VCH, 331-417, and Mesmaeker et al., 1994, Novel Backbone Replacements for Oligonucleotides, in Carbohydrate Modifications in Antisense Research, ACS, 24-39.

The nucleic acids disclosed herein (e.g., synthetic RNAs, including modified mRNAs) can be conjugated to non-nucleic acid molecules. In some embodiments, the nucleic acids disclosed herein (e.g., synthetic RNAs) are conjugated to (or otherwise physically associated with) a moiety that promotes cellular uptake, nuclear entry, and/or nuclear retention. For example, the present disclosure contemplates conjugates of peptide transport moieties and the nucleic acids. In some embodiments, the nucleic acid is conjugated to a peptide transporter moiety, for example a cell-penetrating peptide transport moiety, which is effective to enhance transport of the oligomer into cells. For example, in some embodiments the peptide transporter moiety is an arginine-rich peptide. In further embodiments, the transport moiety is attached to either the 5′ or 3′ terminus of the oligomer. When such peptide is conjugated to either termini, the opposite termini is then available for further conjugation to a modified terminal group as described herein. Peptide transport moieties are generally effective to enhance cell penetration of the nucleic acids. In some embodiments, a glycine (G) or proline (P) amino acid subunit is included between the nucleic acid and the remainder of the peptide transport moiety (e.g., at the carboxy or amino terminus of the carrier peptide) to reduces the toxicity of the conjugate, while maintaining or improving efficacy relative to conjugates with different linkages between the peptide transport moiety and nucleic acid.

A reporter moiety, such as fluorescein or a radiolabeled group, may be attached to nucleic acids disclosed herein for purposes of detection. Alternatively, the reporter label attached to the oligomer may be a ligand, such as an antigen or biotin, capable of binding a labeled antibody or streptavidin. In selecting a moiety for attachment or modification of a nucleic acid molecule, it is generally of course desirable to select chemical compounds of groups that are biocompatible and likely to be tolerated by a subject without undesirable side effects.

In some embodiments, the agent comprises a decoy RNA. As used herein, the term “decoy RNA” refers to an RNA which binds to either the transcription factor or the nascent RNA transcribed from the at least one regulatory element in a manner that interferes with the interaction between the nascent transcribed RNA and the transcription factor. For example, a decoy RNA can bind to the transcription factor in a manner that outcompetes the nascent RNA transcribed from the at least one regulatory element for binding to the transcription factor. In some embodiments, the decoy RNA binds to the transcription factor in a manner that outcompetes the nascent RNA transcribed from the at least one regulatory element for binding to the transcription factor in the absence of directly competing with binding of the transcription factor to the at least one regulatory sequence.

In some embodiments, the decoy RNA comprises a synthetic RNA having a nucleotide sequence that is homologous to the RNA transcribed from the at least one regulatory element. As used herein, the term “synthetic RNA” refers to an RNA molecule that can be generated by in vitro transcription, by direct chemical synthesis or an RNA molecule that is produced in a genetically engineered cell, such as in a bacterial cell, for e.g., in an E. coli cell, but is not produced by that type of cell if it is not genetically engineered. In some contexts, the synthetic RNA molecule contains at least one non-naturally occurring modification compared to its counterpart naturally occurring RNA. As used herein, a synthetic RNA that includes “at least one modification” contains such at least one non-naturally occurring modification. It should appreciate that nucleic acids of use herein that contain at least one modification may, in some embodiments, contain other naturally occurring modifications.

Methods for generating DNA templates for in vitro transcription are well known to those of skill in the art using standard molecular cloning techniques. Approaches to the assembly of DNA templates that do not rely upon the presence of restriction endonuclease cleavage sites are also envisioned, e.g., splint-mediated ligation. The transcribed, synthetic RNA can be modified further post-transcription, e.g., by adding a cap or other functional group. In an aspect, a synthetic RNA comprises a 5′ and/or a 3′-cap structure. Synthetic RNA can be single stranded (e.g., ssRNA) or double stranded (e.g., dsRNA). The 5′ and/or 3′-cap structure can be on only the sense strand, the antisense strand, or both strands. By “cap structure” is meant chemical modifications, which have been incorporated at either terminus of the oligonucleotide (see, for example, Adamic et al., U.S. Pat. No. 5,998,203, incorporated by reference herein). These terminal modifications protect the nucleic acid molecule from exonuclease degradation, and can help in delivery and/or localization within a cell. The cap can be present at the 5′-terminus (5′-cap) or at the 3′-terminal (3′-cap) or can be present on both termini.

Non-limiting examples of the 5′-cap include, but are not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Iyer, 1993, Tetrahedron 49, 1925; incorporated by reference herein).

The synthetic RNA may comprise at least one modified nucleoside, such as pseudouridine, m5U, s2U, m6A, and m5C, N1-methylguanosine, N1-methyladenosine, N7-methylguanosine, 2′-)-methyluridine, and 2′-O-methylcytidine. Polymerases that accept modified nucleosides are known to those of skill in the art. Modified polymerases can be used to generate synthetic, modified RNAs. Thus, for example, a polymerase that tolerates or accepts a particular modified nucleoside as a substrate can be used to generate a synthetic, modified RNA including that modified nucleoside.

In some embodiments, the synthetic RNA provokes a reduced (or absent) innate immune response in vivo or reduced interferon response in vivo by the transfected tissue or cell population. mRNA produced in eukaryotic cells, e.g., mammalian or human cells, is heavily modified, the modifications permitting the cell to detect RNA not produced by that cell. The cell responds by shutting down translation or otherwise initiating an innate immune or interferon response. Thus, to the extent that an exogenously added RNA can be modified to mimic the modifications occurring in the endogenous RNAs produced by a target cell, the exogenous RNA can avoid at least part of the target cell's defense against foreign nucleic acids. Thus, in some embodiments, synthetic RNAs include in vitro transcribed RNAs including modifications as found in eukaryotic/mammalian/human RNA in vivo. Other modifications that mimic such naturally occurring modifications can also be helpful in producing a synthetic RNA molecule that will be tolerated by a cell.

In some embodiments, the synthetic RNA is at least 81% identical to RNA transcribed from the at least one regulatory element. In some embodiments, the synthetic RNA is at least 82% identical to RNA transcribed from the at least one regulatory element. In some embodiments, the synthetic RNA is at least 83% identical to RNA transcribed from the at least one regulatory element. In some embodiments, the synthetic RNA is at least 84% identical to RNA transcribed from the at least one regulatory element. In some embodiments, the synthetic RNA is at least 85% identical to RNA transcribed from the at least one regulatory element. In some embodiments, the synthetic RNA is at least 86% identical to RNA transcribed from the at least one regulatory element. In some embodiments, the synthetic RNA is at least 87% identical to RNA transcribed from the at least one regulatory element. In some embodiments, the synthetic RNA is at least 88% identical to RNA transcribed from the at least one regulatory element. In some embodiments, the synthetic RNA is at least 89% identical to RNA transcribed from the at least one regulatory element. In some embodiments, the synthetic RNA is at least 90% identical to RNA transcribed from the at least one regulatory element. In some embodiments, the synthetic RNA is at least 91% identical to RNA transcribed from the at least one regulatory element. In some embodiments, the synthetic RNA is at least 92% identical to RNA transcribed from the at least one regulatory element. In some embodiments, the synthetic RNA is at least 93% identical to RNA transcribed from the at least one regulatory element. In some embodiments, the synthetic RNA is at least 94% identical to RNA transcribed from the at least one regulatory element. In some embodiments, the synthetic RNA is at least 95% identical to RNA transcribed from the at least one regulatory element. In some embodiments, the synthetic RNA is at least 96% identical to RNA transcribed from the at least one regulatory element. In some embodiments, the synthetic RNA is at least 96% identical to RNA transcribed from the at least one regulatory element. In some embodiments, the synthetic RNA is at least 97% identical to RNA transcribed from the at least one regulatory element. In some embodiments, the synthetic RNA is at least 98% identical to RNA transcribed from the at least one regulatory element. In some embodiments, the synthetic RNA is at least 99% identical to RNA transcribed from the at least one regulatory element. In some embodiments, the synthetic RNA comprises at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the synthetic RNA is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from the at least one regulatory element and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the synthetic RNA consists of, consists essentially of a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from the at least one regulatory element and contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element, and comprises at least one modification.

In some embodiments, the synthetic RNA consists of, consists essentially of, or comprises a nucleotide sequence that comprises an RNA binding site for the transcription factor.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the transcription factor binding site in the RNA transcribed from the at least one regulatory element and contains at least one, two, three, four, five, six, seven, eight, nine, or 10, 11, 12, 13, 14, 15, 16, 17, 18, 19, 20, 25, 30, or more, mismatched nucleotides as compared to the transcription factor binding site in the RNA transcribed from the at least one regulatory element.

In some embodiments, the synthetic RNA consists of, consists essentially of, or comprises a nucleotide sequence that is at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to the transcription factor binding site in the RNA transcribed from the at least one regulatory element and contains at least one, two, three, four, five, six, seven, eight, nine, or 10, 11, 12, 13, 14, 15, 16, 17, 18, 19, 20, 25, 30, or more, mismatched nucleotides as compared to the transcription factor binding site in the RNA transcribed from the at least one regulatory element, and comprises at least one modification.

In some embodiments, the synthetic RNA comprises a length of between 10 nucleotides and 300 nucleotides. In some embodiments, the synthetic RNA comprises a length of between 10 nucleotides and 300 nucleotides and contains at least 1, at least 2, at least 3, at least 4, at least 5, at least 7, at least 8, or at least 9, or at least 10, or more, mismatched nucleotides as compared to the transcription factor binding site of the RNA transcribed from the at least one regulatory element.

In some embodiments, the synthetic RNAs (e.g., decoy RNA) comprise a sequence having a length that is sufficient to target a unique sequence in the transcriptome (e.g., at least 10 nucleotides. In some embodiments, the decoy RNA comprises a sequence having a length that is therapeutically effective (e.g., a length less than 300, e.g., less than 200, e.g., preferably less than about 100 nucleotides). In some embodiments, the synthetic RNAs comprise a sequence having a length of between 12 and 50 nucleotides.

In some embodiments, the presently disclose subject matter contemplates utilizing at least 2, at least 3, at least 4, at least 5, or more synthetic RNAs targeting the same nascent RNA transcribed from the at least one regulatory element but in different regions. In some embodiments, at least 2, at least 3, at least 4, at least 5, or more synthetic RNAs targeting the same nascent RNA transcribed from the at least one regulatory element in different regions each comprise a length of between 10 and 300 nucleotides. In some embodiments, such synthetic RNAs each comprise a length of between about 10 an d100 nucleotides. In some embodiments, such synthetic RNAs each comprise a length of between 12 and 50 nucleotides. In some embodiments, such synthetic RNAs each comprise a length of between 15 and 30 nucleotides. In some embodiments, such synthetic RNAs each comprise a length of about 20, about 21, about 22, about 23, about 24, about 25, about 26, about 27, about 28, or about 29 nucleotides.

Each of such synthetic RNAs can include at least one modification. In some embodiments, the synthetic RNA comprises a length of between 30 and 60 nucleotides. In some embodiments, the synthetic RNA comprises a length of 20 nucleotidesnucleotides. In some embodiments, the synthetic RNA comprises a length of 21 nucleotidesnucleotides. In some embodiments, the synthetic RNA comprises a length of 22 nucleotidesnucleotides. In some embodiments, the synthetic RNA comprises a length of 23 nucleotidesnucleotides. In some embodiments, the synthetic RNA comprises a length of 24 nucleotides. In some embodiments, the synthetic RNA comprises a length of 25 nucleotides. In some embodiments, the synthetic RNA comprises a length of 26 nucleotides. In some embodiments, the synthetic RNA comprises a length of 27 nucleotides. In some embodiments, the synthetic RNA comprises a length of 28 nucleotides. In some embodiments, the synthetic RNA comprises a length of 29 nucleotides. In some embodiments, the synthetic RNA comprises a length of 30 nucleotides. In some embodiments, the synthetic RNA comprises a length of 35 nucleotides. In some embodiments, the synthetic RNA comprises a length of 40 nucleotides. In some embodiments, the synthetic RNA comprises a length of 45 nucleotides. In some embodiments, the synthetic RNA comprises a length of 50 nucleotides. In some embodiments, the synthetic RNA comprises a length of 55 nucleotides. In some embodiments, the synthetic RNA comprises a length of 60 nucleotides.

In some embodiments, the synthetic RNA comprises a length of 20 nucleotides and contains at least one modification. In some embodiments, the synthetic RNA comprises a length of 21 nucleotides and contains at least one modification. In some embodiments, the synthetic RNA comprises a length of 22 nucleotides and contains at least one modification. In some embodiments, the synthetic RNA comprises a length of 23 nucleotides and contains at least one modification. In some embodiments, the synthetic RNA comprises a length of 24 nucleotides and contains at least one modification. In some embodiments, the synthetic RNA comprises a length of 25 nucleotides and contains at least one modification. In some embodiments, the synthetic RNA comprises a length of 26 nucleotides and contains at least one modification. In some embodiments, the synthetic RNA comprises a length of 27 nucleotides and contains at least one modification. In some embodiments, the synthetic RNA comprises a length of 28 nucleotides and contains at least one modification. In some embodiments, the synthetic RNA comprises a length of 29 nucleotides and contains at least one modification. In some embodiments, the synthetic RNA comprises a length of 30 nucleotides and contains at least one modification. In some embodiments, the synthetic RNA comprises a length of 35 nucleotides and contains at least one modification. In some embodiments, the synthetic RNA comprises a length of 40 nucleotides and contains at least one modification. In some embodiments, the synthetic RNA comprises a length of 45 nucleotides and contains at least one modification. In some embodiments, the synthetic RNA comprises a length of 50 nucleotides and contains at least one modification. In some embodiments, the synthetic RNA comprises a length of 55 nucleotides and contains at least one modification. In some embodiments, the synthetic RNA comprises a length of 60 nucleotides and contains at least one modification.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% identical to RNA transcribed from at least one regulatory element occupied by a transcription factor of interest in a cell type of interest within an organism of interest. For example, candidate transcription factors of interest can be identified as noted above, and the methods disclosed herein can be used to design suitable synthetic RNAs that are capable of binding to RNAs transcribed from regulatory elements of target genes regulated by such transcription factors. In some embodiments, such synthetic RNA contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, mismatched nucleotides as compared to the RNA transcribed from the at least one regulatory element.

In some embodiments, the decoy RNA binds to the nascent RNA transcribed from the at least one regulatory element in a manner that prevents the nascent RNA from binding to the transcription factor. In some embodiments, the decoy RNA comprises a synthetic RNA having a sequence that is complementary to the nascent RNA. In some embodiments, the decoy RNA comprises a synthetic RNA having a sequence that is complementary to at least a portion of the nascent RNA. In some embodiments, the decoy RNA comprises a synthetic RNA having a sequence that is complementary to the transcription factor binding site in the nascent RNA transcribed from the at least one regulatory element. In some embodiments, the decoy RNA comprises a synthetic RNA having a sequence that is complementary to at least a portion of the transcription factor binding site in the nascent RNA transcribed from the at least one regulatory element.

In some embodiments, the decoy RNA comprises a synthetic RNA having a length of between 10 and 300 nucleotides and a sequence that is complementary to at least a portion of the nascent RNA transcribed from the at least one regulatory element. In some embodiments, the decoy RNA comprises a synthetic RNA having a length of between 10 and 300 nucleotides and a sequence that is complementary to at least a portion of the transcription factor binding site in the nascent RNA transcribed from the at least one regulatory element. In some embodiments, the synthetic RNA has a length of between 10 and 300 nucleotides and has a sequence that is complementary to at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of a sequence of nascent RNA transcribed from the at least one regulatory element.

In some embodiments, the synthetic RNA has a length of between 30 and 60 nucleotides and has a sequence that is complementary to at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% of a sequence of RNA transcribed from the at least one regulatory element. In some embodiments, the synthetic RNA has a length of between 30 and 60 nucleotides and contains at least one, two, three, four, five, six, seven, eight, nine, 10, 11, 12, 13, 14, 15, 16, 17, 18, 19, 20, 25, 30, 35, 40, 45, 50, 55, or more, nucleotides that are complementary to the nascent RNA transcribed from the at least one regulatory element.

The presently disclosed subject matter also contemplates synthetic RNA consisting of, consisting essentially of, or comprising nucleotide sequences that are at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 95%, at least 96%, at least 97%, at least 98%, or at least 99% complementary to nascent RNA transcribed from at least one regulatory element occupied by a transcription factor of interest in a cell type of interest within an organism of interest. For example, candidate transcription factors of interest can be identified as noted above, and the methods disclosed herein can be used to design suitable synthetic RNAs that are capable of binding to RNAs transcribed from regulatory elements of target genes regulated by such transcription factors. In some embodiments, such synthetic RNA optionally contains at least one, two, three, four, five, six, seven, eight, nine, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 175, 200, 225, 250, or more, nucleotides that are not complementary to the RNA transcribed from the at least one regulatory element.

In some embodiments, the synthetic, modified mRNA (or other synthetic nucleic acid) is capable of evading an innate immune response of a cell, tissue, or subject in which the mRNA is introduced and/or does not induce, or has decreased ability to induce, an innate immune response, e.g., as compared to a corresponding unmodified mRNA. Because the synthetic nucleic acids (e.g., mRNAs) are modified, e.g., to enhance the efficiency of their translation, their intracellular retention, stability, and also possess decreased immunogenicity, the synthetic, modified nucleic acids (e.g., mRNAs) having one or more these properties also may also be referred to in some embodiments as “enhanced nucleic acids.” In some embodiments, the peptide, polypeptide, or protein encoded by the synthetic, modified mRNA comprises one or more post-translational modifications (e.g., those present in mammalian, e.g., human cells).

The modified mRNAs can be engineered to encode a peptide, polypeptide, or protein (e.g., antibody or antibody fragment) that lacks a secretory signal sequence, such that the translated peptide, polypeptide, or protein is not secreted from the target cell in which it is produced. The modified mRNAs can be engineered to encode a peptide, polypeptide, or protein (e.g. antibody or antibody fragment) containing a nuclear localization signal sequence that allows for entrance of the peptide, polypeptide, or protein into the nucleus of a cell of interest (e.g., target cell) where transcription of the target gene regulated by a transcription factor of interest is located. In some embodiments, the nuclear localization signal sequence (NLS) comprises a canonical NLS. In some embodiments, the NLS comprises a single stretch of five to six basic amino acids (e.g., exemplified by the simian virus (SV) 40 large T antigen NLS). In some embodiments, the NLS comprises a bipartite NLS composed of two basic amino acids, a spacer region of 10-12 amino acids, and a cluster in which three of five amino acids must be basic (e.g., as exemplified by nucleoplasmin).

The modified mRNAs can be engineered to encode peptides, polypeptides, or proteins employing NLS-independent mechanisms for passage through the nuclear pore complex into the nucleus of target cells of interest. Examples of such NLS-independent mechanisms include passive diffusion of small proteins (<30-40 kDa), distinct nuclear-directing motifs [D. Christophe, C. Christophe-Hobertus, B. Pichon, Cell Signal 12, 337 (May, 2000), incorporated herein by reference], interaction with NLS-containing proteins, or alternatively, a direct interaction with the nuclear pore proteins (NUPs); [L. Xu, J. Massague, Nat Rev Mol Cell Biol 5, 209 (March, 2004), incorporated herein by reference]. In some embodiments, the mRNA encodes a peptide, polypeptide, or protein that contains nuclear translocation sequences from signaling proteins that translocate into the nucleus upon stimulation, in an NLS-independent manner, so that the peptide, polypeptide, or protein can translocate to the nucleus. Such translocation may occur via direct interaction with NUPs. Examples of such signaling proteins include ERKs, MEKs and SMADs. In some embodiments, the modified mRNAs are engineered to lack consensus sequences that interact with exportin proteins that mediate rapid export of shuttling proteins from the nucleus (e.g., a nuclear export signal (NES), such as the NES consensus sequence of LXXLXXLXL (SEQ ID NO: 1263); identified as having sequence identifier number 36 in U.S. Publication No. 2014/0212438, which is incorporated herein by reference in its entirety)). The peptides, polypeptides, and proteins encoded by the modified mRNAs can be engineered to contain nuclear retention signals that enable the peptides, polypeptides, and proteins encoded by the modified mRNAs to remain in the nucleus once transported there.

In some embodiments, the mRNA encodes a peptide, polypeptide, or protein having nuclear targeting activity that comprises a nuclear targeting sequence less than or equal to 20 amino acids in length comprising X₁, X₂, X₃, wherein X₁ and X₃ are each independently selected from the group consisting of serine, threonine, aspartic acid and glutamic acid, and wherein X₂ is proline, as described in U.S. Publication No. 2014/0212438, which is incorporated herein by reference).

The peptides, polypeptides, and proteins encoded by the modified mRNAs can be engineered to be conjugated to a nuclear localization sequence-binding protein antibody or fragment thereof (i.e., so that when the peptide, polypeptide, or protein is translated in a target cell of interest, the anti-nuclear localization sequence-binding protein antibody portion of the peptide, polypeptide, or protein binds to a nuclear localization sequence and transports the peptide, polypeptide, or protein into the nucleus of the target cell of interest.

It should be appreciated that the modified mRNAs can be engineered to encode peptides, polypeptides, and proteins (e.g., antibodies or antibody fragments) which contain nuclear localization signal sequences, and/or nuclear retention signal sequences, and/or lack secretory signal sequences, and/or nuclear export signal sequences.

The synthetic, modified mRNAs of use herein may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: TRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

“Synthetic, modified mRNA” and “modified mRNA” are used interchangeably herein. Modified mRNAs of use herein (e.g., encoding a peptide, polypeptide, or protein that interferes with binding between the transcribed RNA and a transcription factor of interest need not be uniformly modified along the entire length of the molecule. Different nucleotide modifications and/or backbone structures may exist at various positions in the mRNA. Other components of nucleic acid are optional, and may be beneficial in some embodiments. For example, a 5′ untranslated region (UTR) and/or a 3′UTR may be provided, wherein either or both may independently contain one or more different nucleoside modifications. In such embodiments, nucleoside modifications may also be present in the translatable region. Also contemplated are nucleic acids containing a Kozak sequence. In some embodiments, modified mRNA, e.g., in vitro transcribed mRNA, comprises a polyA tail at its 3′ end. Methods of adding a polyA tail to mRNA are known in the art, e.g., enzymatic addition via polyA polymerase or ligation with a suitable ligase.

One of ordinary skill in the art will appreciate that the nucleotide analogs or other modification(s) may be located at any position(s) of a mRNA such that the function of the nucleic acid is not substantially decreased. A modification may also be a 5′ or 3′terminal modification. The mRNA may contain at a minimum one and at maximum 100% modified nucleotides, or any intervening percentage, such as at least about 50% modified nucleotides, at least about 55% modified nucleotides, at least about 60% modified nucleotides, at least about 65% modified nucleotides, at least about 70% modified nucleotides, at least about 75% modified nucleotides, at least about 80% modified nucleotides, at least about 85% modified nucleotides, or at least about 90% modified nucleotides.

In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of pyridin-4-one ribonucleoside, 5-aza-uridine, 2-thio-5-aza-midine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taulinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudomidine, dihydrouridine, dihydropseudouridine, 2-thio-dihydromidine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N-6-(cis-hydroxyisopentenyl) adenosine, N6-glycinylcarbamoyladenosine, N6-threonylcarbamoyladenosine, 2-methylthio-N-6-threonyl carbamoyladenosine, N6,N6-dimethyladenosine, 7-methyladenine, 2-methylthio-adenine, and 2-methoxy-adenine. In some embodiments, the synthetic, modified mRNA encoding a peptide, polypeptide, or protein that interferes with binding between the RNA transcribed from at least one regulatory element and the transcription factor that binds to the RNA and the at least one regulatory element comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

Generally, the length of a modified mRNA of the present disclosure is suitable for peptide, polypeptide, or protein production in a cell (e.g., a mammalian cell, e.g., human cell). For example, the modified mRNA is of a length sufficient to allow translation of at least a dipeptide in a cell. In one embodiment, the length of the modified mRNA is greater than 30 nucleotides. In another embodiment, the length is greater than 35 nucleotides. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In some embodiments the length is no more than about 500 nucleotides, 750 nucleotides, 1000 nucleotides (1 kB), 2 kB, 3 kB, 4 kB, 5 kB, 6 kB, 7 kB, 8 kB, 9 kB, or 10 kB. In various embodiments the length can range from any lower limit to any upper limit that is greater than the lower limit.

In some embodiments, the modified mRNA encodes a peptide, polypeptide, or protein that binds to the transcription factor in a manner that prevents the transcription factor from binding to the RNA transcribed from the at least one regulatory element. In some embodiments, the peptide, polypeptide, or protein prevents the transcription factor from binding to the RNA transcribed from the at least one regulatory element, but does not prevent the transcription factor from directly binding to the at least one regulatory element (e.g., the peptide, polypeptide, or protein binds to the RNA binding domain or a site in proximity to the RNA binding domain of the transcription factor, but does not bind to the DNA binding domain or a site in proximity to the DNA binding domain of the transcription factor of interest). In some embodiments, the modified mRNA encodes a peptide, polypeptide, or protein that binds to the transcription factor at the same site that the RNA transcribed from at least one regulatory element would bind to the transcription factor. In some embodiments, modified mRNA encodes a peptide, polypeptide, or protein that binds to at least a portion of the same site that the RNA transcribed from at least one regulatory element would bind to the transcription factor (i.e., the agent binds to one or more amino acids of the transcription factor binding site for the RNA transcribed from the at least one regulatory element, but does not bind to all of the amino acids of such site). In some embodiments, the modified mRNA encodes a peptide, polypeptide, or protein that binds to the transcription factor in proximity to where RNA transcribed from at least one regulatory element binds to the transcription factor, but the agent masks the RNA binding site so the RNA can no longer bind to the transcription factor. In some embodiments, the modified mRNA encodes a peptide, polypeptide, or protein that binds to the transcription factor away from where the RNA transcribed from at least one regulatory element binds to the transcription factor, but the agent causes the transcription factor to change its conformation such that the RNA transcribed from at least one regulatory element can no longer bind to the transcription factor. In some embodiments, binding of the peptide, polypeptide, or protein (encoded by the mRNA) to the transcription factor affects another protein or cofactor that interacts with the transcription factor and the other protein or cofactor inhibits the RNA transcribed from at least one regulatory element from binding to the transcription factor.

In some embodiments, the modified mRNA encodes a peptide, polypeptide or protein of interest that binds to the transcription factor and has a length equal to the length of the binding site in the transcribed RNA for the transcription factor of interest. In some embodiments, the modified mRNA encodes a peptide, polypeptide or protein of interest that binds to the transcription factor and has a length equal to a portion of the length of the binding site in the transcribed RNA for the transcription factor of interest.

In some embodiments, the modified mRNA encodes an antibody or antibody fragment thereof that binds to the transcription factor in a manner that prevents the transcription factor from binding to the RNA transcribed from the at least one regulatory element. In some embodiments, the antibody or antibody fragment prevents the transcription factor from binding to the RNA transcribed from the at least one regulatory element, but does not prevent the transcription factor from directly binding to the at least one regulatory element (e.g., the antibody or antibody fragment binds to the RNA binding domain or a site in proximity to the RNA binding domain of the transcription factor, but does not bind to the DNA binding domain or a site in proximity to the DNA binding domain of the transcription factor of interest).

The modified mRNAs may encode full length antibodies or smaller antibodies (e.g., both heavy and light chains). For example, mRNAs may be translated in a cell, tissue, or subject for expression of the heavy and light chains of an immunoglobulin protein (e.g., IgA, IgD, IgE, IgG, and IgM) or antigen-binding fragments thereof (e.g., which bind to a target of interest, e.g., that bind to RNA transcribed from a regulatory element or that bind to a transcription factor of interest and inhibit binding of the TF to RNA transcribed from a regulatory element. The immunoglobulin proteins may be fully human, humanized, or chimeric immunoglobulin proteins. In some embodiments, the mRNA encodes an immunoglobulin protein or an antigen-binding fragment thereof, such as an immunoglobulin heavy chain, an immunoglobulin light chain, a single chain Fv, a fragment of an antibody, such as Fab, Fab′, or (Fab′)₂, or an antigen binding fragment of an immunoglobulin (See, e.g., US Publication No. 2013/0244282, which is incorporated herein by reference in its entirety). It should be appreciated that a single mRNA may be engineered to encode more than one subunit (e.g. in the case of a single-chain Fv antibody). In certain embodiments, separate mRNA molecules encoding the individual subunits may be administered in separate transfer vehicles. In some embodiments, the mRNA may encode full length antibodies (both heavy and light chains of the variable and constant regions) or fragments of antibodies (e.g. Fab, Fv, or a single chain Fv (scFv). In some embodiments the mRNA may encode a single domain antibody or antigen binding fragment thereof.

In some embodiments, the modified mRNA encodes an antibody or antibody fragment thereof that binds to all or a portion of the RNA binding domain of a transcription factor of interest. In some embodiments, the modified mRNA encodes an antibody or antibody fragment that binds to the RNA binding domain of the transcription factor in a manner that interferes with binding of the transcription factor to the RNA transcribed from at least one regulatory element, but does not bind to or block any other portion of the transcription factor (e.g., the DNA binding domain). In some embodiments, the modified mRNA encodes an antibody or an antibody fragment that binds to the transcription factor at a portion of the RNA binding domain that interacts with the binding site in the transcribed RNA for the transcription factor of interest.

In some embodiments, the modified mRNA encodes a peptide, polypeptide, or protein that binds to the RNA transcribed from the at least one regulatory element in a manner that prevents the transcription factor from binding to the RNA transcribed from the at least one regulatory element. In some embodiments, the modified mRNA encodes a peptide, polypeptide, or protein that binds to the RNA in the region that the RNA normally binds to the transcription factor. In some embodiments, the modified mRNA encodes a peptide, polypeptide, or protein that binds to the RNA at a different site from where the RNA binds to the transcription factor, e.g., such that the agent may mask the site on the RNA that binds to the transcription factor. In some embodiments, the modified mRNA encodes an antibody or antibody fragment that binds to the RNA transcribed from the at least one regulatory element in a manner that prevents the transcription factor from binding to the RNA transcribed from the at least one regulatory element.

In some embodiments, the antibody or antibody fragment encoded by the modified mRNA comprises a specific RNA-binding antibody or antibody fragment thereof. In some embodiments, the antibody comprises a specific RNA-binding antibody having a four-amino acid code (see, e.g., Sherman et al., “Specific RNA-binding antibodies with a four-amino-acid code,” J Mol Biol. 2014; 426(10):2145-57, which is incorporated herein by reference in its entirety). Sherman and colleagues describe methods that can be adapted in accordance with the guidance provided herein to construct and screen specific RNA-binding antibodies or antibody fragments which are capable of binding with specificity for and affinity to RNAs transcribed from regulatory elements occupied by transcription factors of interest wherein the RNA-binding antibodies or antibody fragments interfere with binding between the transcribed RNA and the transcription factor of interest, and decrease transcription of the target gene regulated by the regulatory elements occupied by the transcription factor of interest. For example, Sherman and colleagues describe design of an RNA-targeting Fab library with a minimal amino acid composition (e.g., the Fabs comprise complementarity-determining region (CDR) loops consisting of only the amino acids Tyr (Y), Ser (S), Gly (G) and Arg (R), construction of the Fab library (referred to as a “YSGR Min library” using a single Fab framework (P4-P6 binding Fab2) using Kunkel mutagenesis, the selection of antibodies in the YSGR Min library against particular RNA targets, the screening of individual phage clones by enzyme-linked immunosorbent assay, the expression and characterization of the Fabs, specificity assays, DNA constructs of the RNAs, in vitro transcription for the preparation of RNAs, preparation of the stop template for library construction, phage display for the selection for RNAs, phage ELISA for RNAs, native EMSA and PACE, filter binding assays, and competitive filter binding assays, all of which are incorporated herein by reference.

In some embodiments, the specific RNA-binding antibody comprises RNA-binding antibodies comprising complementarity-determining region (CDR) loops consisting of only the amino acids Tyr (Y), Ser (S), Gly (G) and Arg (R). In some embodiments, the specific RNA-binding antibody comprises RNA-binding antibodies comprising complementarity-determining region (CDR) loops consisting of only the amino acids Y, S, G and X, where X is any amino acid (see, e.g., Ye et al., “Synthetic antibodies for specific recognition and crystallization of structured RNA,” Proc Natl Acad Sci USA 2008; 105:82-7, which is incorporated herein by reference). In some embodiments, the specific RNA-binding antibody comprises RNA-binding antibodies comprising complementarity-determining region (CDR) loops consisting of only the amino acids Y,S, G, R, and X, wherein X is any amino acid (see, e.g., Koldobskaya, et al., “A portable RNA sequence whose recognition by a synthetic antibody facilitates structural determination,” Nat StructMol Biol 2011; 18:100-6, which is incorporated herein by reference in its entirety).

In some embodiments, phage display (or another display technology such as ribosome display, yeast display, bacterial display, mRNA display (e.g., using a cell-free system)) may be used to identify antibodies, peptides, or other proteins that bind to the RNA transcribed from a regulatory element or to a transcription factor that binds to RNA transcribed from at least one regulatory element. The presently disclosed subject matter contemplates modified nucleic acids (e.g., DNA, mRNA) encoding such antibodies, peptides, or proteins.

In some embodiments, the synthetic, modified mRNA encodes a variant peptide, polypeptide, or protein that has a certain identity with a reference peptide, polypeptide, or protein sequence. For example, the presently disclosed subject matter contemplates synthetic, modified mRNA encoding variants of a transcription factor of interest, i.e., a transcription factor that binds to RNA transcribed from at least one regulatory element and the at least one regulatory element. The term “identity” as known in the art, refers to a relationship between the sequences of two or more peptides, as determined by comparing the sequences. In the art, “identity” also means the degree of sequence relatedness between peptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Prut 1, Griffin, A. M., and Gtiffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carrillo et al., SIAM J. Applied Math. 48, 1073 (1988).

In some embodiments, the peptide, protein, or polypeptide variant has at least one activity that is the same or similar to an activity as the reference peptide, polypeptide, or protein (e.g., the peptide, protein, or polypeptide encoded by the synthetic, modified mRNA can bind to the same RNA transcribed from the at least one regulatory element as a transcription factor of interest). For example, the sequence of the mRNA encoding the peptide, protein, or polypeptide variant can be identical or similar to the RNA binding domain of a transcription factor of interest. In some embodiments, the peptide, protein, or polypeptide variant has at least one activity that is the same or similar to an activity as the reference peptide, polypeptide, or protein, but lacks at least one other activity of the reference peptide, polypeptide, or protein (e.g., the peptide, protein, or polypeptide encoded by the synthetic, modified mRNA can bind to the same RNA transcribed from the at least one regulatory element as a transcription factor of interest, but is not capable of binding to the at least one regulatory element). For example, the sequence of the mRNA encoding the peptide, protein, or polypeptide variant can be identical or similar to the RNA binding domain of a transcription factor of interest, but lack the DNA binding domain of the transcription factor of interest (e.g., the amino acids comprising the DNA binding domain can be deleted). In some embodiments, the sequence of the mRNA encoding the peptide, polypeptide, or protein variant can be identical or similar to the RNA binding domain of a transcription factor of interest, and the sequence of mRNA encoding the DNA binding domain of the transcription factor of interest can include one or more modifications (e.g., insertions, deletions, mutations) that prevent the DNA binding domain from binding to the at least one regulatory element. In some embodiments, the variant has an altered activity (e.g., increased or decreased) relative to a reference peptide, polypeptide, or protein (e.g., a transcription factor of interest). For example, an mRNA encoding a transcription factor of interest can be designed to exhibit increased affinity for binding to the transcribed RNA relative to the transcription factor of interest and/or decreased affinity for binding to the at least one regulatory element. Generally, variants of a particular peptide, polynucleotide, protein, or polypeptide of the disclosure will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular reference polynucleotide or polypeptide as determined by sequence alignment programs and parameters described herein and known to those skilled in the art.

As recognized by those skilled in the art, protein fragments, functional protein domains, and homologous proteins are also considered to be within the scope of this disclosure. For example, provided herein is any protein fragment of a reference protein (meaning an mRNA encoding a polypeptide sequence at least one amino acid residue shorter than a reference polypeptide sequence but otherwise identical) about 10, 20, 30, 40, 50, 60, 70, 80, 90, 100, or greater than 100 amino acids in length. In another example, any protein that includes a stretch of about 20, about 30, about 40, about 50, or about 100 amino acids, which are about 40%, about 50%, about 60%, about 70%, about 80%, about 90%, about 95%, or about 100% identical to any of the sequences described herein, can be utilized in accordance with the disclosure. In certain embodiments, a protein sequence to be utilized in accordance with the disclosure includes 2, 3, 4, 5, 6, 7, 8, 9, 10, or more mutations as shown in any of the sequences referenced herein.

In some embodiments, the presently disclosed subject matter provides polynucleotide libraries containing nucleoside modifications, wherein the polynucleotides individually contain a first nucleic acid sequence encoding a peptide, polypeptide, or protein, such as an antibody, protein binding partner, scaffold protein, and other polypeptides (e.g., variants of a transcription factor of interest that can bind to RNA transcribed from regulatory elements of their naturally occurring counterparts (i.e., wild type transcription factors) but are unable to bind to the at least one regulatory element from which the RNA is transcribed and/or bind to the at least one regulatory element from which the RNA is transcribed with a lesser affinity compared to the wild type transcription factor). It should be appreciated that the library can comprise any of the modified mRNA described herein. Typically, the polynucleotides are modified mRNA in a form suitable for direct introduction into a target cell host, which in turn synthesizes the encoded peptide, polypeptide, or protein. In certain embodiments, multiple variants of a protein, each with different amino acid modification(s), are produced and tested to determine the best variant in terms of pharmacokinetics, stability, biocompatibility, and/or biological activity, or a biophysical property such as expression level. In some embodiments, the polynucleotides are assessed for their ability to be translated in the target cell host and to interfere with binding between a transcription factor of interest and RNA transcribed from at least one regulatory element occupied by the transcription factor of interest is assessed. Such a library may contain about 10, 10², 10³, 10⁴, 10⁵, 10⁶, 10⁷, 10⁸, 10⁹, or over 10⁹ possible variants (including substitutions, deletions of one or more residues, and insertion of one or more residues (e.g., variants of a transcription factor of interest comprising one or more sequence modifications to an RNA binding domain and/or DNA binding domain of the variant as compared to the transcription factor of interest, e.g., to alter the binding affinity (e.g., increase or decrease) of the RNA binding domain and/or DNA binding domain for its cognate RNA and/or DNA sequence relative to the binding affinity of the DNA binding domain and/or DNA binding domain of the transcription factor of interest.

In some embodiments, a modified mRNA of the presently disclosed subject matter encodes multiple peptides, polypeptides or proteins of interest that are capable of interfering with binding between the transcribed RNA and the transcription factor of interest. For example, the presently disclosed subject matter provides modified mRNAs containing an internal ribosome entry site (IRES). An IRES may act as the sole ribosome binding site, or may serve as one of multiplelibosome binding sites of an mRNA. An mRNA containing more than one functional ribosome binding site may encode several peptides or polypeptides that are translated independently by the ribosomes (“multicistronic mRNA”). When mRNAs are provided with an IRES, further optionally provided is at least a second translatable region. Examples of IRES sequences that can be used according to the disclosure include without limitation, those from picornaviruses (e.g. FMDV), pest viruses (CFFV), polio viruses (PV), encephalomyocarditis viruses (ECMV), foot-and-mouth disease viruses (FMDV), hepatitis C viruses (HCV), classical swine fever viruses (CSFV), murine leukemia virus (MLV), simian immune deficiency viruses (STY) or cricket paralysis viruses (CrPV). In some embodiments a “self-cleaving” 2A peptide may be used instead of an IRES to, e.g., provide polycistronic expression from a single promoter. Self-cleaving 2A peptides were originally identified and characterized in apthovirus foot-and-mouth disease virus (FMDV). 2A oligopeptides are generally approximately 18-22 aa long and contain a highly conserved c-terminal D(V/I)EXNPGP (SEQ ID NO: 1264) motif that mediates “ribosomal skipping” at the terminal 2A proline and subsequent amino acid (glycine). Examples of 2A peptide sequences that can be used according to the disclosure include without limitation, those from FMDV, equine rhinitis A virus (ERAV, porcine teschovirus-1 (PTV-1), and insect Thosea asigna virus (TaV).

In some embodiments, nucleic acids (e.g., enhanced nucleic acids) of interest herein (e.g., DNA constructs, synthetic RNAs, e.g., homologous or complementary RNAs described herein, mRNAs described herein, etc.) herein may be introduced into cells of interest via transfection, electroporation, cationic agents, polymers, or lipid-based delivery molecules well known to those of ordinary skill in the art.

In some embodiments, methods of the present disclosure enhance nucleic acid delivery into a cell population, in vivo, ex vivo, or in culture. For example, a cell culture containing a plurality of host cells (e.g., eukaryotic cells such as yeast or mammalian cells) is contacted with a composition that contains an enhanced nucleic acid having at least one nucleoside modification and, optionally, a translatable region. In some embodiments, the composition also generally contains a transfection reagent or other compound that increases the efficiency of enhanced nucleic acid uptake into the host cells. The enhanced nucleic acid exhibits enhanced retention in the cell population, relative to a corresponding unmodified nucleic acid. The retention of the enhanced nucleic acid is greater than the retention of the unmodified nucleic acid. In some embodiments, it is at least about 50%, 75%, 90%, 95%, 100%, 150%, 200%, or more than 200% greater than the retention of the unmodified nucleic acid. Such retention advantage may be achieved by one round of transfection with the enhanced nucleic acid, or may be obtained following repeated rounds of transfection.

The synthetic RNAs (e.g., modified mRNAs) of the presently disclosed subject matter may be optionally combined with a reporter gene (e.g., upstream or downstream of the coding region of the mRNA) which, for example, facilitates the determination of modified mRNA delivery to the target cells or tissues. Suitable reporter genes may include, for example, Green Fluorescent Protein mRNA (GFP mRNA), Renilla Luciferase mRNA (Luciferase mRNA), Firefly Luciferase mRNA, or any combinations thereof. For example, GFP mRNA may be fused with a mRNA encoding a nuclear localization sequence to facilitate confirmation of mRNA localization in the target cells where the RNA transcribed from the at least one regulatory element is taking place.

As used herein, the terms “transfect” or “transfection” mean the introduction of a nucleic acid, e.g., a synthetic RNA, e.g., modified mRNA into a cell, or preferably into a target cell. The introduced synthetic RNA (e.g., modified mRNA) may be stably or transiently maintained in the target cell. The term “transfection efficiency” refers to the relative amount of synthetic RNA (e.g., modified mRNA) taken up by the target cell which is subject to transfection. In practice, transfection efficiency may be estimated by the amount of a reporter nucleic acid product expressed by the target cells following transfection. Preferred embodiments include compositions with high transfection efficacies and in particular those compositions that minimize adverse effects which are mediated by transfection of non-target cells. In some embodiments, compositions of the present invention that demonstrate high transfection efficacies improve the likelihood that appropriate dosages of the synthetic RNA (e.g., modified mRNA) will be delivered to the target cell, while minimizing potential systemic adverse effects.

In some embodiments a cell may be genetically modified (in vitro or in vivo) (e.g., using a nucleic acid construct, e.g., a DNA construct) to cause it to express (i) an agent that modulates binding between nascent RNA transcribed from at least one regulatory element of a target gene and a transcription factor which binds to both the nascent RNA and the at least one regulatory element or (ii) an mRNA that encodes such an agent. For example, the present disclosure contemplates generating a cell or cell line that transiently or stably expresses an RNA that inhibits binding of the TF to nascent RNA transcribed from a regulatory element to which that TF binds or that transiently stably expresses an mRNA that encodes an antibody (or other protein capable of specific binding) that interferes with binding between a TF and nascent RNA transcribed from a regulatory element to which that TF binds. The genetically modified cells and constructs may be useful, e.g., in gene therapy approaches. For example, in some embodiments, such a nucleic acid construct is administered to an individual in need thereof. In other embodiments, cells (e.g., autologous) that have been contacted ex vivo with such a construct can be administered to an individual in need thereof. The construct may include a promoter operably linked to a sequence that encodes the agent or mRNA.

The synthetic RNA (e.g., modified mRNA) can be formulated with one or more acceptable reagents, which provide a vehicle for delivering such synthetic RNA (e.g., modified mRNA) to target cells. Appropriate reagents are generally selected with regard to a number of factors, which include, among other things, the biological or chemical properties of the synthetic RNA (e.g., modified mRNA), the intended route of administration, the anticipated biological environment to which such synthetic RNA (e.g., modified mRNA) will be exposed and the specific properties of the intended target cells. In some embodiments, transfer vehicles, such as liposomes, encapsulate the synthetic RNA (e.g., modified mRNA) without compromising biological activity. In some embodiments, the transfer vehicle demonstrates preferential and/or substantial binding to a target cell relative to non-target cells. In a preferred embodiment, the transfer vehicle delivers its contents to the target cell such that the synthetic RNA (e.g., modified mRNA) are delivered to the appropriate subcellular compartment, such as the cytoplasm.

In some embodiments, the transfer vehicle in the compositions of the invention is a liposomal transfer vehicle, e.g. a lipid nanoparticle. In one embodiment, the transfer vehicle may be selected and/or prepared to optimize delivery of the nucleic acid (e.g., synthetic RNA (e.g., modified mRNA)) to a target cell. For example, if the target cell is a hepatocyte the properties of the transfer vehicle (e.g., size, charge and/or pH) may be optimized to effectively deliver such transfer vehicle to the target cell, reduce immune clearance and/or promote retention in that target cell. Alternatively, if the target cell is the central nervous system (e.g., for the treatment of neurodegenerative diseases, the transfer vehicle may specifically target brain or spinal tissue), selection and preparation of the transfer vehicle must consider penetration of, and retention within the blood brain barrier and/or the use of alternate means of directly delivering such transfer vehicle to such target cell. In one embodiment, the compositions of the present invention may be combined with agents that facilitate the transfer of exogenous synthetic RNA (e.g., modified mRNA) (e.g., agents which disrupt or improve the permeability of the blood brain barrier and thereby enhance the transfer of exogenous mRNA to the target cells).

The use of liposomal transfer vehicles to facilitate the delivery of nucleic acids to target cells is contemplated by the present disclosure. Liposomes (e.g., liposomal lipid nanoparticles) are generally useful in a variety of applications in research, industry, and medicine, particularly for their use as transfer vehicles of diagnostic or therapeutic compounds in vivo (Lasic, Trends Biotechnol., 16: 307-321, 1998; Drummond et al., Pharmacol. Rev., 51: 691-743, 1999) and are usually characterized as microscopic vesicles having an interior aqua space sequestered from an outer medium by a membrane of one or more bilayers. Bilayer membranes of liposomes are typically formed by amphiphilic molecules, such as lipids of synthetic or natural origin that comprise spatially separated hydrophilic and hydrophobic domains (Lasic, Trends Biotechnol., 16: 307-321, 1998). Bilayer membranes of the liposomes can also be formed by amphiphilic polymers and surfactants (e.g., polymerosomes, niosomes, etc.).

In the context of the present disclosure, a liposomal transfer vehicle typically serves to transport the synthetic RNA (e.g., modified mRNA) to the target cell. For the purposes of the present invention, the liposomal transfer vehicles are prepared to contain the desired nucleic acids. The process of incorporation of a desired entity (e.g., a nucleic acid) into a liposome is often referred to as “loading” (Lasic, et al., FEBS Lett., 312: 255-258, 1992). The liposome-incorporated nucleic acids may be completely or partially located in the interior space of the liposome, within the bilayer membrane of the liposome, or associated with the exterior surface of the liposome membrane. The incorporation of a nucleic acid into liposomes is also referred to herein as “encapsulation” wherein the nucleic acid is entirely contained within the interior space of the liposome. The purpose of incorporating a synthetic RNA (e.g., modified mRNA) into a transfer vehicle, such as a liposome, is often to protect the nucleic acid from an environment which may contain enzymes or chemicals that degrade nucleic acids and/or systems or receptors that cause the rapid excretion of the nucleic acids. Accordingly, in a preferred embodiment of the present invention, the selected transfer vehicle is capable of enhancing the stability of the synthetic RNA (e.g., modified mRNA) contained therein. The liposome can allow the encapsulated synthetic RNA (e.g., modified mRNA) to reach the target cell and/or may preferentially allow the encapsulated synthetic RNA (e.g., modified mRNA) to reach the target cell, or alternatively limit the delivery of such synthetic RNA (e.g., modified mRNA) to other sites or cells where the presence of the administered synthetic RNA (e.g., modified mRNA) may be useless or undesirable. Furthermore, incorporating the synthetic RNA (e.g., modified mRNA) into a transfer vehicle, such as for example, a cationic liposome, also facilitates the delivery of such synthetic RNA (e.g., modified mRNA) into a target cell.

Liposomal transfer vehicles can be prepared to encapsulate one or more desired synthetic RNA (e.g., modified mRNA) such that the compositions demonstrate a high transfection efficiency and enhanced stability. While liposomes can facilitate introduction of nucleic acids into target cells, the addition of polycations (e.g., poly L-lysine and protamine), as a copolymer can facilitate, and in some instances markedly enhance the transfection efficiency of several types of cationic liposomes by 2-28 fold in a number of cell lines both in vitro and in vivo. (See N. J. Caplen, et al., Gene Ther. 1995; 2: 603; S. Li, et al., Gene Ther. 1997; 4, 891.)

In some embodiments, the transfer vehicle is formulated as a lipid nanoparticle. As used herein, the phrase “lipid nanoparticle” refers to a transfer vehicle comprising one or more lipids (e.g., cationic lipids, non-cationic lipids, and PEG-modified lipids). Preferably, the lipid nanoparticles are formulated to deliver one or more synthetic RNAs (e.g., modified mRNAs) to one or more target cells.

Examples of suitable lipids include, for example, the phosphatidyl compounds (e.g., phosphatidylglycerol, phosphatidylcholine, phosphatidylserine, phosphatidylethanolamine, sphingolipids, cerebrosides, and gangliosides). Also contemplated is the use of polymers as transfer vehicles, whether alone or in combination with other transfer vehicles. Suitable polymers may include, for example, polyacrylates, polyalkycyanoacrylates, polylactide, polylactide-polyglycolide copolymers, polycaprolactones, dextran, albumin, gelatin, alginate, collagen, chitosan, cyclodextrins, dendrimers and polyethylenimine. In one embodiment, the transfer vehicle is selected based upon its ability to facilitate the transfection of a synthetic RNA (e.g., modified mRNA) to a target cell.

The present disclosure contemplates the use of lipid nanoparticles as transfer vehicles comprising a cationic lipid to encapsulate and/or enhance the delivery of synthetic RNA (e.g., modified mRNA) into the target cell, e.g., that will act as a depot for production of a peptide, polypeptide, or protein (e.g., antibody or antibody fragment) that interferes with binding between RNA transcribed from at least one regulatory element and a transcription factor that binds to the transcribed RNA and the at least one regulatory element. As used herein, the phrase “cationic lipid” refers to any of a number of lipid species that carry a net positive charge at a selected pH, such as physiological pH. The contemplated lipid nanoparticles may be prepared by including multi-component lipid mixtures of varying ratios employing one or more cationic lipids, non-cationic lipids and PEG-modified lipids. Several cationic lipids have been described in the literature, many of which are commercially available.

Suitable cationic lipids of use in the compositions and methods herein include those described in international patent publication WO 2010/053572, incorporated herein by reference, e.g., C12-200 described at paragraph [00225] of WO 2010/053572. In certain embodiments, the compositions and methods of the invention employ a lipid nanoparticles comprising an ionizable cationic lipid described in U.S. provisional patent application 61/617,468, filed Mar. 29, 2012 (incorporated herein by reference), such as, e.g., (15Z,18Z) N,N-dimethyl-6-(9Z,12Z)-octadeca-9,12-dien-1-yl)tetracosa-15,18-dien-1-amine (HGT5000), (15Z,18Z) N,N-dimethyl-6-((9Z,12Z)-octadeca-9,12-dien-1-yl)tetracosa-4,15,18-trien-1-amine (HGT5001), and (15Z,18Z)—N,N-dimethyl-6-((9Z,12Z)-octadeca-9,12-dien-1-yl)tetracosa-5,15,18-trien-1-amine (HGT5002).

In some embodiments, the cationic lipid N-[1-(2,3-dioleyloxy)propyl]-N,N,N-trimethylammonium chloride or “DOTMA” is used. (Felgner et al. (Proc. Nat'l Acad. Sci. 84, 7413 (1987); U.S. Pat. No. 4,897,355). DOTMA can be formulated alone or can be combined with the neutral lipid, dioleoylphosphatidyl-ethanolamine or “DOPE” or other cationic or non-cationic lipids into a liposomal transfer vehicle or a lipid nanoparticle, and such liposomes can be used to enhance the delivery of nucleic acids into target cells. Other suitable cationic lipids include, for example, 5-carboxyspermylglycinedioctadecylamide or “DOGS,” 2,3-dioleyloxy-N-[2(spermine-carboxamido)ethyl]-N,N-dimethyl-1-propanaminium or “DOSPA” (Behr et al. Proc. Nat.'l Acad. Sci. 86, 6982 (1989); U.S. Pat. Nos. 5,171,678; 5,334,761), 1,2-Dioleoyl-3-Dimethylammonium-Propane or “DODAP”, 1,2-Dioleoyl-3-Trimethylammonium-Propane or “DOTAP”. Contemplated cationic lipids also include 1,2-distearyloxy-N,N-dimethyl-3-aminopropane or “DSDMA”, 1,2-dioleyloxy-N,N-dimethyl-3-aminopropane or “DODMA”, 1,2-dilinoleyloxy-N,N-dimethyl-3-aminopropane or “DLinDMA”, 1,2-dilinolenyloxy-N,N-dimethyl-3-aminopropane or “DLenDMA”, N-dioleyl-N,N-dimethylammonium chloride or “DODAC”, N,N-distearyl-N,N-dimethylammonium bromide or “DDAB”, N-(1,2-dimyristyloxyprop-3-yl)-N,N-dimethyl-N-hydroxyethyl ammonium bromide or “DMRIE”, 3-dimethylamino-2-(cholest-5-en-3-beta-oxybutan-4-oxy)-1-(cis,cis-9,12-octadecadienoxy)propane or “CLinDMA”, 2-[5′-(cholest-5-en-3-beta-oxy)-3′-oxapentoxy)-3-dimethyl-1-(cis,cis-9′, 1-2′-octadecadienoxy)propane or “CpLinDMA”, N,N-dimethyl-3,4-dioleyloxybenzylamine or “DMOBA”, 1,2-N,N′-dioleylcarbamyl-3-dimethylaminopropane or “DOcarbDAP”, 2,3-Dilinoleoyloxy-N,N-dimethylpropylamine or “DLinDAP”, 1,2-N,N′-Dilinoleylcarbamyl-3-dimethylaminopropane or “DLincarbDAP”, 1,2-Dilinoleoylcarbamyl-3-dimethylaminopropane or “DLinCDAP”, 2,2-dilinoleyl-4-dimethylaminomethyl-[1,3]-dioxolane or “DLin-K-DMA”, 2,2-dilinoleyl-4-dimethylaminoethyl-[1,3]-dioxolane or “DLin-K-XTC2-DMA”, and 2-(2,2-di((9Z,12Z)-octadeca-9,12-dien-1-yl)-1,3-dioxolan-4-yl)-N,N-dimethylethanamine (DLin-KC2-DMA)) (See, WO 2010/042877; Semple et al., Nature Biotech. 28:172-176 (2010)), or mixtures thereof. (Heyes, J., et al., J Controlled Release 107: 276-287 (2005); Morrissey, D V., et al., Nat. Biotechnol. 23(8): 1003-1007 (2005); PCT Publication WO2005/121348A1).

The use of cholesterol-based cationic lipids is also contemplated by the present disclosure. Such cholesterol-based cationic lipids can be used, either alone or in combination with other cationic or non-cationic lipids. Suitable cholesterol-based cationic lipids include, for example, DC-Chol (N,N-dimethyl-N-ethylcarboxamidocholesterol), 1,4-bis(3-N-oleylamino-propyl)piperazine (Gao, et al. Biochem. Biophys. Res. Comm. 179, 280 (1991); Wolf et al. BioTechniques 23, 139 (1997); U.S. Pat. No. 5,744,335), or ICE.

The skilled artisan will appreciate that various reagents are commercially available to enhance transfection efficacy. Suitable examples include LIPOFECTIN (DOTMA:DOPE) (Invitrogen, Carlsbad, Calif), LIPOFECTAMINE (DOSPA:DOPE) (Invitrogen), LIPOFECTAMINE2000. (Invitrogen), FUGENE, TRANSFECTAM (DOGS), and EFFECTENE.

Also contemplated are cationic lipids such as the dialkylamino-based, imidazole-based, and guanidinium-based lipids. For example, certain embodiments are directed to a composition comprising one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate, as represented by structure (I) below. In a preferred embodiment, a transfer vehicle for delivery of synthetic RNA (e.g., modified mRNA) may comprise one or more imidazole-based cationic lipids, for example, the imidazole cholesterol ester or “ICE” lipid (3S,10R,13R,17R)-10,13-dimethyl-17-((R)-6-methylheptan-2-yl)-2,3,4,7,8,9,10,11,12,13,14,15,16,17-tetradecahydro-1H-cyclopenta[a]phenanthren-3-yl 3-(1H-imidazol-4-yl)propanoate, as represented by structure (I).

The imidazole-based cationic lipids are also characterized by their reduced toxicity relative to other cationic lipids. The imidazole-based cationic lipids (e.g., ICE) may be used as the sole cationic lipid in the lipid nanoparticle, or alternatively may be combined with traditional cationic lipids, non-cationic lipids, and PEG-modified lipids. The cationic lipid may comprise a molar ratio of about 1% to about 90%, about 2% to about 70%, about 5% to about 50%, about 10% to about 40% of the total lipid present in the transfer vehicle, or preferably about 20% to about 70% of the total lipid present in the transfer vehicle.

In some embodiments, the lipid nanoparticles comprise the HGT4003 cationic lipid 2-((2,3-Bis((9Z,12Z)-octadeca-9,12-dien-1-yloxy)propyl)disulfanyl)-N,N-dimethylethanamine, as represented by structure (II) below, and as further described in U.S. Provisional Application No. 61/494,745, filed Jun. 8, 2011, the entire teachings of which are incorporated herein by reference in their entirety.

In other embodiments the compositions and methods described herein are directed to lipid nanoparticles comprising one or more cleavable lipids, such as, for example, one or more cationic lipids or compounds that comprise a cleavable disulfide (S—S) functional group (e.g., HGT4001, HGT4002, HGT4003, HGT4004 and HGT4005), as further described in U.S. Provisional Application No. 61/494,745, the entire teachings of which are incorporated herein by reference in their entirety.

The use of polyethylene glycol (PEG)-modified phospholipids and derivatized lipids such as derivatized cerarmides (PEG-CER), including N-Octanoyl-Sphingosine-1-[Succinyl(Methoxy Polyethylene Glycol)-2000](C8 PEG-2000 ceramide) is also contemplated by the present invention, either alone or preferably in combination with other lipids together which comprise the transfer vehicle (e.g., a lipid nanoparticle). Contemplated PEG-modified lipids include, but is not limited to, a polyethylene glycol chain of up to 5 kDa in length covalently attached to a lipid with alkyl chain(s) of C₆-C₂₀ length. The addition of such components may prevent complex aggregation and may also provide a means for increasing circulation lifetime and increasing the delivery of the lipid-nucleic acid composition to the target cell, (Klibanov et al. (1990) FEBS Letters, 268 (1): 235-237), or they may be selected to rapidly exchange out of the formulation in vivo (see U.S. Pat. No. 5,885,613). In some embodiments, exchangeable lipids comprise PEG-ceramides having shorter acyl chains (e.g., C14 or C18). The PEG-modified phospholipid and derivatized lipids of the present invention may comprise a molar ratio from about 0% to about 20%, about 0.5% to about 20%, about 1% to about 15%, about 4% to about 10%, or about 2% of the total lipid present in the liposomal transfer vehicle.

The present disclosure also contemplates the use of non-cationic lipids. As used herein, the phrase “non-cationic lipid” refers to any neutral, zwitterionic or anionic lipid. As used herein, the phrase “anionic lipid” refers to any of a number of lipid species that carry a net negative charge at a selected pH, such as physiological pH. Non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. Such non-cationic lipids may be used alone, but are preferably used in combination with other excipients, for example, cationic lipids. When used in combination with a cationic lipid, the non-cationic lipid may comprise a molar ratio of 5% to about 90%, or preferably about 10% to about 70% of the total lipid present in the transfer vehicle.

In some embodiments, the transfer vehicle (e.g., a lipid nanoparticle) is prepared by combining multiple lipid and/or polymer components. For example, a transfer vehicle may be prepared using C12-200, DOPE, chol, DMG-PEG2K at a molar ratio of 40:30:25:5, or DODAP, DOPE, cholesterol, DMG-PEG2K at a molar ratio of 18:56:20:6, or HGT5000, DOPE, chol, DMG-PEG2K at a molar ratio of 40:20:35:5, or HGT5001, DOPE, chol, DMG-PEG2K at a molar ratio of 40:20:35:5. The selection of cationic lipids, non-cationic lipids and/or PEG-modified lipids which comprise the lipid nanoparticle, as well as the relative molar ratio of such lipids to each other, is based upon the characteristics of the selected lipid(s), the nature of the intended target cells, the characteristics of the synthetic RNA (e.g., modified mRNA) to be delivered. Additional considerations include, for example, the saturation of the alkyl chain, as well as the size, charge, pH, pKa, fusogenicity and toxicity of the selected lipid(s). Thus the molar ratios may be adjusted accordingly. For example, in embodiments, the percentage of cationic lipid in the lipid nanoparticle may be greater than 10%, greater than 20%, greater than 30%, greater than 40%, greater than 50%, greater than 60%, or greater than 70%. The percentage of non-cationic lipid in the lipid nanoparticle may be greater than 5%, greater than 10%, greater than 20%, greater than 30%, or greater than 40%. The percentage of cholesterol in the lipid nanoparticle may be greater than 10%, greater than 20%, greater than 30%, or greater than 40%. The percentage of PEG-modified lipid in the lipid nanoparticle may be greater than 1%, greater than 2%, greater than 5%, greater than 10%, or greater than 20%.

In certain embodiments, the lipid nanoparticles of the present disclosure comprise at least one of the following cationic lipids: C12-200, DLin-KC2-DMA, DODAP, HGT4003, ICE, HGT5000, or HGT5001. In embodiments, the transfer vehicle comprises cholesterol and/or a PEG-modified lipid. In some embodiments, the transfer vehicles comprises DMG-PEG2K. In certain embodiments, the transfer vehicle comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, DMG-PEG2K.

The liposomal transfer vehicles for use in the compositions of the disclosure can be prepared by various techniques which are presently known in the art. Multi-lamellar vesicles (MLV) may be prepared conventional techniques, for example, by depositing a selected lipid on the inside wall of a suitable container or vessel by dissolving the lipid in an appropriate solvent, and then evaporating the solvent to leave a thin film on the inside of the vessel or by spray drying. An aqueous phase may then added to the vessel with a vortexing motion which results in the formation of MLVs. Uni-lamellar vesicles (ULV) can then be formed by homogenization, sonication or extrusion of the multi-lamellar vesicles. In addition, unilamellar vesicles can be formed by detergent removal techniques.

In certain embodiments, the compositions of the present disclosure comprise a transfer vehicle wherein the synthetic RNA (e.g., modified mRNA) is associated on both the surface of the transfer vehicle and encapsulated within the same transfer vehicle. For example, during preparation of the compositions of the present invention, cationic liposomal transfer vehicles may associate with the synthetic RNA (e.g., modified mRNA) through electrostatic interactions.

In certain embodiments, the compositions of the invention may be loaded with diagnostic radionuclide, fluorescent materials or other materials that are detectable in both in vitro and in vivo applications. For example, suitable diagnostic materials for use in the present invention may include Rhodamine-dioleoylphospha-tidylethanolamine (Rh-PE), Green Fluorescent Protein mRNA (GFP mRNA), Renilla Luciferase mRNA and Firefly Luciferase mRNA.

Selection of the appropriate size of a liposomal transfer vehicle must take into consideration the site of the target cell or tissue and to some extent the application for which the liposome is being made. In some embodiments, it may be desirable to limit transfection of the synthetic RNA (e.g., modified mRNA) to certain cells or tissues. For example, to target hepatocytes a liposomal transfer vehicle may be sized such that its dimensions are smaller than the fenestrations of the endothelial layer lining hepatic sinusoids in the liver; accordingly the liposomal transfer vehicle can readily penetrate such endothelial fenestrations to reach the target hepatocytes. Alternatively, a liposomal transfer vehicle may be sized such that the dimensions of the liposome are of a sufficient diameter to limit or expressly avoid distribution into certain cells or tissues. For example, a liposomal transfer vehicle may be sized such that its dimensions are larger than the fenestrations of the endothelial layer lining hepatic sinusoids to thereby limit distribution of the liposomal transfer vehicle to hepatocytes. Generally, the size of the transfer vehicle is within the range of about 25 to 250 nm, preferably less than about 250 nm, 175 nm, 150 nm, 125 nm, 100 nm, 75 nm, 50 nm, 25 nm or 10 nm.

A variety of alternative methods known in the art are available for sizing of a population of liposomal transfer vehicles. One such sizing method is described in U.S. Pat. No. 4,737,323, incorporated herein by reference. Sonicating a liposome suspension either by bath or probe sonication produces a progressive size reduction down to small ULV less than about 0.05 microns in diameter. Homogenization is another method that relies on shearing energy to fragment large liposomes into smaller ones. In a typical homogenization procedure, MLV are recirculated through a standard emulsion homogenizer until selected liposome sizes, typically between about 0.1 and 0.5 microns, are observed. The size of the liposomal vesicles may be determined by quasi-electric light scattering (QELS) as described in Bloomfield, Ann. Rev. Biophys. Bioeng., 10:421-450 (1981), incorporated herein by reference. Average liposome diameter may be reduced by sonication of formed liposomes. Intermittent sonication cycles may be alternated with QELS assessment to guide efficient liposome synthesis.

As used herein, the term “target cell” refers to a cell or tissue to which a composition of the invention is to be directed or targeted. For example, where it is desired to deliver a nucleic acid to a hepatocyte, the hepatocyte represents the target cell. In some embodiments, the compositions of the invention transfect the target cells on a discriminatory basis (i.e., do not transfect non-target cells). The compositions of the invention may also be prepared to preferentially target a variety of target cells, which include, but are not limited to, hepatocytes, epithelial cells, hematopoietic cells, epithelial cells, endothelial cells, lung cells, bone cells, stem cells, mesenchymal cells, neural cells (e.g., meninges, astrocytes, motor neurons, cells of the dorsal root ganglia and anterior horn motor neurons), photoreceptor cells (e.g., rods and cones), retinal pigmented epithelial cells, secretory cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes and tumor cells. In some embodiments, the target cells are deficient in a protein or enzyme of interest. In some embodiments the protein or enzyme of interest is encoded by a target gene, and the composition comprises an agent that increases expression of the target gene by stabilizing occupancy of a regulatory element of the target gene by a transcription factor.

The compositions of the invention may be prepared to preferentially distribute to target cells such as in the heart, lungs, kidneys, liver, and spleen. In some embodiments, the compositions of the invention distribute into the cells of the liver to facilitate the delivery and the subsequent expression of the synthetic RNA (e.g., modified mRNA) comprised therein by the cells of the liver (e.g., hepatocytes). The targeted hepatocytes may function as a biological “reservoir” or “depot” capable of producing a functional protein or enzyme (e.g., one that interferes with binding between a transcription factor of interest and a transcribed RNA). Accordingly, in one embodiment of the invention the liposomal transfer vehicle may target hepatocytes and/or preferentially distribute to the cells of the liver upon delivery. Following transfection of the target hepatocytes, the synthetic RNA (e.g., modified mRNA) loaded in the liposomal vehicle are translated and a functional protein product is produced. In other embodiments, cells other than hepatocytes (e.g., lung, spleen, heart, ocular, or cells of the central nervous system) can serve as a depot location for protein production.

The expressed or translated peptides, polypeptides, or proteins may also be characterized by the in vivo inclusion of native post-translational modifications which may often be absent in recombinantly-prepared proteins or enzymes, thereby further reducing the immunogenicity of the translated peptide, polypeptide, or protein.

The present disclosure also contemplates the discriminatory targeting of target cells and tissues by both passive and active targeting means. The phenomenon of passive targeting exploits the natural distributions patterns of a transfer vehicle in vivo without relying upon the use of additional excipients or means to enhance recognition of the transfer vehicle by target cells. For example, transfer vehicles which are subject to phagocytosis by the cells of the reticulo-endothelial system are likely to accumulate in the liver or spleen, and accordingly may provide means to passively direct the delivery of the compositions to such target cells.

The present disclosure contemplates active targeting, which involves the use of additional excipients, referred to herein as “targeting ligands” that may be bound (either covalently or non-covalently) to the transfer vehicle to encourage localization of such transfer vehicle at certain target cells or target tissues. For example, targeting may be mediated by the inclusion of one or more endogenous targeting ligands (e.g., apolipoprotein E) in or on the transfer vehicle to encourage distribution to the target cells or tissues. Recognition of the targeting ligand by the target tissues actively facilitates tissue distribution and cellular uptake of the transfer vehicle and/or its contents in the target cells and tissues (e.g., the inclusion of an apolipoprotein-E targeting ligand in or on the transfer vehicle encourages recognition and binding of the transfer vehicle to endogenous low density lipoprotein receptors expressed by hepatocytes). As provided herein, the composition can comprise a ligand capable of enhancing affinity of the composition to the target cell. Targeting ligands may be linked to the outer bilayer of the lipid particle during formulation or post-formulation. These methods are well known in the art. In addition, some lipid particle formulations may employ fusogenic polymers such as PEAA, hemaglutinin, other lipopeptides (see U.S. patent application Ser. Nos. 08/835,281, and 60/083,294, which are incorporated herein by reference) and other features useful for in vivo and/or intracellular delivery. In other some embodiments, the compositions of the present invention demonstrate improved transfection efficacies, and/or demonstrate enhanced selectivity towards target cells or tissues of interest. Contemplated therefore are compositions which comprise one or more ligands (e.g., peptides, aptamers, oligonucleotides, a vitamin or other molecules) that are capable of enhancing the affinity of the compositions and their nucleic acid contents for the target cells or tissues. Suitable ligands may optionally be bound or linked to the surface of the transfer vehicle. In some embodiments, the targeting ligand may span the surface of a transfer vehicle or be encapsulated within the transfer vehicle. Suitable ligands and are selected based upon their physical, chemical or biological properties (e.g., selective affinity and/or recognition of target cell surface markers or features.) Cell-specific target sites and their corresponding targeting ligand can vary widely. Suitable targeting ligands are selected such that the unique characteristics of a target cell are exploited, thus allowing the composition to discriminate between target and non-target cells. For example, compositions of the invention may include surface markers (e.g., apolipoprotein-B or apolipoprotein-E) that selectively enhance recognition of, or affinity to hepatocytes (e.g., by receptor-mediated recognition of and binding to such surface markers). Additionally, the use of galactose as a targeting ligand would be expected to direct the compositions of the present invention to parenchymal hepatocytes, or alternatively the use of mannose containing sugar residues as a targeting ligand would be expected to direct the compositions of the present invention to liver endothelial cells (e.g., mannose containing sugar residues that may bind preferentially to the asialoglycoprotein receptor present in hepatocytes). (See Hillery A M, et al. “Drug Delivery and Targeting: For Pharmacists and Pharmaceutical Scientists” (2002) Taylor & Francis, Inc.) The presentation of such targeting ligands that have been conjugated to moieties present in the transfer vehicle (e.g., a lipid nanoparticle) therefore facilitate recognition and uptake of the compositions of the present invention in target cells and tissues. Examples of suitable targeting ligands include one or more peptides, proteins, aptamers, small molecules, vitamins and oligonucleotides.

In some embodiments, the synthetic RNAs comprise at least one modification.

In some embodiments, at least 1%, at least 5%, at least 10%, at least 15%, at least 20%, at least 25%, at least 33%, at least 40%, at least 50%, at least 66%, at least 75%, at least 80%, at least 85%, at least 90%, or more of the nucleotides of the synthetic RNA comprise a modification. In some embodiments, the synthetic RNA comprises at least two, at least three, at least four, at least five, at least 10, at least 15, at least 20, at least 30, at least 35, at least 40, at least 45, at least 50, at least 55, at least 60, or more modifications, e.g., which can be the same modification throughout, or a combination of two, three, four, five, or more different modifications throughout.

In some embodiments, the composition comprises an agent which binds to the RNA in a manner that prevents the transcription factor from binding to the RNA. In some embodiments, the agent may bind to the RNA in the region that the RNA normally binds to the transcription factor. In some embodiments, the agent may bind to the RNA at a different site from where the RNA binds to the transcription factor, such that the agent may mask the site on the RNA that binds to the transcription factor or the agent may change the conformation of the RNA so that it no longer binds to the transcription factor.

In some embodiments, the agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the agent is an RNA interfering agent selected from the group consisting of a ribozyme, guide RNA, small interfering RNA (siRNA), short hairpin RNA or small hairpin RNA (shRNA), microRNA (miRNA), post-transcriptional gene silencing RNA (ptgsRNA), short interfering oligonucleotide, antisense oligonucleotide, aptamer, and CRISPR RNA.

In some embodiments, the composition modifies at least one nucleotide of a DNA sequence in a manner that prevents RNA transcribed from the at least one regulatory element from binding to the transcription factor. For example, at least one nucleotide of a DNA sequence that is transcribed to produce RNA can be made such that the modification alters the sequence of the transcribed RNA, such that the transcribed RNA has a reduced affinity for the transcription factor. Of course, it should be appreciated that at least one nucleotide sequence of the DNA sequence encoding the transcription factor could be modified in a way that reduces the affinity of the transcription factor for the transcribed RNA but does not interfere with binding of the transcription factor to the at least one regulatory element. In some embodiments, the modification of at least one nucleotide may decrease the amount of RNA transcribed from the regulatory element such that the amount of RNA becomes limiting for the process of binding of the RNA to the transcription factor. In some embodiments, the modification of at least one nucleotide may essentially stop transcription of the RNA from the regulatory element so that RNA is no longer available for binding to the transcription factor.

In some embodiments, modification of at least one nucleotide may interfere with or not allow binding of at least one of the factors involved in transcription at the regulatory element, such that the amount of RNA transcribed from the regulatory element is reduced and/or the sequence of the RNA is altered such that the RNA binds less tightly to the transcription factor, resulting in a decrease in gene expression of the target gene. In some embodiments, modification of at least one nucleotide may increase binding of at least one of the factors involved in transcription at the regulatory element, such that the amount of RNA transcribed from the regulatory element is increased and/or the sequence of the RNA is altered such that the RNA binds more tightly to the transcription factor, resulting in an increase in gene expression of the target gene.

Non-limiting examples of compositions which modulate binding between the RNA and the transcription factor by modifying at least one nucleotide of a DNA sequence (e.g., a DNA sequence of the at least one regulatory element or DNA sequencing encoding RNA transcribed from the at least one regulatory element) include the CRISPR/Cas system, zinc finger nucleases (ZFNs), transcription activator-like effector nucleases (TALENS), and engineered meganuclease re-engineered homing endonucleases. In some embodiments, the composition comprises a CRISPR\Cas system, which relies upon the nuclease activity of the Cas9 protein (Makarova et al. (2011) Nat. Rev. Microbiol. 9:467-77) coupled with a synthetic guide RNA (gRNA) to make specific modifications in a genome (Barrangou et al. (2007) Science 315:1709-12; Brouns et al. (2008) Science 321:960-64; U.S. Pat. No. 8,771,945). In some embodiments, the composition comprises zinc finger nucleases (ZFNs), which comprise artificial restriction enzymes comprising a zinc finger protein (ZFP) and a nuclease cleavage domain ZFNs can be engineered to bind to a sequence of choice and therefore can be used to target sequences within a genome. (See, for example, Porteus, and Baltimore (2003) Science 300: 763; Miller et al. (2007) Nat. Biotechnol. 25:778-785; Sander et al. (2011) Nature Methods 8:67-69; Wood et al. (2011) Science 333:307); U.S. Patent Publication No. 20080159996). In some embodiments, the composition comprises Transcription Activator-Like Effector Nucleases (TALENs), which comprise TAL effector DNA-binding domains fused to a DNA cleavage domain (Wood et al. (2011) Science 333:307; Boch et al. (2009) Science 326:1509-1512; Moscou and Bogdanove (2009) Science 326:1501; Christian et al. (2010) Genetics 186:757-761; Miller et al. (2011) Nat. Biotechnol. 29:143-148; Zhang et al. (2011) Nat. Biotechnol. 29:149-153; Reyon et al. (2012) Nat. Biotechnol. 30:460-465; U.S. Patent Publication No. 20110145940).In some embodiments, the composition comprises engineered meganuclease re-engineered homing endonucleases.

The genome editing systems described hereinabove use artificially engineered nucleases to cut and create specific double-stranded breaks at a desired location(s) in the genome, which are then repaired by cellular endogenous processes such as, homologous recombination (HR), homology directed repair (HDR) and non-homologous end-joining (NHEJ). NHEJ directly joins the DNA ends in a double-stranded break, while HDR utilizes a homologous sequence as a template for regenerating the missing DNA sequence at the break point. In some embodiments, the regulatory element is modified via specialized nucleic acid replication processes associated with homology-directed repair (HDR). In such embodiments, at least one nucleotide of a DNA sequence to be modified is identified, and then a nucleic acid construct comprising a repair template with the desired modified nucleotide can be used with one of the above editing systems/compositions to modify the at least one nucleotide via homology-directed repair. In some embodiments, integration into the genome occurs through non-homology dependent targeted integration (e.g. “end-capture”). In some embodiments, at least one nucleotide is modified in accordance with the above genomic editing systems/compositions to increase the amount of RNA transcribed from the regulatory element or alter the sequence of the RNA such that it binds more tightly to the transcription factor, for example, to increase transcription of the target gene.

The presently disclosed subject matter also provides methods for screening the modifications of at least one nucleotide of a DNA sequence of at least one regulatory element which decrease binding of the transcription factor to the RNA transcribed from the modified regulatory element. In some embodiments, the presently disclosed subject matter provides methods of screening for a mutation, such as a single nucleotide polymorphism (SNP), in a DNA sequence encoding the at least one regulatory element or the RNA that is transcribed from the at least one regulatory element, whereby the resulting RNA binds to and stabilizes transcription factor occupancy on at least one allele of the at least one regulatory element. In some embodiments, the screening methods comprise identifying the transcription factor that binds both a regulatory element and the RNA transcribed from the regulatory element, and then determining whether the RNA transcribed from the regulatory element from one or both alleles stabilizes occupancy of the transcription factor at the regulatory element. If only one allele stabilizes occupancy of the transcription factor, steps can be performed to compare the two alleles (e.g., sequence alignment, genotyping) to determine whether there are any polymorphisms in one allele relative to another. Further, editing or fixing the polymorphism can be performed to see if that normalizes transcription from the edited allele.

In some embodiments, the presently disclosed subject matter provides methods to identify a disease for which RNA transcribed from a regulatory element increases transcription to cause or exacerbate the disease. In some embodiments, the methods comprise selecting a SNP at one or both alleles of a regulatory element for a target gene that is known to be associated with a disease, such as by searching a disease database (e.g., Online Mendelian Inheritance in Man (OMIM)) or by searching a database of genetic variation such as dbSNP or SNPedia), and then assaying to determine if the SNP increases transcription of the one or both alleles of the regulatory element.

In some embodiments, the presently disclosed subject matter provides methods to identify a disease for which RNA transcribed from a regulatory element decreases transcription to cause or exacerbate the disease. In some embodiments, the methods comprise selecting a SNP at one or both alleles of a regulatory element for a target gene that is known to be associated with a disease, such as by searching a disease database (e.g., Online Mendelian Inheritance in Man (OMIM)) or by searching a database of genetic variation such as dbSNP or SNPedia), and then assaying to determine if the SNP decreases transcription of the one or both alleles of the regulatory element.

In some embodiments, the presently disclosed subject matter provides methods for identifying modifications in a regulatory element that can be introduced to interfere with binding of the RNA transcribed from the regulatory element to the transcription factor. For example, in an embodiment, the DNA sequence is modified in cells using a genomic editing tool such as the CRISPR/Cas system and cross-linking immunoprecipitation (CLIP) and/or CLIP-sequencing is performed. A modification in the DNA sequence of the regulatory element that results in less PCR product as compared to a control in which modification of the DNA sequence did not occur is indicative that the modification decreased binding of the transcription factor to the RNA transcribed from the modified regulatory element.

In some embodiments, the modified regulatory element modulates transcription of a gene involved in a disease or disorder and the modification that decreases binding of the transcription factor to the RNA transcribed from the modified regulatory element can be used to prevent or treat the disease or disorder.

In some embodiments, the agent can bind to more than one component of the presently disclosed methods, such as at least two of RNA, the transcription factor, and at least one regulatory element. In some embodiments, the agent binds to the transcription factor, regulatory element, and/or the RNA via covalent bonding. In some embodiments, the agent binds to the transcription factor, regulatory element, and/or the RNA via non-covalent interactions, such as van der Waals interactions, electrostatic interactions (salt bridges), dipolar interactions (hydrogen bonding), and entropic effects (hydrophobic interactions).

The presently disclosed subject matter contemplates the use of compositions and/or agents that inhibit expression or activity of the exosome complex or a subunit or component thereof. Such agents are useful for therapeutic purposes, e.g., treatment of a disease, condition, or disorder which exhibit aberrantly high expression and/or disease-associated expression. The exosome or exosome complex is an intracellular protein complex that is capable of degrading various types of RNA molecules. In some embodiments, the composition comprises an agent which prevents exosomal degradation of untethered RNA in proximity to the at least one regulatory element or the transcriptional machinery. The term “untethered”, as in untethered RNA, refers to a molecule that is not fastened, bound, or connected to another molecule. In the context of nascent RNA transcribed from at least one regulatory element, untethered RNA refers to RNA that has been transcribed from the at least one regulatory element and is released from RNA polymerase (e.g., RNA Pol II). In some embodiments, methods using an agent which inhibits or prevents exosomal degradation of the untethered RNA result in an increase in untethered RNA and increased binding of the transcription factor to the untethered RNA, thereby titrating the transcription factor away from binding to nascent RNA. As used herein, the term “nascent RNA” refers to RNA that is still being transcribed or has just been transcribed by RNA polymerase. In some embodiments, the nascent RNA transcribed from the regulatory element is bound to RNA polymerase.

In some embodiments, the agent inhibits the expression and/or activity of the exosome or a subunit thereof. Examples of exosome components that can be inhibited include exosome component 1, exosome component 2, exosome component 3 (ExoKD), exosome component 4, exosome component 5, exosome component 6, exosome component 7, exosome component 8, exosome component 9, exosome component 10, and DIS3. In some embodiments, the agent inhibits a component of the exosome via RNA interference. In some embodiments, the agent comprises an shRNA against Exosc3.

In some embodiments, the presently disclosed subject matter provides synthetic RNA hybrid nucleic acids comprising DNA and RNA, e.g., oligonucleotides comprising one or more deoxyribonucleotides at either end or both and/or internally.

In some embodiments, the presently disclosed subject matter provides oligonucleotides that promote RNase H-mediated degradation of the nascent RNA. RNase H degrades RNA in DNA/RNA hybrids. For example, antisense oligonucleotides comprising modifications at both ends (for biostability), e.g., 2′-O-methoxyethyl modifications at both ends, and a central gap of 10 unmodified nucleotides (deoxyribonucleotides) can be utilized to support RNase H activity (see, e.g., Wheeler et al., “Targeting nuclear RNA for in vivo correction of myotonic dystrophy,” Nature. 2012; 488(7409):111-115, which is incorporated herein by reference in its entirety). The deoxyribonucleic acids in the center of the oligonucleotide activate RNAse H and the end modifications stabilize the molecule. In some embodiments, one or more candidate oligonucleotides that are at least partly complementary to a nascent transcribed RNA of interest is tested to identify which of the candidate oligonucleotides effectively promote degradation of the nascent transcribed RNA.

In some embodiments, the presently disclosed subject matter provides a method of increasing transcription of a target gene by increasing the steady state levels of untethered RNA in proximity to the transcription factor, wherein the untethered RNA comprises an RNA which binds to the transcription factor at a site other than the DNA binding domain. In some embodiments, the untethered RNA binds to the transcription factor at a site that is in not in proximity to the DNA binding domain of the transcription factor.

In some embodiments, the presently disclosed subject matter provides methods for identifying agents that can outcompete the nascent RNA being transcribed. In some embodiments, the methods comprise assessing binding between RNA transcribed from at least one regulatory element and a transcription factor which binds to the RNA and to the at least one regulatory element in the presence or absence of a test agent, wherein decreased binding of the transcription factor to the RNA transcribed from the at least one regulatory element in the presence of the test agent as compared to the absence of the test agent indicates that the test agent is capable of outcompeting the nascent RNA being transcribed. Further competition experiments can be performed to determine whether the test agent is actually outcompeting the nascent RNA by binding to the transcription factor or whether the test agent is interfering with binding of the nascent RNA and the transcription factor without binding the transcription factor itself. Such an agent may further be used to destabilize expression of the target gene by being placed in proximity to the transcription factor to compete with the nascent RNA for binding to the transcription factor. In some embodiments, the agent is an RNA molecule. In some embodiments, this method is performed in vivo by growing cells (e.g., ESCs) with and without the agent and performing cross-linking immunoprecipitation (CLIP) and/or CLIP-sequencing. A decrease in PCR product in the presence of the agent as compared to the control without agent is indicative that the agent outcompeted the nascent RNA for binding to the transcription factor.

In some embodiments, the target gene comprises a gene for which increased or aberrant transcription is associated with a disease, condition, or disorder. In some embodiments, the disease, condition, or disorder is selected from the group consisting of cancer; genetic disorders; liver disorders, such as liver fibrosis and liver cancer; neurodegenerative disorders, such as Alzheimer's disease, amyotrophic lateral sclerosis (ALS), etc.; and autoimmune diseases, such as inflammatory bowel disease and rheumatoid arthritis. Cancer as used herein includes, but is not limited to, head cancer, neck cancer, head and neck cancer, lung cancer, breast cancer, prostate cancer, colorectal cancer, esophageal cancer, stomach cancer, leukemia/lymphoma, uterine cancer, skin cancer, endocrine cancer, urinary cancer, pancreatic cancer, gastrointestinal cancer, ovarian cancer, cervical cancer, and adenomas. In some embodiments, the cancer comprises a cancer for which an oncogene comprising a SNP is associated with increased expression (e.g., transcription) of the oncogene. In some embodiments, the cancer comprises a BRCA1-associated cancer. In some embodiments, the cancer comprises breast cancer comprising at least one SNP in at least one allele of the BRCA1 gene. In some embodiments, the cancer comprises ovarian cancer comprising at least one SNP in at least one allele of the BRCA1 gene.

Accordingly, in some embodiments, the presently disclosed subject matter also provides a method for treating a disease, condition, or disorder, the method comprising administering to a subject in need of treatment thereof, an agent that modulates binding between a ribonucleic acid (RNA) transcribed from at least one regulatory element of a target gene and a transcription factor which binds to both the RNA and the at least one regulatory element, wherein modulating binding between the RNA and the transcription factor modulates expression of the target gene. In some embodiments, the agent decreases binding between the RNA and the transcription factor to decrease expression of the target gene. In some embodiments, the agent increases binding between the RNA and the transcription factor to increase expression of the target gene. In some embodiments, the method includes identifying a subject having a disease, condition, or disorder exhibiting increased or aberrant transcription of a target gene driven by stabilization of transcription factor occupancy of at least one regulatory element due to binding of RNA transcribed from the at least one regulatory element to the transcription factor. In some embodiments, the method includes identifying a subject having a disease, condition, or disorder exhibiting decreased transcription of a target gene driven by destabilization of transcription factor occupancy of at least one regulatory element due to weakened or diminished binding of RNA transcribed from at least one regulatory element to the transcription factor. In some embodiments, the method includes identifying such diseases, conditions, or disorders. In some embodiments, the disease, condition, or disorder is selected from the group consisting of cancer, liver disorders, neurodegenerative disorders, metabolic disorders, and autoimmune diseases. As used herein, the term “treating” can include reversing, alleviating, inhibiting the progression of, preventing or reducing the likelihood of the disease, disorder, or condition to which such term applies, or one or more symptoms or manifestations of such disease, disorder or condition.

In some embodiments aberrantly increased expression of the target gene or aberrantly increased activity of a gene product of the target gene causes or contributes to the disease, and the method comprises inhibiting expression of the target gene by interfering with binding of the TF to RNA transcribed from a regulatory element of the target gene, e.g., by administering an agent that decreases such binding to a subject in need of treatment for the disease. In some embodiments aberrantly reduced expression of the target gene or aberrantly reduced activity of a gene product of the target gene causes or contributes to the disease, and the method comprises increasing expression of the target gene by increasing binding of the TF to RNA transcribed from a regulatory element of the target gene, e.g., by administering an agent that increases such binding to a subject in need of treatment for the disease.

Some embodiments involve contacting an agent with a cell that exhibits aberrantly increased or decreased expression of a target gene or aberrantly increased or decreased activity of a gene product of the target gene. In some embodiments, the method decreases the expression in a cell where the expression or activity is aberrantly increased or excessive. In some embodiments, the method increasing the expression in a cell where the expression is aberrantly decreased or insufficient. The cell may be in a subject suffering from a disorder associated with aberrantly increased or excessive expression/activity or aberrantly decreased or insufficient expression/activity.

In some embodiments, the target gene comprises an oncogene. Non-limiting examples of oncogenes include abl, Af4/hrx, akt-2, alk, alk/npm, aml1, amll/mtg8, axl, bcl-2, bcl-3, bcl-6, bcr/abl, c-myc, dbl, dek/can, E2A/pbxl, egfr, enl/hrx, erg/TLS, erbB, erbB-2, ets-1, ews/fli-1, fms, fos, fps, gli, gsp, HER2/neu, hox11, hst, IL-3, int-2, jun, kit, KS3, K-sam, Lbc, lck, lmo1, lmo2, L-myc, lyl-1, lyt-10, lyt-10/C alpha1, mas, mdm-2, mll, mos, mtg8/amll, myb, MYH11/CBFB, neu, N-myc, ost, pax-5, pbxl/E2A, pim-1, PRAD-1, raf, RAR/PML, rasH, rasK, rasN, rel/nrg, ret, rhom1, rhom2, ros, ski, sis, set/can, src, tall, tal2, tan-1, Tiam1, TSC2, and trk.

In some embodiments the target gene encodes a protein. In some embodiments the protein is a transcription factor, a transcriptional co-activator or co-repressor, an enzyme (e.g., a kinase, phosphatase, acetylase, deacetylase, methylase, demethylase, protease), a chaperone, a co-chaperone, a heat shock protein, a receptor, a secreted protein, a transmembrane protein, a peripheral membrane protein, a soluble protein, a nuclear protein, a mitochondrial protein, a lysosomal protein, a growth factor, a cytokine (e.g., an interferon, an interleukin, a chemokine, a tumor necrosis factor), a hormone, an extracellular matrix protein, a motor protein, a cell adhesion molecule, a major or minor histocompatibility (MHC) protein, a transporter, a channel, an immunoglobulin (Ig) superfamily (IgSF) member, an integrin, a cadherin superfamily member, a selectin, a clotting factor, a complement factor, a pluripotency protein, or a tumor suppressor protein. In some embodiments the target gene encodes a protein that is a component of a multiprotein complex such as the ribosome, spliceosome, proteasome, or RNA-induced silencing complex. In some embodiments the target gene encodes a microRNA precursor or an RNA that is a component of a ribonucleoprotein complex.

In some embodiments, the target gene comprises at least one mutation in the at least one regulatory element, wherein the at least one mutation results in the transcription factor binding to RNA transcribed from the at least one regulatory element in a manner that stabilizes occupancy of the transcription factor at the at least one regulatory element, thereby increasing expression of the target gene. In some embodiments, the target gene comprises at least one mutation in the at least one regulatory element, wherein the at least one mutation results in diminished or weakened binding by the transcription factor to RNA transcribed from the at least one regulatory element, thereby decreasing expression of the target gene. In some embodiments, the at least one mutation comprises a single nucleotide polymorphism (SNP). Examples of SNPs can be found in the NCBI database of single nucleotide polymorphisms (dbSNP), SNPedia, and the like. Non-limiting examples of diseases associated with SNPs that are linked to regulatory elements include cancer, such as colorectal and gastric cancer (e.g., BRCA1 associated cancers); diabetes, such as type 2 diabetes; cardiovascular associated disease, such as coronary artery disease; neurodegenerative disorders, such as Parkinson's disease; and autoimmune disorders, such as inflammatory bowel disease.

In some embodiments, the presently disclosed subject matter provides a method for destabilizing the occupancy of the transcription factor at the at least one regulatory element wherein the regulatory element comprises at least one mutation that increases expression of the target gene, the method comprising using an agent that targets the mutated RNA that results from transcription of the regulatory element comprising at least one mutation. In this case, the agent can inhibit the mutated RNA, thereby inhibiting or blocking gene expression by destabilizing the occupancy of the transcription factor. As described hereinabove, a disease or disorder may be caused by increased transcription caused by at least one mutation at a regulatory element. Therefore, in some embodiments, an agent may be used to treat a disease caused by at least one mutation at a regulatory element.

In some embodiments, the presently disclosed subject matter provides a method of identifying a candidate agent that interferes with binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein decreased binding of the transcription factor to the RNA transcribed from the at least one regulatory element in the presence of the test agent as compared to the absence of the test agent indicates that the test agent is a candidate agent that interferes with binding between the RNA and the transcription factor. In some embodiments, the presently disclosed subject matter provides a method of identifying a candidate agent that promotes binding between RNA transcribed from at least one regulatory element and a transcription factor which binds to the RNA and to the at least one regulatory element, the method comprising assessing binding between RNA transcribed from at least one regulatory element and a transcription factor which binds to the RNA and to the at least one regulatory element in the presence and absence of a test agent, wherein increased binding of the transcription factor to the RNA transcribed from the at least one regulatory element in the presence of the test agent as compared to the absence of the test agent indicates that the test agent is a candidate agent that promotes binding between the RNA and the transcription factor. In some embodiments, binding is performed in a cell. In some embodiments, the method comprises performing cross-linking immunoprecipitation (CLIP) with the RNA and the transcription factor. In some embodiments, binding in the cell is assessed using RIP-eq. In some embodiments, binding in the cell is assessed using RIP-Chip.

Those skilled in the art will appreciate that a variety of cell-free binding assays can be used to identify a candidate agent. In some embodiments the method is performed in a cell-free composition comprising a TF that binds to a regulatory element from which RNA is transcribed, RNA whose sequence comprises at least a portion of the sequence of RNA transcribed from the regulatory element, and a candidate agent. The RNA may be incubated with the TF in the absence or presence of the candidate agent. Then, the TF or RNA is isolated from the composition (e.g., using immunoprecipitation). The amount of RNA bound to the TF in the presence of the candidate agent as compared with the amount of RNA bound to the TF in the absence of the candidate agent is determined. In some embodiments the RNA comprises or is conjugated to a detectable label (e.g., a fluorophore, radioactive atom, etc.), and RNA bound to the TF may be detected by detecting the detectable label. In some embodiments the RNA may be synthetically produced using chemical synthesis or an in vitro transcription system. In some embodiments the method comprises performing a high throughput screen to identify an agent that modulates binding between RNA transcribed from at least one regulatory element and a transcription factor which binds to the RNA and to the at least one regulatory element. In some embodiments the test agent is a small molecule, nucleic acid, peptide, etc.

In some embodiments, the methods further comprise identifying a transcription factor that binds to RNA transcribed from at least one regulatory element and to the at least one regulatory element. For example, the transcription factor can be identified by isolating the transcription factor-RNA complex formed from binding between RNA transcribed from at least one regulatory element and the transcription factor which binds to the RNA and to the at least one regulatory element and using a protein identification method such as mass spectrometry or protein sequencing to identify the transcription factor. In some embodiments, the methods further comprise identifying an RNA binding domain of the transcription factor. For example, once the transcription factor has been identified, its amino acid sequence can be compared to known sequences in databases to identify RNA recognition motifs, etc. In some embodiments, the methods further comprise identifying a consensus motif in the RNA transcribed from the at least one regulatory sequence for the RNA binding domain of the transcription factor.

In some embodiments, assessing binding comprises contacting a complex or mixture comprising the transcription factor, the at least one regulatory element, and the RNA transcribed from the at least one regulatory element with the test agent. In some embodiments, the methods further comprise assessing whether the test agent is capable of binding to the transcription factor at a site other than a DNA binding domain of the transcription factor.

In some embodiments, the test agent is selected from the group consisting of small molecules, saccharides, peptides, proteins, peptidomimetics, nucleic acids, an extract made from biological materials selected from the group consisting of bacteria, plants, fungi, animal cells, and animal tissues, and any combination thereof.

In some embodiments, the test agent comprises a decoy RNA as described herein.

In some embodiments, binding is performed in a cell. In some embodiments, the method comprises performing cross-linking immunoprecipitation (CLIP) with the RNA and the transcription factor. In some embodiments, the method comprises performing an EMSA assay. In some embodiments, the method comprises performing an immunoprecipitation assay.

In some aspects, the presently disclosed subject matter contemplates diagnostic and/or prognostic applications, for example, methods of diagnosing diseases, conditions, or disorders associated with aberrant transcription (e.g., increased or decreased) by detecting at least one modification in a DNA sequence encoding at least one regulatory element or the RNA transcribed from the at least one regulatory element, e.g., wherein the alteration of the DNA results in aberrant transcription (e.g., increased transcription, e.g., by stabilizing occupancy of a transcription factor which binds both the RNA and the at least one regulatory element, or decreased transcription, e.g., by destabilizing occupancy of a transcription factor which binds to both the RNA and the at least one regulatory element).

In some embodiments, it is desirable to increase expression of a target gene (e.g., haploinsufficiency disorders) or to decrease expression of a target gene (e.g., disorders associated with gene amplification). The disease or condition is not limited and may be any disease or condition disclosed herein. In some embodiments, modulating expression treats, prevents or reduces the likelihood of a disease or condition associated with a haploinsufficiency. In some embodiments, the disease or condition associated with a haploinsufficiency is a cancer, 1921.1 deletion syndrome, 5q-syndrome in myelodysplastic syndrome (MDS), 22q11.2 deletion syndrome, CHARGE syndrome, Cleidocranial dysostosis, Ehlers-Danlos syndrome, Frontotemporal dementia caused by mutations in progranulin, GLUT1 deficiency (DeVivo syndrome), Haploinsufficiency of A20, Holoprosencephaly caused by haploinsufficiency in the Sonic Hedgehog gene, Holt-Oram syndrome, Marfan syndrome, Phelan-McDermid syndrome, Polydactyly, or Dravet Syndrome. In some embodiments, modulating expression of a gene treats, prevents or reduces the likelihood of a disease or condition associated with gene duplication. In some embodiments, the disease or condition associated with gene duplication is a cancer with an oncogene duplication, Charcot-Marie-Tooth disease type I, or MECP2 duplication syndrome. In some embodiments, modulating of expression of a gene treats, prevents or reduces the likelihood of a disease or condition associated with an eRNA variant (e.g., an eRNA comprising an SNP). In some embodiments, modulating expression of a gene treats, prevents or reduces the likelihood of a disease or condition associated with aberrant transcription (e.g., cancer).

### Pharmaceutical Compositions and Administration

In another aspect, the present disclosure provides a pharmaceutical composition including an agent which interferes with binding between the RNA and the transcription factor alone or in combination with one or more additional therapeutic agents in admixture with a pharmaceutically acceptable excipient. One of skill in the art will recognize that the pharmaceutical compositions include the pharmaceutically acceptable salts of the compounds described above.

In therapeutic and/or diagnostic applications, the agent which interferes with binding between the RNA and the transcription factor for use within the methods of the presently disclosed subject matter can be formulated for a variety of modes of administration, including oral, systemic, and topical or localized administration. Techniques and formulations generally may be found in Remington: The Science and Practice of Pharmacy (20^{th} ed.) Lippincott, Williams & Wilkins (2000). The agents may be delivered, for example, in a timed- or sustained-low release form as is known to those skilled in the art. Techniques for formulation and administration may be found in Remington: The Science and Practice of Pharmacy (20^{th} ed.) Lippincott, Williams & Wilkins (2000).

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

An agent which interferes with binding between the RNA and the transcription factor may be formulated into liquid or solid dosage forms and administered systemically or locally. Suitable routes may include rectal, intestinal, or intraperitoneal delivery. Other suitable routes may include various forms of parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

The compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, camsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). Pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Additional therapeutic agents may be administered together with the agent which interferes with binding between the RNA and the transcription factor within the methods of the presently disclosed subject matter. These additional agents may be administered separately, as part of a multiple dosage regimen, from the inhibitor-containing composition. Alternatively, these agents may be part of a single dosage form, mixed together with the inhibitor in a single composition.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the composition of the encapsulating matrix, the target tissue, and the like.

### Kits

The presently disclosed subject matter also relates to kits for practicing the methods of the presently disclosed subject matter. In general, a presently disclosed kit contains some or all of the components, reagents, supplies, and the like to practice a method according to the presently disclosed subject matter. In some embodiments, the term “kit” refers to any intended article of manufacture (e.g., a package or a container) comprising a composition or agent that modulates binding between RNA transcribed from at least one regulatory element and a transcription factor that binds to both the RNA and the at least one regulatory element, and a set of particular instructions for practicing the methods of the presently disclosed subject matter. The kit can be packaged in a divided or undivided container, such as a carton, bottle, ampule, tube, etc. The presently disclosed compositions can be packaged in dried, lyophilized, or liquid form. Additional components provided can include vehicles for reconstitution of dried components.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### Exemplification

The following exemplification is included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Overview

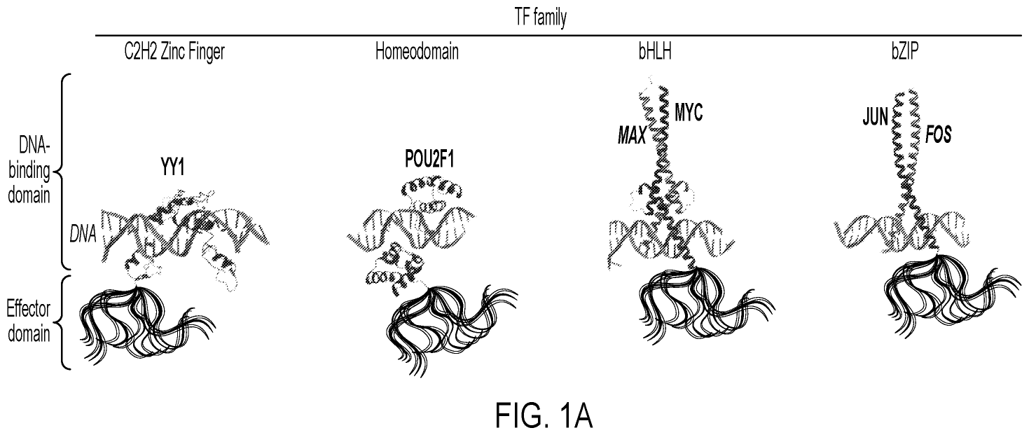

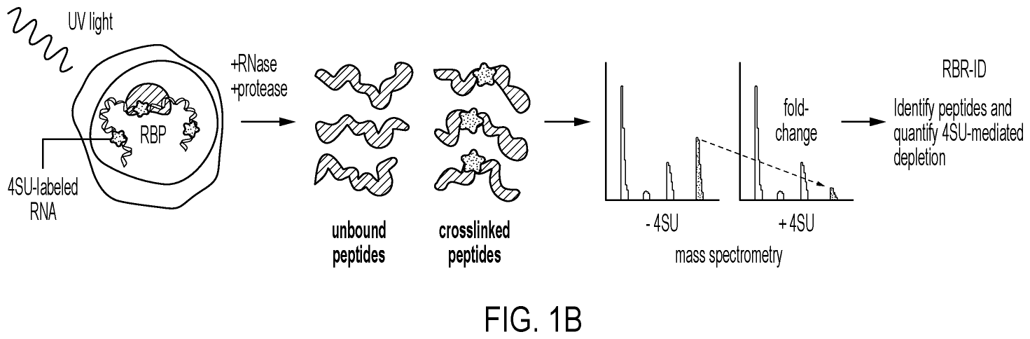

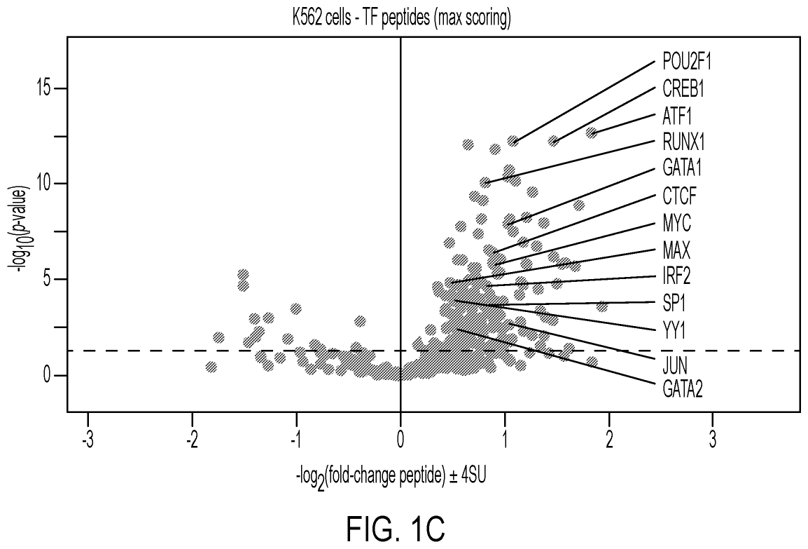

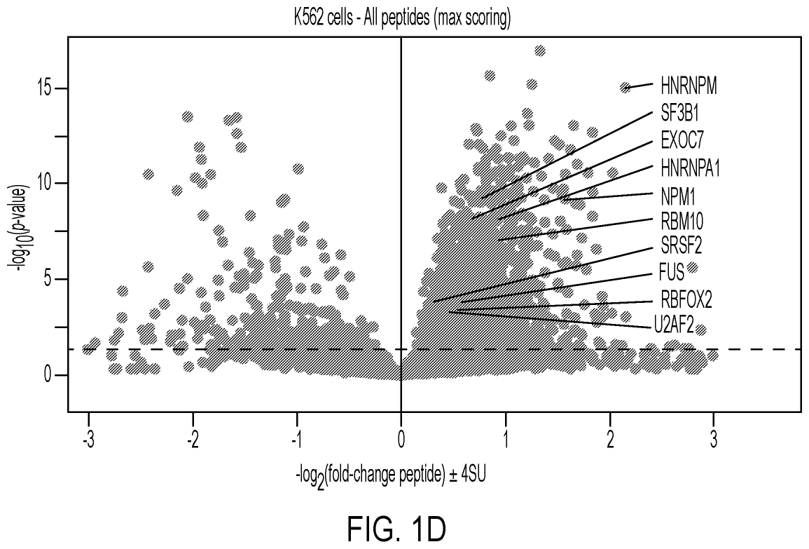

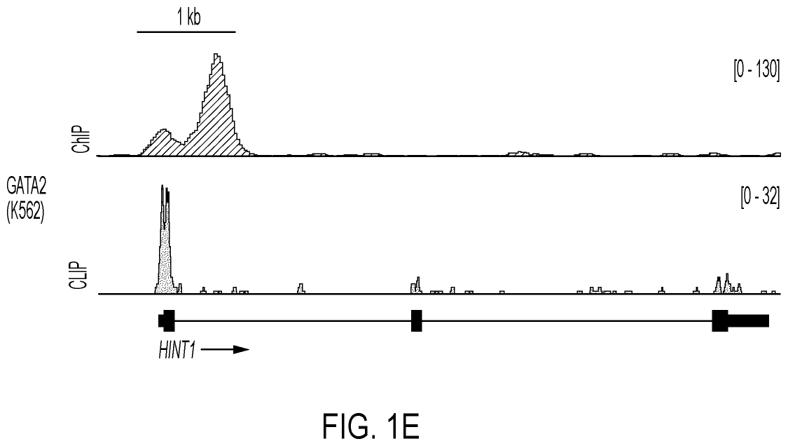

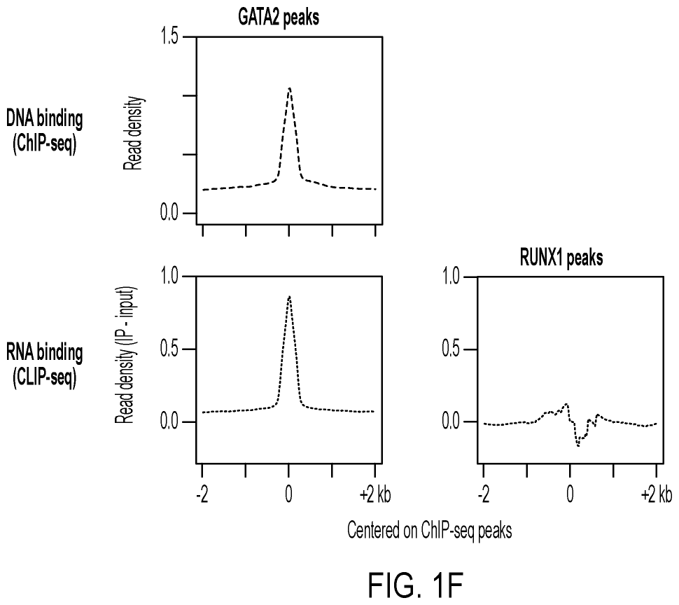

Transcription factors (TFs), which are encoded by ˜1,600 genes in the human genome, comprise the single largest protein family in mammals. Each cell type expresses approximately 150-400 TFs, which together control the gene expression program of the cell¹⁻⁵. TFs typically contain DNA-binding domains that recognize specific sequences and multiple TFs collectively bind to enhancers and promoter-proximal regions of genes^{6,7}. The DNA-binding domains form stable structures whose conserved features are reliably detected by homology and are therefore used to classify TFs (e.g. C2H2 zinc finger, homeodomain, bHLH, bZIP) (FIG. 1A)^{1,2}. TFs also contain effector domains that exhibit less sequence conservation and sample many transient structures that enable multivalent protein interactions⁸⁻¹⁰. These effector domains recruit coactivator or corepressor proteins, which contribute to gene regulation through mechanisms that include mobilizing nucleosomes, modifying chromatin-associated proteins, influencing 30 genome architecture, recruiting transcription apparatus and controlling aspects of transcription initiation and elongation^{11,12}. This canonical view of TFs that function with two domains, one binding DNA and the other protein, has been foundational for models of gene regulation^{13,14}.

RNA molecules are produced at loci where TFs are bound, but their roles in gene regulation are not well-understood^{15,16}. A few TFs and cofactors have been reported to bind RNA¹⁷⁻²⁸, but TFs do not harbor domains characteristic of well-studied RNA binding proteins²⁹. We wondered whether TFs might have evolved to interact with RNA molecules that are pervasively present at gene regulatory regions but harbor a heretofore unrecognized RNA-binding domain. Here we present evidence that a broad spectrum of TFs do bind RNA molecules, that TFs accomplish this with a domain analogous to the RNA-binding arginine-rich motif of the HIV Tat transactivator, and that this domain promotes TF occupancy at regulatory loci. These domains are a conserved feature important for vertebrate development, and they are disrupted in cancer and developmental disorders.

### Transcription Factor Binding to RNA in Cells

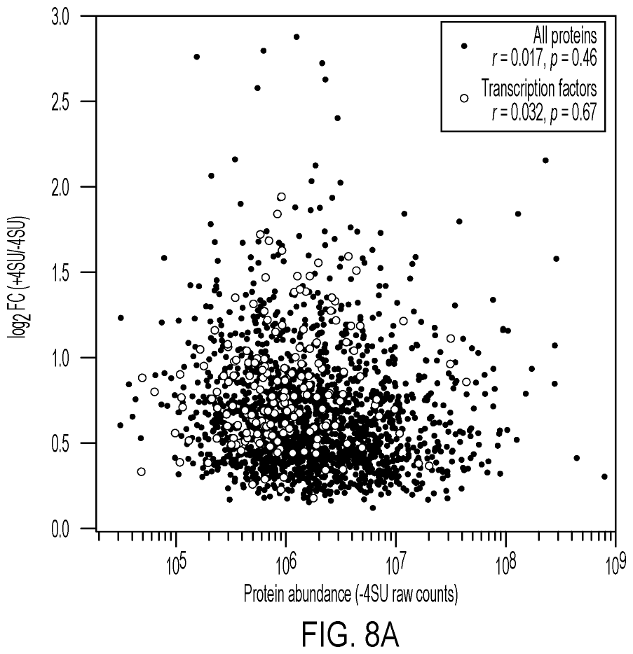

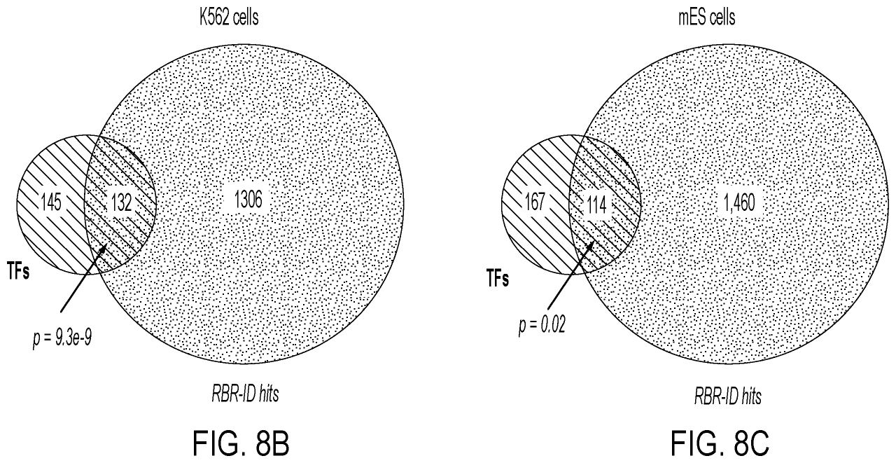

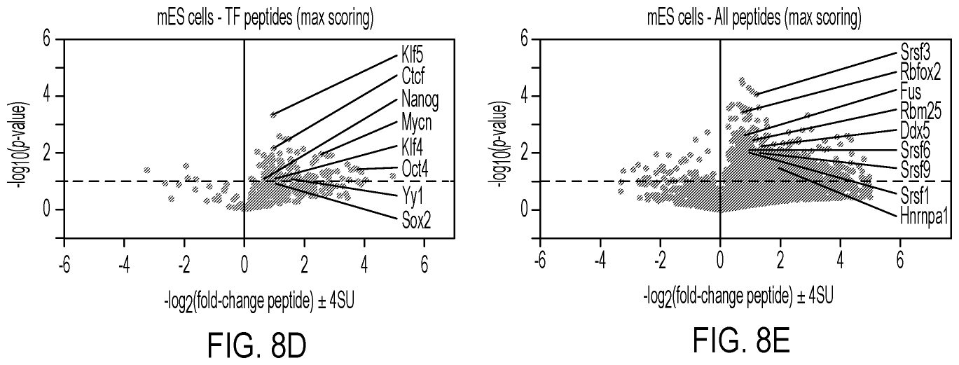

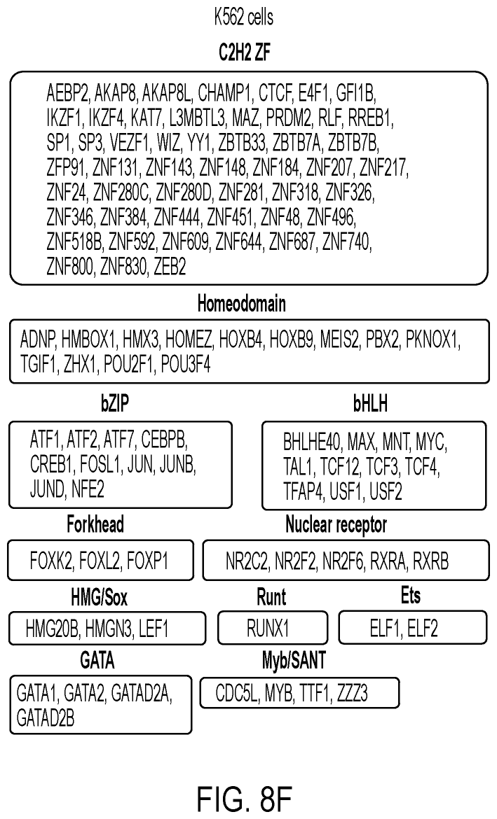

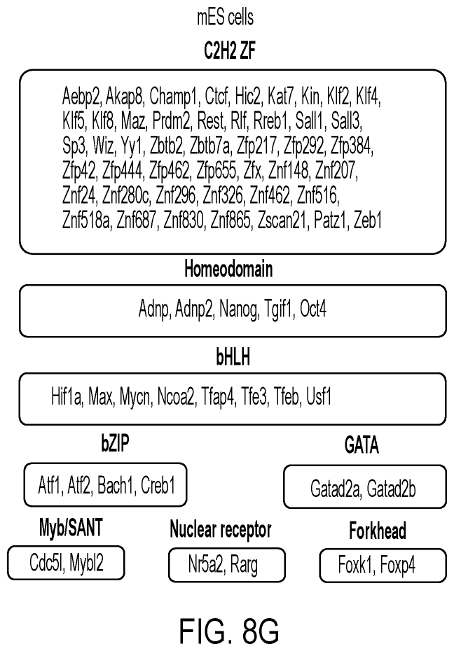

Using human K562 cells, we performed a high throughput RNA-protein crosslinking assay (RNA-binding region identification—RBR-ID), which uses UV crosslinking and mass spectrometry to detect angstrom-scale crosslinks, typically thought to reflect direct interactions³⁰, between protein and RNA molecules in cells³¹ (FIG. 1). The results included the expected distribution of peptides from known RNA-binding proteins (RBPs) and revealed that a broad distribution of TFs had peptides crosslinked to RNA in this assay independent of their cellular abundance (FIGS. 1C, 1D, and 8A). Nearly half (48%) of TFs identified in the RBR-ID dataset showed evidence of RNA binding in K562 cells (FIG. 8B) when the analysis was conducted using thresholds that retain RBPs verified by independent methods³¹. These results prompted a re-examination of previously published RBR-ID data for murine embryonic stem cells (ESCs)³¹ which confirmed that a substantial fraction of TFs (41%) in those cells also bind RNA (FIGS. 8C-E). A meta-analysis of data from multiple studies using proteomics to identify RNA-binding proteins, including data collected in this study, provides an extensive list of RNA-binding TFs (Table 1).

Specific TFs are notable for their roles in control of cell identity and have been subjected to more extensive study than others. Many well-studied TFs that contribute to the control of cell identity were observed among the TFs that showed evidence of RNA binding. In K562 hematopoietic cells, these included GATA1, GATA2, and RUNX1, which play major roles in regulation of hematopoietic cell genes³², as well as MYC and MAX, oncogenic regulators of these tumor cells33 (FIG. 1C). In the ESCs, these included the master pluripotency regulators Oct4, Klf4, and Nanog, as well as the MYC family member that is key to proliferation of these cells, Mycn34 (FIG. 8D). The RNA-binding TFs also included those involved in other important cellular processes, including regulation of chromatin structure (CTCF, YY1) and response to signaling (CREB1, IRF2, ATF1) (FIG. 1C). It was notable that RNA binding was a property of TFs that span many TF families (FIGS. 8F and 8G). These results suggest that RNA binding is a property shared by TFs that participate in diverse cellular processes and that possess diverse DNA-binding domains.

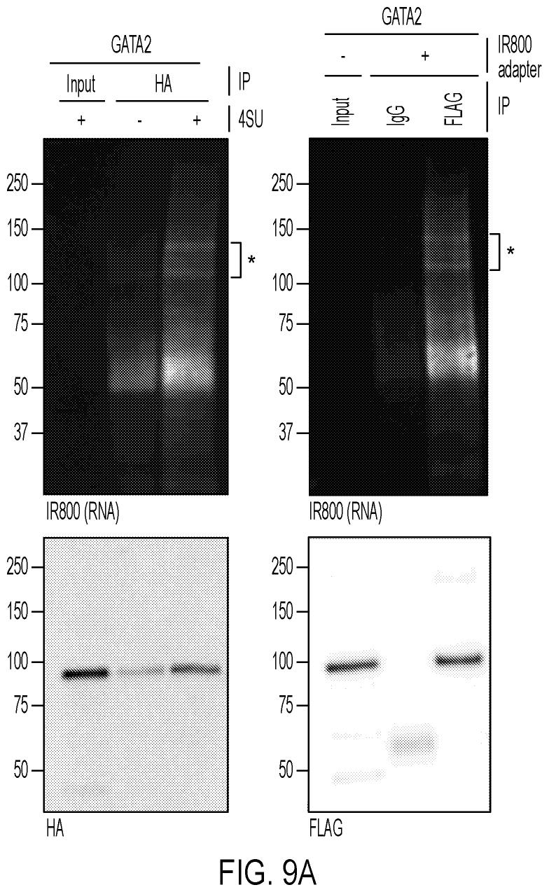

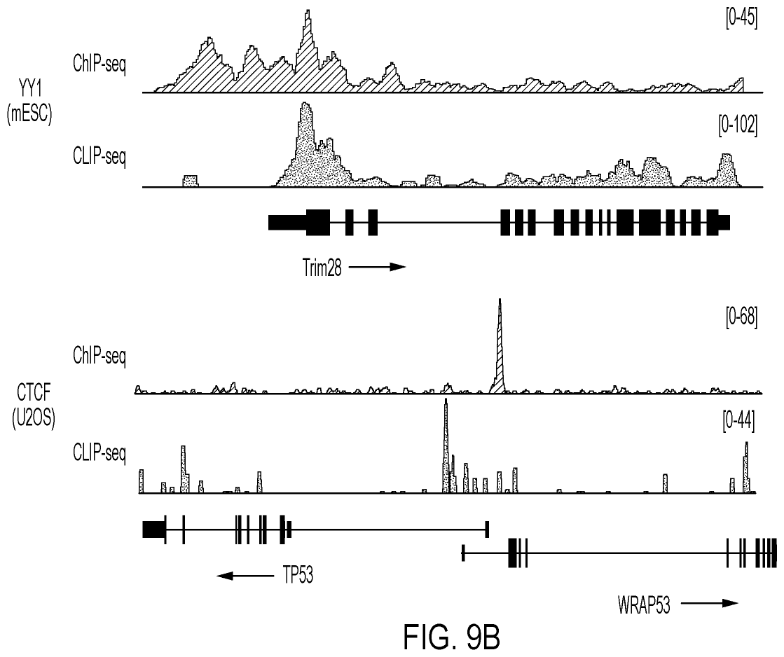

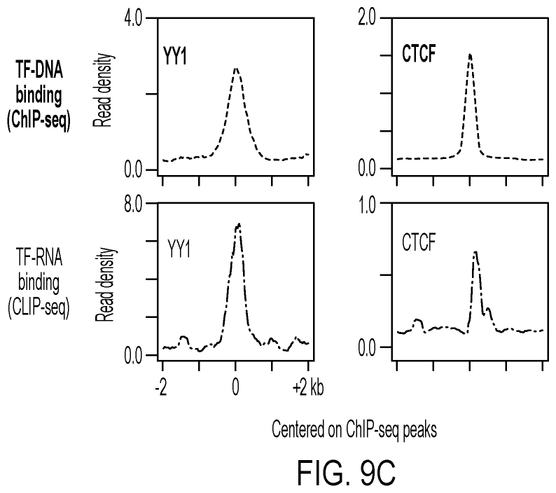

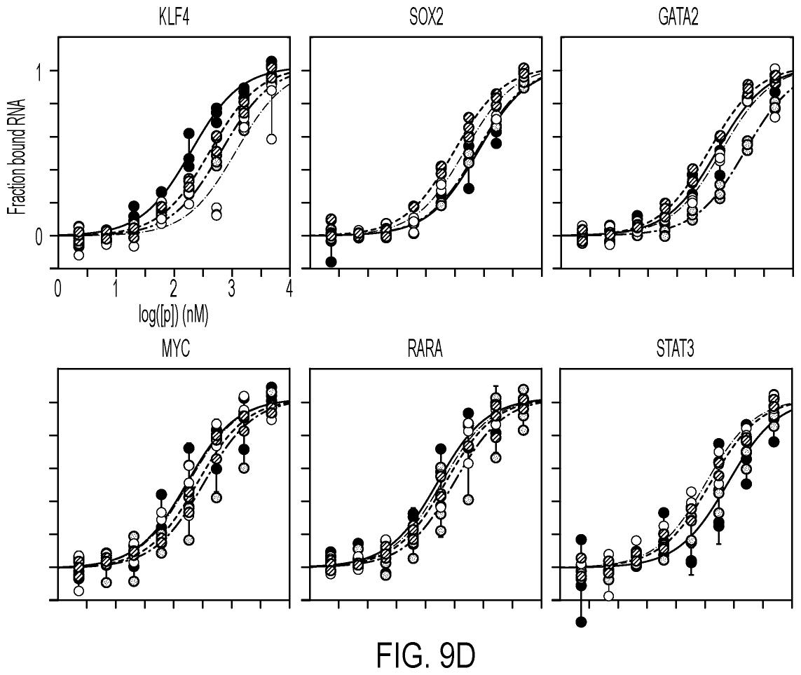

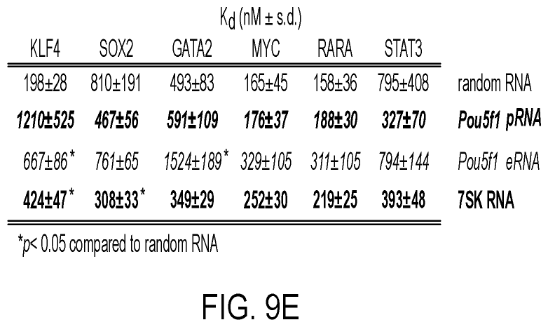

We next sought to identify the RNAs that interact with specific TFs. We conducted CLIP for the TF GATA2, a major regulator of hematopoietic genes in K562 cells that showed evidence of RNA binding in our RBR-ID data (FIG. 1C). Immunoprecipitation of HA- and FLAG-tagged GATA2 in K562 cells subjected to UV cross-linking showed that GATA2 interacts with RNA in cells in a 4SU-dependent manner (FIG. 9A). Interacting RNAs were then sequenced and cross-linked sites were identified with nucleotide resolution (STAR Methods). A diversity of RNA species were bound by GATA2, including many enhancer- and promoter derived RNAs. We reasoned that GATA2 may interact with RNAs transcribed in proximity to regions where GATA2 binds chromatin to regulate genes. Indeed, as illustrated for a specific locus, GATA2 binds chromatin at the HINT1 gene measured by ChIP-seq, and GATA2 interacts with RNA transcribed from the HINT1 gene measured by CLIP-seq (FIG. 1E). A metagene analysis revealed that GATA2 CLIP signal was enriched at GATA2 ChIP-seq peaks (FIG. 1F). Enrichment of GATA2 CLIP signal was not evident at ChIP-seq peaks of RUNX1, another major regulator of hematopoietic genes (FIG. 1F). These results prompted a re-examination of previously published CLIP/ChIP data for RBR-ID+ YY1 and CTCF21,35,36, which also showed that these TFs interact with RNAs transcribed from loci near their chromatin binding sites (FIGS. 9B and 9C). These results suggest that TFs bind to RNAs produced in the vicinity of their DNA-binding sites.

### Transcription Factor Binding to RNA In Vitro

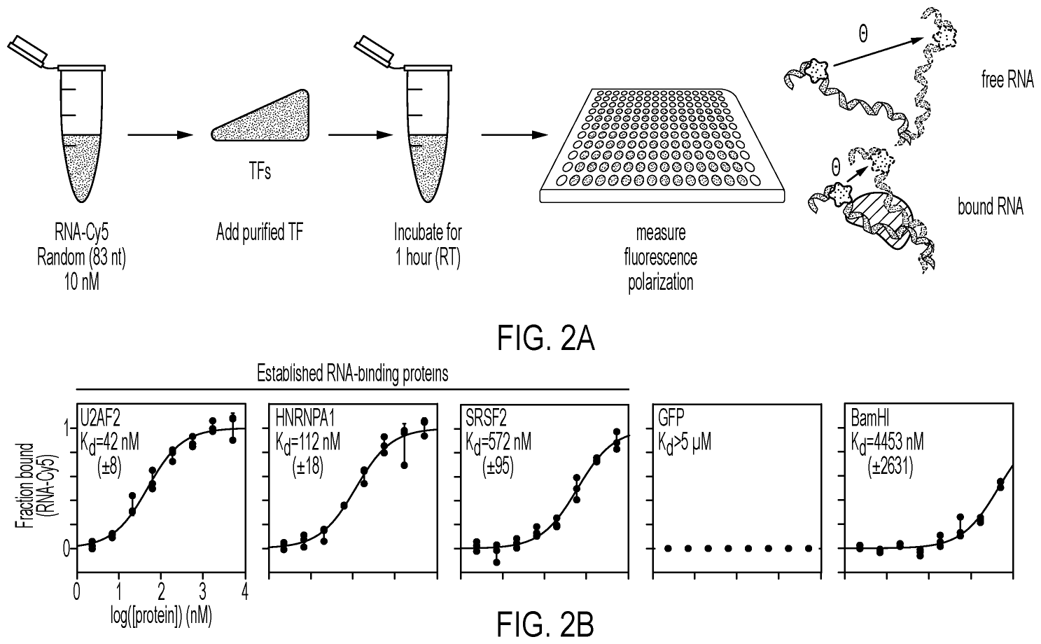

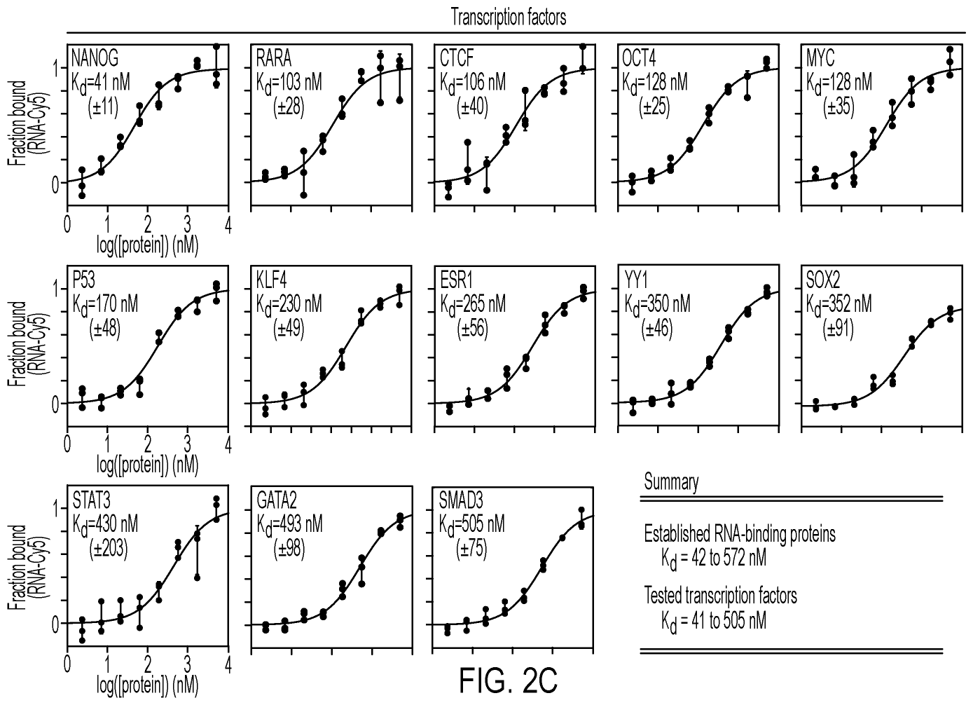

To corroborate evidence that TFs can bind RNA molecules in cells, we sought to confirm that purified TFs bind RNA molecules in vitro using a fluorescence polarization assay (FIG. 2A, STAR Methods). The assay was validated with multiple control proteins with an RNA of random sequence, including three well-studied RNA-binding proteins (U2AF2, HNRNPA1, and SRSF2) and proteins that were not expected to have substantial affinity for RNA (GFP and the DNA-binding restriction enzyme BamHI). The RBPs bound RNA with nanomolar affinities, consistent with previous studies³⁷⁻⁴⁰, whereas GFP and BamHI showed little affinity for RNA (Kd>4 μM) (FIG. 2B). We then selected 13 TFs that showed evidence of crosslinking to RNA in cells, are well-studied for their diverse cellular functions and are members of different TF families, purified them from human cells and measured their RNA-binding affinities. These TFs exhibited a range of binding affinities for the RNA, ranging from 41 to 505 nM, which is remarkably similar to the range of affinities measured for known RBPs (42 to 572 nM) (FIG. 2C). Thus, a diverse set of TFs can bind RNA with affinities similar to proteins with known physiological roles in RNA processing. The thousands of enhancers and promoter-proximal regions where TFs bind have diverse sequences, and thus RNA molecules produced from these sites differ in sequence, so we investigated whether TFs bind diverse RNA sequences. Six TFs were investigated, and the results indicate that these TFs do bind various RNA sequences with similar affinities (FIGS. 9D and 9E).

### An Arginine-Rich Domain in Transcription Factors

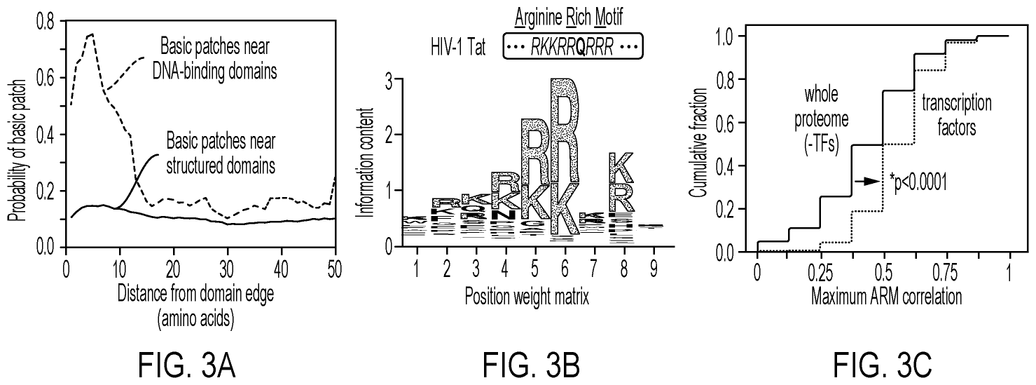

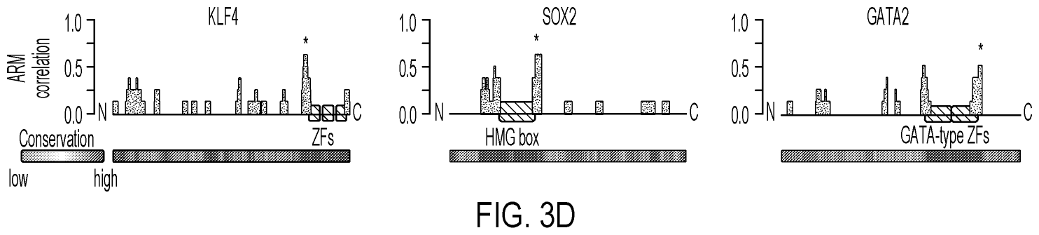

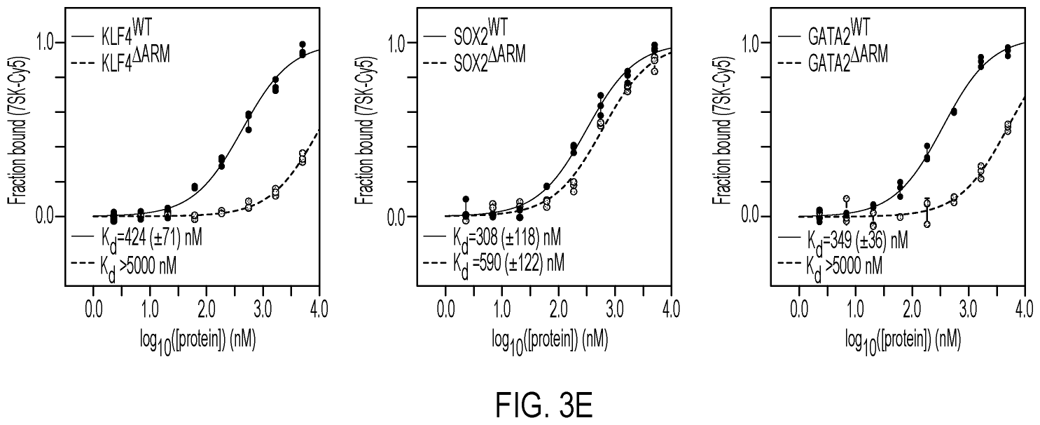

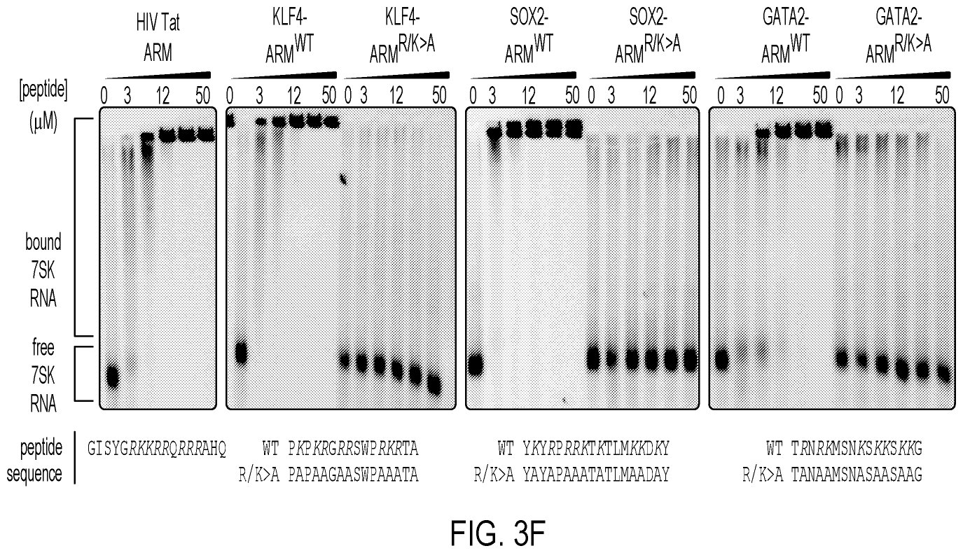

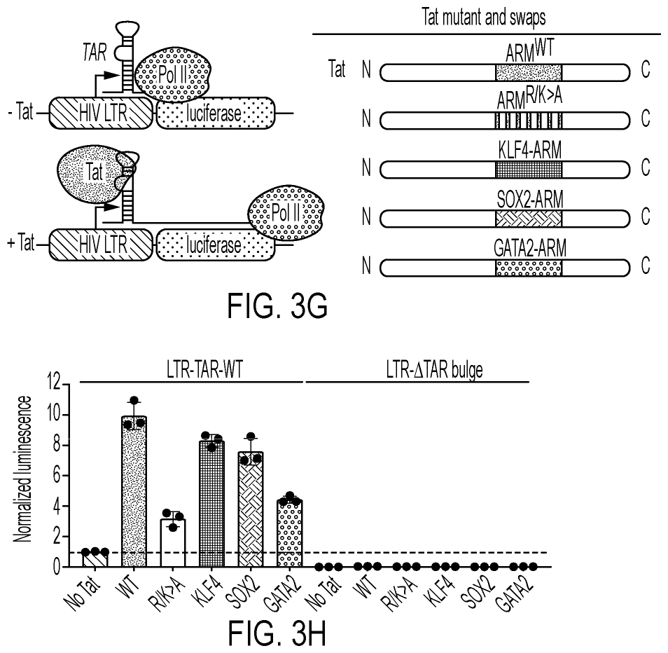

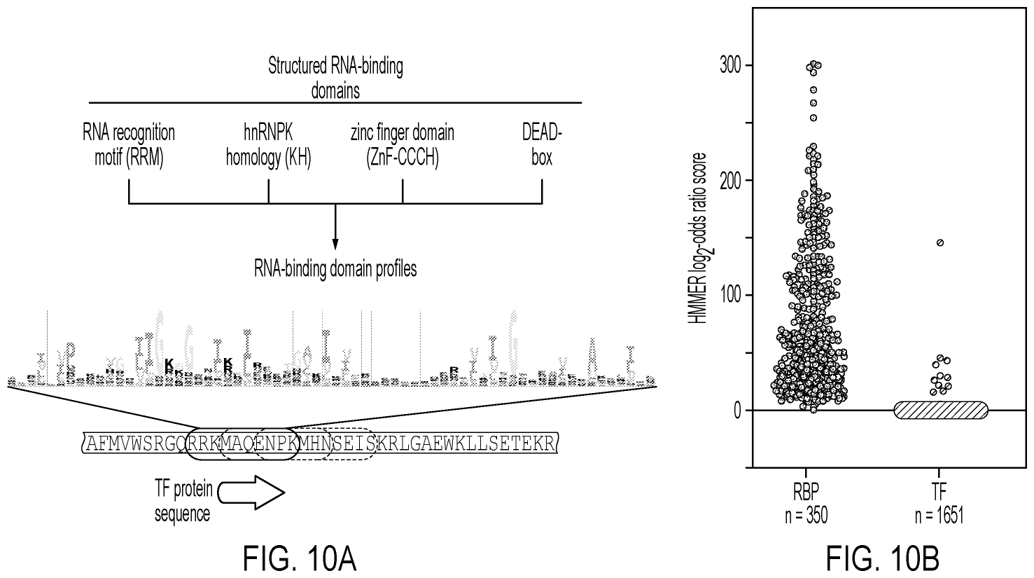

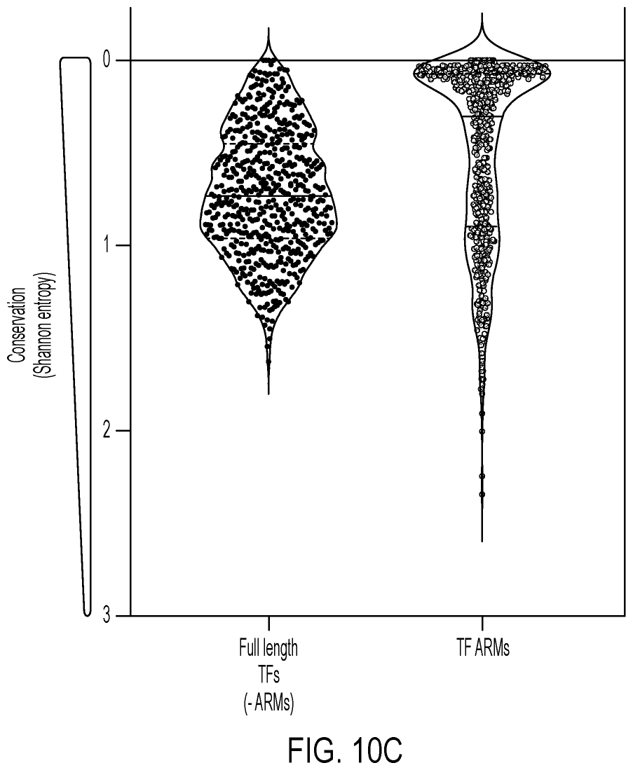

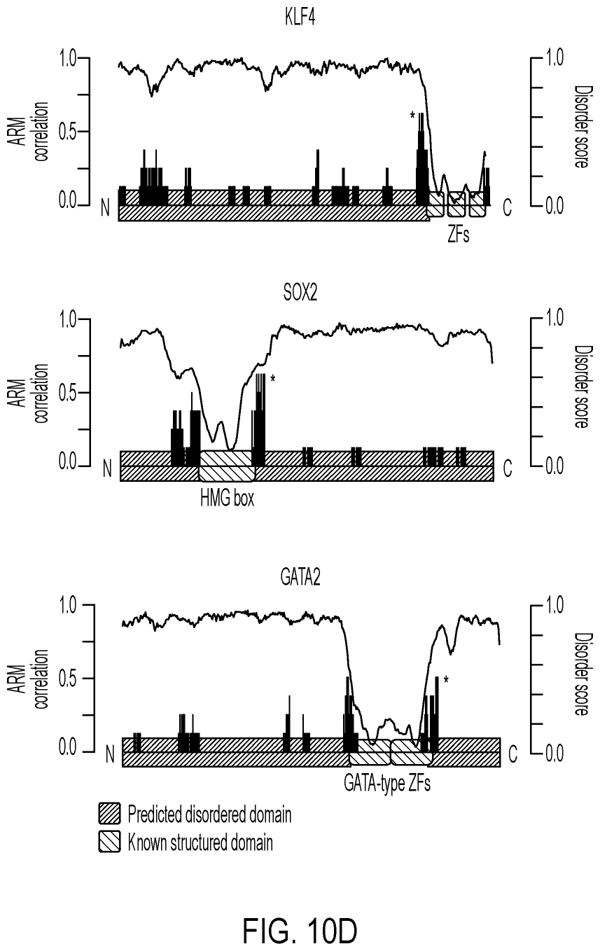

We next sought to identify regions in TFs that contribute to RNA binding. TFs do not contain sequence motifs that resemble those of structured RNA-binding domains^{29,38} (FIGS. 10A and 10B), so we searched for local amino acid features that might be common to TFs. Nearly 80% of TFs were found to have a cluster of basic residues (R/K) adjacent to their DNA-binding domain (FIG. 3A). Derivation of a position-weight matrix from these “basic patches” revealed that they contain a sequence motif similar to the RNA-binding domain of the HIV Tat transactivator, which has been termed the arginine-rich motif (ARM)^{41,42} (FIG. 3B). These ARM-like domains were enriched in TFs compared to the remainder of the proteome (FIG. 3C). Furthermore, the ARM-like domains have sequences that are evolutionarily conserved and appear adjacent to diverse types of DNA-binding domains, as illustrated for KLF4, SOX2, and GATA2 (FIGS. 3D, 10C, and 10D). This analysis suggests that TFs often contain conserved ARM-like domains, which we will refer to hereafter as TF-ARMs.

To investigate whether TF-ARMs are necessary for RNA binding, we purified wild-type and deletion mutant versions of KLF4, SOX2 and GATA2 and compared their RNA binding affinities. The 7SK RNA was used in this assay because it is one of a number of RNA species known to be bound by HIV Tat⁴³. RNA binding by the ARM-deleted proteins was substantially reduced (FIG. 3E). To determine if the TF-ARMs are sufficient for RNA binding, peptides containing the HIV Tat ARM and TF-ARMs were synthesized and their ability to bind 7SK RNA was investigated using an electrophoretic mobility shift assay (EMSA). The results showed that all the TF-ARM peptides can bind 7SK RNA, as did the control HIV Tat ARM peptide (FIG. 3F). This binding was dependent on arginine and lysine residues within the TF-ARMs (FIG. 3F), as has been previously demonstrated for the Tat ARM^{41,43} These results indicate that TF-ARMs are necessary and sufficient for RNA binding.

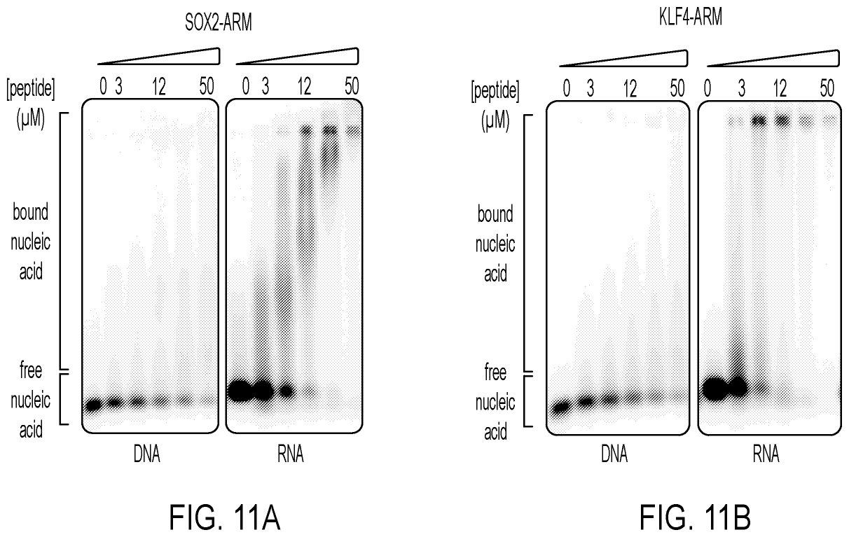

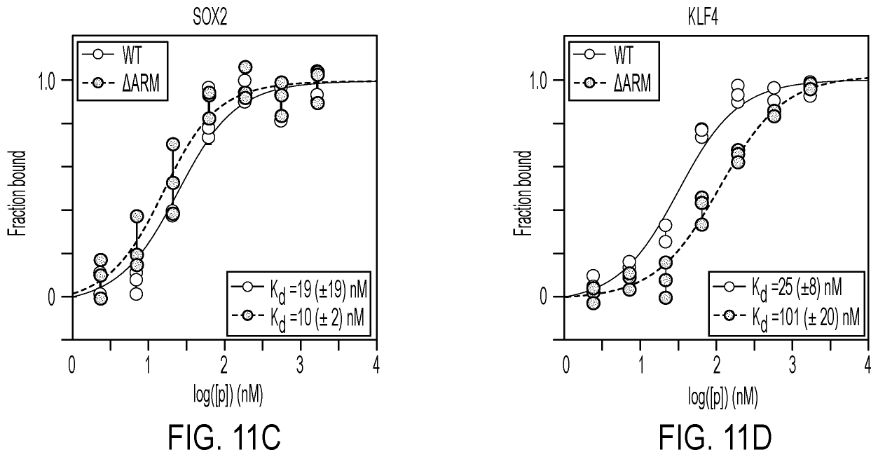

We considered the possibility that the TF-ARM also contributes to DNA-binding. Synthesized peptides of the SOX2 and KLF4 ARMs were tested for binding to either DNA or RNA. The results show that both ARMs bind RNA with greater affinity compared to DNA (FIGS. 11A and 11B). Full-length wildtype and ARM-deleted SOX2 and KLF4 were also tested for binding to motif-containing DNA. The results show that deletion of the SOX2 ARM did not affect DNA-binding (FIG. 11C). Deletion of the KLF4 ARM did affect DNA-binding (FIG. 11D), although not to the extent that it affected RNA binding (FIG. 3E). It thus appears possible that some TF-ARMs can contribute to DNA-binding to some extent whereas others do not.

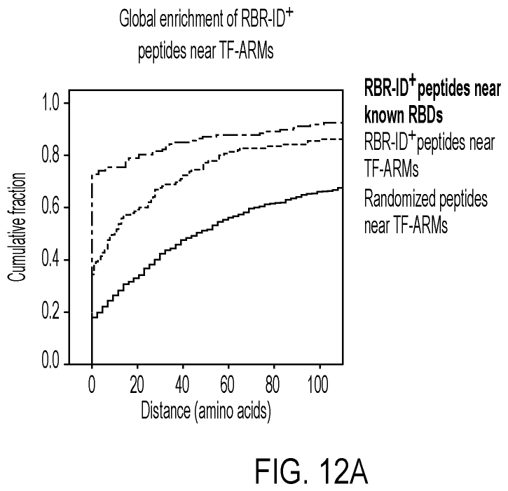

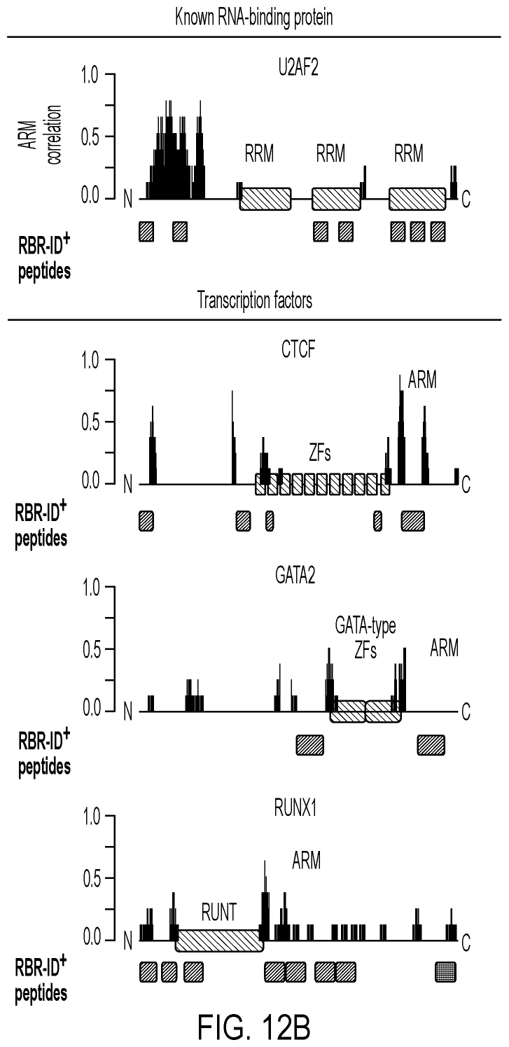

Having found that TF-ARMs bind to RNA in vitro in assays with purified components, we next asked whether TF-ARMs bind RNA in the more complex environment of the cell. To investigate this, we analyzed the RBR-ID data (FIGS. 1B-D), which can provide spatial information on the regions of proteins that bind RNA in cells. If TF-ARMs were binding to RNA in cells, then we would expect an enrichment of RBR-ID⁺ peptides overlapping or adjacent to the TF-ARMs. Global analysis of RBR-ID⁺ peptides in human K562 cells, as well as inspection of RBR-ID+ peptides for individual TFs, confirmed that this was the case (FIGS. 12A-B). These results provide evidence that ARM-like regions in TFs bind to RNA in cells.

To investigate if TF-ARMs could function similarly to the Tat ARM in cells, we tested whether TF-ARMs could replace the Tat ARM in a classical Tat transactivation assay41. In this assay, the HIV-1 5′ long terminal repeat (LTR) is placed upstream of a luciferase reporter gene. Transcription of the LTR generates an RNA stem loop structure called the Trans-activation Response (TAR), and HIV Tat binds to the TAR RNA to stimulate expression of the reporter gene44 (FIG. 3G). We confirmed that expression of full-length Tat stimulates luciferase expression, and that mutation of the lysines and arginines in the Tat ARM reduces this activity (FIG. 3H). Replacing the Tat ARM with the TF-ARMs of KLF4, SOX2, or GATA2 rescued the loss of the Tat ARM (FIG. 3H). In all cases, activation was dependent on the TAR RNA bulge structure, which is required for Tat binding44 (FIG. 3H). These results indicate that the TF-ARMs can perform the functions described for the Tat ARM and activate gene expression in an RNA-dependent manner.

### Tf-ARMs Enhance TF Chromatin Occupancy and Gene Expression

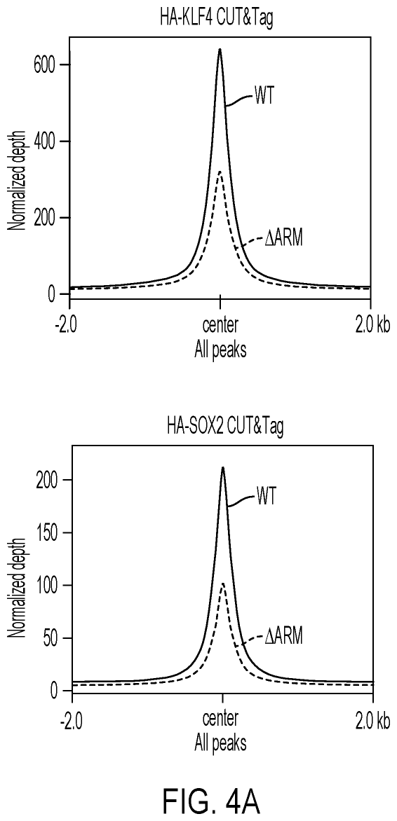

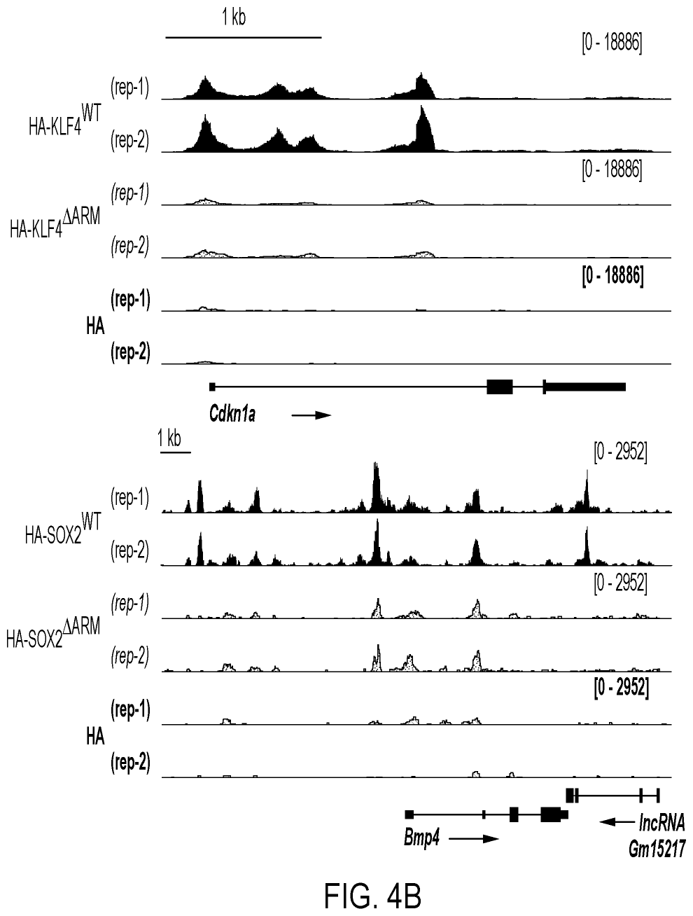

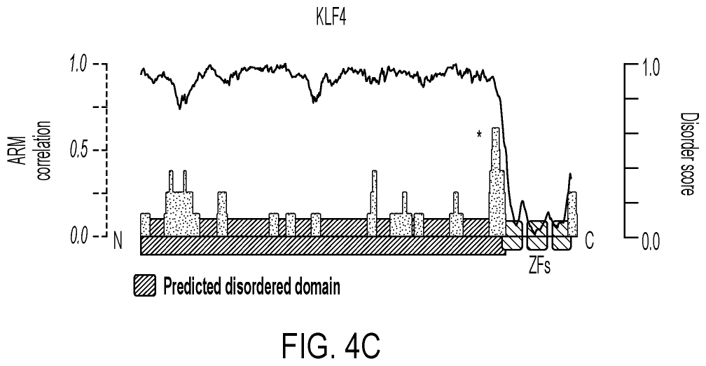

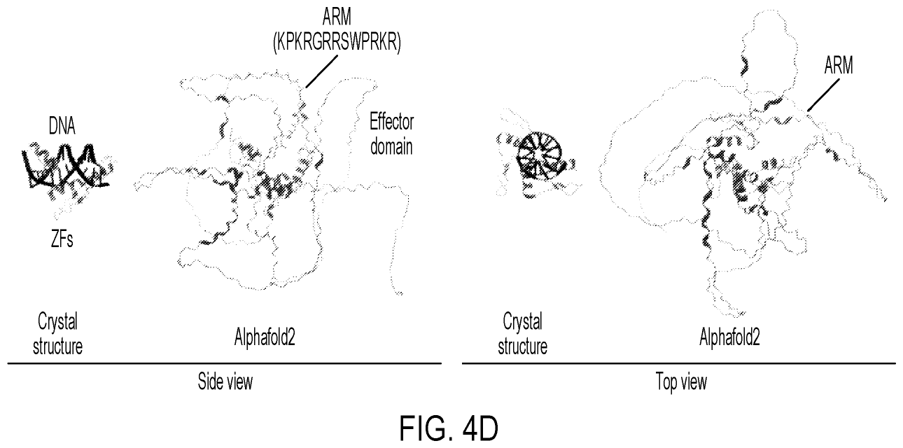

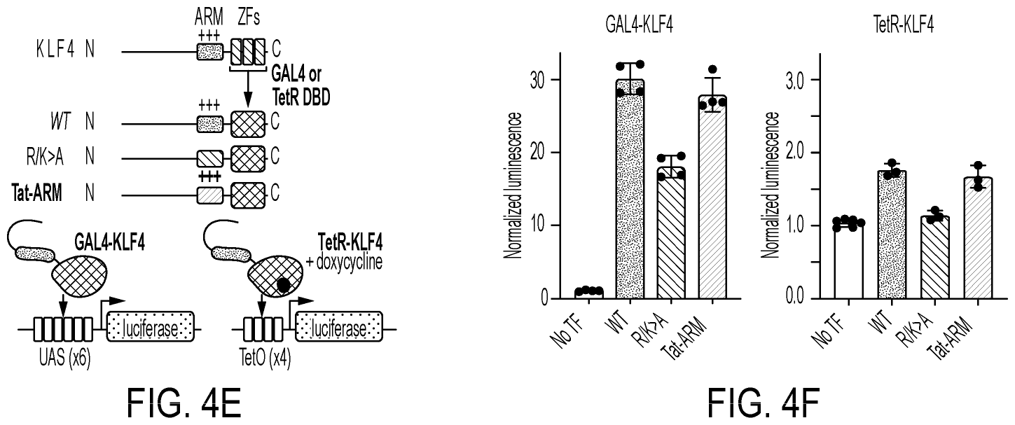

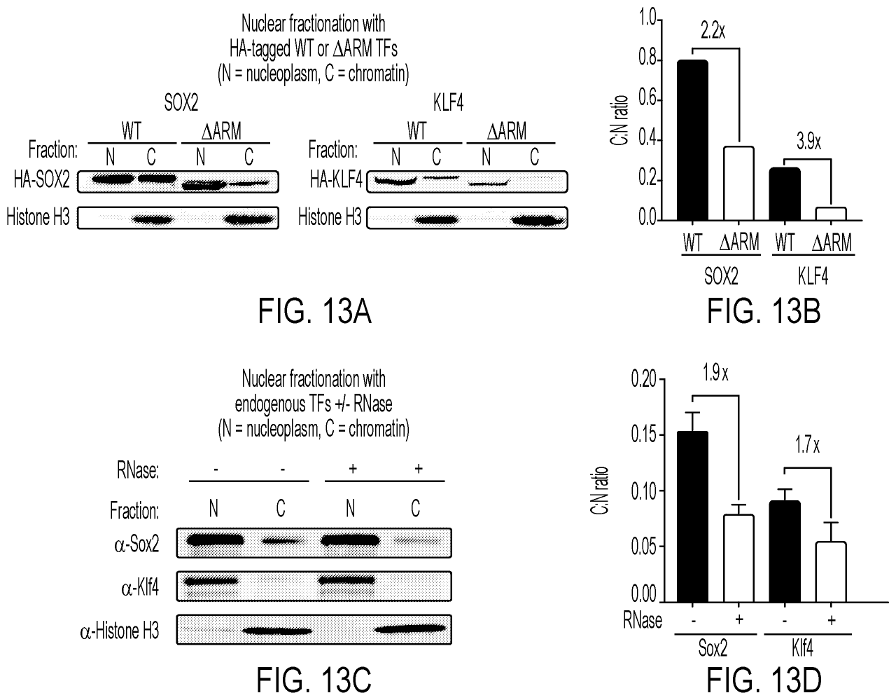

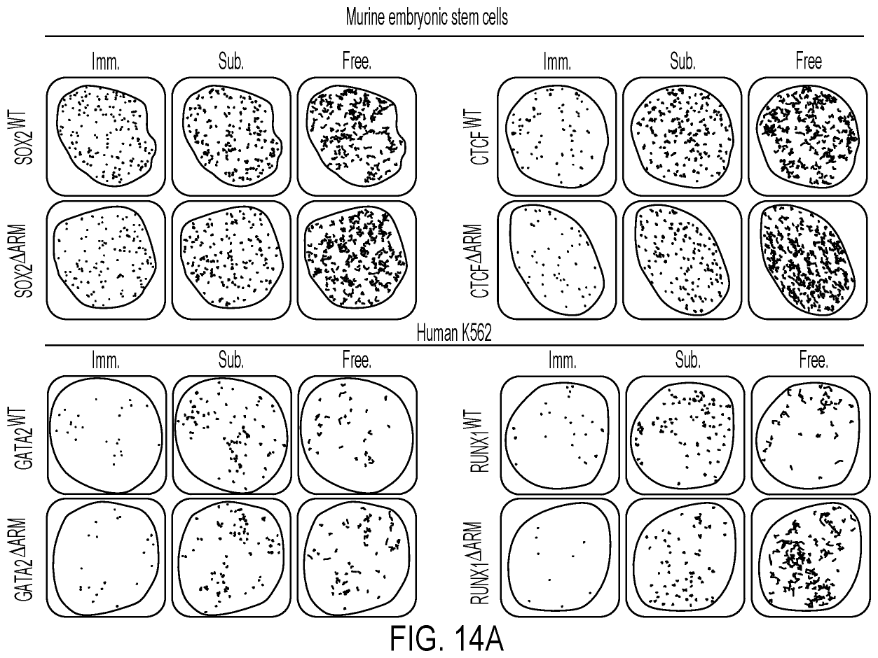

TFs bind enhancer and promoter elements in chromatin and regulate transcriptional output, so it is possible that RNA binding, enabled by TF-ARMs, contributes to chromatin occupancy and gene expression. We investigated whether TF-ARMs contributed to TF association with chromatin by measuring the relative levels of TFs in chromatin and nucleoplasmic fractions from ES cells containing HA-tagged TFs with wild-type and mutant ARMs. Genome-wide localization of KLF4 and SOX2 was globally reduced upon deletion of their ARMs (FIG. 4A) as determined by CUT&Tag and illustrated for specific genes regulated by KLF4 or SOX2 (FIG. 4B). Nuclear fractionation confirmed that deletion of the ARMs reduced the levels of KLF4 and SOX2 in chromatin (FIGS. 13A and 13B), and treatment of the extracts with RNase reduced TF enrichment in the chromatin fraction (FIGS. 13C and 13D). These results are consistent with a model whereby TF-RNA interactions enhance the association of TFs with chromatin.

We next sought to determine whether TF-ARMs contribute to gene output by using a transcriptional reporter assay that has been used extensively to investigate the functions of domains in TFs that contribute to transcriptional output8. KLF4 was selected for study because previous studies have used this assay to study KLF4 function in various cellular contexts45-47, KLF4 has a single ARM-like domain (FIGS. 4C and 4D), it has contiguous effector and DNA-binding domains, and our assays show that deletion of the ARM has a strong effect on RNA binding (FIG. 3E). In this assay, the KLF4 zinc fingers (DBD) were replaced with the yeast GAL4 DBD, and this fusion was tested for its ability to activate expression of a luciferase reporter downstream of GAL4-binding UAS sites (FIG. 4E). GAL4-KLF4WT activated reporter expression, while substitution of arginines and lysines for alanines in the ARM (GAL4-KLF4R/K>A) significantly reduced reporter expression (FIG. 4F). Importantly, this reduction was rescued by replacement of the ARM with the HIV Tat ARM (FIG. 4F). Similar effects were observed with the replacement of KLF4 DBD with the bacterial TetR DBD, which recognizes TetO elements in the presence of doxycycline (FIGS. 4E and 4F). The mutation of the KLF4 ARM caused a reduction in reporter expression rather than complete ablation of expression. These results, taken together with previous studies⁴⁵⁻⁴⁷, suggest that while the DNA and protein binding portions of the TF play major roles in gene activation, TF-RNA binding contributes to fine-tune transcriptional output.

### A Role for TF RNA-Binding Regions in TF Nuclear Dynamics

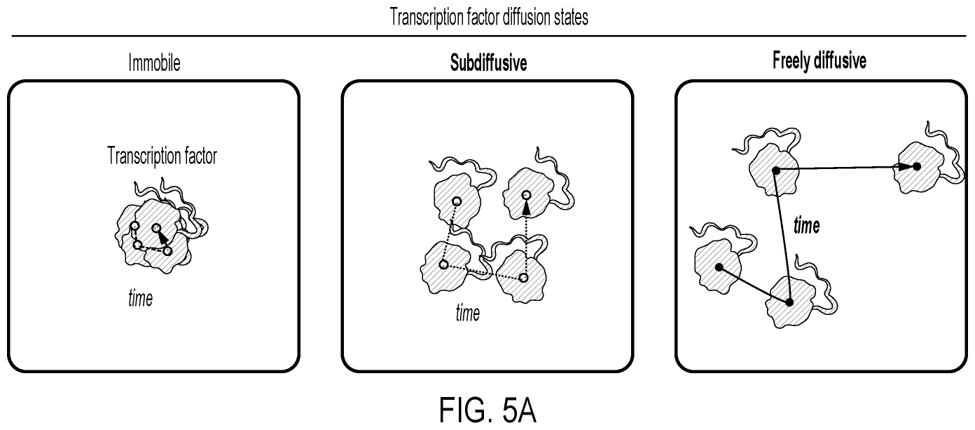

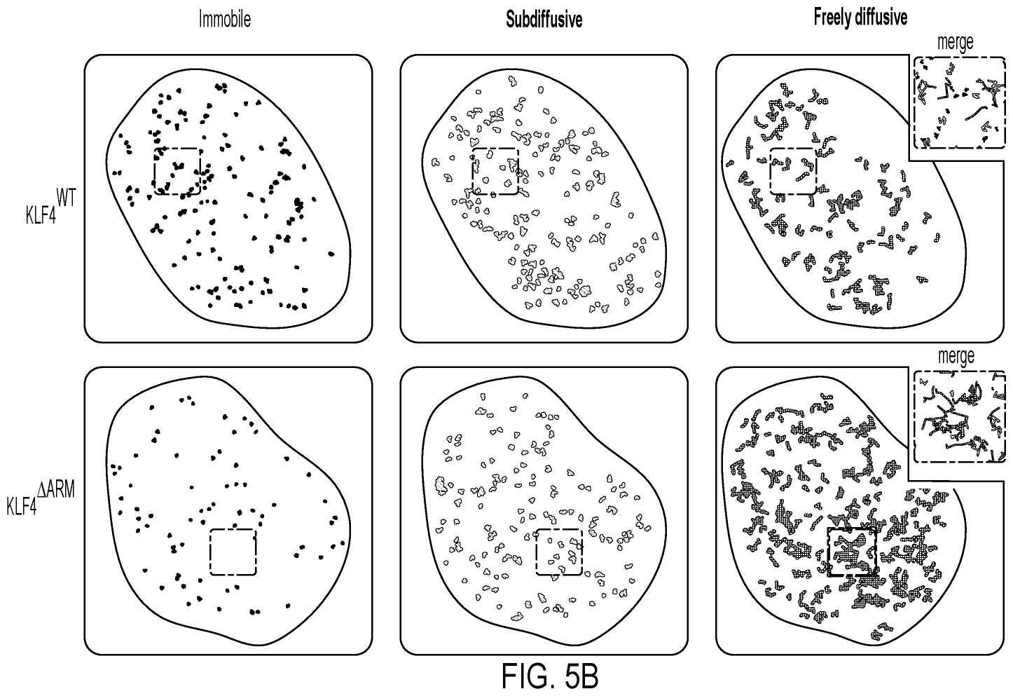

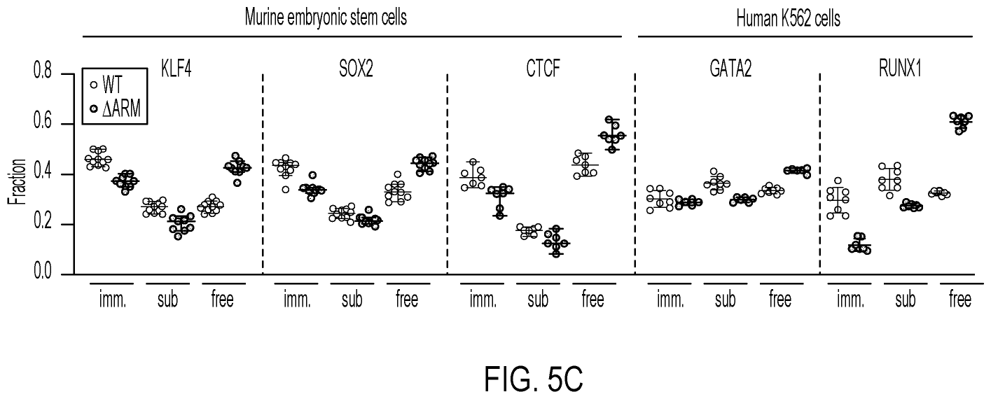

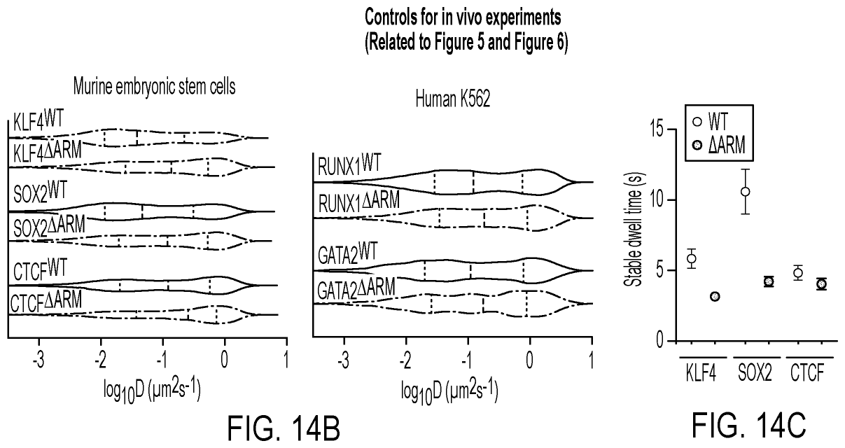

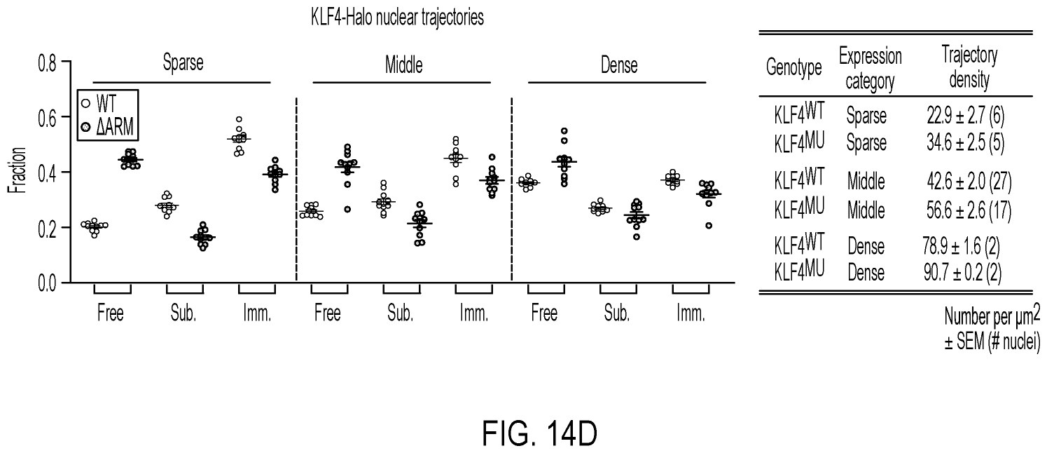

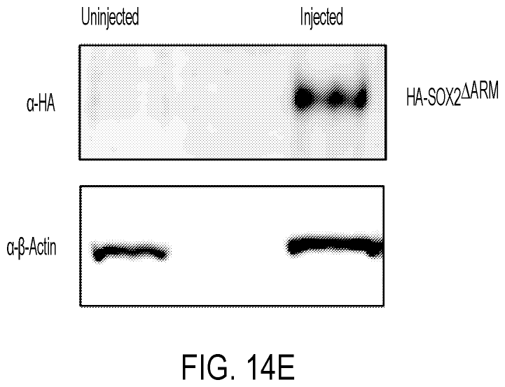

TFs are thought to engage their enhancer and promoter DNA-binding sites through search processes that involve dynamic interactions with diverse components of chromatin. Single molecule image analysis of TF dynamics in cells indicates that TFs conduct a highly dynamic search for their binding sites in chromatin^{48,49}. The tracking data can be fit to a three-state model, where TFs are interpreted to be immobile (potentially DNA-bound), subdiffusive (potentially interacting with chromatin components) and freely diffusing^{50,51}. If TFs interact with chromatin-associated RNA through their ARMs, then we might expect that mutation of their ARMs would reduce the portion of TF molecules in the immobile and sub-diffusive states. To test this, we conducted single-molecule tracking experiments with murine embryonic stem cell (mESC) or human K562 leukemia lines that enable inducible expression of Halo-tagged wildtype or ARM-mutant TFs. For these experiments, we chose the TFs SOX2, KLF4, GATA2, and RUNX1 because of their prominent roles in mES or hematopoietic cells32,34 and our earlier characterization of their RNA-binding regions (FIGS. 3A-H). As a control, we included the deletion of an ARM-like region from CTCF that overlaps the previously described RNA-binding region (RBR)36, which was shown to reduce both the immobile and subdiffusive fractions of CTCF52. Single-molecule imaging data was fit to a three-state model: immobile, subdiffusive, and freely diffusing (FIG. 5A and STAR Methods). Inspection of single-molecule traces for wildtype and ARM-mutant TFs (FIGS. 5B and 14A), as well as global quantification across replicates (FIGS. 5C, 14B, and 14C), showed that deletion of the ARM-like domains in TFs reduces the fraction of molecules in both the immobile and subdiffusive fractions, while increasing the fraction of freely diffusing molecules. Although diffusive fractions changed with expression level, the behavior of the mutant TF was consistent across expression regimes (FIG. 14D). The observed changes in diffusivity upon ARM mutation could reflect changes in binding between TFs and RNA or DNA molecules. The observation that ARM peptides have a preference for RNA binding (FIGS. 11A-D), and evidence that TF chromatin occupancy is reduced upon RNase treatment or ARM mutation (FIGS. 13A-D), is consistent with a role for RNA interactions in TF nuclear dynamics. These results suggest that TF-ARMs enhance the timeframe in which TFs are associated with chromatin.

### TF-ARMs are Essential for Normal Development and Disrupted in Disease

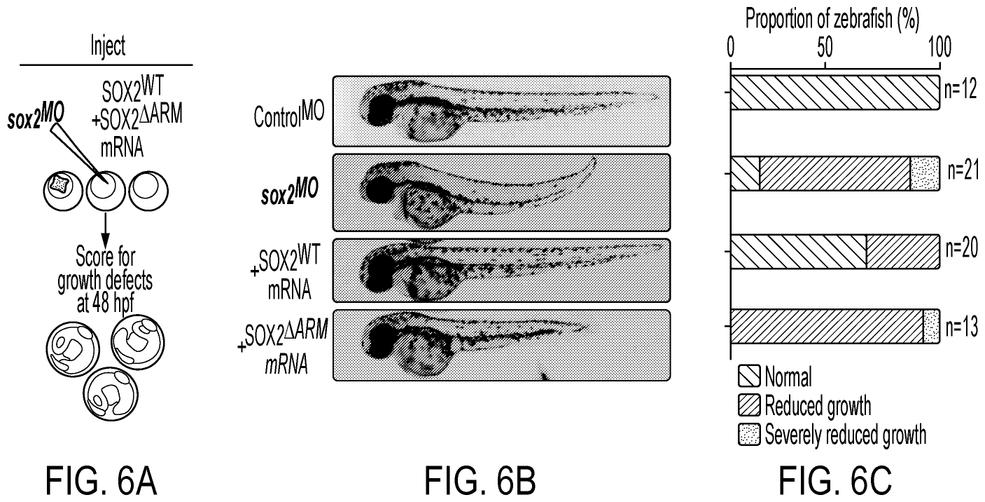

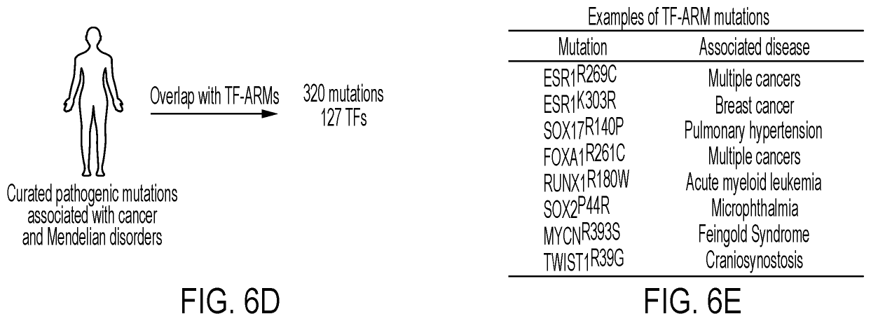

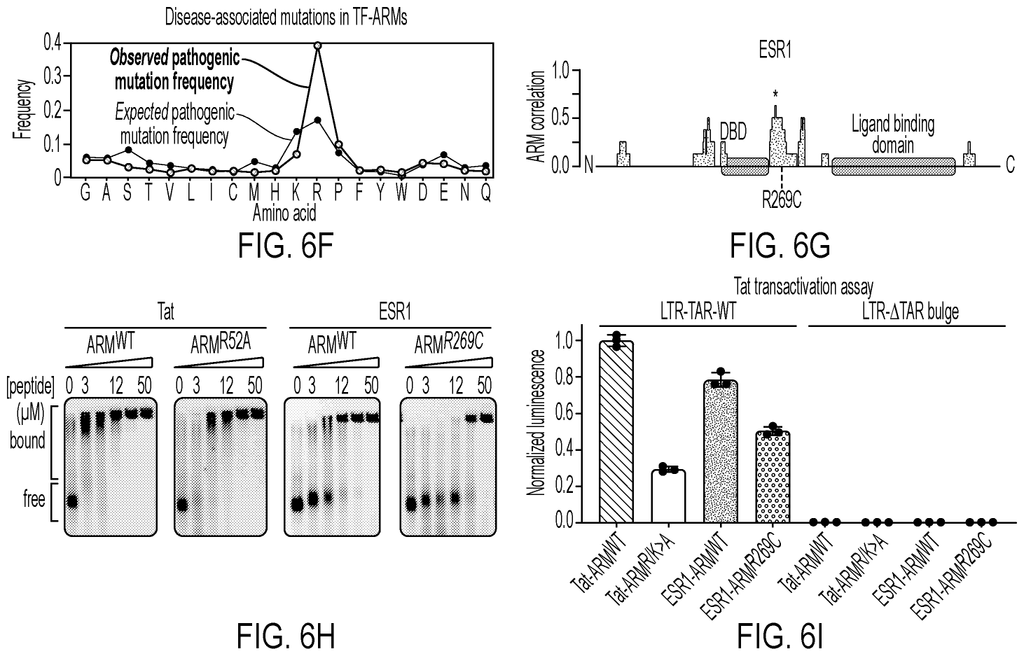

Transcription factors are fundamental controllers of cell-type specific gene expression programs during development, so we next asked whether the TF-ARMs contribute to the factor's role in normal development in vivo. For this purpose, we turned to the zebrafish, which has served as a valuable model system to study and perturb vertebrate development. Previous study showed that knockdown of zebrafish sox2 by injection of antisense morpholinos at the one-cell stage led to growth defects and embryonic lethality, which could be rescued by co-injection with messenger RNA (mRNA) encoding human SOX253. Using this system, we injected zebrafish with the sox2 morpholino while co-injecting mRNA encoding either wildtype or ARM-mutant human SOX2 (FIGS. 6A and 14E), which reduced RNA but not DNA binding in vitro (FIGS. 3E and 11C). Embryos were scored at 48 hours post-fertilization for growth defects by the length of the anterior-posterior axis compared to embryos injected with a non-targeting control morpholino (FIG. 6B). Whereas wildtype human SOX2 could partially rescue the growth defect induced by sox2 knockdown, ARM-mutant SOX2 was unable to do so (FIGS. 6C and 14E). These results indicate that TF-ARMs contribute to proper development.

The presence of ARMs in most TFs, and evidence that they can contribute to TF function in a developmental system, prompted us to investigate whether pathological mutations occur in these sequences in human disease. Analysis of curated datasets of pathogenic mutations revealed hundreds of disease-associated missense mutations in TF-ARMs (FIG. 6D, Table 2, STAR Methods). These mutations are associated with both germline and somatic disorders, including multiple cancers and developmental syndromes, that affect a range of tissue types (FIG. 6E). Variants that mutate arginine residues were the most enriched compared to the other amino acid residues in ARMs (STAR Methods), which is consistent with their importance in RNA binding (FIG. 6F)⁴². To confirm that such mutations could affect RNA binding, we selected for further study the estrogen receptor (ESR1) R269C mutation (FIG. 6G), which is found in multiple cancers and is particularly enriched in a subset of patients with pancreatic cancer54. An EMSA assay showed that RNA binding was reduced with an ESR1 ARM peptide containing the R269C mutation (FIG. 6H). Furthermore, when the Tat ARM was replaced with wildtype and mutant versions of the ESR1 ARM in the Tat transactivation assay, the mutation caused reduced reporter expression compared to wildtype (FIG. 6I). These results support the hypothesis that disease-associated mutations in TF-ARMs can disrupt TF RNA binding.

### Discussion

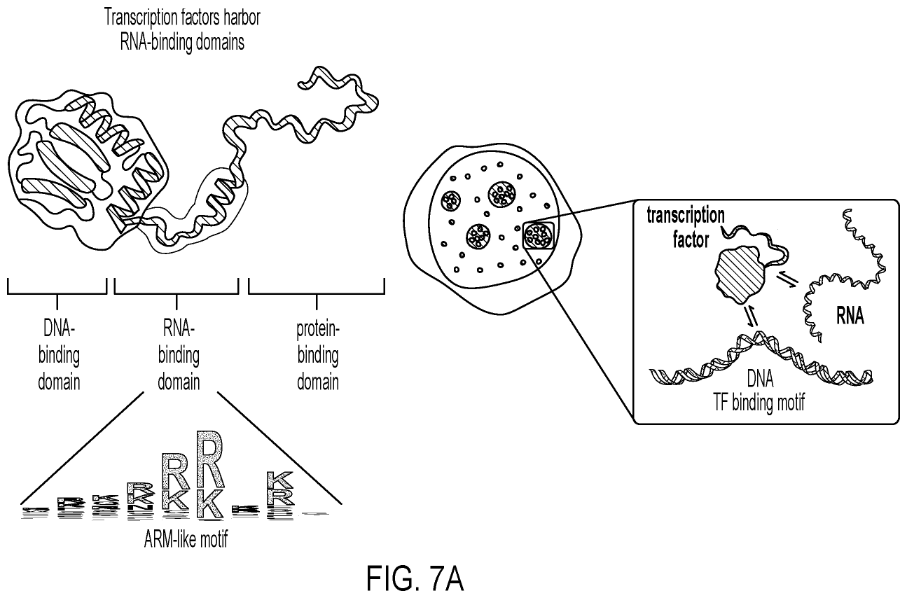

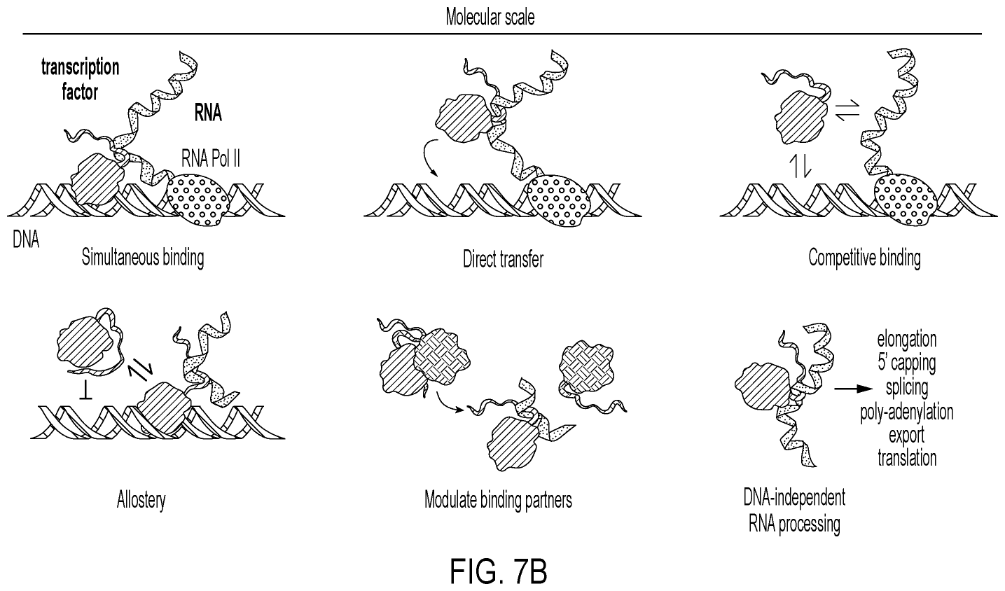

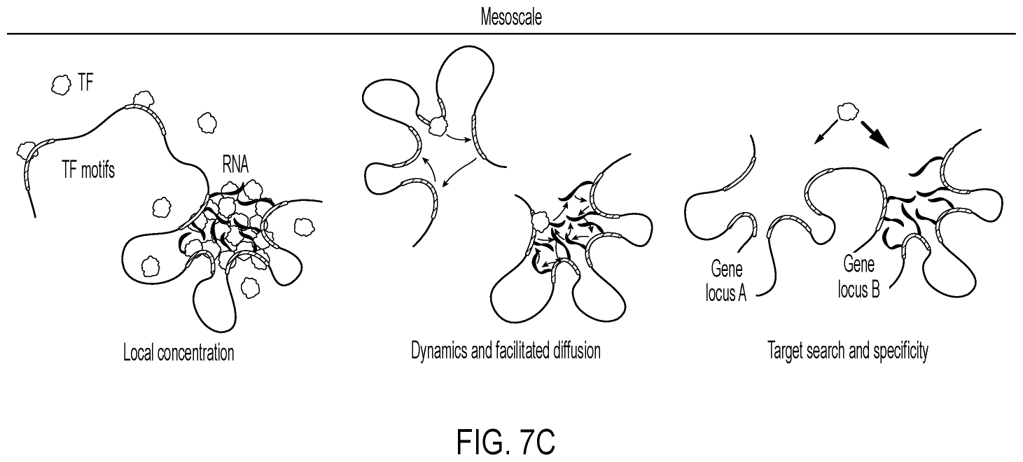

The canonical view of transcription factors is that they guide the transcription apparatus to genes and control transcriptional output through the concerted function of domains that bind DNA and protein molecules^{1,3,55,56}. The evidence presented here suggests that many transcription factors also harbor RNA-binding domains that contribute to gene regulation (FIG. 7A). Given the large portion of TFs that showed evidence of RNA interaction in cells and the presence of an ARM-like sequence in nearly 80% of TFs, it is possible that the majority of TFs engage in RNA binding.

RNA molecules are pervasive components of active transcriptional regulatory loci^{15,16,57-59} and have been implicated in the formation and regulation of spatial compartments⁶⁰. The noncoding RNAs produced from enhancers and promoters are known to affect gene expression¹⁵, and plausible mechanisms by which these RNA species could influence gene regulation have been proposed to include binding to cofactors and chromatin regulators⁶¹⁻⁶⁴, and electrostatic regulation of condensate compartments⁵⁸. The evidence that TFs bind RNA suggests additional functions for RNA molecules at enhancers and promoters (FIGS. 7B and 7C). These RNA molecules serve to enhance the recruitment and dynamic interaction of TFs with active regulatory DNA loci.

The observation that many TFs can bind DNA, RNA and protein molecules offers new opportunities to further advance our understanding of gene regulation and its dysregulation in disease. Knowledge that TFs can interact with both DNA and RNA molecules may help with efforts to decipher the “code” by which multiple TFs collectively bind to specific regulatory regions of the genome and inspire novel hypotheses that may provide additional insight into gene regulatory mechanisms. It might also provide new clues to the pathogenic mechanisms that accompany GWAS variants in enhancers, where those variations occur in both DNA and RNA.

### Limitations of the Study

This study shows that many transcription factors bind RNA and harbor RNA-binding domains that resemble the HIV Tat ARM. Our results demonstrate for a few tested examples that these domains contribute to the dynamic association of TFs with chromatin, which may provide a mechanism by which TF-RNA interactions contribute to gene control. There are several ways in which the binding of TFs to RNA could affect their function (FIGS. 7B and 7C), and these mechanisms could result in positive or negative effects on transcriptional output. It is also possible that these domains have additional RNA-dependent functions, some of which may be general and some TF-specific65. Another limitation of the study is the extent to which cellular and organismal phenotypes observed upon deletion of ARM-like domains can be attributed to RNA binding. We believe that characterization of these domains in TFs, including systematic identification of the precise residues required for RNA binding and RNA sequence preferences, will inspire investigation of their roles in many aspects of TF function, including but not limited to locus-specific chromatin association, chromatin architecture, transcriptional output, splicing, translational control, and RNA polymerase II pausing. A key challenge will be to delineate these functions in cells and explore how these functions are related to cooperative or competitive interactions of these domains with RNA, DNA or proteins.

### STAR Methods

### Data Code Availability

The RBR-ID mass spectrometry proteomics data have been deposited to the ProteomeXchange Consortium via the PRIDE partner repository with the dataset identifier PXD035484.

### Structures of Known DNA-Binding Domains in TFs

TF-DNA X-ray structures were obtained from the RCSB Protein Data Bank (Accession numbers: YY1=1UBD, MYC/MAX=1NKP, POU2F1=1CQT, JUN/FOS=1FOS). These entries were modified using ChimeraX^{66,67}, and the effector domains, which are not included in the X-ray structures, are depicted as cartoons highlighting their dynamic and transient structure.

### RNA Binding Region Identification (RBR-ID)

K562 cells were cultured in suspension flasks containing culture medium [RPMI-1640 medium with GlutaMAX™ (ThermoFisher Cat. 72400047) supplemented with 10% FBS (ThermoFisher Cat. 10437028), 2 mM L-glutamine (Sigma-Aldrich Cat. G7513), 50 U/mL penicillin and 50 [μg/mL streptomycin]. For each biological replicate of RBR-ID, 4 million K562 cells from actively proliferating cultures were aliquoted into 2×T25 flasks. 4-thiouridine (4SU) was added to one of the two flasks for each replicate at a final concentration of 50 μM and incubated for 2 hrs at 37° C. with 5% C02. Cells from each flask were collected and resuspended in 600 μL 1×PBS [137 mM NaCl, 2.7 mM KCl, 10 mM Na2HPO4, 1.8 mM KH2PO4] and transferred to 6-well plates.

Plates were placed on ice with their lids removed and protein-RNA complexes were crosslinked with 1 J/cm2 UVB (312 nm) light. Cells were lysed in Buffer A (10 mM Tris pH 7.94° C., 1.5 mM MgCl2, 10 mM KCl, 0.5 mM DTT, 0.2 mM PMSF) with 0.2% IGEPAL CA-630 for 5 min at 4° C., then centrifuged at 2,500 g for 5 min at 4° C. to pellet nuclei. Nuclei were washed 3× with 1 mL cold Buffer A (without IGEPAL) and lysed at room temperature in 100 L denaturing lysis buffer [9 M urea, 100 mM Tris pH 8RT, 1× complete protease inhibitor, EDTA free (Roche Cat. 4693132001)]. Lysates were sonicated using a BioRuptor instrument (Diagenode) as follows: (energy: high, cycle: 15 sec ON, 15 sec OFF, duration: 5 min), centrifuged at 12,000 g for 10 min and supernatant was collected. Extracts were quantified using Pierce BCA assay kit (ThermoFisher Cat. 23225). 5 mM DTT was added to extracts and incubated at room temperature for one hr to reduce proteins, and then alkylated with 10 mM iodoacetamide in the dark for one hr. Samples were then diluted to 1.5 M urea with 50 mM ammonium bicarbonate and treated with 1 μL of 10,000U/μL molecular grade benzonase (Millipore Sigma Cat. E8263) and incubated at room temperature for 30 min. Sequencing grade trypsin (Promega Cat. V5117) was then added to samples at a ratio of 1:50 (trypsin:protein) by mass and incubated at room temperature for 16 hrs. The digested samples were loaded onto Hamilton C18 spin columns, washed twice with 0.1% formic acid, and eluted in 60% acetonitrile in 0.1% formic acid. Samples were dried using a speed vacuum apparatus and reconstituted in 0.1% formic acid, then measured via A205 quantification and diluted to 0.333 g/μL.

For the proximity analysis in FIGS. 12A-B, the nearest distance was calculated for each detected protein between RBR-ID+ peptides (p-val<0.05, log 2FC<0) and either (1) TF-ARMs (cross-correlation to Tat ARM>0.5, described below), (2) Known RNA-binding domains (RRM: IPR000504, KH: IPR004087, dsRBD: IPR014720). We required that at least 3 peptides were detected for each protein considered. As a control for the TF-ARM nearest distance analysis, the label (RBR-ID+ or RBR-ID−) of each peptide was randomly shuffled 100 times for all detected RBR-ID peptides for each protein, which provides the null distribution of the dataset.

The RBR-ID mass spectrometry proteomics data have been deposited to the ProteomeXchange Consortium via the PRIDE partner repository with the dataset identifier PXD035484.

### LC-MS/MS

Peptide samples were batch randomized and separated using a Thermo Fisher Dionex 3000 nanoLC with a binary gradient consisting of 0.1% formic acid aqueous for mobile phase A and 80% acetonitrile with 0.1% formic acid for mobile phase B. 3 μL of each sample were injected onto a Pepmax C18 trap column and washed with a 0.05% trifluoroacetic acid 2% acetonitrile loading buffer. The linear gradient was 3 minutes until switching the valve at 2% mobile phase B and increasing to 25% by 90 minutes and 45% by 120 minutes at a flow rate of 300 nL/minute. Peptides were separated on a laser-pulled 75 m ID and 30 cm length analytical column packed with 2.4 μm C18 resin. Peptides were analyzed on a Thermo Fisher QE HF using a DIA method.

The precursor scan range was a 385 to 1015 m/z window at a resolution of 60 k with an automatic gain control (AGC) target of 106 and a maximum inject time (MIT) of 60 ms. The subsequent product ion scans were 25 windows of 24 m/z at 30 k resolution with an AGC target of 106 and MIT of 60 ms and fragmentation of 27 normalized collision energy (NCE). All samples were acquired by LC-MS/MS in three technical replicates. Thermo .raw files were converted to indexed mzML format using ThermoRawFileParser utility (https://github.com/compomics/ThermoRawFileParser). To detect and quantify peptides, indexed mzML files from each set of technical replicates were searched together using Dia-NN v1.8.168 against a FASTA file of the Homo sapiens UniProtKB database (release 2022_02, containing Swiss-Prot+TrEMBL and alternative isoforms). Precursor and fragment m/z ranges of 300-1800 and 200-3000 were considered, respectively with peptides lengths from 6-40. Fixed and variable modifications included carbamidomethyl, N-term acetylation and methionine oxidation. A 0.01 q value cutoff was applied, and the options --peak-translation and --peak-center were enabled, while all other Dia-NN parameters were left as default.

### Bioinformatic Analysis of the RBR-ID Data

After removal of suspected contaminants, identified peptides were re-mapped to an updated human proteome reference (UniProtKB release 2022_02, Swiss-Prot+TrEMBL+isoforms) to reannotate matching proteins. Where multiple protein matches were identified, peptides were assigned to a single protein annotation by first defaulting to Swiss-Prot accessions, where available, then by the accession with the most matching peptides in the dataset and therefore the most likely protein group69. Abundances of the different charge states of the same peptide were summed, and all abundances were normalized by the median peptide intensity in each run. To assess depletion mediated by RNA crosslinking, normalized abundances for each peptide in cells treated or not with 4SU were analyzed by unpaired, two-sided Student's t tests. For peptides that were missing across all 5×3 technical replicates in one of the treatments, Fisher's exact tests were used comparing the frequency of peptide detection between cells treated with or without 4SU. Statistical significance was determined by adjusting p values from both tests using the Benjamini-Hochberg method70. For mESC RBR-ID data from previous study31, all peptides were re-mapped to an updated mouse reference proteome (UniProtKBrelease 2021_04) as described above while keeping original quantification and Pvalues. A relaxed p-value threshold (0.10) was used in the original study because it was validated to include additional RBPs31. Peptides were annotated using the InterPro database (release 87, accessed 28 Feb. 2022) to identify functional domains. For volcano plots, outliers were removed and each marker represents the peptide with maximum RBR-ID score31 for each protein. Transcription factors annotated in this dataset are from a previous census study1.

### Generating List of RNA-Binding TFs

RNA-binding proteins identified in the current and previous studies using various methods were collected18,23,31,71-77. The list of RNA-binding proteins from these studies was overlapped with the list of transcription factors from a previous census study1 using merge function in R. Transcription factors that are found at least in one dataset were reported in Table 1.

### Clip

CLIP experiments were performed as previously described78 with minor modifications (see below for details).

### Protein-RNA Crosslinking

K562 cells were treated for 24 hours with 10 μM of 4-Thiouridine (4SU) (Sigma-Aldrich T4509) prior to cell collection. Cells were resuspended in 1×PBS and transferred to a 6-well plate for crosslinking. Plates were placed on ice with lids removed and crosslinked at 365 nm at 0.3 J/cm². Cell suspension was transferred to microcentrifuge tubes and plates were washed with 1×PBS.

### Lysate Preparation

Cells were washed in 1×PBS and cell pellets were lysed in eCLIP lysis buffer [20 mM HEPESNaOH pH 7.4, 1 mM EDTA, 100 mM NaCl, 1% NP-40, 0.1% SDS, 0.5% sodium deoxycholate, 1×cOmplete□ EDTA-free protease inhibitor cocktail (Roche 4693132001)]. Samples were sonicated in a Diagenode Bioruptor (30 s ON/OFF) on medium for 5 minutes. RNase I (ThermoFisher AM2294) was added to lysates for a final concentration of 0.4 U/μL and incubated at 37° C. at 1200 rpm for 5 min. EDTA was immediately added at a final concentration of 21 mM. Lysates were clarified at 15,000 g for 10 minutes at 4° C. and supernatant was transferred to fresh tubes. Protein concentration was measured using Protein Assay Dye Reagent (Bio-Rad 5000006).

### Labeling of Crosslinked Protein-RNA Complexes

Dynabeads™ were washed in eCLIP binding buffer (20 mM HEPES-NaOH pH 7.4, 20 mM EDTA, 100 mM NaCl, 1% NP-40, 0.1% SDS, 0.5% sodium deoxycholate). Antibody was added to bead mixture and incubated, rotating at room temperature for 45 min. Antibody-bead mixture was washed in eCLIP binding buffer and mixed with calculated amount of lysate. Tubes were incubated overnight rotating at 4° C. 2% of lysate-bead mixture was transferred to a new tube to serve as input sample. IP samples were washed with CLIP wash buffer (20 mM HEPES-NaOH pH 7.4, 20 mM EDTA, 5 mM NaCl, 0.2% Tween-20) and IP50 (20 mM Tris pH 7.3RT, 0.2 mM EDTA, 50 mM KCl, 0.05% NP-40). Samples were treated with TURBO□ DNase (ThermoFisher AM2238) and 0.1 U/μL final concentration of RNase I (in some cases, 1 U/μL final concentration was used for better visualization of bands, e.g. Fig. S2A). IP samples were washed in CLIP wash buffer and FastAP buffer (10 mM Tris-Cl pH 7.5RT, 5 mM MgCl2, 100 mM KCl, 0.02% Triton X-100). IP RNA was dephosphorylated using FastAP phosphatase reaction FastAP Thermosensitive Alkaline Phosphotase (ThermoFisher EF0652), and T4 PNK (NEB M0201S).

IP samples were washed in CLIP wash buffer and 1×RNA Ligase buffer (50 mM Tris-Cl pH 7.5RT, 10 mM MgCl2]. A 3′ IR-800 fluorescent adaptor was ligated using T4 RNA Ligase 1 high concentration (NEB M0437M). Samples were washed in eCLIP high-salt wash buffer (50 mM Tris-HCl pH 7.4RT, 1M NaCl, 1 mM EDTA, 1% NP-40, 0.1% SDS, 0.5% sodium deoxycholate) and CLIP wash buffer. IP and input samples were eluted with 4×LDS Sample Buffer (ThermoFisher NP0007), run on an 8% bis-tris gel, and transferred overnight to a nitrocellulose membrane.

### Library Preparation and Sequencing

The transferred membrane was cut ˜0-50 kDa above protein size and incubated with Proteinase K (ThermoFisher AM2548) to isolate crosslinked RNA. Remaining steps were performed as per the seCLIP protocol79, with some modifications. RNA was purified and concentrated with phenol:chloroform:IAA (ThermoFisher AM9732) and ethanol precipitation. 3′ and 5′ adapters were designed to include an IR800 fluorophore and an 8-nt UMI for cDNA ligation, respectively. We did not include 5′ deadenylase enzyme in our 5′ ligation reactions and we used the AffinityScript RT (Agilent 600107) for crosslinking-induced truncation. Libraries were sequenced on an Illumina NextSeq 500 in paired-end mode for 47:8:8:29 cycles (read 1 index 1:index2:read 2).

### CLIP Analysis

### Generating CLIP-Seq Peaks

Raw CLIP-seq reads were trimmed using Cutadapt80. The adapter sequence AGATCGGAAGAGCACACGTCTGAA (SEQ ID NO: 1) was trimmed from the 5′ end of the reads, AGATCGGAAGAGCGTCGTGTAGGGAAAGAGTGT (SEQ ID NO: 2) adapter sequence from the 3′ end, and a universal four nucleotide UMI from the 3′ end. Prior to mapping, UMIs were extracted from the 5′ end of the reads using UMI-tools version 1.0.0 with the argument --bc-patter=NNNNNNNN81.

Bowtie2 was used to map all trimmed reads to the hg19 human genome using parameters -p 40 -end-to-end -no-discordant82,83. Trimmed and mapped reads were then sorted using the samtools sort function and indexed using the bedtools index function84,85. Lastly, reads were collapsed to account for PCR duplicates using the extracted UMIs with the UMI-tools dedup function. These trimmed, mapped, and collapsed reads were then used for downstream analysis. To call CLIP-seq peaks, .bed files were generated using MACS with parameters -g hs --keep-dup auto -nomodel86.

### Identifying Crosslinked Nucleotides

The site of the expected crosslink is first nucleotide in the DNA template upstream of position 1 (or the −1 position) of the 5′ end of the + strand mapped reads (see CLIP methods). Reads containing crosslinked nucleotides were defined as the reads containing a U in the −1 position nucleotide of the 5′ end of the + strand mapped reads. As expected, there was an enrichment of U nucleotides as compared to Gs, Cs, and As at this position within the reads.

### Generating CLIP-Seq Metaplots

Fastq files from GATA2 ChIP-seq⁸⁷ (GSM467648) and RUNX1 ChIP-seq⁸⁸ (GSM2423457) experiments in K562 cells were downloaded from Gene Omnibus Expression database (GEO) and aligned to the hg19 human genome using Bowtie2. ChIP-seq peaks were called using MACS with parameters -g hs --keep-dup auto --nomodel. Regions for metaplot analysis were generated using +/−2000 bases from the center of the called peaks. Normalized CLIP-seq densities within these regions were calculated using bamToGFF89. Input-corrected meta-gene plots were generated by subtracting the mean read density per bin of the input CLIP at ChTP peaks from the the HA pull down CLIP at ChTP peaks. R matplot function was used to plot the density values across the 4 Kb region.

### Protein Purification

To purify transcription factors, a mammalian purification system using Freestyle HEK 293F cells (gift from Sabatini lab) were used. HEK cells were grown in FreeStyle 293 Expression Medium (Gibco) on an orbital shaker. Coding sequence of desired genes were synthesized by IDT as gBlock fragments (Table 3) containing proper Gibson overhangs. TF-ARM deletion mutants were generated by removal of a stretch of peptide adjacent to DNA binding domains that contain ARMs. The amino acid sequences that are removed in TF-ARM mutants are shown in parentheses as follows: hsKLF4_ΔARM (aa 355-386), hsSOX2_ΔARM (aa 118-178), hsGATA2_ΔARM (aa 360-395), and hsCTCF_ΔARM (576-611). To reduce sequence complexity for gBlock synthesis, codon optimization using the IDT codon optimization tool was applied when needed. The fragments are then cloned into a mammalian expression vector containing Flag and mEGFP (N- or C-terminal) (modified from Addgene #32104) using NEBuilder HiFi DNA Assembly kit (E2611). These vectors were transiently transfected into 293F cells at a concentration of 1 million/ml with 1 g of DNA per million cells using branched polyethylenimine (PEI) (Polysciences). 60-72 hours post-transfection, cells were resuspended in 45 ml HMSD50 buffer (20 mM HEPES pH 7.5, 5 mM MgCl2, 250 mM sucrose, 1 mM DTT, 50 mM NaCl, supplemented with 0.2 mM PMSF and 5 mM sodium butyrate) and incubated for 30 min at 4° C. with gentle agitation. After a spin down at 3500 rpm at 4° C. for 10 min, the supernatant was discarded and the pellet containing nuclei were resuspended in 35 ml of BD450 buffer (10 mM HEPES pH 7.5, 5% Glycerol, 450 mM NaCl, and protease and phosphatase inhibitors) and incubated for 30 min at 4° C. with agitation. The solution was spun down at 3500 rpm at 4° C. for 10 min to clear the nuclear extract. The supernatant was transferred into fresh tube and the pellet containing chromatin was passed through 18G 12 syringe 5 times. The chromatin containing lysate was spun down at 8000 rpm at 4° C. for 10 min and supernatant is combined with the previously collected supernatant. Then the combined supernatants were spun down again at 8000 rpm at 4° C. for 10 min to clear the lysate. 500 ul of Flag-M2 beads (Sigma) were added to the cleared lysates and incubated overnight at 4° C. The Flag-M2 beads were washed 2 times with 45 ml BD450 buffer and they were transferred into a purification column (Biorad). The beads on the column were washed 2 more times with 10 ml BD450 buffer and 5 ml Elution buffer (20 mM HEPES pH 7.5, 10% Glycerol, 300 mM NaCl). Elutions were performed by incubating the beads overnight at 4° C. with 800 elution buffer and 200 ul of 5 mg/ml flag peptide (Sigma). The buffer exchange (into elution buffer) and concentration of proteins were performed using spin columns (Milipore). Proteins were aliquoted and stored at −80° C.

### In Vitro RNA Synthesis and Purification

To synthesize labeled RNA for fluorescence polarization measurements, in vitro transcription templates were generated from ssDNA oligos (for the random RNA template, Integrated DNA Technologies), gBlocks (for 7SK template, Integrated DNA Technologies), or PCR amplification of genomic DNA from V6.5 murine embryonic stem cells (for Pou5f1 enhancer and promoter RNAs)⁵⁸. Templates were amplified by PCR with primers containing T7 (sense) or SP6 (antisense) promoters:
T7 (added to 5′ of sense): 5′ TAATACGACTCACTATAGGG 3′ (SEQ ID NO: 3)
SP6 (added to 5′ of antisense): 5′ ATTTAGGTGACACTATAGAA 3′ (SEQ ID NO: 4)

Templates were amplified using Phusion polymerase (NEB), and the products were gel-purified using the Monarch Gel Purification Kit (NEB) following the manufacturer's instructions and eluted in 40 μL H2O. Each template was transcribed using the MEGAscript T7 kit using 200 ng total template according to the manufacturer's instructions. Reactions included a Cy5-labeled UTP (Enzo LifeSciences ENZ-42506) at a ratio of 1:10 labeled UTP:unlabeled UTP. The transcription reaction was incubated overnight at 37° C., and then it was incubated with 1 μL TURBO DNase (supplied in kit) for 15 minutes at 37° C. Transcribed RNA was purified by the MEGAclear Transcription Clean-Up Kit (Invitrogen) following the manufacturer's instructions and eluting in 40 μL H2O. The RNA was diluted to 2 μM and aliquoted to limit freeze/thaw cycles. Transcribed RNA was analyzed by gel electrophoresis to verify a single band of correct size.

### Fluorescence Polarization Assay

To determine the binding affinity of a protein with RNA, we conducted the fluorescence polarization assay as previously described with some minor modifications18 (Holmes et al 2020)., The concentration of protein is serially diluted from 5000 nM down to 2 nM by a 3-fold dilution factor. The series of protein concentrations is then mixed with a buffer containing 10 nM Cy5-labeled RNA, 10 mM Tris pH 7.5, 8% Ficoll PM70 (Sigma F2878), 0.05% NP-40 (Sigma), 150 mM NaCl, 1 mM DTT, 0.1 mg/mL non-acetylated BSA (Invitrogen AM2616), and 1 μM ZnCl2. The reactions were performed in triplicates in a 20 L reaction volume. After incubating the reactions 1 hr at room temperature, they are transferred into flat bottom black 384 well-plate (Corning 3575). Anisotropy was measured by a Tecan i-control infinite M1000 with the following parameters. Excitation Wavelength: 635 nm; Emission Wavelength: 665; Excitation/Emission Bandwidth: 5 nm; Gain: Auto; Number of Flashes: 20; Settle Time: 200 ms; G-Factor: 1. To account for instrument error, the plate was measured 3 times and the mean of the values are used in the affinity calculations. Reagents used for established RNA-binding proteins were generated previously90 and BamHI was purchased from New England Biolabs.

To determine the binding affinity of a protein with DNA, the same buffer conditions and incubation times were used, as described above. The series of protein concentrations from 0.76-1666 nM (3-fold serial dilution) and 10 nM cy5-labeled DNA were used. The motif containing DNA sequences that have been shown to bind SOX218 and KLF491 were ordered from IDT. To prepare motif-containing DNA sequences, 5 μM of oligos with complementary sequences (one unlabeled and the other labeled with cy5) (Table 3) were annealed in TE+100 mM NaCl buffer by ramping down the temperature from 98° C. to 4° C. on a thermocycler. Then the annealed DNA fragments were diluted to appropriate concentrations with water for the assay.

Binding curves were fit to fluorescence anisotropy data via nonlinear regression with the Levenberg-Marquardt-based ‘curve_fit’ function in scipy (v. 1.7.3). Curve fitting was performed using a monovalent reversible equilibrium binding model accounting for ligand depletion, given by the equation below:

$$A = {A}_{0} + ( {A}_{1} - {A}_{0} ) [ \frac{{P}_{0} + {L}_{0} + {K}_{d} - \sqrt{{( {P}_{0} + {L}_{0} + {K}_{d} )}^{2} - 4 {P}_{0} {L}_{0}}}{2 {L}_{0}} ]$$

where P0 is the total protein concentration, L0 is the total ligand (RNA) concentration, and A0, A1, and Kd are fit parameters. The measured anisotropy value A for each condition was determined by first averaging raw anisotropy measurements across three subsequent reads of the same well, then averaging these values across three technical replicates from separate wells. To calculate the bound fraction of RNA, A values were normalized to the range between the upper and lower anisotropy asymptotes A0 and A1. Error bars were computed from the standard deviation of RNA bound fraction across three technical replicates. The script used to calculate the affinities are available on GitHub (https://github.com/uberholzer/2022_Oksuz_et_al_TF_RNA).

### Electrophoretic Mobility Shift Assay

To determine the binding affinity of a TF-ARM peptides (synthesized by Genscript) (Table 3) with 7SK RNA, we conducted the electrophoretic mobility shift assay as previously described with some minor modifications19,36. The concentration of peptides was serially diluted from 50000 nM down to 3.125 nM by a 2-fold dilution factor in buffer containing 20 mM HEPES, 300 mM NaCl, and 10% Glycerol. The series of protein concentrations was then mixed 1:1 with a buffer containing an initial concentration of 20 nM Cy5-labeled RNA, 20 mM Tris pH 8.0, 5% glycerol, 0.1% NP40 (Sigma), 0.02 mM ZnCl2, 1 mM MgCl2, 2 mM DTT, and 0.2 mg/mL nonacetylated BSA (Invitrogen AM2616). For DNA-binding assays, 20 nM Cy5-labeled dsDNA or 20 nM Cy5-labeled ssRNA were used (Table 3). The reactions were performed in a 20 μL reaction volume. After incubating the reactions in the dark for 1 hr at room temperature, they were loaded into a 2.5% agarose gel that is pre-run for at least 30 min at 4° C. The samples then ran for 1.5 hr at 150V at 4° C. The gel is imaged using Typhoon FLA95 imager with a Cy5 fluorescence module.

### Homology Search for RNA-Binding Domains in TFs

We retrieved hidden Markov model based profiles (HMM-profiles) for RNA-binding domains corresponding to the following Pfam92 entries using hmmfetch from the HMMER package (hmmer.org)—RRM_1, RRM_2, RRM_3, RRM_5, RRM_7, RRM_8, RRM_9, DEAD, zf-CCCH, zf-CCCH 2, zf-CCCH_3, zf-CCCH 4, zf-CCCH 6, zf-CCCH_7, zf-CCCH_8, KH_1, KH_2, KH_4, KH_5, KH_6, KH_7, KH_8, KH_9. These domains represent the largest families of RNA-binding domains. We searched for these profiles using hmmsearch form the HMMER package with ‘-T 0’ as a parameter in fasta files with sequences corresponding to TFs1 or RNA-binding proteins93. The log 2-odds ratio score from the hmmsearch output was plotted for RBPs with score >0 (n=350, to provide scores that one would expect if these domains were in the protein) and for all 1651 TFs1. If a TF was not in the output, it was assigned a score of 0.

### Analysis of ARM-Like Regions in TFs

We used an approach based on analogous functions in localCIDER94 and on a previously applied procedure95 used to map basic patches. For each TF, amino acid compositions of Lys and Arg in sliding 5-residue windows were computed. Basic patches were defined as regions of ≥5 consecutive residues that consisted of Lys and Arg occurring at a frequency of >0.5. This threshold was based on optimizing this approach against previously described basic patches in MECP295. All identified basic patches were filtered for those that occurred within predicted IDRs (metapredict), determined as described above. For the adjacency analysis, DNA-binding domains were defined based on domains with annotations of DNA-binding in Interpro96. Probabilities of basic patch occurrence in all TFs were computed starting from the N-terminal edge of the first DNA-binding domain and moving N-terminally, or the C-terminal edge of the last DNA-binding domain and moving C-terminally. These probabilities were summed to arrive at the total probability as a function of distance from the bounds of the DNA-binding regions.

A consensus motif for bioinformatically identified basic patches (FIG. 3B) was created using MEME (v. 4.11.4)97. Briefly, 963 basic patches found in TFs were padded by appending the 10 amino acid residues upstream and downstream of each the region. Next, a zero-order Markov model was created from 1,290 full sequences of annotated TFs using the ‘fasta_get_markov’ function to generate a background for the motif search. The TF basic patch sequences were input to the ‘MEME’ function using the TF background model, specifying a 890 constraint to identify exactly one site per sequence, a minimum motif width of 5, a maximum motif width of 13, and defaults for the unspecified parameters.

A charge-based cross-correlation method was employed to identify ARMs in TF disordered regions similar to the HIV Tat ARM. Extensive in vitro and cellular analyses of the Tat ARM have mapped the critical residues responsible for Tat RNA-binding and HIV transactivation^{41,42}. To properly function, the Tat ARM requires an arginine positioned near the motif center flanked by an enrichment of basic residues (R/K). The Tat ARM sequence “RKKRRQRRR” (SEQ ID NO: 5) was digitized to the amino acid charge pattern “11110111” to create a 9-mer search kernel. A protein target sequence was created by first digitizing the sequence of the protein of interest to “1” for R/K amino acid residues and “0” otherwise, then refining the sequence by setting residues to “0” if they fell outside of disordered regions assessed through the metapredict package⁹⁸ (v. 2.2) with a disorder threshold of 0.2. The target sequence was further refined by setting all entries to “0” in 9-mer windows where no R's were originally present. The cross correlation between the search kernel and the target sequence was then computed using the ‘correlate’ function in scipy using the “direct” method. Maximum cross-correlations were computed as the maximum of the returned array for each protein tested. This method was applied iteratively to all sequences from the UniProt database to generate distributions for TFs and the proteome.

### Evolutionary Conservation of TF-ARMs

Evolutionary conservation of specific human TFs was assessed using the ConSurf online server99. TF sequences were downloaded from UniProt and run without specifying a 3D structure or MSA, with automatic detection of homologs from the “NR_PROT_DB” database. Defaults were used for all other running parameters. Amino acid conservation scores from the ConSurf GRADES output were re-normalized between 0 and 1 for each protein, such that a score of 1 corresponded to the of the most conserved amino acid in a given protein.

To evaluate the extent of evolutionary conservation for a larger cohort of TF ARMs, the degree of conservation of TF ARMs was compared to non-ARM regions across vertebrates. The OrthoDB v10 database was used to identify the set of vertebrate orthologs for each protein in a list of annotated human TFs. For each TF, a multiple sequence alignment (MSA) of the retrieved vertebrate orthologs was generated using Clustal Omega (v. 1.2.4) with default parameters. The output ALN format MSA files were converted directly to FASTA format. TFs with an ARM maximum cross-correlation score of 5 or above were retained for further analysis. Each MSA file was parsed via the “prody” package (v. 2.3.1)¹⁰⁰ in Python using the ‘parseMSA’ command. Reference coordinates for the MSA were set with respect to the human TF of interest by using the ‘refineMSA’ command and specifying the ID of the human TF. The degree of conservation of each amino acid residue in the human TF was quantified by computing the Shannon entropy (H) for each residue via the ‘calcShannonEntropy’ function. Higher values of H represent more sequence variation at a specific residue position and therefore a lower degree of evolutionary conservation. To define ARM regions for the purpose of Shannon entropy analysis, the union of 9-mer regions with an ARM cross-correlation score of 5 or above was used. For each TF analyzed (N=580), the median value of H in the ARM region and the median value of H in the remainder of the sequence (non-ARM region) were calculated and plotted. Distributions of these paired data were compared via a Wilcoxon signed-rank test.

### HIV Tat Transactivation Assay

To generate the HIV LTR luciferase reporter, the HIV 5′ LTR from the pNL4-3 isolate (Genbank AF324493) was cloned into pGL3-Basic (Promega) via Gibson assembly (NEB 2×HiFi) with a HindIII-digested pGL3-Basic and a gBlock (Integrated DNA Technologies) containing the HIV 5′ LTR with compatible overhangs (Table 3). A mutant version of this reporter lacking the Tat activation site (TAR RNA bulge structure)⁴⁴ was also generated in a similar fashion. Mammalian expression vectors encoding Tat, an R/K>A mutant of Tat, and replacements of the Tat ARM with TF-ARMs from KLF4, SOX2, GATA2, and ESR1 were generated by Gibson assembly with a NotI-XhoI-digested pcDNA3 (Invitrogen) and gBlocks encoding these variants with compatible overhangs (Table 3).

For transfections, HEK293T cells were cultured in DMEM (Gibco) supplemented with 10% fetal bovine serum (Sigma F4135), 50 U/mL penicillin and 50 g/mL streptomycin (Life Technologies 15140163). Transfections were conducted in triplicate. 24-well plastic plates were first coated with poly-L-lysine (Sigma) for 30 minutes at 37° C., washed once with 1×PBS, and then allowed to air dry. Cells were seeded in 500 μL of media in coated wells at a density of 2×10⁵ cells per well. The next day, each well was transfected using Lipofectamine 3000 (Life Technologies) (total reaction 50 μL Optimem, 1.5 μL Lipo-3000, 0.6 μL P3000, and the appropriate volume of DNA) with 100 ng of the HIV 5′ LTR reporter vector, 150 ng of the pcDNA3 expression vector (encoding Tat or the variants), and 50 ng of a renilla luciferase plasmid (pRL-SV40, Promega) to normalize transfection efficiency. As a control, we included a pcDNA3 vector expressing LacImCherry (labeled as “No Tat” in FIG. 3). After 6 hours of incubation, luciferase activity was quantified by the Dual Luciferase Assay kit (Promega) following the manufacturer's instructions and a Safire II plate reader. The luminescence values were first normalized to the renilla luciferase luminescence for each well, and then all conditions were normalized to the average value of the “No Tat” control condition.

### CUT&Tag Experimental Procedure

CUT&Tag sequencing was performed using the CUT&Tag-IT Assay Kit (Active Motif 53160) according to manufacturer's instructions. Stable mESC lines expressing HA-tagged versions of WT and ARM-mutant SOX2 and KLF4 were induced with doxycycline (1 g/mL) for 6 hours, and 4×105 mESCs were collected. The nuclei of the cells were extracted and incubated with 1 g of HA antibody (Abcam ab9110). After incubation with a rabbit secondary antibody and pA-Tn5 Transposomes, DNA was extracted and amplified with i7/i5 indexed primer combinations. SPRI Bead clean-up of the amplified DNA fragments were performed, and libraries were pooled, subjected to gel-based clean up and sequenced by Novaseq (50×50).

### CUT&Tag Analysis

Reads were first trimmed by adapter sequence (CTGTCTCTTATACACATCT (SEQ ID NO: 6)) in the forward and reverse directions using Cutadapt with default parameters. Subsequent analysis of the data was conducted according to a published protocol with no modification101. Reads were aligned to the mm10 mouse genome, and samples were spike-in normalized according to the protocol by calculating a scale factor from reads aligning to the E. coli genome. Peak calling for both WT and ARM-mutant samples was conducted using the Seacr algorithm using the “non” (nonnormalized) and “stringent” parameters102. For meta-gene plots, raw read density was calculated by centering on called peaks for both WT and ARM-mutant TFs that were merged using bedTools merge with default parameters.

### TF Reporter Assays

For KLF4 reporter assays, constructs were designed that replaced the 3 zinc fingers of KLF4 with either the yeast GAL4 DNA-binding domain or the bacterial TetR DNA-binding domain. Plasmids were cloned via Gibson assembly with gBlocks (IDT) encoding wildtype, mutant, or Tat-ARM-swap versions of KLF4, and expression of the KLF4 fusions were driven by the human UbiC promoter. Reporter constructs contained either 6×UAS sites or 4×TetO sites upstream of a minimal CMV promoter driving firefly luciferase. For GAL4 experiments, HEK293 cells were plated at 2×10⁵ cells per well in a 24-well plate in triplicate. Cells were transfected with 100 ng reporter, 166 ng KLF4 expression construct, and 50 ng of a renilla luciferase transfection control (pRL-SV40, Promega) the following day using Lipofectamine 3000 following the manufacturer's instructions. As a control, we included a pcDNA3 vector expressing LacI-mCherry (labeled as “No TF”). After 4 hours of incubation, luciferase activity was quantified by the Dual Luciferase Assay Kit (Promega) following the manufacturer's instructions and a Safire II plate reader. The luminescence values were first normalized to the renilla luciferase luminescence for each well, and then all conditions were normalized to the average value of the “No TF” control condition. For TetR assays, HEK293 cells were plated at 1×105 cells per well in a 24-well plate in triplicate in media containing tetracycline-free serum. The following day, cells were transfected with 100 ng reporter, 100 ng KLF4 expression construct, and 50 ng of renilla luciferase. After 2 hours of incubation, the media was removed and replaced with a media containing 1 μg/mL doxycycline. After 4 hours in dox, the cells were processed for luminescence readings in an identical fashion to the GAL4 assays.

### Single-Molecule Tracking

### Cell Line Generation

Murine embryonic stem cells were cultured in 2i/LIF media on tissue culture plates coated with 0.2% gelatin (Sigma, G1890). The 2i/LIF media contained: 960 mL DMEM/F12 (Life Technologies, 11320082), 5 mL N2 supplement (Life Technologies, 17502048; stock 100×), 10 mL B27 supplement (Life Technologies, 17504044; stock 50×), 5 mL additional L-glutamine (GIBCO 25030-081; stock 200 mM), 10 mL MEM nonessential amino acids (GIBCO 11140076; stock 100×), 10 mL penicillin-streptomycin (Life Technologies, 15140163; stock 10\hat{ }4 U/mL), 333 mL BSA fraction V (GIBCO 15260037; stock 7.50%), 7 mL b-mercaptoethanol (Sigma M6250; stock 14.3 M), 100 mL LIF (Chemico, ESG1107; stock 10\hat{ }7 U/mL), 100 mL PD0325901 (Stemgent, 04-0006-10; stock 10 mM), and 300 mL CHIR99021 (Stemgent, 04-0004-10; stock 10 mM). Cells were passaged by washing once with 1×PBS (Life Technologies, AM9625) and incubating with TrypLE (Life Technologies, 12604021) for 3-5 minutes, then quenched with serum-containing media made by the following recipe: 500 mL DMEM KO (GIBCO 10829-018), MEM nonessential amino acids (GIBCO 11140076; stock 100×), penicillin-streptomycin (Life Technologies, 15140163; stock 10\hat{ }4 U/mL), 5 mL L-glutamine (GIBCO 25030-081; stock 100×), 4 mL b-mercaptoethanol (Sigma M6250; stock 14.3 M), 50 mL LIF (Chemico, ESG1107; stock 10\hat{ }7 U/mL), and 75 mL of fetal bovine serum (Sigma, F4135). Cells were passaged every 2 days.

A piggyBac compatible base vector was assembled containing two tandem gene cassettes: (1) an insertion site downstream of a doxycycline-inducible promoter allowing for the expression of a Flag-HA-Halo-tagged ORF with SV40 NLS and bGH polyA termination sequence, and (2) the Tet-On 3G rtta element driven by the EF1a promoter that also produces hygromycin resistance via a 2A self-cleaving peptide. This base vector was generated by Gibson assembly. Plasmids encoding Halo-tagged versions of TFs (WT and ARM-deletion) were generated by Gibson assembly with BamHI-digested base vector and gBlocks (Integrated DNA Technologies) encoding the WT and ARM-deletion TFs.

To generate cell lines, 5×106 mESCs per well were transfected in 6-well plates with 1 μg of the Halo-TF vector and 1 g of the piggyBac transposase (Systems Biosciences) in serum containing media (described above) using Lipofectamine-3000 for at least 4 hours. After transfection, the cells were passaged into 10 cm plates in 2i media containing 500 ng/mL Hygromycin-B (Gibco 10687010). After 2-4 days of selection, cells were maintained as described above.

### Sample Preparation

Cells were plated on glass bottom dishes (Cellvis D35-20-1.5-N) coated with 5 μg/ml of poly-Lornithine (Sigma-Aldrich P4957) for 2 hrs min at 37° C. and with 5 μg/ml of Laminin (Corning® 354232) for 2 hrs-24 hrs at 37° C., growing from 20% confluency in 2i for one day. Doxycycline=10 ng/mL was added to dishes for 1 hr, followed by adding 5 nM of HaloTag-(PA) JF549 for another 3 hrs. Cells were then rinsed once with PBS and washed in fresh 2i for hr. Dishes were refilled with 2 mL prewarmed Leibovitz's L-15 Medium, no phenol red (ThermoFisher 21083027) and brought for imaging.

### Imaging

Cells were imaged on an inverted, widefield setup with a Nikon Eclipse Ti microscope and a 100× oil immersion objective as previously described⁵⁸. Images were acquired with an EMCCD camera (EM gain 1000, exposure time 10 ms, conjugated pixel-size on sample 160 nm). A 561 nm laser beam of 150 mW (attenuated with 50% AOTF) was 2× expanded for a uniform illumination across around 200×200 pixel region. 10,000 frames were recorded for each ROI (including 2-4 cells), and the 405 nm activation was kept very low to guarantee the molecule sparsity needed for robust reconnection.

### Analyses

Particle trajectories were detected and reconnected with customized MATLAB code from MTT103. Detection settings: false-positive threshold=24, window-size 7×7pixel, and Gaussian width fitting allowed. Reconnection settings: Toff=10 ms, Tcut=20 ms, and rmax=270 nm. A collection of trajectories from each ROI were fitted to a 3-state model in Spot-on104. Spot-on settings: detection slice dZ=950 nm, 8 delays to consider, and only first 10 jumps to consider for each trajectory. The final outputs include fractions and apparent diffusion coefficients of each state (immobile, sub-diffusive, and free, respectively). For expression dependence testing in FIG. 12B, trajectories of the same genotype from different nuclei with similar trajectory density were gathered together first and resampled ten times (2,000 trajectories for each resampling) for ten independent Spot-on fittings, respectively. In this way, the accuracy of each fitting and the distributions across different conditions are comparable.

For dwell time analyses in FIG. 14C, sparse detections from slow tracking mode were generated with the same MTT settings as for those in the fast tracking. The detections were then grouped to different spatial clusters by running a Density-based spatial clustering of applications with noise (DBSCAN) with short radius. Within each spatial cluster, the time-correlated detections were further grouped into the same trajectory (two dark frames at maximum). In this manner, only immobile (i.e., bound) trajectories will be collected, whose duration (tₗₐₛₜ−t_{first}) were the apparent dwelling time. The survival probabilities of apparent dwelling time distributions were fitted to a biexponential model for both fixed and live cell samples, where a short dwelling time scale and a long dwelling time scale were fitted. The stable dwell time of each live cell sample was based on the long dwelling time scale, which was calibrated by the long dwelling time scale of a fixed sample with the exact imaging condition as following:

$$\frac{1}{{\hat{τ}}_{cali}} = \frac{1}{{τ}_{live}} - \frac{1}{{τ}_{fix}} ,$$

where τₗᵢᵥₑ is the “apparent” long dwelling time scale of the live sample, τ_{fix} is the “apparent” long dwelling time scale of a fixed sample on the same date in the same imaging buffer, and τ_{cali} is the calibrated stable dwell time actually reported in final figures.

### Sub-Nuclear Fractionation

mESCs with exogenous expression for SOX2 and KLF4 wild type and ARM deletion mutations expressing HA tag were used for nuclei sub fractionation. To extract nuclei, cells were resuspended in 10 ml HMSD50 buffer (20 mM HEPES pH 7.5, 5 mM MgCl2, 250 mM sucrose, 1 mM DTT, 50 mM NaCl, supplemented with 0.2 mM PMSF and 5 mM sodium butyrate) and incubated for 30 min at 4° C. with gentle agitation. After a spin down at 3500 rpm at 4° C. for 10 min, the supernatant was discarded and the pellet containing nuclei were subjected to subcellular protein fractionation for nucleoplasm and chromatin fractions using the Subcellular Protein Fractionation Kit for Cultured Cells (ThermoScientific, Ref 78840) according to manufacturer's instructions. For RNase treatment in wild type mESCs, nuclei were treated with RNase A (1:100, Thermo Fisher EN0531) and the initial 30-minute incubation at 4° C. was adjusted to 20 minutes at 4° C. and 10 minutes at 37° C. The pH of the buffer remained the same (˜7.5) after RNase A treatment. SDS Page was run on 12% Bis-Tris gel (Criterion XT, BioRad) and western blotting was performed on the subfractions using anti Histone H3 antibody from Abcam (ab1791) and anti HA antibody from Abcam (ab9110) with secondary antibody against Rabbit (TRDye 800CW Goat anti-rabbit LI-COR 926-32211). For wild type transcription factor detection, antibody for Sox2 (R&D Systems, MAB2018) and Klf4 (R&D Systems, AF3158) with secondary antibody anti-mouse for Sox2 (TRDye 680CW goat anti-mouse LI-COR 926-32211) and anti-goat for Klf4 (IRDye 800CW donkey anti-goat LI-COR 926-32214), were used. Fluorescence was assessed using Odyssey CLX LiCOR and quantified using ImageJ.

Zebrafish Knockdown and Rescue of sox2

Morpholinos (MO, GeneTools) were resuspended in nuclease free water, heated to 65° C. for 5 minutes, and stored at room temperature. Wildtype AB zebrafish embryos were injected into the yolk at the 1-cell stage with 7 ng of sox2-MO (TCTTGAAAGTCTACCCCACCAGCCG (SEQ ID NO: 7))⁵³, either alone or in combination with 25 μg of human wildtype or ARM-deletion SOX2 mRNA. Messenger RNA was synthesized using the T7 mMessage mMachine (Invitrogen) kit with templates generated from gBlocks (IDT). The mRNA was purified with the MEGAclear Clean-Up Kit (Invitrogen), run on a TBE agarose gel to confirm purity and size, aliquoted, and stored at −80° C. Embryos injected with 7 ng of Standard Control MO (CCTCTTACCTCAGTTACAATTTATA (SEQ ID NO: 8)) were used as controls. At 48 hours post fertilization (hpf), MO injected embryos were dechorionated using forceps, anaesthetized using 0.16 mg/ml Tricaine, then visually assessed for growth impairment using a Nikon SMZ18 stereoscope with DS-Ri2 camera and NIS-Elements software. Embryos were scored based on rescue of growth impairment in the presence of wildtype or mutant sox2 mRNA.

To assure that mutant SOX2 was expressed as protein, we conducted Western blots (FIG. 14C). Protein extraction for zebrafish embryos (n=20 per tube) that were uninjected or injected with mRNA encoding HA-tagged ARM-mutant SOX2 was performed with Urea Chaps lysis buffer. Cells were resuspended in Urea Chaps (1% Chaps, 8M Urea, 50 mM Tris-Cl pH 7.5 containing protease inhibitors (Thermo Fisher)) and incubated for 30′ at 4° C. with gentle agitation. After a spin down at 14,000 rpm for 10′ at 4° C., the supernatant was used for SDSPage. SDS-Page was run on a 10% Bis-Tris (Criterion XT, BioRad) and western blotting was performed on uninjected and injected samples using anti HA antibody from Abcam (ab9110) and anti beta actin (Sigma A5441) with secondary antibody against Rabbit (TRDye 800CW Goat anti-rabbit LI-COR 926-32211 and IRDye 680RD Goat anti-mouse 926-68070). Fluorescence was assessed using Odyssey CLX LiCOR.

### Overlap of Pathogenic Mutations in TF-ARMs

Pathogenic nonsynonmous substitution mutations were obtained from a prior dataset of pathogenic mutations that integrated multiple databases of somatic and germline variation associated with cancer and Mendelian disorders, including ClinVar (accessed Jan. 29, 2021) and HGMD v2020.4 in hg38. Cancer variants were obtained from AACR Project GENIE v8.1 (AACR Project GENIE Consortium, 2017) and various TCGA and TARGET studies via cBioPortal105. Mutations were subsetted for those affecting TF-ARMs. For mutation frequency analysis, the expected mutation frequency for each amino acid type within TF-ARMs was estimated using the average nucleotide substitution rates within the entire mutation dataset and the frequency of nucleotide types encoding each amino acid type within TF-ARMs. It is important to note that this analysis does not take into account disease-specific mutational signatures, which could introduce potential biases. Enrichment was defined as a significantly higher pathogenic mutation frequency compared to the aforementioned expected amino acid mutation frequency. Statistical significance of the enrichment was determined using a one-sided binomial test, and p-values were corrected for the multiple tests across the twenty amino acids using the Benjamini-Hochberg method.

### Statistical Information

Confidence intervals for Kd estimates from fluorescence polarization data were computed by multiplying the standard deviation of the Kd curve fit parameter with the Student's t-value corresponding to the 95% confidence interval with degrees of freedom equal to the number of data points in the concentration curve minus the number of fit parameters. Statistical comparisons between the Kd's of two fluorescence polarization curves (for FIGS. 3E, 9C, and 11A-D) were assessed using a two-tailed Student's t-test based on the standard errors of the Kd parameters calculated from the diagonals of the covariance matrix returned by ‘curve_fit’ in scipy, with the degrees of freedom as specified above.

The distributions of ARM correlation scores (FIG. 3C) for whole proteome (−TFs) vs TFs were compared using a two-tailed Mann Whitney U test, n1=1287, n2=20238.

The Tat reporter assays were conducted on 3 biological replicates per genotype, and luminescence readings were measured in technical duplicates. Each condition was compared to the Tat R/K>A condition using a Sidak multiple comparisons test (DF=24, t statistics were as follow: TAR-WT—WT=20.15, KLF4=15.3, SOX2=13.17, GATA2=3.805, NoTat=6.419; ΔTARbulge—WT=9.263, KLF4=9.319, SOX2=9.329, GATA2=9.315, Tat R/K>A=9.302, No-Tat=9.364).

For comparison of the diffusive fractions reported in FIG. 4C, multiple fields of cells were imaged per genotype (KLF4-WT n=11, KLF4-ΔARM n=9, SOX2-WT n=10, SOX2-ΔARM n=9, CTCF-WT n=7, CTCF-ΔARM n=7). The diffusive fractions were compared by 2-tailed Student t-test. The data was confirmed to have equal variance via F test, and the degrees of freedom and t statistics were as follows: KLF4-free (t=13.47, df=18), SOX2-free (t=8.297, df=18), CTCF-free (t=6.044, df=12), KLF4-sub (t=5.152, df=18), SOX2-sub (2.908, df=18), CTCF-sub (t=3.051, df=12), KLF4-imm (t=7.824, df=18), SOX2-imm (t=6.203, df=18), CTCF-imm (t=3.639, df=12).

TABLE 1

| EnsemblID_{—} | Gene_{—} | | Gene_{—} | | |
|---|---|---|---|---|---|
| Human | Human | EnsemblID_Mouse | Mouse | Gene | DBD |
| ENSG00000101126 | ADNP | ENSMUSG00000051149 | Adnp | ADNP | Homeodomain |
| ENSG00000101544 | ADNP2 | ENSMUSG00000053950 | Adnp2 | ADNP2 | Homeodomain |
| ENSG00000139154 | AEBP2 | ENSMUSG00000030232 | Aebp2 | AEBP2 | C2H2 ZF |
| ENSG00000153207 | AHCTF1 | ENSMUSG00000026491 | Ahctf1 | AHCTF1 | AT hook |
| ENSG00000126705 | AHDC1 | ENSMUSG00000037692 | Ahdc1 | AHDC1 | AT hook |
| ENSG00000105127 | AKAP8 | ENSMUSG00000024045 | Akap8 | AKAP8 | C2H2 ZF |
| ENSG00000011243 | AKAP8L | ENSMUSG00000002625 | Akap8l | AKAP8L | C2H2 ZF |
| ENSG00000163516 | ANKZF1 | ENSMUSG00000026199 | Ankzf1 | ANKZF1 | C2H2 ZF |
| ENSG00000189079 | ARID2 | ENSMUSG00000033237 | Arid2 | ARID2 | ARID/BRIGHT; |
| | | | | | RFX |
| ENSG00000179361 | ARID3B | ENSMUSG00000004661 | Arid3b | ARID3B | ARID/BRIGHT |
| ENSG00000150347 | ARID5B | ENSMUSG00000019947 | Arid5b | ARID5B | ARID/BRIGHT |
| ENSG00000123268 | ATF1 | ENSMUSG00000023027 | Atf1 | ATF1 | bZIP |
| ENSG00000115966 | ATF2 | ENSMUSG00000027104 | Atf2 | ATF2 | bZIP |
| ENSG00000170653 | ATF7 | Not Found | Not Found | ATF7 | bZIP |
| ENSG00000156273 | BACH1 | ENSMUSG00000025612 | Bach1 | BACH1 | bZIP |
| ENSG00000076108 | BAZ2A | Not Found | Not Found | BAZ2A | MBD; AT hook |
| ENSG00000123636 | BAZ2B | ENSMUSG00000026987 | Baz2b | BAZ2B | MBD |
| ENSG00000134107 | BHLHE40 | ENSMUSG00000030103 | Bhlhe40 | BHLHE40 | bHLH |
| ENSG00000171634 | BPTF | ENSMUSG00000040481 | Bptf | BPTF | Unknown |
| ENSG00000173894 | CBX2 | ENSMUSG00000025577 | Cbx2 | CBX2 | AT hook |
| ENSG00000132024 | CC2D1A | ENSMUSG00000036686 | Cc2d1a | CC2D1A | Unknown |
| ENSG00000096401 | CDC5L | ENSMUSG00000023932 | Cdc5l | CDC5L | Myb/SANT |
| ENSG00000096401 | CDC5L | ENSMUSG00000112252 | Gm32802 | CDC5L | Myb/SANT |
| ENSG00000096401 | CDC5L | ENSMUSG00000112027 | Gm9045 | CDC5L | Myb/SANT |
| ENSG00000096401 | CDC5L | ENSMUSG00000112919 | Gm9049 | CDC5L | Myb/SANT |
| ENSG00000096401 | CDC5L | ENSMUSG00000112495 | Gm9048 | CDC5L | Myb/SANT |
| ENSG00000096401 | CDC5L | ENSMUSG00000112419 | Gm9044 | CDC5L | Myb/SANT |
| ENSG00000096401 | CDC5L | ENSMUSG00000112781 | Gm9046 | CDC5L | Myb/SANT |
| ENSG00000096401 | CDC5L | ENSMUSG00000112814 | Gm9040 | CDC5L | Myb/SANT |
| ENSG00000096401 | CDC5L | ENSMUSG00000112216 | Gm32717 | CDC5L | Myb/SANT |
| ENSG00000168564 | CDKN2AIP | ENSMUSG00000038069 | Cdkn2aip | NA | NA |
| ENSG00000172216 | CEBPB | ENSMUSG00000056501 | Cebpb | CEBPB | bZIP |
| ENSG00000115816 | CEBPZ | ENSMUSG00000024081 | Cebpz | CEBPZ | Unknown |
| ENSG00000125817 | CENPB | ENSMUSG00000068267 | Cenpb | CENPB | CENPB |
| ENSG00000175279 | CENPS | ENSMUSG00000073705 | Cenps | CENPS | Unknown |
| ENSG00000102901 | CENPT | ENSMUSG00000036672 | Cenpt | CENPT | Unknown |
| ENSG00000169689 | CENPX | Not Found | Not Found | CENPX | Unknown |
| ENSG00000163320 | CGGBP1 | ENSMUSG00000054604 | Cggbp1 | CGGBP1 | Unknown |
| ENSG00000198824 | CHAMP1 | ENSMUSG00000047710 | Champ1 | CHAMP1 | C2H2 ZF |
| ENSG00000106554 | CHCHD3 | ENSMUSG00000053768 | Chchd3 | CHCHD3 | Unknown |
| ENSG00000079432 | CIC | ENSMUSG00000005442 | Cic | CIC | HMG/Sox |
| ENSG00000118260 | CREB1 | ENSMUSG00000025958 | Creb1 | CREB1 | bZIP |
| ENSG00000102974 | CTCF | ENSMUSG00000005698 | Ctcf | CTCF | C2H2 ZF |
| ENSG00000257923 | CUX1 | ENSMUSG00000029705 | Cux1 | CUX1 | CUT; |
| | | | | | Homeodomain |
| ENSG00000257923 | CUX1 | ENSMUSG00000029705 | Cux1 | CUX1 | CUT; |
| | | | | | Homeodomain |
| ENSG00000154832 | CXXC1 | ENSMUSG00000024560 | Cxxc1 | CXXC1 | CxxC |
| ENSG00000171604 | CXXC5 | ENSMUSG00000046668 | Cxxc5 | CXXC5 | CxxC |
| ENSG00000130816 | DNMT1 | ENSMUSG00000004099 | Dnmt1 | DNMT1 | CxxC |
| ENSG00000104885 | DOT1L | ENSMUSG00000061589 | Dot1l | DOT1L | AT hook |
| ENSG00000112242 | E2F3 | ENSMUSG00000016477 | E2f3 | E2F3 | E2F |
| ENSG00000205250 | E2F4 | ENSMUSG00000014859 | E2f4 | E2F4 | E2F |
| ENSG00000169016 | E2F6 | ENSMUSG00000057469 | E2f6 | E2F6 | E2F |
| ENSG00000167967 | E4F1 | ENSMUSG00000024137 | E4f1 | E4F1 | C2H2 ZF |
| ENSG00000102189 | EEA1 | ENSMUSG00000036499 | Eea1 | EEA1 | C2H2 ZF |
| ENSG00000120690 | ELF1 | ENSMUSG00000036461 | Elf1 | ELF1 | Ets |
| ENSG00000109381 | ELF2 | ENSMUSG00000037174 | Elf2 | ELF2 | Ets |
| ENSG00000091831 | ESR1 | ENSMUSG00000019768 | Esr1 | ESR1 | Nuclear |
| | | | | | receptor |
| ENSG00000059122 | FLYWCH1 | ENSMUSG00000040097 | Flywch1 | FLYWCH1 | FLYWCH |
| ENSG00000175592 | FOSL1 | ENSMUSG00000024912 | Fosl1 | FOSL1 | bZIP |
| ENSG00000164916 | FOXK1 | ENSMUSG00000056493 | Foxk1 | FOXK1 | Forkhead |
| ENSG00000141568 | FOXK2 | ENSMUSG00000039275 | Foxk2 | FOXK2 | Forkhead |
| ENSG00000183770 | FOXL2 | ENSMUSG00000050397 | Foxl2 | FOXL2 | Forkhead |
| ENSG00000114861 | FOXP1 | ENSMUSG00000030067 | Foxp1 | FOXP1 | Forkhead |
| ENSG00000137166 | FOXP4 | ENSMUSG00000023991 | Foxp4 | FOXP4 | Forkhead |
| ENSG00000102145 | GATA1 | ENSMUSG00000031162 | Gata1 | GATA1 | GATA |
| ENSG00000179348 | GATA2 | ENSMUSG00000015053 | Gata2 | GATA2 | GATA |
| ENSG00000107485 | GATA3 | ENSMUSG00000015619 | Gata3 | GATA3 | GATA |
| ENSG00000167491 | GATAD2A | ENSMUSG00000036180 | Gatad2a | GATAD2A | GATA |
| ENSG00000143614 | GATAD2B | ENSMUSG00000042390 | Gatad2b | GATAD2B | GATA |
| ENSG00000165702 | GFI1B | ENSMUSG00000026815 | Gfi1b | GFI1B | C2H2 ZF |
| ENSG00000140632 | GLYR1 | ENSMUSG00000022536 | Glyr1 | GLYR1 | AT hook |
| ENSG00000101216 | GMEB2 | ENSMUSG00000038705 | Gmeb2 | GMEB2 | SAND |
| ENSG00000137947 | GTF2B | ENSMUSG00000028271 | Gtf2b | GTF2B | Unknown |
| ENSG00000263001 | GTF2I | ENSMUSG00000060261 | Gtf2i | GTF2I | GTF2I-like |
| ENSG00000006704 | GTF2IRD1 | ENSMUSG00000023079 | Gtf2ird1 | GTF2IRD1 | GTF2I-like |
| ENSG00000169635 | HIC2 | ENSMUSG00000050240 | Hic2 | HIC2 | C2H2 ZF |
| ENSG00000100644 | HIF1A | ENSMUSG00000021109 | Hif1a | HIF1A | bHLH |
| ENSG00000147421 | HMBOX1 | ENSMUSG00000021972 | Hmbox1 | HMBOX1 | Homeodomain |
| ENSG00000064961 | HMG20B | ENSMUSG00000020232 | Hmg20b | HMG20B | HMG/Sox |
| ENSG00000137309 | HMGA1 | ENSMUSG00000046711 | Hmga1 | HMGA1 | AT hook |
| ENSG00000118418 | HMGN3 | ENSMUSG00000066456 | Hmgn3 | HMGN3 | HMG/Sox |
| ENSG00000215271 | HOMEZ | ENSMUSG00000057156 | Homez | HOMEZ | Homeodomain |
| ENSG00000182742 | HOXB4 | ENSMUSG00000038692 | Hoxb4 | HOXB4 | Homeodomain |
| ENSG00000108511 | HOXB6 | ENSMUSG00000000690 | Hoxb6 | HOXB6 | Homeodomain |
| ENSG00000170689 | HOXB9 | ENSMUSG00000020875 | Hoxb9 | HOXB9 | Homeodomain |
| ENSG00000185811 | IKZF1 | ENSMUSG00000018654 | Ikzf1 | IKZF1 | C2H2 ZF |
| ENSG00000123411 | IKZF4 | Not Found | Not Found | IKZF4 | C2H2 ZF |
| ENSG00000168310 | IRF2 | ENSMUSG00000031627 | Irf2 | IRF2 | IRF |
| ENSG00000177606 | JUN | ENSMUSG00000052684 | Jun | JUN | bZIP |
| ENSG00000171223 | JUNB | ENSMUSG00000052837 | Junb | JUNB | bZIP |
| ENSG00000130522 | JUND | ENSMUSG00000071076 | Jund | JUND | bZIP |
| ENSG00000136504 | KAT7 | ENSMUSG00000038909 | Kat7 | KAT7 | C2H2 ZF |
| ENSG00000173120 | KDM2A | ENSMUSG00000054611 | Kdm2a | KDM2A | CxxC |
| ENSG00000117139 | KDM5B | ENSMUSG00000042207 | Kdm5b | KDM5B | ARID/BRIGHT |
| ENSG00000151657 | KIN | ENSMUSG00000037262 | Kin | KIN | C2H2 ZF |
| ENSG00000127528 | KLF2 | ENSMUSG00000055148 | Klf2 | KLF2 | C2H2 ZF |
| ENSG00000136826 | KLF4 | ENSMUSG00000003032 | Klf4 | KLF4 | C2H2 ZF |
| ENSG00000102554 | KLF5 | ENSMUSG00000005148 | Klf5 | KLF5 | C2H2 ZF |
| ENSG00000102349 | KLF8 | ENSMUSG00000041649 | Klf8 | KLF8 | C2H2 ZF |
| ENSG00000118058 | KMT2A | ENSMUSG00000002028 | Kmt2a | KMT2A | CxxC; AT |
| | | | | | hook |
| ENSG00000272333 | KMT2B | ENSMUSG00000006307 | Kmt2b | KMT2B | CxxC; AT |
| | | | | | hook |
| ENSG00000198945 | L3MBTL3 | ENSMUSG00000039089 | L3mbt13 | L3MBTL3 | C2H2 ZF |
| ENSG00000196233 | LCOR | ENSMUSG00000025019 | Lcor | LCOR | Pipsqueak |
| ENSG00000138795 | LEF1 | ENSMUSG00000027985 | Lef1 | LEF1 | HMG/Sox |
| ENSG00000131914 | LIN28A | ENSMUSG00000050966 | Lin28a | LIN28A | CSD |
| ENSG00000187772 | LIN28B | ENSMUSG00000063804 | Lin28b | LIN28B | CSD |
| ENSG00000189308 | LIN54 | ENSMUSG00000118665 | LIN54 | LIN54 | TCR/CxC |
| ENSG00000125952 | MAX | ENSMUSG00000059436 | Max | MAX | bHLH |
| ENSG00000103495 | MAZ | ENSMUSG00000030678 | Maz | MAZ | C2H2 ZF |
| ENSG00000141644 | MBD1 | ENSMUSG00000024561 | Mbd1 | MBD1 | MBD; CxxC |
| | | | | | ZF |
| ENSG00000134046 | MBD2 | ENSMUSG00000024513 | Mbd2 | MBD2 | MBD |
| ENSG00000071655 | MBD3 | ENSMUSG00000035478 | Mbd3 | MBD3 | MBD |
| ENSG00000129071 | MBD4 | ENSMUSG00000030322 | Mbd4 | MBD4 | MBD |
| ENSG00000139793 | MBNL2 | ENSMUSG00000022139 | Mbnl2 | MBNL2 | CCCH ZF |
| ENSG00000169057 | MECP2 | ENSMUSG00000031393 | Mecp2 | MECP2 | MBD; AT |
| | | | | | hook |
| ENSG00000134138 | MEIS2 | ENSMUSG00000027210 | Meis2 | MEIS2 | Homeodomain |
| ENSG00000174197 | MGA | ENSMUSG00000033943 | Mga | MGA | T-box |
| ENSG00000070444 | MNT | ENSMUSG00000000282 | Mnt | MNT | bHLH |
| ENSG00000127989 | MTERF1 | ENSMUSG00000053178 | Mterf1b | MTERF1 | mTERF |
| ENSG00000127989 | MTERF1 | ENSMUSG00000040429 | Mterf1a | MTERF1 | mTERF |
| ENSG00000156469 | MTERF3 | ENSMUSG00000021519 | Mterf3 | MTERF3 | mTERF |
| ENSG00000122085 | MTERF4 | ENSMUSG00000026273 | Mterf4 | MTERF4 | mTERF |
| ENSG00000118513 | MYB | Not Found | Not Found | MYB | Myb/SANT |
| ENSG00000101057 | MYBL2 | ENSMUSG00000017861 | Mybl2 | MYBL2 | Myb/SANT |
| ENSG00000136997 | MYC | ENSMUSG00000022346 | Myc | MYC | bHLH |
| ENSG00000134323 | MYCN | ENSMUSG00000037169 | Mycn | MYCN | bHLH |
| ENSG00000111704 | NANOG | ENSMUSG00000012396 | Nanog | NANOG | Homeodomain |
| ENSG00000255192 | NANOGP8 | ENSMUSG00000012396 | Nanog | NANOGP8 | Homeodomain |
| ENSG00000123405 | NFE2 | Not Found | Not Found | NFE2 | bZIP |
| ENSG00000162599 | NFIA | ENSMUSG00000028565 | Nfia | NFIA | SMAD |
| ENSG00000141905 | NFIC | ENSMUSG00000055053 | Nfic | NFIC | SMAD |
| ENSG00000109320 | NFKB1 | ENSMUSG00000028163 | Nfkb1 | NFKB1 | Rel |
| ENSG00000077150 | NFKB2 | ENSMUSG00000025225 | Nfkb2 | NFKB2 | Rel |
| ENSG00000086102 | NFX1 | ENSMUSG00000028423 | Nfx1 | NFX1 | NFX |
| ENSG00000170448 | NFXL1 | ENSMUSG00000072889 | Nfxl1 | NFXL1 | NFX |
| ENSG00000001167 | NFYA | ENSMUSG00000023994 | Nfya | NFYA | CBF/NF-Y |
| ENSG00000066136 | NFYC | ENSMUSG00000032897 | Nfyc | NFYC | Unknown |
| ENSG00000186416 | NKRF | ENSMUSG00000044149 | Nkrf | NKRF | Unknown |
| ENSG00000243678 | NME2 | ENSMUSG00000020857 | Nme2 | NME2 | Unknown |
| ENSG00000177463 | NR2C2 | ENSMUSG00000005893 | Nr2c2 | NR2C2 | Nuclear |
| | | | | | receptor |
| ENSG00000185551 | NR2F2 | ENSMUSG00000030551 | Nr2f2 | NR2F2 | Nuclear |
| | | | | | receptor |
| ENSG00000160113 | NR2F6 | ENSMUSG00000002393 | Nr2f6 | NR2F6 | Nuclear |
| | | | | | receptor |
| ENSG00000113580 | NR3C1 | ENSMUSG00000024431 | Nr3c1 | NR3C1 | Nuclear |
| | | | | | receptor |
| ENSG00000116833 | NR5A2 | ENSMUSG00000026398 | Nr5a2 | NR5A2 | Nuclear |
| | | | | | receptor |
| ENSG00000172939 | OXSR1 | ENSMUSG00000036737 | Oxsr1 | NA | NA |
| ENSG00000170515 | PA2G4 | Not Found | Not Found | PA2G4 | Unknown |
| ENSG00000100105 | PATZ1 | ENSMUSG00000020453 | Patz1 | PATZ1 | C2H2 ZF; |
| | | | | | AT hook |
| ENSG00000204304 | PBX2 | ENSMUSG00000034673 | Pbx2 | PBX2 | Homeodomain |
| ENSG00000277258 | PCGF2 | ENSMUSG00000018537 | Pcgf2 | PCGF2 | Unknown |
| ENSG00000156374 | PCGF6 | ENSMUSG00000025050 | Pcgf6 | PCGF6 | Unknown |
| ENSG00000141456 | PELP1 | ENSMUSG00000018921 | Pelp1 | NA | NA |
| ENSG00000112511 | PHF1 | ENSMUSG00000024193 | Phf1 | PHF1 | Unknown |
| ENSG00000025293 | PHF20 | ENSMUSG00000038116 | Phf20 | PHF20 | AT hook |
| ENSG00000135365 | PHF21A | ENSMUSG00000058318 | Phf21a | PHF21A | AT hook |
| ENSG00000127445 | PIN1 | ENSMUSG00000032171 | Pin1 | PIN1 | MBD |
| ENSG00000127445 | PIN1 | ENSMUSG00000074997 | Pin1rt1 | PIN1 | MBD |
| ENSG00000160199 | PKNOX1 | ENSMUSG00000006705 | Pknox1 | PKNOX1 | Homeodomain |
| ENSG00000143190 | POU2F1 | ENSMUSG00000026565 | Pou2f1 | POU2F1 | Homeodomain; |
| | | | | | POU |
| ENSG00000196767 | POU3F4 | ENSMUSG00000056854 | Pou3f4 | POU3F4 | Homeodomain; |
| | | | | | POU |
| ENSG00000204531 | POU5F1 | ENSMUSG00000024406 | Pou5f1 | POU5F1 | Homeodomain; |
| | | | | | POU |
| ENSG00000212993 | POU5F1B | ENSMUSG00000024406 | Pou5f1 | POU5F1B | Homeodomain; |
| | | | | | POU |
| ENSG00000116731 | PRDM2 | ENSMUSG00000057637 | Prdm2 | PRDM2 | C2H2 ZF |
| ENSG00000138073 | PREB | ENSMUSG00000045302 | Preb | PREB | Unknown |
| ENSG00000180228 | PRKRA | ENSMUSG00000002731 | Prkra | NA | NA |
| ENSG00000185238 | PRMT3 | ENSMUSG00000030505 | Prmt3 | PRMT3 | C2H2 ZF |
| ENSG00000126464 | PRR12 | ENSMUSG00000046574 | Prr12 | PRR12 | AT hook |
| ENSG00000185129 | PURA | ENSMUSG00000043991 | Pura | PURA | Unknown |
| ENSG00000146676 | PURB | ENSMUSG00000094483 | Purb | PURB | Unknown |
| ENSG00000172733 | PURG | ENSMUSG00000049184 | Purg | PURG | Unknown |
| ENSG00000172819 | RARG | ENSMUSG00000001288 | Rarg | RARG | Nuclear |
| | | | | | receptor |
| ENSG00000168214 | RBPJ | ENSMUSG00000039191 | Rbpj | RBPJ | CSL |
| ENSG00000214022 | REPIN1 | ENSMUSG00000052751 | Repin1 | REPIN1 | C2H2 ZF |
| ENSG00000084093 | REST | ENSMUSG00000029249 | Rest | REST | C2H2 ZF |
| ENSG00000148300 | REXO4 | ENSMUSG00000052406 | Rexo4 | REXO4 | Unknown |
| ENSG00000132005 | RFX1 | ENSMUSG00000031706 | Rfx1 | RFX1 | RFX |
| ENSG00000117000 | RLF | ENSMUSG00000049878 | Rlf | RLF | C2H2 ZF |
| ENSG00000124782 | RREB1 | ENSMUSG00000039087 | Rreb1 | RREB1 | C2H2 ZF |
| ENSG00000159216 | RUNX1 | ENSMUSG00000022952 | Runx1 | RUNX1 | Runt |
| ENSG00000186350 | RXRA | ENSMUSG00000015846 | Rxra | RXRA | Nuclear |
| | | | | | receptor |
| ENSG00000204231 | RXRB | ENSMUSG00000039656 | Rxrb | RXRB | Nuclear |
| | | | | | receptor |
| ENSG00000160633 | SAFB | ENSMUSG00000071054 | Safb | SAFB | Unknown |
| ENSG00000130254 | SAFB2 | ENSMUSG00000042625 | Safb2 | SAFB2 | Unknown |
| ENSG00000103449 | SALL1 | ENSMUSG00000031665 | Sall1 | SALL1 | C2H2 ZF |
| ENSG00000256463 | SALL3 | ENSMUSG00000024565 | Sall3 | SALL3 | C2H2 ZF |
| ENSG00000119042 | SATB2 | ENSMUSG00000038331 | Satb2 | SATB2 | CUT; |
| | | | | | Homeodomain |
| ENSG00000143379 | SETDB1 | ENSMUSG00000015697 | Setdb1 | SETDB1 | MBD |
| ENSG00000157933 | SKI | ENSMUSG00000029050 | Ski | SKI | Unknown |
| ENSG00000165684 | SNAPC4 | ENSMUSG00000036281 | Snapc4 | SNAPC4 | Myb/SANT |
| ENSG00000159140 | SON | ENSMUSG00000022961 | Son | SON | Unknown |
| ENSG00000181449 | SOX2 | ENSMUSG00000074637 | Sox2 | SOX2 | HMG/Sox |
| ENSG00000185591 | SP1 | ENSMUSG00000001280 | Sp1 | SP1 | C2H2 ZF |
| ENSG00000172845 | SP3 | ENSMUSG00000027109 | Sp3 | SP3 | C2H2 ZF |
| ENSG00000065526 | SPEN | ENSMUSG00000040761 | Spen | SPEN | Unknown |
| ENSG00000080603 | SRCAP | ENSMUSG00000107023 | Gm42715 | SRCAP | AT hook |
| ENSG00000080603 | SRCAP | ENSMUSG00000053877 | Srcap | SRCAP | AT hook |
| ENSG00000115415 | STAT1 | ENSMUSG00000026104 | Stat1 | STAT1 | STAT |
| ENSG00000168610 | STAT3 | ENSMUSG00000004040 | Stat3 | STAT3 | STAT |
| ENSG00000166888 | STAT6 | ENSMUSG00000002147 | Stat6 | STAT6 | STAT |
| ENSG00000162367 | TAL1 | ENSMUSG00000028717 | Tal1 | TAL1 | bHLH |
| ENSG00000112592 | TBP | ENSMUSG00000014767 | Tbp | TBP | TBP |
| ENSG00000135111 | TBX3 | ENSMUSG00000018604 | Tbx3 | TBX3 | T-box |
| ENSG00000140262 | TCF12 | ENSMUSG00000032228 | Tcf12 | TCF12 | bHLH |
| ENSG00000100207 | TCF20 | ENSMUSG00000071262 | Zfp957 | TCF20 | Unknown |
| ENSG00000100207 | TCF20 | ENSMUSG00000041852 | Tcf20 | TCF20 | Unknown |
| ENSG00000071564 | TCF3 | ENSMUSG00000020167 | Tcf3 | TCF3 | bHLH |
| ENSG00000196628 | TCF4 | ENSMUSG00000053477 | Tcf4 | TCF4 | bHLH |
| ENSG00000187079 | TEAD1 | ENSMUSG00000055320 | Tead1 | TEAD1 | TEA |
| ENSG00000138336 | TET1 | ENSMUSG00000047146 | Tet1 | TET1 | CxxC |
| ENSG00000168769 | TET2 | ENSMUSG00000040943 | Tet2 | TET2 | Unknown |
| ENSG00000090447 | TFAP4 | ENSMUSG00000005718 | Tfap4 | TFAP4 | bHLH |
| ENSG00000135457 | TFCP2 | ENSMUSG00000009733 | Tfcp2 | TFCP2 | Grainyhead |
| ENSG00000068323 | TFE3 | ENSMUSG00000000134 | Tfe3 | TFE3 | bHLH |
| ENSG00000112561 | TFEB | ENSMUSG00000023990 | Tfeb | TFEB | bHLH |
| ENSG00000177426 | TGIF1 | ENSMUSG00000047407 | Tgif1 | TGIF1 | Homeodomain |
| ENSG00000144747 | TMF1 | ENSMUSG00000030059 | Tmf1 | TMF1 | Unknown |
| ENSG00000141510 | TP53 | ENSMUSG00000059552 | Trp53 | TP53 | p53 |
| ENSG00000125482 | TTF1 | ENSMUSG00000026803 | Ttf1 | TTF1 | Myb/SANT |
| ENSG00000153560 | UBP1 | ENSMUSG00000009741 | Ubp1 | UBP1 | Grainyhead |
| ENSG00000158773 | USF1 | ENSMUSG00000026641 | Usf1 | USF1 | bHLH |
| ENSG00000105698 | USF2 | ENSMUSG00000058239 | Usf2 | USF2 | bHLH |
| ENSG00000136451 | VEZF1 | ENSMUSG00000018377 | Vezf1 | VEZF1 | C2H2 ZF |
| ENSG00000011451 | WIZ | ENSMUSG00000024050 | Wiz | WIZ | C2H2 ZF |
| ENSG00000100219 | XBP1 | ENSMUSG00000020484 | Xbp1 | XBP1 | bZIP |
| ENSG00000136936 | XPA | ENSMUSG00000028329 | Xpa | XPA | Unknown |
| ENSG00000065978 | YBX1 | ENSMUSG00000028639 | Ybx1 | YBX1 | CSD |
| ENSG00000006047 | YBX2 | ENSMUSG00000018554 | Ybx2 | YBX2 | CSD |
| ENSG00000060138 | YBX3 | ENSMUSG00000030189 | Ybx3 | YBX3 | CSD |
| ENSG00000100811 | YY1 | ENSMUSG00000021264 | Yy1 | YY1 | C2H2 ZF |
| ENSG00000181472 | ZBTB2 | ENSMUSG00000075327 | Zbtb2 | ZBTB2 | C2H2 ZF |
| ENSG00000177485 | ZBTB33 | ENSMUSG00000048047 | Zbtb33 | ZBTB33 | C2H2 ZF |
| ENSG00000178951 | ZBTB7A | ENSMUSG00000035011 | Zbtb7a | ZBTB7A | C2H2 ZF |
| ENSG00000160685 | ZBTB7B | ENSMUSG00000028042 | Zbtb7b | ZBTB7B | C2H2 ZF |
| ENSG00000144161 | ZC3H8 | ENSMUSG00000027387 | Zc3h8 | ZC3H8 | CCCH ZF |
| ENSG00000148516 | ZEB1 | ENSMUSG00000024238 | Zeb1 | ZEB1 | C2H2 ZF; |
| | | | | | Homeodomain |
| ENSG00000169554 | ZEB2 | ENSMUSG00000026872 | Zeb2 | ZEB2 | C2H2 ZF; |
| | | | | | Homeodomain |
| ENSG00000179059 | ZFP42 | ENSMUSG00000051176 | Zfp42 | ZFP42 | C2H2 ZF |
| ENSG00000186660 | ZFP91 | ENSMUSG00000024695 | Zfp91 | ZFP91 | C2H2 ZF |
| ENSG00000186660 | ZFP91 | ENSMUSG00000118491 | Gm44505 | ZFP91 | C2H2 ZF |
| ENSG00000005889 | ZFX | ENSMUSG00000079509 | Zfx | ZFX | C2H2 ZF |
| ENSG00000197114 | ZGPAT | ENSMUSG00000027582 | Zgpat | ZGPAT | CCCH ZF |
| ENSG00000165156 | ZHX1 | ENSMUSG00000022361 | Zhx1 | ZHX1 | Homeodomain |
| ENSG00000269699 | ZIM2 | Not Found | Not Found | ZIM2 | C2H2 ZF |
| ENSG00000103994 | ZNF106 | ENSMUSG00000027288 | Zfp106 | NA | NA |
| ENSG00000172262 | ZNF131 | ENSMUSG00000094870 | Zfp131 | ZNF131 | C2H2 ZF |
| ENSG00000166478 | ZNF143 | ENSMUSG00000061079 | Zfp143 | ZNF143 | C2H2 ZF |
| ENSG00000163848 | ZNF148 | ENSMUSG00000022811 | Zfp148 | ZNF148 | C2H2 ZF |
| ENSG00000096654 | ZNF184 | Not Found | Not Found | ZNF184 | C2H2 ZF |
| ENSG00000010244 | ZNF207 | ENSMUSG00000017421 | Zfp207 | ZNF207 | C2H2 ZF |
| ENSG00000171940 | ZNF217 | ENSMUSG00000052056 | Zfp217 | ZNF217 | C2H2 ZF |
| ENSG00000172466 | ZNF24 | ENSMUSG00000051469 | Zfp24 | ZNF24 | C2H2 ZF |
| ENSG00000198839 | ZNF277 | ENSMUSG00000055917 | Zfp277 | ZNF277 | C2H2 ZF; |
| | | | | | BED ZF |
| ENSG00000169548 | ZNF280A | Not Found | Not Found | ZNF280A | C2H2 ZF |
| ENSG00000056277 | ZNF280C | ENSMUSG00000036916 | Zfp280c | ZNF280C | C2H2 ZF |
| ENSG00000137871 | ZNF280D | ENSMUSG00000038535 | Zfp280d | ZNF280D | C2H2 ZF |
| ENSG00000162702 | ZNF281 | ENSMUSG00000041483 | Zfp281 | ZNF281 | C2H2 ZF |
| ENSG00000188994 | ZNF292 | ENSMUSG00000039967 | Zfp292 | ZNF292 | C2H2 ZF |
| ENSG00000170684 | ZNF296 | ENSMUSG00000011267 | Zfp296 | ZNF296 | C2H2 ZF |
| ENSG00000171467 | ZNF318 | ENSMUSG00000015597 | Zfp318 | ZNF318 | C2H2 ZF |
| ENSG00000162664 | ZNF326 | ENSMUSG00000029290 | Zfp326 | ZNF326 | C2H2 ZF |
| ENSG00000196378 | ZNF34 | ENSMUSG00000078497 | Zfp978 | ZNF34 | C2H2 ZF |
| ENSG00000088876 | ZNF343 | Not Found | Not Found | ZNF343 | C2H2 ZF |
| ENSG00000113761 | ZNF346 | ENSMUSG00000021481 | Zfp346 | ZNF346 | C2H2 ZF |
| ENSG00000126746 | ZNF384 | ENSMUSG00000038346 | Zfp384 | ZNF384 | C2H2 ZF |
| ENSG00000161642 | ZNF385A | Not Found | Not Found | ZNF385A | C2H2 ZF |
| ENSG00000167685 | ZNF444 | ENSMUSG00000044876 | Zfp444 | ZNF444 | C2H2 ZF |
| ENSG00000112200 | ZNF451 | ENSMUSG00000042197 | Zfp451 | ZNF451 | C2H2 ZF |
| ENSG00000148143 | ZNF462 | ENSMUSG00000060206 | Zfp462 | ZNF462 | C2H2 ZF |
| ENSG00000180035 | ZNF48 | ENSMUSG00000045598 | Zfp553 | ZNF48 | C2H2 ZF |
| ENSG00000162714 | ZNF496 | ENSMUSG00000020472 | Zkscan17 | ZNF496 | C2H2 ZF |
| ENSG00000178163 | ZNF518B | ENSMUSG00000046572 | Zfp518b | ZNF518B | C2H2 ZF |
| ENSG00000218891 | ZNF579 | ENSMUSG00000051550 | Zfp579 | ZNF579 | C2H2 ZF |
| ENSG00000166716 | ZNF592 | ENSMUSG00000005621 | Zfp592 | ZNF592 | C2H2 ZF |
| ENSG00000167962 | ZNF598 | ENSMUSG00000041130 | Zfp598 | ZNF598 | C2H2 ZF |
| ENSG00000180357 | ZNF609 | ENSMUSG00000040524 | Zfp609 | ZNF609 | C2H2 ZF |
| ENSG00000122482 | ZNF644 | ENSMUSG00000049606 | Zfp644 | ZNF644 | C2H2 ZF |
| ENSG00000197343 | ZNF655 | ENSMUSG00000007812 | Zfp655 | ZNF655 | C2H2 ZF |
| ENSG00000167394 | ZNF668 | ENSMUSG00000049728 | Zfp668 | ZNF668 | C2H2 ZF |
| ENSG00000143373 | ZNF687 | ENSMUSG00000019338 | Zfp687 | ZNF687 | C2H2 ZF |
| ENSG00000139651 | ZNF740 | ENSMUSG00000046897 | Zfp740 | ZNF740 | C2H2 ZF |
| ENSG00000169957 | ZNF768 | ENSMUSG00000047371 | Zfp768 | ZNF768 | C2H2 ZF |
| ENSG00000179965 | ZNF771 | ENSMUSG00000054716 | Zfp771 | ZNF771 | C2H2 ZF |
| ENSG00000048405 | ZNF800 | ENSMUSG00000039841 | Zfp800 | ZNF800 | C2H2 ZF |
| ENSG00000198783 | ZNF830 | ENSMUSG00000046010 | Zfp830 | ZNF830 | C2H2 ZF |
| ENSG00000166529 | ZSCAN21 | ENSMUSG00000037017 | Zscan21 | ZSCAN21 | C2H2 ZF |
| ENSG00000036549 | ZZZ3 | ENSMUSG00000039068 | Zzz3 | ZZZ3 | Myb/SANT |

TABLE 2

| Gene_Name | ARM | Mutation | Associated_Diseases |
|---|---|---|---|
| AHDC1 | GRRNKTTYK (SEQ ID NO: | Arg487Trp | SCHIZOPHRENIA |
| | 18), KIPVSLGRR (SEQ ID NO: | | |
| | 19), RRNKTTYKV (SEQ ID NO: | | |
| | 20), SLGRRNKTT (SEQ ID NO: 21) | | |
| AIRE | KRKASEEAR (SEQ ID NO: | Arg132Ser | AUTOIMMUNE DISEASE, AUTOIMMUNE |
| | 22), PPPRLPTKR (SEQ ID NO: | | POLYENDOCRINE SYNDROME, TYPE I, WITH |
| | 23), PPRLPTKRK (SEQ ID NO: | | OR WITHOUT REVERSIBLE METAPHYSEAL |
| | 24), PRLPTKRKA (SEQ ID NO: | | DYSPLASIA, HYPOADRENOCORTICISM, |
| | 25), PTKRKASEE (SEQ ID NO: | | FAMILIAL |
| | 26), RKASEEARA (SEQ ID NO: | | |
| | 27), RLPTKRKAS (SEQ ID NO: | | |
| | 28), TKRKASEEA (SEQ ID NO: 29) | | |
| AIRE | PPPRLPTKR (SEQ ID NO: 30) | Pro124Leu | AUTOIMMUNE DISEASE, AUTOIMMUNE |
| | | | POLYENDOCRINE SYNDROME, TYPE I, WITH |
| | | | OR WITHOUT REVERSIBLE METAPHYSEAL |
| | | | DYSPLASIA, HYPOADRENOCORTICISM, |
| | | | FAMILIAL |
| AIRE | RPGTGLRCR (SEQ ID NO: 31) | Arg471Cys | AUTOIMMUNE DISEASE, AUTOIMMUNE |
| | | | POLYENDOCRINE SYNDROME, TYPE I, WITH |
| | | | OR WITHOUT REVERSIBLE METAPHYSEAL |
| | | | DYSPLASIA, HYPOADRENOCORTICISM, |
| | | | FAMILIAL |
| ASH1L | AKRTKKPPK (SEQ ID NO: | Pro99Thr | AUTISM |
| | 32), KRTKKPPKN (SEQ ID NO: | | |
| | 33), LQAKRTKKP (SEQ ID NO: | | |
| | 34), QAKRTKKPP (SEQ ID NO: | | |
| | 35), RTKKPPKNL (SEQ ID NO: 36) | | |
| ASH1L | ASKGRRRLS (SEQ ID NO: | Arg1160Gln | AUTISM |
| | 37), GRRRLSPPT (SEQ ID NO: | | |
| | 38), KASKGRRRL (SEQ ID NO: | | |
| | 39), KGRRRLSPP (SEQ ID NO: | | |
| | 40), KKASKGRRR (SEQ ID NO: | | |
| | 41), RRRLSPPTL (SEQ ID NO: | | |
| | 42), SKGRRRLSP (SEQ ID NO: 43) | | |
| ASH1L | AVGERYKHK (SEQ ID NO: | Tyr1528Cys | AUTISM, GILLES DE LA TOURETTE SYNDROME |
| | 44), ERYKHKEKH (SEQ ID NO: | | |
| | 45), GERYKHKEK (SEQ ID NO: | | |
| | 46), KDAVGERYK (SEQ ID NO: | | |
| | 47), RYKHKEKHR (SEQ ID NO: | | |
| | 48), YKHKEKHRC (SEQ ID NO: 49) | | |
| ASH1L | AVGERYKHK (SEQ ID NO: | Ala1523Thr | AUTISM |
| | 50), KDAVGERYK (SEQ ID NO: | | |
| | 51), KRYRFGKDA (SEQ ID NO: 52) | | |
| ASH1L | DIYKPKRGR (SEQ ID NO: | Lys825Glu | AUTISM |
| | 53), IYKPKRGRP (SEQ ID NO: | | |
| | 54), KPKRGRPKS (SEQ ID NO: | | |
| | 55), SDIYKPKRG (SEQ ID NO: | | |
| | 56), YKPKRGRPK (SEQ ID NO: 57) | | |
| ASH1L | EHKKGLKRK (SEQ ID NO: | Lys1915Arg | AUTISM, GILLES DE LA TOURETTE SYNDROME |
| | 58), GLKRKGWLL (SEQ ID NO: | | |
| | 59), HKKGLKRKG (SEQ ID NO: | | |
| | 60), KGLKRKGWL (SEQ ID NO: | | |
| | 61), KKGLKRKGW (SEQ ID NO: | | |
| | 62), KRKGWLLEE (SEQ ID NO: | | |
| | 63), LKRKGWLLE (SEQ ID NO: | | |
| | 64), PEHKKGLKR (SEQ ID NO: 65) | | |
| ASH1L | ESLKRYRFG (SEQ ID NO: | Arg1516Cys | GILLES DE LA TOURETTE SYNDROME |
| | 66), KRYRFGKDA (SEQ ID NO: | | |
| | 67), LKRYRFGKD (SEQ ID NO: | | |
| | 68), RSVLESLKR (SEQ ID NO: | | |
| | 69), SLKRYRFGK (SEQ ID NO: 70) | | |
| ASH1L | ESLKRYRFG (SEQ ID NO: | Arg1518Gly | AUTISM |
| | 71), KRYRFGKDA (SEQ ID NO: | | |
| | 72), LKRYRFGKD (SEQ ID NO: | | |
| | 73), SLKRYRFGK (SEQ ID NO: 74) | | |
| ASH1L | GRRRLSPPT (SEQ ID NO: | Pro1164Leu | AUTISM |
| | 75), KGRRRLSPP (SEQ ID NO: | | |
| | 76), RRRLSPPTL (SEQ ID NO: 77) | | |
| ASH1L | HSKDRTLGK (SEQ ID NO: | Arg1757Gln | AUTISM |
| | 78), KDRTLGKPD (SEQ ID NO: | | |
| | 79), PGRSHSKDR (SEQ ID NO: | | |
| | 80), RSHSKDRTL (SEQ ID NO: | | |
| | 81), SKDRTLGKP (SEQ ID NO: 82) | | |
| ASH1L | KREVELEKN (SEQ ID NO: | Lys34Asn | AUTISM |
| | 83), SKREVELEK (SEQ ID NO: 84) | | |
| ASH1L | KRNRERNIE (SEQ ID NO: | Glu59Lys | AUTISM |
| | 85), RERNIEAGK (SEQ ID NO: | | |
| | 86), RNRERNIEA (SEQ ID NO: 87) | | |
| ASH1L | KVVARSTCR (SEQ ID NO: | Ala724Ser | MENTAL RETARDATION, AUTOSOMAL |
| | 88), PRWTKVVAR (SEQ ID NO: | | DOMINANT 52 |
| | 89), RWTKVVARS (SEQ ID NO: 90) | | |
| ASH1L | KVVARSTCR (SEQ ID NO: | Arg729Gln | AUTISM |
| | 91), RSTCRSPKG (SEQ ID NO: 92) | | |
| ASH1L | MKPMSNRER (SEQ ID NO: 93) | Asn2354Ser | AUTISM |
| ASH1L | PGRSHSKDR (SEQ ID NO: | Arg1751Cys | AUTISM |
| | 94), RSHSKDRTL (SEQ ID NO: 95) | | |
| ASH1L | RSTCRSPKG (SEQ ID NO: 96) | Pro731Arg | AUTISM |
| ASH1L | SRDPDLKDR (SEQ ID NO: 97) | Asp203Asn | AUTISM |
| ATF6 | AIRRRGDTF (SEQ ID NO: | Asp564Gly | CONE-ROD DYSTROPHY 2 |
| | 98), IRRRGDTFY (SEQ ID NO: | | |
| | 99),RRRGDTFYV (SEQ ID NO: 100) | | |
| ATF6 | IRRRGDTFY (SEQ ID NO: | Tyr567Asn | ACHROMATOPSIA 7 |
| | 101), RRRGDTFYV (SEQ ID NO: 102) | | |
| BARX1 | ESPTKPKGR (SEQ ID NO: | Thr211Ile | TETRALOGY OF FALLOT |
| | 103), PTKPKGRPK (SEQ ID NO: | | |
| | 104), TKPKGRPKK (SEQ ID NO: 105) | | |
| | MSRRKQGNP (SEQ ID NO: | Arg3Ser | CRANIOSYNOSTOSIS 1 |
| | 106), RRKQGNPQH (SEQ ID NO: | | |
| BCL11B | 107), SRRKQGNPQ (SEQ ID NO: 108) | | |
| BHLHA9 | KARRMAANV (SEQ ID NO: | Asn71Asp | SYNDACTYLY, MESOAXIAL SYNOSTOTIC, |
| | 109), SKARRMAAN (SEQ ID NO: 110) | | WITH PHALANGEAL REDUCTION |
| BNC1 | ALPKKKSRK (SEQ ID NO: | Leu532Pro | PREMATURE OVARIAN FAILURE 1 |
| | 111), DALPKKKSR (SEQ ID NO: | | |
| | 112), LPKKKSRKS (SEQ ID NO: 113) | | |
| BNC2 | HRKLLTKEL (SEQ ID NO: | His888Arg | LOWER URINARY TRACT OBSTRUCTION, |
| | 114), LHRKLLTKE (SEQ ID NO: | | CONGENITAL |
| | 115), NLHRKLLTK (SEQ ID NO: 116) | | |
| BPTF | EISRLSTKK (SEQ ID NO: | Arg823Gln | LUNG CANCER |
| | 117), ISRLSTKKE (SEQ ID NO: 118) | | |
| CBX2 | RRSKLKEPD (SEQ ID NO: | Pro98Leu | 46, XY SEX REVERSAL 5 |
| | 119), RSKLKEPDA (SEQ ID NO: | | |
| | 120), SRRSKLKEP (SEQ ID NO: 121) | | |
| CBX2 | RTAPGEARK (SEQ ID NO: 122) | Arg443Pro | 46, XY SEX REVERSAL 5 |
| CEBPA | HSRQQEKAK (SEQ ID NO: 123) | His84Leu | LEUKEMIA, ACUTE MYELOID |
| CTCF | CPRRSNLDR (SEQ ID NO: | Arg283His | MENTAL RETARDATION, AUTOSOMAL |
| | 124), PRRSNLDRH (SEQ ID NO: | | DOMINANT 21 |
| | 125), RRSNLDRHM (SEQ ID NO: 126) | | |
| CTCF | FTRRNTMAR (SEQ ID NO: | Arg567Trp | MENTAL RETARDATION, AUTOSOMAL |
| | 127), RRNTMARHA (SEQ ID NO: | | DOMINANT 21 |
| | 128), TRRNTMARH (SEQ ID NO: 129) | | |
| CTCF | PRRSNLDRH (SEQ ID NO: | His284Arg | Breast Invasive Lobular Carcinoma (ILC) |
| | 130), RRSNLDRHM (SEQ ID NO: 131) | | |
| CTCF | PRRSNLDRH (SEQ ID NO: | His284Asn | Breast Invasive Ductal Carcinoma (IDC) |
| | 132), RRSNLDRHM (SEQ ID NO: 133) | | |
| CTCF | PRRSNLDRH (SEQ ID NO: | His284Asp | Breast Invasive Ductal Carcinoma (IDC) |
| | 134), RRSNLDRHM (SEQ ID NO: 135) | | |
| CTCF | PRRSNLDRH (SEQ ID NO: | His284Gln | Breast Invasive Ductal Carcinoma (IDC) |
| | 136), RRSNLDRHM (SEQ ID NO: 137) | | |
| CTCF | PRRSNLDRH (SEQ ID NO: | His284Leu | Breast Invasive Ductal Carcinoma (IDC) |
| | 138), RRSNLDRHM (SEQ ID NO: 139) | | |
| CTCF | PRRSNLDRH (SEQ ID NO: | His284Pro | Breast Invasive Ductal Carcinoma (IDC) |
| | 140), RRSNLDRHM (SEQ ID NO: 141) | | |
| CTCF | PRRSNLDRH (SEQ ID NO: | His284Tyr | Breast Invasive Carcinoma, NOS (BRCNOS), |
| | 142), RRSNLDRHM (SEQ ID NO: 143) | | Breast Invasive Ductal Carcinoma (IDC) |
| CXXC1 | EERYKRHRQ (SEQ ID NO: | Arg347Trp | AUTISM |
| | 144), ERYKRHRQK (SEQ ID NO: | | |
| | 145), KEERYKRHR (SEQ ID NO: | | |
| | 146), KKEERYKRH (SEQ ID NO: | | |
| | 147), KKKEERYKR (SEQ ID NO: | | |
| | 148), KRHRQKQKH (SEQ ID NO: | | |
| | 149), RHRQKQKHK (SEQ ID NO: | | |
| | 150), RYKRHRQKQ (SEQ ID NO: | | |
| | 151), YKRHRQKQK (SEQ ID NO: 152) | | |
| DPF3 | ARGSAGGRR (SEQ ID NO: | Gly182Asp | AUTISM |
| | 153), GGRRRHDAA (SEQ ID NO: | | |
| | 154), GRRRHDAAS (SEQ ID NO: | | |
| | 155), RARGSAGGR (SEQ ID NO: | | |
| | 156), RGSAGGRRR (SEQ ID NO: 157) | | |
| E2F1 | KRRLDLETD (SEQ ID NO: | Asp93Glu | SCHIZOPHRENIA |
| | 158), PVKRRLDLE (SEQ ID NO: | | |
| | 159), RPPVKRRLD (SEQ ID NO: | | |
| | 160), VKRRLDLET (SEQ ID NO: 161) | | |
| EGR2 | ARSDERKRH (SEQ ID NO: | Glu412Gly | CHARCOT-MARIE-TOOTH DISEASE, |
| | 162), DERKRHTKI (SEQ ID NO: | | DEMYELINATING, TYPE 1B |
| | 163), ERKRHTKIH (SEQ ID NO: | | |
| | 164), FARSDERKR (SEQ ID NO: | | |
| | 165), KFARSDERK (SEQ ID NO: | | |
| | 166), RSDERKRHT (SEQ ID NO: | | |
| | 167), SDERKRHTK (SEQ ID NO: 168) | | |
| EGR2 | ARSDERKRH (SEQ ID NO: | Glu412Lys | CHARCOT-MARIE-TOOTH DISEASE, |
| | 169), DERKRHTKI (SEQ ID NO: | | DEMYELINATING, TYPE 1B, HYPERTROPHIC |
| | 170), ERKRHTKIH (SEQ ID NO: | | NEUROPATHY OF DEJERINE-SOTTAS |
| | 171), FARSDERKR (SEQ ID NO: | | |
| | 172), KFARSDERK (SEQ ID NO: | | |
| | 173), RSDERKRHT (SEQ ID NO: | | |
| | 174), SDERKRHTK (SEQ ID NO: 175) | | |
| EGR2 | ARSDERKRH (SEQ ID NO: | Asp411Gly | CHARCOT-MARIE-TOOTH DISEASE, |
| | 176), DERKRHTKI (SEQ ID NO: | | DEMYELINATING, TYPE 1B, HYPERTROPHIC |
| | 177), FARSDERKR (SEQ ID NO: | | NEUROPATHY OF DEJERINE-SOTTAS |
| | 178), KFARSDERK (SEQ ID NO: | | |
| | 179), RSDERKRHT (SEQ ID NO: | | |
| | 180), SDERKRHTK (SEQ ID NO: 181) | | |
| EGR2 | ARSDERKRH (SEQ ID NO: | Arg409Gln | CHARCOT-MARIE-TOOTH DISEASE, |
| | 182), FARSDERKR (SEQ ID NO: | | DEMYELINATING, TYPE 1B |
| | 183), KFARSDERK (SEQ ID NO: | | |
| | 184), RSDERKRHT (SEQ ID NO: 185) | | |
| EGR2 | ARSDERKRH (SEQ ID NO: | Arg409Trp | CHARCOT-MARIE-TOOTH DISEASE, |
| | 186), FARSDERKR (SEQ ID NO: | | DEMYELINATING, TYPE 1B, CHARCOT-MARIE- |
| | 187), KFARSDERK (SEQ ID NO: | | TOOTH DISEASE, DEMYELINATING, TYPE ID |
| | 188), RSDERKRHT (SEQ ID NO: 189) | | |
| EGR2 | CDRRFSRSD (SEQ ID NO: | Arg353Gly | CHARCOT-MARIE-TOOTH DISEASE, |
| | 190), GCDRRFSRS (SEQ ID NO: | | DEMYELINATING, TYPE 1B |
| | 191), RRFSRSDEL (SEQ ID NO: 192) | | |
| EGR2 | CDRRFSRSD (SEQ ID NO: | Asp355Val | CHARCOT-MARIE-TOOTH DISEASE, |
| | 193), RRFSRSDEL (SEQ ID NO: 194) | | DEMYELINATING, TYPE 1B |
| EGR2 | LRPILRPRK (SEQ ID NO: | Arg324His | CHARCOT-MARIE-TOOTH DISEASE, |
| | 195), LRPRKYPNR (SEQ ID NO: | | DEMYELINATING, TYPE 1B |
| | 196), PLRPILRPR (SEQ ID NO: | | |
| | 197), PRKYPNRPS (SEQ ID NO: | | |
| | 198), RKYPNRPSK (SEQ ID NO: | | |
| | 199), RPILRPRKY (SEQ ID NO: | | |
| | 200), RPRKYPNRP (SEQ ID NO: 201) | | |
| EGR2 | RRFSRSDEL (SEQ ID NO: 202) | Glu356Lys | CHARCOT-MARIE-TOOTH DISEASE, |
| | | | DEMYELINATING, TYPE 1B |
| ERF | FKRRWSEDC (SEQ ID NO: | Arg487His | SCHIZOPHRENIA |
| | 203), KLRFKRRWS (SEQ ID NO: | | |
| | 204), KRRWSEDCR (SEQ ID NO: | | |
| | 205), LKLRFKRRW (SEQ ID NO: | | |
| | 206), LRFKRRWSE (SEQ ID NO: | | |
| | 207), PLKLRFKRR (SEQ ID NO: | | |
| | 208), RFKRRWSED (SEQ ID NO: | | |
| | 209), RRWSEDCRL (SEQ ID NO: 210) | | |
| ERG | ARRWGERKS (SEQ ID NO: | Arg354Gln | B-Lymphoblastic Leukemia/Lymphoma (BLL), |
| | 211), ERKSKPNMN (SEQ ID NO: | | Chronic Lymphocytic Leukemia/Small |
| | 212), EVARRWGER (SEQ ID NO: | | Lymphocytic |
| | 213), RKSKPNMNY (SEQ ID NO: | | |
| | 214), RRWGERKSK (SEQ ID NO: | | Lymphoma (CLLSLL) |
| | 215), RWGERKSKP (SEQ ID NO: | | |
| | 216), VARRWGERK (SEQ ID NO: | | |
| | 217), WGERKSKPN (SEQ ID NO: 218) | | |
| ERG | ARRWGERKS (SEQ ID NO: | Arg354Trp | Prostate Adenocarcinoma (PRAD) |
| | 219), ERKSKPNMN (SEQ ID NO: | | |
| | 220), EVARRWGER (SEQ ID NO: | | |
| | 221), RKSKPNMNY (SEQ ID NO: | | |
| | 222), RRWGERKSK (SEQ ID NO: | | |
| | 223), RWGERKSKP (SEQ ID NO: | | |
| | 224), VARRWGERK (SEQ ID NO: | | |
| | 225), WGERKSKPN (SEQ ID NO: 226) | | |
| ERG | ERKSKPNMN (SEQ ID NO: | Arg361Gln | B-Lymphoblastic Leukemia/Lymphoma (BLL), |
| | 227), RKSKPNMNY (SEQ ID NO: 228) | | Chronic Lymphocytic Leukemia/Small |
| | | | Lymphocytic Lymphoma (CLLSLL) |
| ERG | ERKSKPNMN (SEQ ID NO: | Arg361Trp | Prostate Adenocarcinoma (PRAD) |
| | 229), RKSKPNMNY (SEQ ID NO: 230) | | |
| ESR1 | IKRSKKNSL (SEQ ID NO: | Lys303Arg | BREAST CANCER |
| | 231), KRSKKNSLA (SEQ ID NO: | | |
| | 232), LMIKRSKKN (SEQ ID NO: | | |
| | 233), MIKRSKKNS (SEQ ID NO: | | |
| | 234), PLMIKRSKK (SEQ ID NO: | | |
| | 235), RSKKNSLAL (SEQ ID NO: 236) | | |
| ETV3 | LMPPKLRLK (SEQ ID NO: | Pro471Arg | AUTISM |
| | 237), MPPKLRLKR (SEQ ID NO: | | |
| | 238), PPKLRLKRR (SEQ ID NO: 239) | | |
| ETV6 | IRRLSPAER (SEQ ID NO: | Pro214Leu | B-Lymphoblastic Leukemia/Lymphoma (BLL), |
| | 240), RRLSPAERA (SEQ ID NO: 241) | | Chronic Myelomonocytic Leukemia (CMML), |
| | | | Colon Adenocarcinoma (COAD), Colorectal |
| | | | Adenocarcinoma (COADREAD), Cutaneous |
| | | | Squamous Cell Carcinoma (CSCC), Diffuse |
| | | | Large B-Cell Lymphoma, NOS (DLBCLNOS), |
| | | | Head and Neck Squamous Cell Carcinoma |
| | | | (HNSC), Melanoma (MEL), Myeloid Neoplasm |
| | | | (MNM), Nasopharyngeal Carcinoma (NPC), |
| | | | Oropharynx Squamous Cell Carcinoma |
| | | | (OPHSC), Poorly Differentiated Thyroid |
| | | | Cancer (THPD), Rectal Adenocarcinoma |
| | | | (READ), Stomach Adenocarcinoma (STAD), |
| | | | THROMBOCYTOPENIA 5, Thymoma (THYM), |
| | | | Urethral Squamous Cell Carcinoma (USCC), |
| | | | Uterine Clear Cell Carcinoma (UCCC) |
| ETV6 | LKQRKPRIL (SEQ ID NO: | Arg127Gln | LEUKEMIA, CHRONIC MYELOID |
| | 242), QRKPRILFS (SEQ ID NO: | | |
| | 243), RKPRILFSP (SEQ ID NO: 244) | | |
| FEZF1 | RGSPNAKPK (SEQ ID NO: 245) | Arg250Gly | HYPOGONADOTROPIC HYPOGONADISM 1 |
| | | | WITH OR WITHOUT ANOSMIA |
| FLI1 | ARRWGERKS (SEQ ID NO: | Arg324Trp | BLEEDING DISORDER, PLATELET-TYPE, 21 |
| | 246), ERKSKPNMN (SEQ ID NO: | | |
| | 247), EVARRWGER (SEQ ID NO: | | |
| | 248), RKSKPNMNY (SEQ ID NO: | | |
| | 249), RRWGERKSK (SEQ ID NO: | | |
| | 250), RWGERKSKP (SEQ ID NO: | | |
| | 251), VARRWGERK (SEQ ID NO: | | |
| | 252), WGERKSKPN (SEQ ID NO: 253) | | |
| FOXA1 | CYLRRQKRF (SEQ ID NO: | Arg261Cys | Bladder Urothelial Carcinoma (BLCA), |
| | 254), GCYLRRQKR (SEQ ID NO: | | Breast Invasive Ductal Carcinoma (IDC), |
| | 255), LRRQKRFKC (SEQ ID NO: | | Prostate Adenocarcinoma (PRAD) |
| | 256), RRQKRFKCE (SEQ ID NO: | | |
| | 257), YLRRQKRFK (SEQ ID NO: 258) | | |
| FOXA1 | CYLRRQKRF (SEQ ID NO: | Arg261Gly | Breast Invasive Lobular Carcinoma (ILC), |
| | 259), GCYLRRQKR (SEQ ID NO: | | Prostate Adenocarcinoma (PRAD) |
| | 260), LRRQKRFKC (SEQ ID NO: | | |
| | 261), RRQKRFKCE (SEQ ID NO: | | |
| | 262), YLRRQKRFK (SEQ ID NO: 263) | | |
| FOXA1 | CYLRRQKRF (SEQ ID NO: | Arg261His | Breast Invasive Lobular Carcinoma (ILC), |
| | 264), GCYLRRQKR (SEQ ID NO: | | Mucinous Adenocarcinoma of the Colon and |
| | 265), LRRQKRFKC (SEQ ID NO: | | Rectum (MACR), Prostate Adenocarcinoma |
| | 266), RRQKRFKCE (SEQ ID NO: | | (PRAD) |
| | 267), YLRRQKRFK (SEQ ID NO: 268) | | |
| FOXA1 | CYLRRQKRF (SEQ ID NO: | Arg261Ser | Breast Invasive Carcinoma, NOS (BRCNOS), |
| | 269), GCYLRRQKR (SEQ ID NO: | | Breast Invasive Ductal Carcinoma (IDC), |
| | 270), LRRQKRFKC (SEQ ID NO: | | Breast Mixed Ductal and Lobular Carcinoma |
| | 271), RRQKRFKCE (SEQ ID NO: | | (MDLC), Prostate Adenocarcinoma (PRAD), |
| | 272), YLRRQKRFK (SEQ ID NO: 273) | | Uterine Endometrioid Carcinoma (UEC) |
| FOXA1 | CYLRRQKRF (SEQ ID NO: | Tyr259Asn | Prostate Adenocarcinoma (PRAD) |
| | 274), GCYLRRQKR (SEQ ID NO: | | |
| | 275), YLRRQKRFK (SEQ ID NO: 276) | | |
| FOXA1 | CYLRRQKRF (SEQ ID NO: | Tyr259Cys | Breast Invasive Ductal Carcinoma (IDC), |
| | 277), GCYLRRQKR (SEQ ID NO: | | Prostate Adenocarcinoma (PRAD) |
| | 278), YLRRQKRFK (SEQ ID NO: 279) | | |
| FOXA1 | CYLRRQKRF (SEQ ID NO: | Tyr259Ser | Breast Invasive Ductal Carcinoma (IDC), |
| | 280), GCYLRRQKR (SEQ ID NO: | | Invasive Breast Carcinoma (BRCA), Prostate |
| | 281), YLRRQKRFK (SEQ ID NO: 282) | | Adenocarcinoma (PRAD) |
| FOXA1 | CYLRRQKRF (SEQ ID NO: | Phe266Cys | Prostate Adenocarcinoma (PRAD) |
| | 283), KRFKCEKQP (SEQ ID NO: | | |
| | 284), LRRQKRFKC (SEQ ID NO: | | |
| | 285), QKRFKCEKQ (SEQ ID NO: | | |
| | 286), RQKRFKCEK (SEQ ID NO: | | |
| | 287), RRQKRFKCE (SEQ ID NO: | | |
| | 288), YLRRQKRFK (SEQ ID NO: 289) | | |
| FOXA1 | CYLRRQKRF (SEQ ID NO: | Phe266Ile | Breast Invasive Lobular Carcinoma (ILC) |
| | 290), KRFKCEKQP (SEQ ID NO: | | |
| | 291), LRRQKRFKC (SEQ ID NO: | | |
| | 292), QKRFKCEKQ (SEQ ID NO: | | |
| | 293), RQKRFKCEK (SEQ ID NO: | | |
| | 294), RRQKRFKCE (SEQ ID NO: | | |
| | 295), YLRRQKRFK (SEQ ID NO: 296) | | |
| FOXA1 | CYLRRQKRF (SEQ ID NO: | Phe266Leu | Bladder Urothelial Carcinoma (BLCA), |
| | 297), KRFKCEKQP (SEQ ID NO: | | Breast Invasive Cancer, NOS (BRCANOS), |
| | 298), LRRQKRFKC (SEQ ID NO: | | Breast Invasive Ductal Carcinoma (IDC), |
| | 299), QKRFKCEKQ (SEQ ID NO: | | Breast Invasive Lobular Carcinoma (ILC), |
| | 300), RQKRFKCEK (SEQ ID NO: | | Breast Mixed Ductal and Lobular Carcinoma |
| | 301), RRQKRFKCE (SEQ ID NO: | | (MDLC), Invasive Breast Carcinoma |
| | 302), YLRRQKRFK (SEQ ID NO: 303) | | (BRCA), Prostate Adenocarcinoma (PRAD) |
| FOXA1 | CYLRRQKRF (SEQ ID NO: | Phe266Ser | Prostate Adenocarcinoma (PRAD) |
| | 304), KRFKCEKQP (SEQ ID NO: | | |
| | 305), LRRQKRFKC (SEQ ID NO: | | |
| | 306), QKRFKCEKQ (SEQ ID NO: | | |
| | 307), RQKRFKCEK (SEQ ID NO: | | |
| | 308), RRQKRFKCE (SEQ ID NO: | | |
| | 309), YLRRQKRFK (SEQ ID NO: 310) | | |
| FOXA1 | CYLRRQKRF (SEQ ID NO: | Phe266Tyr | Prostate Adenocarcinoma (PRAD) |
| | 311), KRFKCEKQP (SEQ ID NO: | | |
| | 312), LRRQKRFKC (SEQ ID NO: | | |
| | 313), QKRFKCEKQ (SEQ ID NO: | | |
| | 314), RQKRFKCEK (SEQ ID NO: | | |
| | 315), RRQKRFKCE (SEQ ID NO: | | |
| | 316), YLRRQKRFK (SEQ ID NO: 317) | | |
| FOXA1 | CYLRRQKRF (SEQ ID NO: | Phe266Val | Breast Invasive Ductal Carcinoma (IDC), |
| | 318), KRFKCEKQP (SEQ ID NO: | | Prostate Adenocarcinoma (PRAD) |
| | 319), LRRQKRFKC (SEQ ID NO: | | |
| | 320), QKRFKCEKQ (SEQ ID NO: | | |
| | 321), RQKRFKCEK (SEQ ID NO: | | |
| | 322), RRQKRFKCE (SEQ ID NO: | | |
| | 323), YLRRQKRFK (SEQ ID NO: 324) | | |
| FOXE3 | DNGSFLRRR (SEQ ID NO: | Arg164Ser | AORTIC ANEURYSM, FAMILIAL THORACIC 1 |
| | 325), FLRRRKRFK (SEQ ID NO: | | PYLORIC STENOSIS, INFANTILE |
| | 326), GSFLRRRKR (SEQ ID NO: | | |
| | 327), LRRRKRFKR (SEQ ID NO: | | |
| | 328), NGSFLRRRK (SEQ ID NO: | | |
| | 329), RKRFKRAEL (SEQ ID NO: | | |
| | 330), RRKRFKRAE (SEQ ID NO: | | |
| | 331), RRRKRFKRA (SEQ ID NO: | | |
| | 332), SFLRRRKRF (SEQ ID NO: 333) | | |
| FOXF1 | EGSFRRRPR (SEQ ID NO: | Arg139Gln | HYPERTROPHIC, 5 |
| | 334), FEEGSFRRR (SEQ ID NO: | | |
| | 335), FRRRPRGFR (SEQ ID NO: | | |
| | 336), GSFRRRPRG (SEQ ID NO: | | |
| | 337), RRPRGFRRK (SEQ ID NO: | | |
| | 338), RRRPRGFRR (SEQ ID NO: | | |
| | 339), SFRRRPRGF (SEQ ID NO: 340) | | |
| FOXF1 | EGSFRRRPR (SEQ ID NO: | Arg138Pro | ALVEOLAR CAPILLARY DYSPLASIA WITH |
| | 341), FEEGSFRRR (SEQ ID NO: | | MISALIGNMENT OF PULMONARY VEINS |
| | 342), FRRRPRGFR (SEQ ID NO: | | |
| | 343), GSFRRRPRG (SEQ ID NO: | | |
| | 344), RRRPRGFRR (SEQ ID NO: | | |
| | 1265), SFRRRPRGF (SEQ ID NO: | | |
| | 1266) | | |
| FOXI1 | DNGNFRRKR (SEQ ID NO: | Arg213Pro | EAR MALFORMATION, RENAL TUBULAR |
| | 1267), FDNGNFRRK (SEQ ID NO: | | ACIDOSIS, DISTAL, 1 |
| | 1268), FRRKRKRKS (SEQ ID NO: | | |
| | 1269), GNFRRKRKR (SEQ ID NO: | | |
| | 1270), NFRRKRKRK (SEQ ID NO: | | |
| | 1271), NGNFRRKRK (SEQ ID NO: | | |
| | 1272), RRKRKRKSD (SEQ ID NO: | | |
| | 1273) | | |
| FOXP3 | KRSQRPSRC (SEQ ID NO: | Cys424Tyr | IMMUNODYSREGULATION, |
| | 1274), RSQRPSRCS (SEQ ID | | POLYENDOCRINOPATHY, AND |
| | NO: 345) | | ENTEROPATHY, X-LINKED |
| GATA2 | GIQTRNRKM (SEQ ID NO: | Arg396Gln | IMMUNODEFICIENCY 21, LEUKEMIA, ACUTE |
| | 346), KEGIQTRNR (SEQ ID NO: | | MYELOID, LYMPHEDEMA, PRIMARY, WITH |
| | 347), KKEGIQTRN (SEQ ID NO: | | MYELODYSPLASIA, MYELODYSPLASTIC |
| | 348), MKKEGIQTR (SEQ ID NO: | | SYNDROME |
| | 349), RNRKMSNKS (SEQ ID NO: | | |
| | 350), TRNRKMSNK (SEQ ID NO: 351) | | |
| GATA2 | GIQTRNRKM (SEQ ID NO: | Arg396Leu | IMMUNODEFICIENCY 21 |
| | 352), KEGIQTRNR (SEQ ID NO: | | |
| | 353), KKEGIQTRN (SEQ ID NO: | | |
| | 354), MKKEGIQTR (SEQ ID NO: | | |
| | 355), RNRKMSNKS (SEQ ID NO: | | |
| | 356), TRNRKMSNK (SEQ ID NO: 357) | | |
| GATA2 | GIQTRNRKM (SEQ ID NO: | Arg396Trp | IMMUNODEFICIENCY 21, MYELODYSPLASTIC |
| | 358), KEGIQTRNR (SEQ ID NO: | | SYNDROME |
| | 359), KKEGIQTRN (SEQ ID NO: | | |
| | 360), MKKEGIQTR (SEQ ID NO: | | |
| | 361), RNRKMSNKS (SEQ ID NO: | | |
| | 362), TRNRKMSNK (SEQ ID NO: 363) | | |
| GATA2 | GIQTRNRKM (SEQ ID NO: | Arg398Gln | IMMUNODEFICIENCY 21, MYELODYSPLASTIC |
| | 364), KEGIQTRNR (SEQ ID NO: | | SYNDROME |
| | 365), NRKMSNKSK (SEQ ID NO: | | |
| | 366), RKMSNKSKK (SEQ ID NO: | | |
| | 367), RNRKMSNKS (SEQ ID NO: | | |
| | 368), TRNRKMSNK (SEQ ID NO: 369) | | |
| GATA2 | GIQTRNRKM (SEQ ID NO: | Arg398Trp | Acute Myeloid Leukemia (AML), |
| | 370), KEGIQTRNR (SEQ ID NO: | | IMMUNODEFICIENCY 21, LEUKEMIA, ACUTE |
| | 371), NRKMSNKSK (SEQ ID NO: | | MYELOID, MYELODYSPLASTIC SYNDROME, |
| | 372), RKMSNKSKK (SEQ ID NO: | | Myelodysplastic Syndromes (MDS), |
| | 373), RNRKMSNKS (SEQ ID NO: | | Pancreatic Adenocarcinoma (PAAD) |
| | 374), TRNRKMSNK (SEQ ID NO: 375) | | |
| GATA2 | KARSCSEGR (SEQ ID NO: | Ala286Val | LEUKEMIA, ACUTE MYELOID |
| | 376), KQRSKARSC (SEQ ID NO: | | |
| | 377), PKQRSKARS (SEQ ID NO: | | |
| | 378), RSKARSCSE (SEQ ID NO: | | |
| | 379), TPKQRSKAR (SEQ ID NO: 380) | | |
| GATA2 | KEGIQTRNR (SEQ ID NO: | Lys390Glu | MYELODYSPLASTIC SYNDROME |
| | 381), KKEGIQTRN (SEQ ID NO: | | |
| | 382), MKKEGIQTR (SEQ ID NO: | | |
| | 383), NRPLTMKKE (SEQ ID NO: | | |
| | 384), VNRPLTMKK (SEQ ID NO: 385) | | |
| GATA2 | NRPLTMKKE (SEQ ID NO: | Pro385Gln | IMMUNODEFICIENCY 21 |
| | 386), VNRPLTMKK (SEQ ID NO: 387) | | |
| GATA3 | GIQTRNRKM (SEQ ID NO: | Arg364Gly | Breast Invasive Ductal Carcinoma (IDC), |
| | 388), KEGIQTRNR (SEQ ID NO: | | Invasive Breast Carcinoma (BRCA) |
| | 389), KKEGIQTRN (SEQ ID NO: | | |
| | 390), MKKEGIQTR (SEQ ID NO: | | |
| | 391), RNRKMSSKS (SEQ ID NO: | | |
| | 392), TRNRKMSSK (SEQ ID NO: 393) | | |
| GATA3 | GIQTRNRKM (SEQ ID NO: | Arg364Ile | Breast Invasive Ductal Carcinoma (IDC) |
| | 394), KEGIQTRNR (SEQ ID NO: | | |
| | 395), KKEGIQTRN (SEQ ID NO: | | |
| | 396), MKKEGIQTR (SEQ ID NO: | | |
| | 397), RNRKMSSKS (SEQ ID NO: | | |
| | 398), TRNRKMSSK (SEQ ID NO: 399) | | |
| GATA3 | GIQTRNRKM (SEQ ID NO: | Arg364Lys | Breast Invasive Ductal Carcinoma (IDC) |
| | 400), KEGIQTRNR (SEQ ID NO: | | |
| | 401), KKEGIQTRN (SEQ ID NO: | | |
| | 402), MKKEGIQTR (SEQ ID NO: | | |
| | 403), RNRKMSSKS (SEQ ID NO: | | |
| | 404), TRNRKMSSK (SEQ ID NO: 405) | | |
| GATA3 | GIQTRNRKM (SEQ ID NO: | Arg364Ser | Breast Invasive Ductal Carcinoma (IDC) |
| | 406), KEGIQTRNR (SEQ ID NO: | | |
| | 407), KKEGIQTRN (SEQ ID NO: | | |
| | 408), MKKEGIQTR (SEQ ID NO: | | |
| | 409), RNRKMSSKS (SEQ ID NO: | | |
| | 410), TRNRKMSSK (SEQ ID NO: 411) | | |
| GATA3 | GIQTRNRKM (SEQ ID NO: | Arg364Thr | Breast Invasive Ductal Carcinoma (IDC), |
| | 412), KEGIQTRNR (SEQ ID NO: | | Breast Mixed Ductal and Lobular Carcinoma |
| | 413), KKEGIQTRN (SEQ ID NO: | | (MDLC), Breast Neoplasm, NOS (BNNOS) |
| | 414), MKKEGIQTR (SEQ ID NO: | | |
| | 415), RNRKMSSKS (SEQ ID NO: | | |
| | 416), TRNRKMSSK (SEQ ID NO: 417) | | |
| GATA3 | INRPLTMKK (SEQ ID NO: | Arg352Ser | HYPOPARATHYROIDISM, SENSORINEURAL |
| | 418), NRPLTMKKE (SEQ ID NO: 419) | | DEAFNESS, AND RENAL DISEASE |
| GATA3 | INRPLTMKK (SEQ ID NO: | Arg352Thr | HYPOPARATHYROIDISM, SENSORINEURAL |
| | 420), NRPLTMKKE (SEQ ID NO: 421) | | DEAFNESS, AND RENAL DISEASE |
| GATA4 | EGIQTRKRK (SEQ ID NO: | Gln316Glu | ATRIAL SEPTAL DEFECT 2 |
| | 422), GIQTRKRKP (SEQ ID NO: | | |
| | 423), IQTRKRKPK (SEQ ID NO: | | |
| | 424), KEGIQTRKR (SEQ ID NO: | | |
| | 425), MRKEGIQTR (SEQ ID NO: | | |
| | 426), QTRKRKPKN (SEQ ID NO: | | |
| | 427), RKEGIQTRK (SEQ ID NO: 428) | | |
| GATA4 | MRKEGIQTR (SEQ ID NO: | Met310Val | ATRIAL SEPTAL DEFECT 2 |
| | 429), PRPLAMRKE (SEQ ID NO: | | |
| | 430), VPRPLAMRK (SEQ ID NO: 431) | | |
| GATA5 | ESIQTRKRK (SEQ ID NO: | Thr289Ala | ATRIOVENTRICULAR SEPTAL DEFECT |
| | 432), IQTRKRKPK (SEQ ID NO: | | |
| | 433), KESIQTRKR (SEQ ID NO: | | |
| | 434), KKESIQTRK (SEQ ID NO: | | |
| | 435), MKKESIQTR (SEQ ID NO: | | |
| | 436), QTRKRKPKT (SEQ ID NO: | | |
| | 437), SIQTRKRKP (SEQ ID NO: | | |
| | 438), TRKRKPKTI (SEQ ID NO: 439) | | |
| GATA5 | KRLSSSRRA (SEQ ID NO: | Leu233Pro | AORTIC VALVE DISEASE 1, AORTIC VALVE |
| | 440), PQKRLSSSR (SEQ ID NO: | | DISEASE 2 |
| | 441), QKRLSSSRR (SEQ ID NO: 442) | | |
| GLI1 | RKHVKTVHG (SEQ ID NO: | Arg380Gln | Colon Adenocarcinoma (COAD), Cutaneous |
| | 443), SLRKHVKTV (SEQ ID NO: | | Squamous Cell Carcinoma (CSCC), Lung |
| | 444), SSLRKHVKT (SEQ ID NO: 445) | | Adenocarcinoma (LUAD), Mucinous |
| | | | Adenocarcinoma of the Colon and Rectum |
| | | | (MACR), Rectal Adenocarcinoma (READ), |
| | | | Uterine Carcinosarcoma/Uterine Malignant |
| | | | Mixed Mullerian Tumor (UCS), Uterine Clear |
| | | | Cell Carcinoma (UCCC), Uterine |
| | | | Endometrioid Carcinoma (UEC) |
| GLI2 | CPRPLGPRR (SEQ ID NO: | Pro932Ser | HOLOPROSENCEPHALY 1 |
| | 446), PRPLGPRRG (SEQ ID NO: 447) | | |
| GLI2 | DRAKHQNRT (SEQ ID NO: | Ala551Thr | CRANIOSYNOSTOSIS 1 |
| | 448), RAKHQNRTH (SEQ ID NO: | | |
| | 449), SDRAKHQNR (SEQ ID NO: 450) | | |
| GLI2 | KIPGCTKRY (SEQ ID NO: 451) | Tyr575His | PITUITARY HORMONE DEFICIENCY, |
| | | | COMBINED, 2 |
| GLI2 | PRLSRKRAL (SEQ ID NO: | Arg226His | HOLOPROSENCEPHALY 1 |
| | 452), RLSRKRALS (SEQ ID NO: | | |
| | 453), RVTPRLSRK (SEQ ID NO: | | |
| | 454), TPRLSRKRA (SEQ ID NO: | | |
| | 455), VTPRLSRKR (SEQ ID NO: 456) | | |
| GPBP1 | DKRERKQFE (SEQ ID NO: | Arg129Cys | AUTISM |
| | 457), EDKRERKQF (SEQ ID NO: | | |
| | 458), GRKEDKRER (SEQ ID NO: | | |
| | 459), KEDKRERKQ (SEQ ID NO: | | |
| | 460), KRERKQFEA (SEQ ID NO: | | |
| | 461), RERKQFEAE (SEQ ID NO: | | |
| | 462), RKEDKRERK (SEQ ID NO: 463) | | |
| GPBP1 | KLTRMRTDK (SEQ ID NO: | Arg292Gln | AUTISM |
| | 464), LTRMRTDKK (SEQ ID NO: | | |
| | 465), PRLTKLTRM (SEQ ID NO: | | |
| | 466), RLTKLTRMR (SEQ ID NO: | | |
| | 467), RMRTDKKSE (SEQ ID NO: | | |
| | 468), TKLTRMRTD (SEQ ID NO: | | |
| | 469), TRMRTDKKS (SEQ ID NO: 470) | | |
| GRHL3 | DDERKQFRR (SEQ ID NO: | Asp410Gly | CLEFT PALATE, ISOLATED |
| | 471), ERKMRDDER (SEQ ID NO: | | |
| | 472), GAERKMRDD (SEQ ID NO: | | |
| | 473), KMRDDERKQ (SEQ ID NO: | | |
| | 474), RDDERKQFR (SEQ ID NO: | | |
| | 475), RKMRDDERK (SEQ ID NO: 476) | | |
| GRHL3 | PKEKRILSS (SEQ ID NO: | Pro70Thr | NEURAL TUBE DEFECTS, SUSCEPTIBILITY TO |
| | 477), YMGPKEKRI (SEQ ID NO: 478) | | |
| GSC | EKREEEGKS (SEQ ID NO: | Glu247Gly | EAR MALFORMATION |
| | 479), KREEEGKSD (SEQ ID NO: | | |
| | 480), PEKREEEGK (SEQ ID NO: 481) | | |
| HES7 | EKRRRDRIN (SEQ ID NO: | Arg25Trp | SPONDYLOCOSTAL DYSOSTOSIS 1, |
| | 482), KPLVEKRRR (SEQ ID NO: | | AUTOSOMAL RECESSIVE, SPONDYLOCOSTAL |
| | 483), KRRRDRINR (SEQ ID NO: | | DYSOSTOSIS 4, AUTOSOMAL RECESSIVE, |
| | 484), VEKRRRDRI (SEQ ID NO: 485) | | SPONDYLOCOSTAL DYSOSTOSIS 5 |
| HES7 | EKRRRDRIN (SEQ ID NO: | Asn29Ser | SPONDYLOCOSTAL DYSOSTOSIS 1, |
| | 486), KRRRDRINR (SEQ ID NO: 487) | | AUTOSOMAL RECESSIVE, SPONDYLOCOSTAL |
| | | | DYSOSTOSIS 4, AUTOSOMAL RECESSIVE |
| HESX1 | KRELSWYRG (SEQ ID NO: | Trp105Gly | PITUITARY HORMONE DEFICIENCY, |
| | 488), LKRELSWYR (SEQ ID NO: | | COMBINED, 1, SEPTOOPTIC DYSPLASIA |
| | 489), LSWYRGRRP (SEQ ID NO: | | |
| | 490), RELSWYRGR (SEQ ID NO: | | |
| | 491), SWYRGRRPR (SEQ ID NO: | | |
| | 492), WYRGRRPRT (SEQ ID NO: 493) | | |
| HESX1 | KRELSWYRG (SEQ ID NO: | Glu102Gly | PITUITARY HORMONE DEFICIENCY, |
| | 494), LKRELSWYR (SEQ ID NO: | | COMBINED, 2 |
| | 495), RELSWYRGR (SEQ ID NO: | | |
| | 496), SERLSLKRE (SEQ ID NO: 497) | | |
| HIVEP1 | GCHREMRRT (SEQ ID NO: | Arg1365Leu | AUTISM |
| | 498), PGCHREMRR (SEQ ID NO: | | |
| | 499), REMRRTASE (SEQ ID NO: 500) | | |
| HIVEP1 | GCHREMRRT (SEQ ID NO: | Met1363Ile | ATTENTION DEFICIT-HYPERACTIVITY |
| | 501), PGCHREMRR (SEQ ID NO: | | DISORDER, EAR MALFORMATION |
| | 502), REMRRTASE (SEQ ID NO: 503) | | |
| HNF4A | ERDRISTRR (SEQ ID NO: | Arg136Gln | AUTOIMMUNE DISEASE |
| | 504), NERDRISTR (SEQ ID NO: | | |
| | 505), RDRISTRRS (SEQ ID NO: 506) | | |
| IRX5 | NARRRLKKE (SEQ ID NO: 507) | Asn166Lys | HAMAMY SYNDROME |
| KDM2A | CAPRKDRQV (SEQ ID NO: | Arg449Lys | AUTISM |
| | 508), PRKDRQVHL (SEQ ID NO: | | |
| | 509), RKDRQVHLT (SEQ ID NO: 510) | | |
| KDM2B | EGQEPAKRR (SEQ ID NO: | Pro763Thr | AUTISM |
| | 511), KEGQEPAKR (SEQ ID NO: | | |
| | 512), PAKRRSECE (SEQ ID NO: 513) | | |
| KDM5B | LRRRMGCPT (SEQ ID NO: | Pro263Ser | MENTAL RETARDATION, AUTOSOMAL |
| | 514), NLRRRMGCP (SEQ ID NO: | | RECESSIVE 65 |
| | 515), RRMGCPTPK (SEQ ID NO: | | |
| | 516), RRRMGCPTP (SEQ ID NO: 517) | | |
| KLF12 | SHLKAHRRT (SEQ ID NO: | Ser332Cys | SCHIZOPHRENIA |
| | 518), SSHLKAHRR (SEQ ID NO: 519) | | |
| KMT2A | AAVALGRKR (SEQ ID NO: | Arg1083Gln | COLOBOMA, OCULAR, AUTOSOMAL |
| | 520), GRKRAVFPD (SEQ ID NO: | | DOMINANT |
| | 521), LGRKRAVFP (SEQ ID NO: | | |
| | 522), RKRAVFPDD (SEQ ID NO: 523) | | |
| MAX | ALERKRRDH (SEQ ID NO: | Arg36Thr | Lung Adenocarcinoma (LUAD) |
| | 524), ERKRRDHIK (SEQ ID NO: | | |
| | 525), HNALERKRR (SEQ ID NO: | | |
| | 526), KRRDHIKDS (SEQ ID NO: | | |
| | 527), LERKRRDHI (SEQ ID NO: | | |
| | 528), NALERKRRD (SEQ ID NO: | | |
| | 529), RKRRDHIKD (SEQ ID NO: 530) | | |
| MAX | HHNALERKR (SEQ ID NO: | His28Arg | Endometrial Carcinoma (UCEC), Mucinous |
| | 531), HNALERKRR (SEQ ID NO: 532) | | Adenocarcinoma of the Colon and Rectum |
| | | | (MACR), Prostate Adenocarcinoma (PRAD), |
| | | | Seminoma (SEM), Uterine Endometrioid |
| | | | Carcinoma (UEC), Uterine Serous |
| | | | Carcinoma/Uterine Papillary Serous |
| | | | Carcinoma (USC) |
| MAX | RFQSAADKR (SEQ ID NO: 533) | Arg25Trp | PARAGANGLIOMAS 1, PHEOCHROMOCYTOMA |
| MAX | RFQSAADKR (SEQ ID NO: 534) | Asp23Asn | PARAGANGLIOMAS 1, PHEOCHROMOCYTOMA |
| MBD1 | ARRKGGCDS (SEQ ID NO: | Arg269Cys | AUTISM |
| | 535), CLRGKHARR (SEQ ID NO: | | |
| | 536), GKHARRKGG (SEQ ID NO: | | |
| | 537), HARRKGGCD (SEQ ID NO: | | |
| | 538), KHARRKGGC (SEQ ID NO: | | |
| | 539), LRGKHARRK (SEQ ID NO: | | |
| | 540), RGKHARRKG (SEQ ID NO: | | |
| | 541), RRKGGCDSK (SEQ ID NO: 542) | | |
| MBD4 | EALSPPRRK (SEQ ID NO: | Arg432His | AUTISM |
| | 543), KEALSPPRR (SEQ ID NO: | | |
| | 544), PPRRKAFKK (SEQ ID NO: | | |
| | 545), PRRKAFKKW (SEQ ID NO: | | |
| | 546), RKAFKKWTP (SEQ ID NO: | | |
| | 547), RRKAFKKWT (SEQ ID NO: | | |
| | 548), SPPRRKAFK (SEQ ID NO: 549) | | |
| MBD6 | ARGRKPGSR (SEQ ID NO: | Arg883Trp | AUTISM |
| | 550), GRKPGSRRE (SEQ ID NO: | | |
| | 551), GSRREPGRL (SEQ ID NO: | | |
| | 552), PGSRREPGR (SEQ ID NO: | | |
| | 553), RGRKPGSRR (SEQ ID NO: | | |
| | 554), RKPGSRREP (SEQ ID NO: | | |
| | 555), SRREPGRLA (SEQ ID NO: 556) | | |
| MBD6 | KVPPGVVRK (SEQ ID NO: | Pro943Arg | AUTISM |
| | 557), PGVVRKSRR (SEQ ID NO: 558) | | |
| MEF2B | DILETLKRR (SEQ ID NO: 559) | Asp83Ala | Follicular Lymphoma (FL) |
| MEF2B | DILETLKRR (SEQ ID NO: 560) | Asp83Asn | Dedifferentiated Liposarcoma (DDLS) |
| MEF2B | DILETLKRR (SEQ ID NO: 561) | Asp83Gly | Follicular Lymphoma (FL) |
| MEF2B | DILETLKRR (SEQ ID NO: 562) | Asp83Val | Diffuse Large B-Cell Lymphoma, NOS |
| | | | (DLBCLNOS), Follicular Lymphoma (FL), |
| | | | Glioblastoma Multiforme (GBM), Histiocytic |
| | | | Dendritic Cell Sarcoma (HDCS) |
| MEF2D | APSRKPDLR (SEQ ID NO: | Arg266Cys | AUTISM |
| | 563), PSRKPDLRV (SEQ ID NO: | | |
| | 564), SRKPDLRVI (SEQ ID NO: 565) | | |
| MESP2 | REKLRMRTL (SEQ ID NO: | Lys91Glu | SPONDYLOCOSTAL DYSOSTOSIS 2, |
| | 566), RQSASEREK (SEQ ID NO: | | AUTOSOMAL RECESSIVE |
| | 567), SASEREKLR (SEQ ID NO: | | |
| | 568), SEREKLRMR (SEQ ID NO: 569) | | |
| MGA | KKISGDMRG (SEQ ID NO: | Gly2753Glu | AUTISM |
| | 570), MRGIQYKWK (SEQ ID NO: 571) | | |
| MITF | AKERQKKDN (SEQ ID NO: | Glu309Lys | TIETZ ALBINISM-DEAFNESS SYNDROME |
| | 572), ALAKERQKK (SEQ ID NO: | | |
| | 573), EARALAKER (SEQ ID NO: | | |
| | 574), ERQKKDNHN (SEQ ID NO: | | |
| | 575), KERQKKDNH (SEQ ID NO: | | |
| | 576), LAKERQKKD (SEQ ID NO: | | |
| | 577), RALAKERQK (SEQ ID NO: 578) | | |
| MITF | AKERQKKDN (SEQ ID NO: | Lys313Asn | COLOBOMA, OSTEOPETROSIS, |
| | 579), ALAKERQKK (SEQ ID NO: | | MICROPHTHALMIA, MACROCEPHALY, |
| | 580), ERQKKDNHN (SEQ ID NO: | | ALBINISM, AND DEAFNESS |
| | 581), KERQKKDNH (SEQ ID NO: | | |
| | 582), LAKERQKKD (SEQ ID NO: | | |
| | 583), RQKKDNHNL (SEQ ID NO: 584) | | |
| MITF | ERQKKDNHN (SEQ ID NO: | Asn317Lys | TIETZ ALBINISM-DEAFNESS SYNDROME |
| | 585), HNLIERRRR (SEQ ID NO: | | |
| | 586), NHNLIERRR (SEQ ID NO: | | |
| | 587), NLIERRRRF (SEQ ID NO: | | |
| | 588), RQKKDNHNL (SEQ ID NO: 589) | | |
| MITF | ERRRRFNIN (SEQ ID NO: | Arg324Gly | COLOBOMA, OSTEOPETROSIS, |
| | 590), HNLIERRRR (SEQ ID NO: | | MICROPHTHALMIA, MACROCEPHALY, |
| | 591), IERRRRFNI (SEQ ID NO: | | ALBINISM, AND DEAFNESS |
| | 592), LIERRRRFN (SEQ ID NO: | | |
| | 593), NLIERRRRF (SEQ ID NO: | | |
| | 594), RRRFNINDR (SEQ ID NO: | | |
| | 595), RRRRFNIND (SEQ ID NO: 596) | | |
| MITF | ERRRRFNIN (SEQ ID NO: | Arg324Ile | TIETZ ALBINISM-DEAFNESS SYNDROME |
| | 597), HNLIERRRR (SEQ ID NO: | | |
| | 598), IERRRRFNI (SEQ ID NO: | | |
| | 599), LIERRRRFN (SEQ ID NO: | | |
| | 600), NLIERRRRF (SEQ ID NO: | | |
| | 601), RRRFNINDR (SEQ ID NO: | | |
| | 602), RRRRFNIND (SEQ ID NO: 603) | | |
| MITF | GASKTSSRR (SEQ ID NO: 604) | Gly506Arg | EAR MALFORMATION |
| MITF | HNLIERRRR (SEQ ID NO: | Ile319Met | TIETZ ALBINISM-DEAFNESS SYNDROME |
| | 605), IERRRRFNI (SEQ ID NO: | | |
| | 606), LIERRRRFN (SEQ ID NO: | | |
| | 607), NHNLIERRR (SEQ ID NO: | | |
| | 608), NLIERRRRF (SEQ ID NO: 609) | | |
| MITF | HNLIERRRR (SEQ ID NO: | Leu318Pro | EAR MALFORMATION |
| | 610), LIERRRRFN (SEQ ID NO: | | |
| | 611), NHNLIERRR (SEQ ID NO: | | |
| | 612), NLIERRRRF (SEQ ID NO: | | |
| | 613), RQKKDNHNL (SEQ ID NO: 614) | | |
| MSX1 | RFSPPPARR (SEQ ID NO: 615) | Pro153Gln | OROFACIAL CLEFT 5 |
| MYB | GKTRWTREE (SEQ ID NO: | Arg45Gln | AUTISM |
| | 616), LGKTRWTRE (SEQ ID NO: 617) | | |
| MYCN | DSEDSERRR (SEQ ID NO: | Arg382His | FEINGOLD SYNDROME 1 |
| | 618), ERRRNHNIL (SEQ ID NO: | | |
| | 619), RRRNHNILE (SEQ ID NO: | | |
| | 620), SERRRNHNI (SEQ ID NO: 621) | | |
| MYCN | ERQRRNDLR (SEQ ID NO: | Arg393Cys | FEINGOLD SYNDROME 1 |
| | 622), NILERQRRN (SEQ ID NO: | | |
| | 623), QRRNDLRSS (SEQ ID NO: | | |
| | 624), RQRRNDLRS (SEQ ID NO: 625) | | |
| MYCN | ERQRRNDLR (SEQ ID NO: | Arg393His | FEINGOLD SYNDROME 1 |
| | 626), NILERQRRN (SEQ ID NO: | | |
| | 627), QRRNDLRSS (SEQ ID NO: | | |
| | 628), RQRRNDLRS (SEQ ID NO: 629) | | |
| MYCN | ERQRRNDLR (SEQ ID NO: | Arg393Ser | FEINGOLD SYNDROME 1 |
| | 630), NILERQRRN (SEQ ID NO: | | |
| | 631), QRRNDLRSS (SEQ ID NO: | | |
| | 632), RQRRNDLRS (SEQ ID NO: 633) | | |
| MYCN | ERQRRNDLR (SEQ ID NO: | Arg394His | FEINGOLD SYNDROME 1 |
| | 634), NILERQRRN (SEQ ID NO: | | |
| | 635), QRRNDLRSS (SEQ ID NO: | | |
| | 636), RQRRNDLRS (SEQ ID NO: 637) | | |
| MYCN | ERQRRNDLR (SEQ ID NO: | Arg394Leu | FEINGOLD SYNDROME 1 |
| | 638), NILERQRRN (SEQ ID NO: | | |
| | 639), QRRNDLRSS (SEQ ID NO: | | |
| | 640), RQRRNDLRS (SEQ ID NO: 641) | | |
| MYCN | ERQRRNDLR (SEQ ID NO: | Arg391Cys | FEINGOLD SYNDROME 1 |
| | 642), NILERQRRN (SEQ ID NO: | | |
| | 643), RQRRNDLRS (SEQ ID NO: | | |
| | 644), RRNHNILER (SEQ ID NO: 645) | | |
| MYCN | ERQRRNDLR (SEQ ID NO: | Arg391Ser | FEINGOLD SYNDROME 1 |
| | 646), NILERQRRN (SEQ ID NO: | | |
| | 647), RQRRNDLRS (SEQ ID NO: | | |
| | 648), RRNHNILER (SEQ ID NO: 649) | | |
| MYF6 | CKRKSAPTD (SEQ ID NO: | Ala90Asp | MYOSITIS |
| | 650), CKTCKRKSA (SEQ ID NO: | | |
| | 651), KRKSAPTDR (SEQ ID NO: | | |
| | 652), KSAPTDRRK (SEQ ID NO: | | |
| | 653), KTCKRKSAP (SEQ ID NO: | | |
| | 654), RKSAPTDRR (SEQ ID NO: | | |
| | 655), TCKRKSAPT (SEQ ID NO: 656) | | |
| MYTIL | DYTKMKPRR (SEQ ID NO: | Arg830Lys | AUTISM |
| | 657), KMKPRRIDE (SEQ ID NO: | | |
| | 658), KPRRIDEDE (SEQ ID NO: | | |
| | 659), MKPRRIDED (SEQ ID NO: | | |
| | 660), RRIDEDESK (SEQ ID NO: | | |
| | 661), TKMKPRRID (SEQ ID NO: | | |
| | 662), YTKMKPRRI (SEQ ID NO: 663) | | |
| MYTIL | RTEKKESKC (SEQ ID NO: 664) | Cys506Arg | AUTISM, MENTAL RETARDATION, |
| | | | AUTOSOMAL DOMINANT 39 |
| NCOA2 | RMQPRPGLR (SEQ ID NO: 1275) | Met1170Thr | ENDOMETRIAL CANCER |
| NCOA3 | PRNRGSPKI (SEQ ID NO: | Arg485Cys | AUTOIMMUNE DISEASE |
| | 665), RNRGSPKIA (SEQ ID NO: | | |
| | 666), SPRNRGSPK (SEQ ID NO: 667) | | |
| NEUROD2 | RQKANARER (SEQ ID NO: | Glu130Gln | DEVELOPMENTAL AND EPILEPTIC |
| | 668), RRQKANARE (SEQ ID NO: 669) | | ENCEPHALOPATHY 72 |
| NEUROG3 | KANDRERNR (SEQ ID NO: | Arg93Leu | DIARRHEA 4, MALABSORPTIVE, CONGENITAL |
| | 670), KKANDRERN (SEQ ID NO: | | |
| | 671), NDRERNRMH (SEQ ID NO: | | |
| | 672), RERNRMHNL (SEQ ID NO: | | |
| | 673), RKKANDRER (SEQ ID NO: 674) | | |
| NFE2 | PVRAKPTAR (SEQ ID NO: | Arg219Gln | Adrenocortical Carcinoma (ACC), Cutaneous |
| | 675), RAKPTARGE (SEQ ID NO: | | Melanoma (SKCM) |
| | 676), VRAKPTARG (SEQ ID NO: 677) | | |
| NFE2 | PVRAKPTAR (SEQ ID NO: | Arg219Leu | Cutaneous Melanoma (SKCM) |
| | 678), RAKPTARGE (SEQ ID NO: | | |
| | 679), VRAKPTARG (SEQ ID NO: 680) | | |
| NR1D2 | RDAVRFGRI (SEQ ID NO: | Arg175Trp | ATRIOVENTRICULAR SEPTAL DEFECT |
| | 681), RFGRIPKRE (SEQ ID NO: | | |
| | 682), VRFGRIPKR (SEQ ID NO: 683) | | |
| NR3C2 | AKPLYFHRK (SEQ ID NO: 684) | Leu979Pro | PSEUDOHYPOALDOSTERONISM, TYPE I, |
| | | | AUTOSOMAL DOMINANT |
| NR5A1 | ADRMRGGRN (SEQ ID NO: | Arg92Gln | 46, XX SEX REVERSAL 4, 46, XY SEX REVERSAL |
| | 685), DRMRGGRNK (SEQ ID NO: | | 3, HYPOADRENOCORTICISM, FAMILIAL |
| | 686), MRGGRNKFG (SEQ ID NO: | | |
| | 687), RADRMRGGR (SEQ ID NO: | | |
| | 688), RMRGGRNKF (SEQ ID NO: | | |
| | 689), RNKFGPMYK (SEQ ID NO: 690) | | |
| NR5A1 | ADRMRGGRN (SEQ ID NO: | Gly91Ser | 46, XY SEX REVERSAL 3 |
| | 691), DRMRGGRNK (SEQ ID NO: | | |
| | 692), MRGGRNKFG (SEQ ID NO: | | |
| | 693), RADRMRGGR (SEQ ID NO: | | |
| | 694), RMRGGRNKF (SEQ ID NO: | | |
| | 695), VRADRMRGG (SEQ ID NO: 696) | | |
| NR5A1 | EAVRADRMR (SEQ ID NO: | Arg84His | 46, XY SEX REVERSAL 3, SPERMATOGENIC |
| | 697), RADRMRGGR (SEQ ID NO: | | FAILURE 1 |
| | 698), VRADRMRGG (SEQ ID NO: 699) | | |
| NR5A1 | KQQKKAQIR (SEQ ID NO: | Arg114Gln | 46, XY SEX REVERSAL 11 |
| | 700), QKKAQIRAN (SEQ ID NO: | | |
| | 701), QQKKAQIRA (SEQ ID NO: 702) | | |
| PAX6 | RAIGGSKPR (SEQ ID NO: | Gly72Arg | FOVEAL HYPOPLASIA 1 |
| | 703), RPRAIGGSK (SEQ ID NO: 704) | | |
| PAX6 | RPRAIGGSK (SEQ ID NO: 705) | Pro68Ser | COLOBOMA OF OPTIC NERVE |
| PHF21A | GTRKRGRPP (SEQ ID NO: | Gly429Ser | INTELLECTUAL DEVELOPMENTAL DISORDER |
| | 706), HPGTRKRGR (SEQ ID NO: | | WITH BEHAVIORAL ABNORMALITIES AND |
| | 707), KRGRPPKYN (SEQ ID NO: | | CRANIOFACIAL DYSMORPHISM WITH OR |
| | 708), PGTRKRGRP (SEQ ID NO: | | WITHOUT SEIZURES |
| | 709), RKRGRPPKY (SEQ ID NO: | | |
| | 710), TRKRGRPPK (SEQ ID NO: 711) | | |
| POU3F4 | CNRRQKEKR (SEQ ID NO: | Lys334Glu | DEAFNESS, X-LINKED 2 |
| | 712), FCNRRQKEK (SEQ ID NO: | | |
| | 713), KEKRMTPPG (SEQ ID NO: | | |
| | 714), NRRQKEKRM (SEQ ID NO: | | |
| | 715), QKEKRMTPP (SEQ ID NO: | | |
| | 716), RQKEKRMTP (SEQ ID NO: | | |
| | 717), RRQKEKRMT (SEQ ID NO: 718) | | |
| POU3F4 | CNRRQKEKR (SEQ ID NO: | Gln331Pro | EAR MALFORMATION |
| | 719), FCNRRQKEK (SEQ ID NO: | | |
| | 720), NRRQKEKRM (SEQ ID NO: | | |
| | 721), QKEKRMTPP (SEQ ID NO: | | |
| | 722), RQKEKRMTP (SEQ ID NO: | | |
| | 723), RRQKEKRMT (SEQ ID NO: 724) | | |
| POU3F4 | CNRRQKEKR (SEQ ID NO: | Arg330Lys | EAR MALFORMATION |
| | 725), FCNRRQKEK (SEQ ID NO: | | |
| | 726), NRRQKEKRM (SEQ ID NO: | | |
| | 727), RQKEKRMTP (SEQ ID NO: | | |
| | 728), RRQKEKRMT (SEQ ID NO: 729) | | |
| POU3F4 | CNRRQKEKR (SEQ ID NO: | Arg330Ser | DEAFNESS, X-LINKED 2 |
| | 730), FCNRRQKEK (SEQ ID NO: | | |
| | 731), NRRQKEKRM (SEQ ID NO: | | |
| | 732), RQKEKRMTP (SEQ ID NO: | | |
| | 733), RRQKEKRMT (SEQ ID NO: 734) | | |
| POU4F3 | ERKRKRTSI (SEQ ID NO: | Ile281Val | EAR MALFORMATION |
| | 735), KRKRTSIAA (SEQ ID NO: | | |
| | 736), RKRKRTSIA (SEQ ID NO: | | |
| | 737), RKRTSIAAP (SEQ ID NO: 738) | | |
| POU4F3 | QKQKRMKYS (SEQ ID NO: 739) | Lys328Glu | EAR MALFORMATION |
| PRDM16 | ALKEKYLRP (SEQ ID NO: | Pro889Leu | CARDIAC CONDUCTION DEFECT, WOLFF- |
| | 740), LKEKYLRPS (SEQ ID NO: 741) | | PARKINSON-WHITE SYNDROME |
| PRDM16 | KGKERYTCR (SEQ ID NO: | Thr952Met | CARDIAC CONDUCTION DEFECT, WOLFF- |
| | 742), LRKGKERYT (SEQ ID NO: | | PARKINSON-WHITE SYNDROME |
| | 743), RKGKERYTC (SEQ ID NO: 744) | | |
| PRRX1 | IANLRLKAK (SEQ ID NO: | Ala231Pro | AGNATHIA-OTOCEPHALY COMPLEX |
| | 745), KAKEYSLQR (SEQ ID NO: | | |
| | 746), NLRLKAKEY (SEQ ID NO: | | |
| | 747), RLKAKEYSL (SEQ ID NO: 748) | | |
| RAG1 | FKLFRVRSF (SEQ ID NO: | Ser37Tyr | AUTOIMMUNE DISEASE |
| | 749), FRVRSFEKT (SEQ ID NO: | | |
| | 750), KFKLFRVRS (SEQ ID NO: | | |
| | 751), LFRVRSFEK (SEQ ID NO: | | |
| | 752), RVRSFEKTP (SEQ ID NO: 753) | | |
| RBPJ | KSYGNEKRF (SEQ ID NO: 754) | Phe66Val | ADAMS-OLIVER SYNDROME 1, ADAMS- |
| | | | OLIVER SYNDROME 3, APLASIA CUTIS |
| | | | CONGENITA, NONSYNDROMIC |
| RBPJ | KSYGNEKRF (SEQ ID NO: | Arg65Gly | ADAMS-OLIVER SYNDROME 1, ADAMS- |
| | 755), QKSYGNEKR (SEQ ID NO: 756) | | OLIVER SYNDROME 3, APLASIA CUTIS |
| | | | CONGENITA, NONSYNDROMIC |
| RBPJ | KSYGNEKRF (SEQ ID NO: | Glu63Gly | ADAMS-OLIVER SYNDROME 1, ADAMS- |
| | 757), QKSYGNEKR (SEQ ID NO: 758) | | OLIVER SYNDROME 3, APLASIA CUTIS |
| | | | CONGENITA, NONSYNDROMIC |
| REPIN1 | KQLRAHLRR (SEQ ID NO: | Ala162Thr | AUTISM |
| | 759), QLRAHLRRC (SEQ ID NO: 760) | | |
| RUNX1 | AGKLRSGDR (SEQ ID NO: | Gly42Arg | LEUKEMIA, ACUTE MYELOID |
| | 761), GKLRSGDRS (SEQ ID NO: 762) | | |
| RUNX1 | EPRRHRQKL (SEQ ID NO: | Arg180Gln | LEUKEMIA, ACUTE MYELOID |
| | 763), GPREPRRHR (SEQ ID NO: | | |
| | 764), PREPRRHRQ (SEQ ID NO: | | |
| | 765), PRRHRQKLD (SEQ ID NO: | | |
| | 766), REPRRHRQK (SEQ ID NO: | | |
| | 767), RHRQKLDDQ (SEQ ID NO: | | |
| | 768), RQKLDDQTK (SEQ ID NO: | | |
| | 769), RRHRQKLDD (SEQ ID NO: 770) | | |
| RUNX1 | EPRRHRQKL (SEQ ID NO: | Arg180Trp | LEUKEMIA, ACUTE MYELOID |
| | 771), GPREPRRHR (SEQ ID NO: | | |
| | 772), PREPRRHRQ (SEQ ID NO: | | |
| | 773), PRRHRQKLD (SEQ ID NO: | | |
| | 774), REPRRHRQK (SEQ ID NO: | | |
| | 775), RHRQKLDDQ (SEQ ID NO: | | |
| | 776), RQKLDDQTK (SEQ ID NO: | | |
| | 777), RRHRQKLDD (SEQ ID NO: 778) | | |
| RUNX3 | RFGDLERLR (SEQ ID NO: 779) | Arg197His | SCHIZOPHRENIA |
| SATB2 | ILRKEEDPR (SEQ ID NO: | Arg399His | GLASS SYNDROME |
| | 780), LRKEEDPRT (SEQ ID NO: | | |
| | 781), RKEEDPRTA (SEQ ID NO: 782) | | |
| SATB2 | KKPRSRTKI (SEQ ID NO: | Ile621Phe | AUTISM |
| | 783), KPRSRTKIS (SEQ ID NO: | | |
| | 784), RSRTKISLE (SEQ ID NO: 785) | | |
| SATB2 | RDRIYQDER (SEQ ID NO: 786) | Arg429Gln | GLASS SYNDROME |
| SATB2 | RDRIYQDER (SEQ ID NO: | Tyr433Ser | GLASS SYNDROME |
| | 787), RIYQDERER (SEQ ID NO: 788) | | |
| SETDB1 | MRNEQYRGK (SEQ ID NO: | Glu592Gln | EAR MALFORMATION |
| | 789), RPMRNEQYR (SEQ ID NO: | | |
| | 790), SRVRPMRNE (SEQ ID NO: 791) | | |
| SETDB2 | IFSKKRKLE (SEQ ID NO: | Ile425Thr | AUTISM |
| | 792), KNIFSKKRK (SEQ ID NO: | | |
| | 793), MKNIFSKKR (SEQ ID NO: | | |
| | 794), NIFSKKRKL (SEQ ID NO: 795) | | |
| SIM1 | EKSKNAART (SEQ ID NO: | Lys4Met | OBESITY |
| | 796), KEKSKNAAR (SEQ ID NO: | | |
| | 797), KSKNAARTR (SEQ ID NO: 798) | | |
| SIX3 | DRAAAAKNR (SEQ ID NO: 799) | Arg269Met | HOLOPROSENCEPHALY 1 |
| SIX3 | DRAAAAKNR (SEQ ID NO: 800) | Arg269Ser | HOLOPROSENCEPHALY 1 |
| SIX3 | DRAAAAKNR (SEQ ID NO: 801) | Arg269Thr | HOLOPROSENCEPHALY 1 |
| SKI | KDKPSSWLR (SEQ ID NO: 802) | Arg357Trp | AORTIC ANEURYSM, FAMILIAL THORACIC 1 |
| SOX10 | ADPKRDGRS (SEQ ID NO: | Arg258Gln | AUTISM |
| | 803), DPKRDGRSM (SEQ ID NO: | | |
| | 804), KADPKRDGR (SEQ ID NO: | | |
| | 805), KRDGRSMGE (SEQ ID NO: | | |
| | 806), QSGKADPKR (SEQ ID NO: | | |
| | 807), SGKADPKRD (SEQ ID NO: 808) | | |
| SOX10 | DYKYQPRRR (SEQ ID NO: | Arg177Gln | HYPOGONADOTROPIC HYPOGONADISM 1 |
| | 809), KYQPRRRKN (SEQ ID NO: | | WITH OR WITHOUT ANOSMIA |
| | 810), PDYKYQPRR (SEQ ID NO: | | |
| | 811), PRRRKNGKA (SEQ ID NO: | | |
| | 812), QPRRRKNGK (SEQ ID NO: | | |
| | 813), RRKNGKAAQ (SEQ ID NO: | | |
| | 814), RRRKNGKAA (SEQ ID NO: | | |
| | 815), YKYQPRRRK (SEQ ID NO: | | |
| | 816), YQPRRRKNG (SEQ ID NO: 817) | | |
| SOX10 | PDYKYQPRR (SEQ ID NO: 818) | Pro169Leu | EAR MALFORMATION |
| SOX17 | AKGESRIRR (SEQ ID NO: | Arg70Gln | PULMONARY HYPERTENSION, PRIMARY, 1 |
| | 819), KGESRIRRP (SEQ ID NO: | | |
| | 820), RIRRPMNAF (SEQ ID NO: | | |
| | 821), SRIRRPMNA (SEQ ID NO: 822) | | |
| SOX17 | HPNYKYRPR (SEQ ID NO: 823) | His132Asp | PULMONARY HYPERTENSION, PRIMARY, 1 |
| SOX17 | HPNYKYRPR (SEQ ID NO: | Arg140Pro | PULMONARY HYPERTENSION, PRIMARY, 1 |
| | 824), KYRPRRRKQ (SEQ ID NO: | | |
| | 825), NYKYRPRRR (SEQ ID NO: | | |
| | 826), PNYKYRPRR (SEQ ID NO: | | |
| | 827), PRRRKQVKR (SEQ ID NO: | | |
| | 828), RPRRRKQVK (SEQ ID NO: | | |
| | 829), RRRKQVKRL (SEQ ID NO: | | |
| | 830), YKYRPRRRK (SEQ ID NO: | | |
| | 831), YRPRRRKQV (SEQ ID NO: 832) | | |
| SOX17 | HPNYKYRPR (SEQ ID NO: | Arg140Trp | PULMONARY HYPERTENSION, PRIMARY, 1 |
| | 833), KYRPRRRKQ (SEQ ID NO: | | |
| | 834), NYKYRPRRR (SEQ ID NO: | | |
| | 835), PNYKYRPRR (SEQ ID NO: | | |
| | 836), PRRRKQVKR (SEQ ID NO: | | |
| | 837), RPRRRKQVK (SEQ ID NO: | | |
| | 838), RRRKQVKRL (SEQ ID NO: | | |
| | 839), YKYRPRRRK (SEQ ID NO: | | |
| | 840), YRPRRRKQV (SEQ ID NO: 841) | | |
| SOX17 | HPNYKYRPR (SEQ ID NO: | Pro133Ala | PULMONARY HYPERTENSION, PRIMARY, 1 |
| | 842), PNYKYRPRR (SEQ ID NO: 843) | | |
| SOX17 | HPNYKYRPR (SEQ ID NO: | Pro133Leu | PULMONARY HYPERTENSION, PRIMARY, 1 |
| | 844), PNYKYRPRR (SEQ ID NO: 845) | | |
| SOX17 | HPNYKYRPR (SEQ ID NO: | Pro133Ser | PULMONARY HYPERTENSION, PRIMARY, 1 |
| | 846), PNYKYRPRR (SEQ ID NO: 847) | | |
| SOX2 | DRVKRPMNA (SEQ ID NO: | Pro44Arg | MICROPHTHALMIA, SYNDROMIC 3 |
| | 848), NSPDRVKRP (SEQ ID NO: | | |
| | 849), RVKRPMNAF (SEQ ID NO: 850) | | |
| SOX2 | DRVKRPMNA (SEQ ID NO: | Asn46Lys | MICROPHTHALMIA, SYNDROMIC 3 |
| | 851), RVKRPMNAF (SEQ ID NO: 852) | | |
| SOX2 | GQRRKMAQE (SEQ ID NO: | Arg56Gly | MICROPHTHALMIA, SYNDROMIC 3 |
| | 853), MVWSRGQRR (SEQ ID NO: | | |
| | 854), QRRKMAQEN (SEQ ID NO: | | |
| | 855), RGQRRKMAQ (SEQ ID NO: | | |
| | 856), RRKMAQENP (SEQ ID NO: | | |
| | 857), SRGQRRKMA (SEQ ID NO: | | |
| | 858), VWSRGQRRK (SEQ ID NO: | | |
| | 859), WSRGQRRKM (SEQ ID NO: 860) | | |
| SOX2 | RVKRPMNAF (SEQ ID NO: 861) | Phe48Ser | MICROPHTHALMIA, SYNDROMIC 3 |
| SOX3 | MVWSRGQRR (SEQ ID NO: | Ser150Tyr | COLOBOMA, OCULAR, AUTOSOMAL |
| | 862), SRGQRRKMA (SEQ ID NO: | | DOMINANT, MENTAL RETARDATION, X- |
| | 863), VWSRGQRRK (SEQ ID NO: | | LINKED, WITH PANHYPOPITUITARISM |
| | 864), WSRGQRRKM (SEQ ID NO: 865) | | |
| SOX5 | DYKYKPRPK (SEQ ID NO: | Tyr623Cys | LAMB-SHAFFER SYNDROME |
| | 866), YKYKPRPKR (SEQ ID NO: | | |
| | 867), YPDYKYKPR (SEQ ID NO: 868) | | |
| SOX5 | KPRPKRTCL (SEQ ID NO: | Thr632Asn | LAMB-SHAFFER SYNDROME |
| | 869), KYKPRPKRT (SEQ ID NO: | | |
| | 870), PRPKRTCLV (SEQ ID NO: | | |
| | 871), RPKRTCLVD (SEQ ID NO: | | |
| | 872), YKPRPKRTC (SEQ ID NO: 873) | | |
| SP110 | PSDKKGKKR (SEQ ID NO: 874) | Pro272Ser | AUTISM |
| SPEN | EDARVLSKK (SEQ ID NO: 875) | Glu1010Lys | AUTISM |
| SPEN | LEKDEPRKS (SEQ ID NO: | Asp329Ala | AUTISM |
| | 876), SLEKDEPRK (SEQ ID NO: 877) | | |
| SRCAP | EEKELVRRR (SEQ ID NO: | Val2764Gly | ALZHEIMER DISEASE |
| | 878), EKELVRRRR (SEQ ID NO: | | |
| | 879), ELVRRRRQQ (SEQ ID NO: | | |
| | 880), KELVRRRRQ (SEQ ID NO: | | |
| | 881), LVRRRRQQR (SEQ ID NO: | | |
| | 882), VEEKELVRR (SEQ ID NO: | | |
| | 883), VRRRRQQRG (SEQ ID NO: 884) | | |
| SRCAP | PPGPKVLRK (SEQ ID NO: 885) | Pro2741Arg | ALZHEIMER DISEASE |
| TBX4 | CLKRRDGTR (SEQ ID NO: | Asp341His | PULMONARY HYPERTENSION, PRIMARY, 1 |
| | 886), KRRDGTRHL (SEQ ID NO: | | |
| | 887), LKRRDGTRH (SEQ ID NO: 888) | | |
| TBX4 | CLKRRDGTR (SEQ ID NO: | Gly342Cys | PULMONARY HYPERTENSION, PRIMARY, 1 |
| | 889), KRRDGTRHL (SEQ ID NO: | | |
| | 890), LKRRDGTRH (SEQ ID NO: 891) | | |
| TBX4 | ISKSIMRQR (SEQ ID NO: 892) | Ile270Ser | PULMONARY HYPERTENSION, PRIMARY, 1 |
| TBX4 | LRVARLQSK (SEQ ID NO: | Arg261Gln | PULMONARY HYPERTENSION, PRIMARY, 1 |
| | 893), RVARLQSKE (SEQ ID NO: 894) | | |
| TBX4 | RHLDLPCKR (SEQ ID NO: 895) | Arg352Leu | PULMONARY HYPERTENSION, PRIMARY, 1 |
| TBX5 | HRMSRMQSK (SEQ ID NO: | Ser252Ile | CARDIAC CONDUCTION DEFECT, HOLT-ORAM |
| | 896), RMSRMQSKE (SEQ ID NO: 897) | | SYNDROME |
| TBX5 | HRMSRMQSK (SEQ ID NO: | Ser252Thr | HOLT-ORAM SYNDROME |
| | 898), RMSRMQSKE (SEQ ID NO: 899) | | |
| TBX5 | RSTVRQKVA (SEQ ID NO: | Ser261Cys | CARDIAC CONDUCTION DEFECT, HOLT-ORAM |
| | 900), VPRSTVRQK (SEQ ID NO: 901) | | SYNDROME |
| TBX5 | RSTVRQKVA (SEQ ID NO: | Val263Met | AORTIC VALVE DISEASE 1, AORTIC VALVE |
| | 902), VPRSTVRQK (SEQ ID NO: 903) | | DISEASE 2 |
| TCF12 | CLKRREEEK (SEQ ID NO: | Glu651Ala | CRANIOSYNOSTOSIS 1 |
| | 904), KRREEEKVS (SEQ ID NO: | | |
| | 905), LKRREEEKV (SEQ ID NO: 906) | | |
| TCF12 | EKERRMANN (SEQ ID NO: | Arg579Gln | CRANIOSYNOSTOSIS 3 |
| | 907), EREKERRMA (SEQ ID NO: | | |
| | 908), IEREKERRM (SEQ ID NO: | | |
| | 909), KERRMANNA (SEQ ID NO: | | |
| | 910), KIEREKERR (SEQ ID NO: | | |
| | 911), REKERRMAN (SEQ ID NO: 912) | | |
| TCF12 | PEQKIEREK (SEQ ID NO: 913) | Pro568Ser | CRANIOSYNOSTOSIS 1 |
| TCF4 | AEREKERRM (SEQ ID NO: | Arg565Trp | CORNEAL DYSTROPHY, FUCHS ENDOTHELIAL, |
| | 914), EKERRMANN (SEQ ID NO: | | 3 |
| | 915), EREKERRMA (SEQ ID NO: | | |
| | 916), ERRMANNAR (SEQ ID NO: | | |
| | 917), KAEREKERR (SEQ ID NO: | | |
| | 918), KERRMANNA (SEQ ID NO: | | |
| | 919), QKAEREKER (SEQ ID NO: | | |
| | 920), REKERRMAN (SEQ ID NO: | | |
| | 921), RRMANNARE (SEQ ID NO: 922) | | |
| TCF4 | NPRRRPLHS (SEQ ID NO: | Pro156Thr | SCHIZOPHRENIA |
| | 923), PRRRPLHSS (SEQ ID NO: | | |
| | 924), YSSNNPRRR (SEQ ID NO: 925) | | |
| TCF4 | RIQSKTERG (SEQ ID NO: 926) | Ser102Cys | SCHIZOPHRENIA |
| TET2 | EEKKRSGAI (SEQ ID NO: | Ala1443Val | Lung Adenocarcinoma (LUAD) |
| | 927), EKKRSGAIQ (SEQ ID NO: | | |
| | 928), KKRSGAIQV (SEQ ID NO: 929) | | |
| TET2 | EEKKRSGAI (SEQ ID NO: | Arg1440Gln | Colon Adenocarcinoma (COAD), Cutaneous |
| | 930), EKKRSGAIQ (SEQ ID NO: | | Melanoma (SKCM), Large Cell Neuroendocrine |
| | 931), KKRSGAIQV (SEQ ID NO: | | Carcinoma (LUNE), Melanoma of Unknown |
| | 932), VEAQEEKKR (SEQ ID NO: 933) | | Primary (MUP) |
| TET2 | EEKKRSGAI (SEQ ID NO: | Arg1440Trp | Mucinous Ovarian Cancer (MOV), Uterine |
| | 934), EKKRSGAIQ (SEQ ID NO: | | Mixed Endometrial Carcinoma (UMEC) |
| | 935), KKRSGAIQV (SEQ ID NO: | | |
| | 936), VEAQEEKKR (SEQ ID NO: 937) | | |
| TET2 | EEKKRSGAI (SEQ ID NO: | Lys1438Arg | Glioblastoma (GB) |
| | 938), EKKRSGAIQ (SEQ ID NO: | | |
| | 939), KKRSGAIQV (SEQ ID NO: | | |
| | 940), VEAQEEKKR (SEQ ID NO: 941) | | |
| TET2 | EEKKRSGAI (SEQ ID NO: | Glu1436Gln | Acute Myeloid Leukemia (AML), Bladder |
| | 942), VEAQEEKKR (SEQ ID NO: 943) | | Urothelial Carcinoma (BLCA) |
| TET2 | EEKKRSGAI (SEQ ID NO: | Glu1436Gly | Uterine Endometrioid Carcinoma (UEC) |
| | 944), VEAQEEKKR (SEQ ID NO: 945) | | |
| TET2 | GRDKEQTRD (SEQ ID NO: | Asp551Ala | PROSTATE CANCER |
| | 946), RDKEQTRDL (SEQ ID NO: 947) | | |
| TET2 | VEAQEEKKR (SEQ ID NO: 948) | Ala1434Val | Colorectal Adenocarcinoma (COADREAD), |
| | | | Prostate Adenocarcinoma (PRAD) |
| TET2 | VEAQEEKKR (SEQ ID NO: 949) | Gln1435His | Mucinous Adenocarcinoma of the Colon and |
| | | | Rectum (MACR), Small Intestinal Carcinoma |
| | | | (SIC), Uterine Endometrioid Carcinoma |
| | | | (UEC) |
| TET2 | VEAQEEKKR (SEQ ID NO: 950) | Val1432Ala | High-Grade Serous Ovarian Cancer (HGSOC) |
| TFAP2B | AKSKNGGRS (SEQ ID NO: | Lys276Arg | |
| | 951), GVLRRAKSK (SEQ ID NO: | | |
| | 952), KSKNGGRSL (SEQ ID NO: | | |
| | 953), LRRAKSKNG (SEQ ID NO: | | |
| | 954), RAKSKNGGR (SEQ ID NO: | | CRANIOSYNOSTOSIS 1 |
| | 955), RRAKSKNGG (SEQ ID NO: | | |
| | 956), VLRRAKSKN (SEQ ID NO: 957) | | |
| THAP1 | AAVRRKNFK (SEQ ID NO: | Ala39Thr | DYSTONIA 6, TORSION |
| | 958), AVRRKNFKP (SEQ ID NO: | | |
| | 959), EWEAAVRRK (SEQ ID NO: | | |
| | 960), KEWEAAVRR (SEQ ID NO: 961) | | |
| THAP1 | CKNRYDKDK (SEQ ID NO: | Arg13His | DYSTONIA 6, TORSION |
| | 962), GCKNRYDKD (SEQ ID NO: | | |
| | 963), KNRYDKDKP (SEQ ID NO: | | |
| | 964), NRYDKDKPV (SEQ ID NO: 965) | | |
| THAP1 | CKNRYDKDK (SEQ ID NO: | Asn12Lys | DYSTONIA 6, TORSION |
| | 966), GCKNRYDKD (SEQ ID NO: | | |
| | 967), KNRYDKDKP (SEQ ID NO: | | |
| | 968), NRYDKDKPV (SEQ ID NO: 969) | | |
| THAP1 | CKNRYDKDK (SEQ ID NO: | Lys16Glu | DYSTONIA 6, TORSION |
| | 970), GCKNRYDKD (SEQ ID NO: | | |
| | 971), KNRYDKDKP (SEQ ID NO: | | |
| | 972), NRYDKDKPV (SEQ ID NO: 973) | | |
| THAP1 | GCKNRYDKD (SEQ ID NO: 974) | Gly9Cys | DYSTONIA 6, TORSION |
| THAP1 | GCKNRYDKD (SEQ ID NO: 975) | Gly9Ser | DYSTONIA 6, TORSION |
| THRB | GSHWKQKRK (SEQ ID NO: | Arg243Gln | HYPOTHYROIDISM, CONGENITAL, |
| | 976), HWKQKRKFL (SEQ ID NO: | | NONGOITROUS, 2, THYROID HORMONE |
| | 977), KQKRKFLPE (SEQ ID NO: | | RESISTANCE, GENERALIZED, AUTOSOMAL |
| | 978), KRKFLPEDI (SEQ ID NO: | | DOMINANT |
| | 979), QKRKFLPED (SEQ ID NO: | | |
| | 980), SHWKQKRKF (SEQ ID NO: | | |
| | 981), WKQKRKFLP (SEQ ID NO: 982) | | |
| THRB | GSHWKQKRK (SEQ ID NO: | Arg243Trp | HYPOTHYROIDISM, CONGENITAL, |
| | 983), HWKQKRKFL (SEQ ID NO: | | NONGOITROUS, 2, THYROID HORMONE |
| | 984), KQKRKFLPE (SEQ ID NO: | | RESISTANCE, GENERALIZED, AUTOSOMAL |
| | 985), KRKFLPEDI (SEQ ID NO: | | DOMINANT |
| | 986), QKRKFLPED (SEQ ID NO: | | |
| | 987), SHWKQKRKF (SEQ ID NO: | | |
| | 988), WKQKRKFLP (SEQ ID NO: 989) | | |
| THRB | KQKRKFLPE (SEQ ID NO: | Pro247Leu | HYPOTHYROIDISM, CONGENITAL, |
| | 990), KRKFLPEDI (SEQ ID NO: | | NONGOITROUS, 2 |
| | 991), QKRKFLPED (SEQ ID NO: | | |
| | 992), WKQKRKFLP (SEQ ID NO: 993) | | |
| THRB | KRKFLPEDI (SEQ ID NO: 994) | Ile250Thr | HYPOTHYROIDISM, CONGENITAL, |
| | | | NONGOITROUS, 2 |
| TOPORS | ESSRPRGRR (SEQ ID NO: | Pro637Leu | AUTISM |
| | 995), PRGRRDKKR (SEQ ID NO: | | |
| | 996), RESSRPRGR (SEQ ID NO: | | |
| | 997), RPRGRRDKK (SEQ ID NO: | | |
| | 998), RSRESSRPR (SEQ ID NO: | | |
| | 999), SRPRGRRDK (SEQ ID NO: | | |
| | 1000), SRSRESSRP (SEQ ID NO: | | |
| | 1001), SSRPRGRRD (SEQ ID NO: | | |
| | 1002) | | |
| TP53 | KGQSTSRHK (SEQ ID NO: | Gly374Arg | BREAST CANCER |
| | 1003), KKGQSTSRH (SEQ ID NO: | | |
| | 1004), SKKGQSTSR (SEQ ID NO: | | |
| | 1005) | | |
| TP53 | LRKKGEPHH (SEQ ID NO: | Gly293Trp | BREAST CANCER, GLIOMA SUSCEPTIBILITY 1 |
| | 1006), NLRKKGEPH (SEQ ID NO: | | Adenocarcinoma of the Gastroesophageal |
| | 1007), RKKGEPHHE (SEQ ID NO: | | Junction (GEJ), Adenoid Cystic Carcinoma |
| | 1008) | | (ACYC), Anaplastic Astrocytoma (AASTR), |
| | | | Anorectal Mucosal Melanoma (ARMM), |
| | | | Astrocytoma (ASTR), Breast Invasive |
| | | | Carcinoma, NOS (BRCNOS), Breast Invasive |
| | | | Ductal Carcinoma (IDC), Breast Invasive |
| | | | Lobular Carcinoma |
| TP53 | LRKKGEPHH (SEQ ID NO: | Arg290His | (ILC), COLORECTAL CANCER, Cancer of |
| | 1009), NLRKKGEPH (SEQ ID NO: | | Unknown Primary (CUP), Collecting Duct |
| | 1010), RKKGEPHHE (SEQ ID NO: | | Renal Cell Carcinoma (CDRCC), Colon |
| | 1011), RRTEEENLR (SEQ ID NO: | | Adenocarcinoma (COAD), Cutaneous Melanoma |
| | 1012), RTEEENLRK (SEQ ID NO: | | (SKCM), ENDOMETRIAL CANCER, Glioblastoma |
| | 1013), TEEENLRKK (SEQ ID NO: | | Multiforme (GBM), LI-FRAUMENI SYNDROME, |
| | 1014) | | Lung Adenocarcinoma (LUAD), Medullary |
| | | | Thyroid Cancer (THME), Melanoma (MEL), |
| | | | OVARIAN CANCER, Oral Cavity Squamous Cell |
| | | | Carcinoma (OCSC), Papillary Thyroid Cancer |
| | | | (THPA), Pleural Mesothelioma (PLMESO), |
| | | | Upper Tract Urothelial Carcinoma (UTUC) |
| TP53 | LRKKGEPHH (SEQ ID NO: | Arg290Leu | Bladder Urothelial Carcinoma (BLCA), LI- |
| | 1015), NLRKKGEPH (SEQ ID NO: | | FRAUMENI SYNDROME, MDS with Ring |
| | 1016), RKKGEPHHE (SEQ ID NO: | | Sideroblasts and Multilineage Dysplasia |
| | 1017), RRTEEENLR (SEQ ID NO: | | (MDSRSMD) |
| | 1018), RTEEENLRK (SEQ ID NO: | | |
| | 1019), TEEENLRKK (SEQ ID NO: | | |
| | 1020) | | |
| TP53 | LRKKGEPHH (SEQ ID NO: | Arg290Pro | Lung Adenocarcinoma (LUAD) |
| | 1021), NLRKKGEPH (SEQ ID NO: | | |
| | 1022), RKKGEPHHE (SEQ ID NO: | | |
| | 1023), RRTEEENLR (SEQ ID NO: | | |
| | 1024), RTEEENLRK (SEQ ID NO: | | |
| | 1025), TEEENLRKK (SEQ ID NO: | | |
| | 1026) | | |
| TP53 | LRKKGEPHH (SEQ ID NO: | Lys291Arg | Uterine Endometrioid Carcinoma (UEC) |
| | 1027), NLRKKGEPH (SEQ ID NO: | | |
| | 1028), RKKGEPHHE (SEQ ID NO: | | |
| | 1029), RTEEENLRK (SEQ ID NO: | | |
| | 1030), TEEENLRKK (SEQ ID NO: | | |
| | 1031) | | |
| TP53 | LRKKGEPHH (SEQ ID NO: | Lys291Asn | Bladder Urothelial Carcinoma (BLCA), |
| | 1032), NLRKKGEPH (SEQ ID NO: | | Invasive Breast Carcinoma (BRCA) |
| | 1033), RKKGEPHHE (SEQ ID NO: | | |
| | 1034), RTEEENLRK (SEQ ID NO: | | |
| | 1035), TEEENLRKK (SEQ ID NO: | | |
| | 1036) | | |
| TP53 | LRKKGEPHH (SEQ ID NO: | Lys291Gln | High-Grade Serous Ovarian Cancer (HGSOC) |
| | 1037), NLRKKGEPH (SEQ ID NO: | | |
| | 1038), RKKGEPHHE (SEQ ID NO: | | |
| | 1039), RTEEENLRK (SEQ ID NO: | | |
| | 1040), TEEENLRKK (SEQ ID NO: | | |
| | 1041) | | |
| TP53 | LRKKGEPHH (SEQ ID NO: | Lys291Glu | Gallbladder Adenocarcinoma, NOS (GBAD), |
| | 1042), NLRKKGEPH (SEQ ID NO: | | Uterine Carcinosarcoma/Uterine Malignant |
| | 1043), RKKGEPHHE (SEQ ID NO: | | Mixed Mullerian Tumor (UCS) |
| | 1044), RTEEENLRK (SEQ ID NO: | | |
| | 1045), TEEENLRKK (SEQ ID NO: | | |
| | 1046) | | |
| TP53 | LRKKGEPHH (SEQ ID NO: | Lys292Asn | Colon Adenocarcinoma (COAD) |
| | 1047), NLRKKGEPH (SEQ ID NO: | | |
| | 1048), RKKGEPHHE (SEQ ID NO: | | |
| | 1049), TEEENLRKK (SEQ ID NO: | | |
| | 1050) | | |
| TP53 | LRKKGEPHH (SEQ ID NO: | Lys292Ile | LI-FRAUMENI SYNDROME |
| | 1051), NLRKKGEPH (SEQ ID NO: | | |
| | 1052), RKKGEPHHE (SEQ ID NO: | | |
| | 1053), TEEENLRKK (SEQ ID NO: | | |
| | 1054) | | |
| TP53 | LRKKGEPHH (SEQ ID NO: | Leu289Phe | Bladder Urothelial Carcinoma (BLCA), |
| | 1055), NLRKKGEPH (SEQ ID NO: | | Breast Invasive Ductal Carcinoma (IDC), |
| | 1056), RRTEEENLR (SEQ ID NO: | | Cutaneous Squamous Cell Carcinoma (CSCC), |
| | 1057), RTEEENLRK (SEQ ID NO: | | Larynx Squamous Cell Carcinoma (LXSC), |
| | 1058), TEEENLRKK (SEQ ID NO: | | Uterine Endometrioid Carcinoma (UEC) |
| | 1059) | | |
| TP53 | LRKKGEPHH (SEQ ID NO: | Leu289Pro | Lung Adenocarcinoma (LUAD) |
| | 1060), NLRKKGEPH (SEQ ID NO: | | |
| | 1061), RRTEEENLR (SEQ ID NO: | | |
| | 1062), RTEEENLRK (SEQ ID NO: | | |
| | 1063), TEEENLRKK (SEQ ID NO: | | |
| | 1064) | | |
| TP53 | LRKKGEPHH (SEQ ID NO: | Leu289Val | Bladder Urothelial Carcinoma (BLCA), |
| | 1065), NLRKKGEPH (SEQ ID NO: | | Plasmacytoid/Signet Ring Cell Bladder |
| | 1066), RRTEEENLR (SEQ ID NO: | | Carcinoma (SRCBC) |
| | 1067), RTEEENLRK (SEQ ID NO: | | |
| | 1068), TEEENLRKK (SEQ ID NO: | | |
| | 1069) | | |
| TP53 | SKKGQSTSR (SEQ ID NO: 1070) | Ser371Phe | BREAST CANCER |
| TP63 | DGTKRPFRQ (SEQ ID NO: | Arg379His | Colon Adenocarcinoma (COAD), Large Cell |
| | 1071), GTKRPFRQN (SEQ ID NO: | | Neuroendocrine Carcinoma (LUNE), Signet |
| | 1072), KRPFRONTH (SEQ ID NO: | | Ring Cell Carcinoma of the Stomach (SSRCC) |
| | 1073) | | |
| TP63 | DGTKRPFRQ (SEQ ID NO: | Arg379Leu | Lung Adenocarcinoma (LUAD) |
| | 1074), GTKRPFRQN (SEQ ID NO: | | |
| | 1075), KRPFRQNTH (SEQ ID NO: | | |
| | 1076) | | |
| TP63 | DGTKRPFRQ (SEQ ID NO: | Arg379Ser | Cutaneous Melanoma (SKCM), Poorly |
| | 1077), GTKRPFRQN (SEQ ID NO: | | Differentiated Carcinoma of the Uterus |
| | 1078), KRPFRQNTH (SEQ ID NO: | | (UPDC) |
| | 1079) | | |
| TTF1 | EKKNKKHQR (SEQ ID NO: | Gln172Arg | HYPOTHYROIDISM, CONGENITAL, |
| | 1080), KHQRKAASW (SEQ ID NO: | | NONGOITROUS, 2 |
| | 1081), KKHQRKAAS (SEQ ID NO: | | |
| | 1082), KKNKKHQRK (SEQ ID NO: | | |
| | 1083), KNKKHQRKA (SEQ ID NO: | | |
| | 1084), NKKHQRKAA (SEQ ID NO: | | |
| | 1085), REKKNKKHQ (SEQ ID NO: | | |
| | 1086) | | |
| TTF1 | EQSQITRRK (SEQ ID NO: | Lys53Arg | AUTISM |
| | 1087), ITRRKKRKK (SEQ ID NO: | | |
| | 1088), KKRKKDFQH (SEQ ID NO: | | |
| | 1089), QITRRKKRK (SEQ ID NO: | | |
| | 1090), QSQITRRKK (SEQ ID NO: | | |
| | 1091), RKKRKKDFQ (SEQ ID NO: | | |
| | 1092), RRKKRKKDF (SEQ ID NO: | | |
| | 1093), SQITRRKKR (SEQ ID NO: | | |
| | 1094), TRRKKRKKD (SEQ ID NO: | | |
| | 1095) | | |
| TTF1 | KKRKKRRYS (SEQ ID NO: | Arg90Lys | HYPOTHYROIDISM, CONGENITAL, |
| | 1096), KKRRYSALE (SEQ ID NO: | | NONGOITROUS, 2 |
| | 1097), KRKKRRYSA (SEQ ID NO: | | |
| | 1098), KRRYSALEV (SEQ ID NO: | | |
| | 1099), LKKRKKRRY (SEQ ID NO: | | |
| | 1100), RKKRRYSAL (SEQ ID NO: | | |
| | 1101), TLKKRKKRR (SEQ ID NO: | | |
| | 1102) | | |
| TWIST1 | GGRKRRSSR (SEQ ID NO: | Arg39Gly | CRANIOSYNOSTOSIS 1 |
| | 1103), GKRGGRKRR (SEQ ID NO: | | |
| | 1104), GRKRRSSRR (SEQ ID NO: | | |
| | 1105), KRGGRKRRS (SEQ ID NO: | | |
| | 1106), KRRSSRRSA (SEQ ID NO: | | |
| | 1107), RGGRKRRSS (SEQ ID NO: | | |
| | 1108), RKRRSSRRS (SEQ ID NO: | | |
| | 1109), RRSSRRSAG (SEQ ID NO: | | |
| | 1110), SGKRGGRKR (SEQ ID NO: | | |
| | 1111) | | |
| TWIST1 | GKRGGRKRR (SEQ ID NO: | Gly32Ser | CRANIOSYNOSTOSIS 1 |
| | 1112), PSGKRGGRK (SEQ ID NO: | | |
| | 1113), RQQPPSGKR (SEQ ID NO: | | |
| | 1114), SGKRGGRKR (SEQ ID NO: | | |
| | 1115) | | |
| USF1 | PRTTRDEKR (SEQ ID NO: | Arg196Trp | HYPERLIPIDEMIA, FAMILIAL COMBINED, 3 |
| | 1116), RDEKRRAQH (SEQ ID NO: | | |
| | 1117), RTTRDEKRR (SEQ ID NO: | | |
| | 1118), TRDEKRRAQ (SEQ ID NO: | | |
| | 1119), TTRDEKRRA (SEQ ID NO: | | |
| | 1120) | | |
| ZBTB21 | EGTRPNKKF (SEQ ID NO: | Gly539Arg | AUTISM |
| | 1121), GTRPNKKFK (SEQ ID NO: | | |
| | 1122), LEGTRPNKK (SEQ ID NO: | | |
| | 1123) | | |
| ZEB2 | CKRRKQANP (SEQ ID NO: | Pro20Leu | AUTISM |
| | 1124), KQANPRRKN (SEQ ID NO: | | |
| | 1125), KRRKQANPR (SEQ ID NO: | | |
| | 1126), NPRRKNVVN (SEQ ID NO: | | |
| | 1127), PRRKNVVNY (SEQ ID NO: | | |
| | 1128), RKQANPRRK (SEQ ID NO: | | |
| | 1129), RRKQANPRR (SEQ ID NO: | | |
| | 1130) | | |
| ZEB2 | FAKRKLEER (SEQ ID NO: | Phe148Leu | FETAL AKINESIA DEFORMATION SEQUENCE 1 |
| | 1131), FEEYFAKRK (SEQ ID NO: | | |
| | 1132) | | |
| ZFPM2 | ERTTTSPKR (SEQ ID NO: | Thr843Ala | DIAPHRAGMATIC HERNIA 3, DIAPHRAGMATIC |
| | 1133), RTTTSPKRL (SEQ ID NO: | | HERNIA, CONGENITAL |
| | 1134) | | |
| ZFPM2 | ERTTTSPKR (SEQ ID NO: | Thr843Met | TETRALOGY OF FALLOT |
| | 1135), RTTTSPKRL (SEQ ID NO: | | |
| | 1136) | | |
| ZFPM2 | KKCLSQSER (SEQ ID NO: 1137) | Lys834Arg | DIAPHRAGMATIC HERNIA, CONGENITAL |
| ZFPM2 | KRRKMYEMC (SEQ ID NO: | Lys737Glu | CONOTRUNCAL HEART MALFORMATIONS |
| | 1138), MQRTMRTRK (SEQ ID NO: | | |
| | 1139), MRTRKRRKM (SEQ ID NO: | | |
| | 1140), QRTMRTRKR (SEQ ID NO: | | |
| | 1141), RKRRKMYEM (SEQ ID NO: | | |
| | 1142), RTMRTRKRR (SEQ ID NO: | | |
| | 1143), RTRKRRKMY (SEQ ID NO: | | |
| | 1144), TMRTRKRRK (SEQ ID NO: | | |
| | 1145), TRKRRKMYE (SEQ ID NO: | | |
| | 1146) | | |
| ZIC3 | RKHMKVHES (SEQ ID NO: | Lys405Glu | HETEROTAXY, VISCERAL, 1, X-LINKED |
| | 1147), SLRKHMKVH (SEQ ID NO: | | |
| | 1148), SSLRKHMKV (SEQ ID NO: | | |
| | 1149) | | |
| ZKSCAN5 | DRKQGIPMK (SEQ ID NO: | Ile516Thr | AUTISM |
| | 1150), KLDRKQGIP (SEQ ID NO: | | |
| | 1151), RKQGIPMKE (SEQ ID NO: | | |
| | 1152) | | |
| ZNF148 | HDKKLNRCA (SEQ ID NO: | Asp282Gly | GLOBAL DEVELOPMENTAL DELAY, ABSENT |
| | 1153), NHDKKLNRC (SEQ ID NO: | | OR HYPOPLASTIC CORPUS CALLOSUM, AND |
| | 1154) | | DYSMORPHIC FACIES |
| ZNF174 | DFHRASKKP (SEQ ID NO: | Ser128Phe | AUTISM |
| | 1155), EDFHRASKK (SEQ ID NO: | | |
| | 1156), FHRASKKPK (SEQ ID NO: | | |
| | 1157), HRASKKPKQ (SEQ ID NO: | | |
| | 1158), RASKKPKQW (SEQ ID NO: | | |
| | 1159) | | |
| ZNF217 | KRPETKLKP (SEQ ID NO: | Leu861Ser | AUTISM |
| | 1160), KTKRPETKL (SEQ ID NO: | | |
| | 1161), RPETKLKPL (SEQ ID NO: | | |
| | 1162), TKRPETKLK (SEQ ID NO: | | |
| | 1163) | | |
| ZNF292 | AAMKPLRRL (SEQ ID NO: | Leu609Phe | AUTISM |
| | 1164), KPLRRLGRP (SEQ ID NO: | | |
| | 1165), LRRLGRPPK (SEQ ID NO: | | |
| | 1166), MKPLRRLGR (SEQ ID NO: | | |
| | 1167), PLRRLGRPP (SEQ ID NO: | | |
| | 1168), RLGRPPKIT (SEQ ID NO: | | |
| | 1169), RRLGRPPKI (SEQ ID NO: | | |
| | 1170) | | |
| ZNF292 | DEKQKKREI (SEQ ID NO: | Arg519Gly | AUTISM |
| | 1171), EKQKKREIK (SEQ ID NO: | | |
| | 1172), GDEKQKKRE (SEQ ID NO: | | |
| | 1173), IGDEKQKKR (SEQ ID NO: | | |
| | 1174), KKREIKQLR (SEQ ID NO: | | |
| | 1175), KQKKREIKQ (SEQ ID NO: | | |
| | 1176), KREIKQLRE (SEQ ID NO: | | |
| | 1177), QKKREIKQL (SEQ ID NO: | | |
| | 1178), REIKQLRER (SEQ ID NO: | | |
| | 1179) | | |
| ZNF292 | DRGRGPNGK (SEQ ID NO: | Arg1349Pro | AUTISM |
| | 1180), EKVKKDRGR (SEQ ID NO: | | |
| | 1181), GRGPNGKER (SEQ ID NO: | | |
| | 1182), KDRGRGPNG (SEQ ID NO: | | |
| | 1183), KKDRGRGPN (SEQ ID NO: | | |
| | 1184), KVKKDRGRG (SEQ ID NO: | | |
| | 1185), RGPNGKERK (SEQ ID NO: | | |
| | 1186), RGRGPNGKE (SEQ ID NO: | | |
| | 1187), VKKDRGRGP (SEQ ID NO: | | |
| | 1188) | | |
| ZNF292 | EEKKRKKPV (SEQ ID NO: | Pro2097Ala | AUTISM |
| | 1189), EKKRKKPVS (SEQ ID NO: | | |
| | 1190), KEEKKRKKP (SEQ ID NO: | | |
| | 1191), KKRKKPVSQ (SEQ ID NO: | | |
| | 1192), KRKKPVSQS (SEQ ID NO: | | |
| | 1193), RKKPVSQSL (SEQ ID NO: | | |
| | 1194) | | |
| ZNF292 | IKRPYGRKS (SEQ ID NO: | Pro1987Thr | AUTISM |
| | 1195), KIKRPYGRK (SEQ ID NO: | | |
| | 1196), KLKIKRPYG (SEQ ID NO: | | |
| | 1197), KRPYGRKSQ (SEQ ID NO: | | |
| | 1198), LKIKRPYGR (SEQ ID NO: | | |
| | 1199), MVKLKIKRP (SEQ ID NO: | | |
| | 1200), VKLKIKRPY (SEQ ID NO: | | |
| | 1201) | | |
| ZNF292 | KRVNKEKNV (SEQ ID NO: | Val2533Asp | AUTISM |
| | 1202), LKRVNKEKN (SEQ ID NO: | | |
| | 1203), NLKRVNKEK (SEQ ID NO: | | |
| | 1204), QKASNLKRV (SEQ ID NO: | | |
| | 1205), SNLKRVNKE (SEQ ID NO: | | |
| | 1206) | | |
| ZNF292 | RKKVAPPLI (SEQ ID NO: 1207) | Ile1639Thr | AUTISM |
| ZNF335 | AAGKKGRLR (SEQ ID NO: | Arg286Gln | AUTISM |
| | 1208), AGKKGRLRK (SEQ ID NO: | | |
| | 1209), GKKGRLRKW (SEQ ID NO: | | |
| | 1210), GRLRKWSTS (SEQ ID NO: | | |
| | 1211), KGRLRKWST (SEQ ID NO: | | |
| | 1212), KKGRLRKWS (SEQ ID NO: | | |
| | 1213), LRKWSTSTK (SEQ ID NO: | | |
| | 1214), RKWSTSTKS (SEQ ID NO: | | |
| | 1215), RLRKWSTST (SEQ ID NO: | | |
| | 1216) | | |
| ZNF335 | HMRERHFRP (SEQ ID NO: | Arg265Trp | AUTISM |
| | 1217), LRHMRERHF (SEQ ID NO: | | |
| | 1218), MRERHFRPV (SEQ ID NO: | | |
| | 1219), RHMRERHFR (SEQ ID NO: | | |
| | 1220), TLLRHMRER (SEQ ID NO: | | |
| | 1221) | | |
| ZNF335 | RFNRNGHLK (SEQ ID NO: 1222) | Arg1111His | MICROCEPHALY 10, PRIMARY, AUTOSOMAL |
| | | | RECESSIVE |
| ZNF335 | RFNRNGHLK (SEQ ID NO: 1223) | Arg1111Leu | MICROCEPHALY 10, PRIMARY, AUTOSOMAL |
| | | | RECESSIVE |
| ZNF385A | ISSRRHRDG (SEQ ID NO: | Arg284Gln | GILLES DE LA TOURETTE SYNDROME |
| | 1224), KQHISSRRH (SEQ ID NO: | | |
| | 1225), LKQHISSRR (SEQ ID NO: | | |
| | 1226), RHRDGVAGK (SEQ ID NO: | | |
| | 1227), RRHRDGVAG (SEQ ID NO: | | |
| | 1228), SRRHRDGVA (SEQ ID NO: | | |
| | 1229) | | |
| ZNF407 | RMYMKHLRT (SEQ ID NO: 1230) | Tyr460Cys | AUTISM |
| ZNF574 | ARRRGLECS (SEQ ID NO: | Arg734His | AUTISM |
| | 1231), PARRRGLEC (SEQ ID NO: | | |
| | 1232), RRRGLECSE (SEQ ID NO: | | |
| | 1233), SPAAPARRR (SEQ ID NO: | | |
| | 1234) | | |
| ZNF644 | GHLKRLGKT (SEQ ID NO: | Gly1059Val | AUTISM |
| | 1235), HVRGHLKRL (SEQ ID NO: | | |
| | 1236), NHVRGHLKR (SEQ ID NO: | | |
| | 1237), RGHLKRLGK (SEQ ID NO: | | |
| | 1238) | | |
| ZNF644 | SSFSKIHKR (SEQ ID NO: 1239) | Ser672Gly | MYOPIA 21, AUTOSOMAL DOMINANT |
| ZNF687 | EPPRPAKRP (SEQ ID NO: | Pro937Arg | PAGET DISEASE OF BONE 6 |
| | 1240), PEPPRPAKR (SEQ ID NO: | | |
| | 1241), PPRPAKRPR (SEQ ID NO: | | |
| | 1242) | | |
| ZNF804A | KALQRLHKL (SEQ ID NO: | Arg116Cys | AUTISM |
| | 1243), KQEKALQRL (SEQ ID NO: | | |
| | 1244), RKQEKALQR (SEQ ID NO: | | |
| | 1245), RLHKLAELR (SEQ ID NO: | | |
| | 1246) | | |
| ZNF831 | LRASRLRTP (SEQ ID NO: | Arg1310Cys | INFLAMMATORY BOWEL DISEASE (CROHN |
| | 1247), LRTPTWVRR (SEQ ID NO: | | DISEASE) 1 |
| | 1248), RLRTPTWVR (SEQ ID NO: | | |
| | 1249), RTPTWVRRR (SEQ ID NO: | | |
| | 1250), VQLRASRLR (SEQ ID NO: | | |
| | 1251) | | |
| ZSCAN30 | RDGRMVAGK (SEQ ID NO: 1252) | Gly186Ser | AUTISM |

TABLE 3

| Oligo ID | Sequence | Notes |
|---|---|---|
| KR298_fp-cy5-SOX2-motif-F | /5Cy5/CGCGCCATTGTGCCCGGGT (SEQ ID | |
| | NO: 1253) | |
| KR297_fp-NL-SOX2-motif-R | ACCCGGGCACAATGGCGCG (SEQ ID NO: 1254) | |
| KR294_fp-cy5-KLF4-1X- | /5Cy5/AGGGGGTGTGCCCGCCAGGAGGGGTGGGTC | |
| motif-F | (SEQ ID NO: 1255) | |
| KR279_KLF4-1X-motif-R | GACCCACCCCTCCTGGCGGGCACACCCCCT (SEQ ID | |
| | NO: 1256) | |
| JH440_trx_T7_prom | TAATACGACTCACTATAGGG (SEQ ID NO: 1257) | |
| JH441_trx_SP6_prom | ATTTAGGTGACACTATAGAA (SEQ ID NO: 1258) | |
| KR290_DNA_F | AGGATTCTAATTTCGATCA (SEQ ID NO: 1259) | Used for the EMSA in FIG. |
| | | 11A-D |
| KR291_DNA_R | TGATCGAAATTAGAATCCT (SEQ ID NO: 1260) | Used for the EMSA in FIG. |
| | | 11A-D |
| CLIP_RT v5 | TTCAGACGTGTGCTCTTCCG (SEQ ID NO: 1261) | |
| CLIP_5_link_v5 (C5) | /5phos/NNNNNNNN | |
| | AGATCGGAAGAGCGTCGTGTAGGG/3ddC/ (SEQ ID | |
| | NO: 1262) | |

### REFERENCES

1. Lambert, S. A., Jolma, A., Campitelli, L. F., Das, P. K., Yin, Y., Albu, M., Chen, X., Taipale, J., Hughes, T. R., and Weirauch, M. T. (2018). The Human Transcription Factors. Cell 172, 650-665. 10.1016/j.cell.2018.01.029.
2. Vaquerizas, J. M., Kummerfeld, S. K., Teichmann, S. A., and Luscombe, N. M. (2009). A census of human transcription factors: function, expression and evolution. Nat. Rev. Genet. 10, 252-263. 10.1038/nrg2538.
3. Cramer, P. (2019). Organization and regulation of gene transcription. Nature 573, 45-54. 10.1038/s41586-019-1517-4.
4. Lee, T. I., and Young, R. A. (2013). Transcriptional regulation and its misregulation in disease. Cell 152, 1237-1251. 10.1016/j.cell.2013.02.014.
5. Stadhouders, R., Filion, G. J., and Graf, T. (2019). Transcription factors and 3D genome conformation in cell-fate decisions. Nature 569, 345-354. 10.1038/s41586-019-1182-7.
6. Panne, D., Maniatis, T., and Harrison, S. C. (2007). An Atomic Model of the Interferon-β Enhanceosome. Cell 129, 1111-1123. 10.1016/j.cell.2007.05.019.
7. Avsec, Ž., Weilert, M., Shrikumar, A., Krueger, S., Alexandari, A., Dalal, K., Fropf, R., McAnany, C., Gagneur, J., Kundaje, A., et al. (2021). Base-resolution models of transcription factor binding reveal soft motif syntax. Nat. Genet. 53, 354-366. 10.1038/s41588-021-00782-6.
8. Arnold, C. D., Nemčko, F., Woodfin, A. R., Wienerroither, S., Vlasova, A., Schleiffer, A., Pagani, M., Rath, M., and Stark, A. (2018). A high-throughput method to identify transactivation domains within transcription factor sequences. EMBO J. 37, e98896. 10.15252/embj.201798896.
9. Boija, A., Klein, I. A., Sabari, BR., Dall'Agnese, A., Coffey, E. L., Zamudio, A. V., Li, C. H., Shrinivas, K., Manteiga, J. C., Hannett, N. M., et al. (2018). Transcription Factors Activate Genes through the Phase-Separation Capacity of Their Activation Domains. Cell 175, 1842-1855.e16. 10.1016/j.cell.2018.10.042.
10. Soto, L. F., Li, Z., Santoso, C. S., Berenson, A., Ho, I., Shen, V. X., Yuan, S., and Fuxman Bass, J. I. (2022). Compendium of human transcription factor effector domains. Mol. Cell 82, 514-526. 10.1016/j.molcel.2021.11.007.
11. Richter, W. F., Nayak, S., Iwasa, J., and Taatjes, D. J. (2022). The Mediator complex as a master regulator of transcription by RNA polymerase II. Nat. Rev. Mol. Cell Biol., 1-18. 10.1038/s41580-022-00498-3.
12. Vos, S. M. (2021). Understanding transcription across scales: From base pairs to chromosomes. Mol. Cell 81, 1601-1616. 10.1016/j.molcel.2021.03.002.
13. Lelli, K. M., Slattery, M., and Mann, R. S. (2012). Disentangling the many layers of eukaryotic transcriptional regulation. Annu. Rev. Genet. 46, 43-68. 10.1146/annurev-genet-110711-155437.
14. Spitz, F., and Furlong, E. E. M. (2012). Transcription factors: from enhancer binding to developmental control. Nat. Rev. Genet. 13, 613-626. 10.1038/nrg3207.
15. Kaikkonen, M. U., and Adelman, K. (2018). Emerging Roles of Non-Coding RNA Transcription. Trends Biochem. Sci. 43, 654-667. 10.1016/j.tibs.2018.06.002.
16. Seila, A. C., Calabrese, J. M., Levine, S. S., Yeo, G. W., Rahl, P. B., Flynn, R. A., Young, R. A., and Sharp, P. A. (2008). Divergent Transcription from Active Promoters. Science 322, 1849-1851. 10.1126/science.1162253.
17. Cassiday, L. A., and Maher, L. J. (2002). Having it both ways: transcription factors that bind DNA and RNA. Nucleic Acids Res. 30, 4118-4126. 10.1093/nar/gkf512.
18. Holmes, Z. E., Hamilton, D. J., Hwang, T., Parsonnet, N. V., Rinn, J. L., Wuttke, D. S., and Batey, R. T. (2020). The Sox2 transcription factor binds RNA. Nat. Commun. 11, 1805. 10.1038/s41467-020-15571-8.
19. Hou, L., Wei, Y., Lin, Y., Wang, X., Lai, Y., Yin, M., Chen, Y., Guo, X., Wu, S., Zhu, Y., et al. (2020). Concurrent binding to DNA and RNA facilitates the pluripotency reprogramming activity of Sox2. Nucleic Acids Res. 48, 3869-3887. 10.1093/nar/gkaa067.
20. Saldaña-Meyer, R., Rodriguez-Hernaez, J., Escobar, T., Nishana, M., Jácome-López, K., Nora, E. P., Bruneau, B. G., Tsirigos, A., Furlan-Magaril, M., Skok, J., et al. (2019). RNA Interactions Are Essential for CTCF-Mediated Genome Organization. Mol. Cell 76, 412-422.e5. 10.1016/j.molcel.2019.08.015.
21. Sigova, A. A., Abraham, B. J., Ji, X., Molinie, B., Hannett, N. M., Guo, Y. E., Jangi, M., Giallourakis, C. C., Sharp, P. A., and Young, R. A. (2015). Transcription factor trapping by RNA in gene regulatory elements. Science 350, 978-981. 10.1126/science.aad3346.
22. Theunissen, O., Rudt, F., Guddat, U., Mentzel, H., and Pieler, T. (1992). RNA and DNA binding zinc fingers in Xenopus TFIIIA. Cell 71, 679-690. 10.1016/0092-8674(92)90601-8.
23. Xu, Y., Huangyang, P., Wang, Y., Xue, L., Devericks, E., Nguyen, H. G., Yu, X., Oses-Prieto, J. A., Burlingame, A. L., Miglani, S., et al. (2021). ERa is an RNA-binding protein sustaining tumor cell survival and drug resistance. Cell 0. 10.1016/j.cell.2021.08.036.
24. Jeon, Y., and Lee, J. T. (2011). YY1 tethers Xist RNA to the inactive X nucleation center. Cell 146, 119-133. 10.1016/j.cell.2011.06.026.
25. Yoshida, Y., Izumi, H., Torigoe, T., Ishiguchi, H., Yoshida, T., Itoh, H., and Kohno, K. (2004). Binding of RNA to p53 regulates its oligomerization and DNA-binding activity. Oncogene 23, 4371-4379. 10.1038/sj.onc.1207583.
26. Steiner, H. R., Lammer, N. C., Batey, R. T., and Wuttke, D. S. (2022). An Extended DNA Binding Domain of the Estrogen Receptor Alpha Directly Interacts with RNAs in Vitro. Biochemistry 61, 2490-2494. 10.1021/acs.biochem.2c00536.
27. Niessing, D., Driever, W., Sprenger, F., Taubert, H., Jäckle, H., and Rivera-Pomar, R. (2000). Homeodomain Position 54 Specifies Transcriptional versus Translational Control by Bicoid. Mol. Cell 5, 395-401. 10.1016/S1097-2765(00)80434-7.
28. Dvir, S., Argoetti, A., Lesnik, C., Roytblat, M., Shriki, K., Amit, M., Hashimshony, T., and Mandel-Gutfreund, Y. (2021). Uncovering the RNA-binding protein landscape in the pluripotency network of human embryonic stem cells. Cell Rep. 35. 10.1016/j.celrep.2021.109198.
29. Lunde, B. M., Moore, C., and Varani, G. (2007). RNA-binding proteins: modular design for efficient function. Nat. Rev. Mol. Cell Biol. 8, 479-490. 10.1038/nrm2178.
30. Wheeler, E. C., Van Nostrand, E. L., and Yeo, G. W. (2018). Advances and challenges in the detection of transcriptome-wide protein-RNA interactions. Wiley Interdiscip. Rev. RNA 9, e1436. 10.1002/wrna.1436.
31. He, C., Sidoli, S., Warneford-Thomson, R., Tatomer, D. C., Wilusz, J. E., Garcia, B. A., and Bonasio, R. (2016). High-Resolution Mapping of RNA-Binding Regions in the Nuclear Proteome of Embryonic Stem Cells. Mol. Cell 64, 416-430. 10.1016/j.molcel.2016.09.034.
32. Orkin, S. H., and Zon, L. I. (2008). Hematopoiesis: An Evolving Paradigm for Stem Cell Biology. Cell 132, 631-644. 10.1016/j.cell.2008.01.025.
33. Delgado, M. D., Lerga, A., Cañelles, M., Gómez-Casares, M. T., and León, J. (1995). Differential regulation of Max and role of c-Myc during erythroid and myelomonocytic differentiation of K562 cells. Oncogene 10, 1659-1665.
34. Young, R. A. (2011). Control of the embryonic stem cell state. Cell 144, 940-954. 10.1016/j.cell.2011.01.032.
35. Ibarra, A., Benner, C., Tyagi, S., Cool, J., and Hetzer, M. W. (2016). Nucleoporin-mediated regulation of cell identity genes. Genes Dev. 30, 2253-2258. 10.1101/gad.287417.116.
36. Saldaña-Meyer, R., González-Buendía, E., Guerrero, G., Narendra, V., Bonasio, R., Recillas-Targa, F., and Reinberg, D. (2014). CTCF regulates the human p53 gene through direct interaction with its natural antisense transcript, Wrap53. Genes Dev. 28, 723-734. 10.1101/gad.236869.113.
37. Burd, C. G., and Dreyfuss, G. (1994). RNA binding specificity of hnRNP A1: significance of hnRNP A1 high-affinity binding sites in pre-mRNA splicing. EMBO J. 13, 1197-1204.
38. Corley, M., Burns, M. C., and Yeo, G. W. (2020). How RNA-Binding Proteins Interact with RNA: Molecules and Mechanisms. Mol. Cell 78, 9-29. 10.1016/j.molcel.2020.03.011.
39. Maji, D., Glasser, E., Henderson, S., Galardi, J., Pulvino, M. J., Jenkins, J. L., and Kielkopf, C. L. (2020). Representative cancer-associated U2AF2 mutations alter RNA interactions and splicing. J. Biol. Chem. 295, 17148-17157. 10.1074/jbc.RA120.015339.
40. Zhang, J., Lieu, Y. K., Ali, A. M., Penson, A., Reggio, K. S., Rabadan, R., Raza, A., Mukherjee, S., and Manley, J. L. (2015). Disease-associated mutation in SRSF2 misregulates splicing by altering RNA-binding affinities. Proc. Natl. Acad. Sci. U.S.A. 112, E4726-E4734. 10.1073/pnas.1514105112.
41. Calnan, B. J., Biancalana, S., Hudson, D., and Frankel, A. D. (1991). Analysis of arginine-rich peptides from the HIV Tat protein reveals unusual features of RNA-protein recognition. Genes Dev. 5, 201-210. 10.1101/gad.5.2.201.
42. Calnan, B. J., Tidor, B., Biancalana, S., Hudson, D., and Frankel, A. D. (1991). Arginine-Mediated RNA Recognition: the Arginine Fork. Science 252, 1167-1171. 10.1126/science.252.5009.1167.
43. Pham, V. V., Salguero, C., Khan, S. N., Meagher, J. L., Brown, W. C., Humbert, N., de Rocquigny, H., Smith, J. L., and D'Souza, V. M. (2018). HIV-1 Tat interactions with cellular 7SK and viral TAR RNAs identifies dual structural mimicry. Nat. Commun. 9, 4266. 10.1038/s41467-018-06591-6.
44. Jakobovits, A., Smith, D. H., Jakobovits, E. B., and Capon, D. J. (1988). A discrete element 3′ of human immunodeficiency virus 1 (HIV-1) and HIV-2 mRNA initiation sites mediates transcriptional activation by an HIV trans activator. Mol. Cell. Biol. 8, 2555-2561. 10.1128/mcb.8.6.2555-2561.1988.
45. Ghaleb, A. M., and Yang, V. W. (2017). Kruppel-like factor 4 (KLF4): What we currently know. Gene 611, 27-37. 10.1016/j.gene.2017.02.025.
46. Geiman, D. E., Ton-That, H., Johnson, J. M., and Yang, V. W. (2000). Transactivation and growth suppression by the gut-enriched Kruppel-like factor (Kruppel-like factor 4) are dependent on acidic amino acid residues and protein-protein interaction. Nucleic Acids Res. 28, 1106-1113. 10.1093/nar/28.5.1106.
47. Yet, S. F., McA'Nulty, M. M., Folta, S. C., Yen, H. W., Yoshizumi, M., Hsieh, C. M., Layne, M. D., Chin, M. T., Wang, H., Perrella, M. A., et al. (1998). Human EZF, a Kruppel-like zinc finger protein, is expressed in vascular endothelial cells and contains transcriptional activation and repression domains. J. Biol. Chem. 273, 1026-1031. 10.1074/jbc.273.2.1026.
48. Chen, J., Zhang, Z., Li, L., Chen, B.-C., Revyakin, A., Hajj, B., Legant, W., Dahan, M., Lionnet, T., Betzig, E., et al. (2014). Single-molecule dynamics of enhanceosome assembly in embryonic stem cells. Cell 156, 1274-1285. 10.1016/j.cell.2014.01.062.
49. Nguyen, V. Q., Ranjan, A., Liu, S., Tang, X., Ling, Y. H., Wisniewski, J., Mizuguchi, G., Li, K. Y., Jou, V., Zheng, Q., et al. (2021). Spatiotemporal coordination of transcription preinitiation complex assembly in live cells. Mol. Cell, S1097276521005918. 10.1016/j.molcel.2021.07.022.
50. Garcia, D. A., Johnson, T. A., Presman, D. M., Fettweis, G., Wagh, K., Rinaldi, L., Stavreva, D. A., Paakinaho, V., Jensen, R. A. M., Mandrup, S., et al. (2021). An intrinsically disordered region-mediated confinement state contributes to the dynamics and function of transcription factors. Mol. Cell 81, 1484-1498.e6. 10.1016/j.molcel.2021.01.013.
51. Garcia, D. A., Fettweis, G., Presman, D. M., Paakinaho, V., Jarzynski, C., Upadhyaya, A., and Hager, G. L. (2021). Power-law behavior of transcription factor dynamics at the single-molecule level implies a continuum affinity model. Nucleic Acids Res. 49, 6605-6620. 10.1093/nar/gkab072.
52. Hansen, A. S., Amitai, A., Cattoglio, C., Tjian, R., and Darzacq, X. (2020). Guided nuclear exploration increases CTCF target search efficiency. Nat. Chem. Biol. 16, 257-266. 10.1038/s41589-019-0422-3.
53. Pavlou, S., Astell, K., Kasioulis, I., Gakovic, M., Baldock, R., Heyningen, V. van, and Coutinho, P. (2014). Pleiotropic Effects of Sox2 during the Development of the Zebrafish Epithalamus. PLOS ONE 9, e87546. 10.1371/journal.pone.0087546.
54. Boldes, T., Merenbakh-Lamin, K., Journo, S., Shachar, E., Lipson, D., Yeheskel, A., Pasmanik-Chor, M., Rubinek, T., and Wolf, I. (2020). R269C variant of ESR1: high prevalence and differential function in a subset of pancreatic cancers. BMC Cancer 20, 531. 10.1186/s12885-020-07005-x.
55. Keegan, L., Gill, G., and Ptashne, M. (1986). Separation of DNA binding from the transcription-activating function of a eukaryotic regulatory protein. Science 231, 699-704. 10.1126/science.3080805.
56. Tjian, R., and Maniatis, T. (1994). Transcriptional activation: a complex puzzle with few easy pieces. Cell 77, 5-8. 10.1016/0092-8674(94)90227-5.
57. Asimi, V., Sampath Kumar, A., Niskanen, H., Riemenschneider, C., Hetzel, S., Naderi, J., Fasching, N., Popitsch, N., Du, M., Kretzmer, H., et al. (2022). Hijacking of transcriptional condensates by endogenous retroviruses. Nat. Genet., 1-10. 10.1038/s41588-022-01132-w.
58. Henninger, J. E., Oksuz, O., Shrinivas, K., Sagi, I., LeRoy, G., Zheng, M. M., Andrews, J. O., Zamudio, A. V., Lazaris, C., Hannett, N. M., et al. (2021). RNA-Mediated Feedback Control of Transcriptional Condensates. Cell 184, 207-225.e24. 10.1016/j.cell.2020.11.030.
59. Sharp, P. A., Chakraborty, A. K., Henninger, J. E., and Young, R. A. (2022). RNA in formation and regulation of transcriptional condensates. RNA N. Y. N 28, 52-57. 10.1261/rna.078997.121.
60. Quinodoz, S. A., Jachowicz, J. W., Bhat, P., Ollikainen, N., Banerjee, A. K., Goronzy, I. N., Blanco, M. R., Chovanec, P., Chow, A., Markaki, Y., et al. (2021). RNA promotes the formation of spatial compartments in the nucleus. Cell 184, 5775-5790.e30. 10.1016/j.cell.2021.10.014.
61. Bose, D. A., Donahue, G., Reinberg, D., Shiekhattar, R., Bonasio, R., and Berger, S. L. (2017). RNA Binding to CBP Stimulates Histone Acetylation and Transcription. Cell 168, 135-149.e22. 10.1016/j.cell.2016.12.020.
62. Lai, F., Orom, U. A., Cesaroni, M., Beringer, M., Taatjes, D. J., Blobel, G. A., and Shiekhattar, R. (2013). Activating RNAs associate with Mediator to enhance chromatin architecture and transcription. Nature 494, 497-501. 10.1038/nature11884.
63. Long, Y., Wang, X., Youmans, D. T., and Cech, T. R. (2017). How do lncRNAs regulate transcription? Sci. Adv. 3, eaao2110. 10.1126/sciadv.aao2110.
64. Hemphill, W. O., Voong, C. K., Fenske, R., Goodrich, J. A., and Cech, T. R. (2022). RNA- and DNA-binding proteins generally exhibit direct transfer of polynucleotides: Implications for target site search. 2022.11.30.518605. 10.1101/2022.11.30.518605.
65. Han, H., Braunschweig, U., Gonatopoulos-Pournatzis, T., Weatheritt, R. J., Hirsch, C. L., Ha, K. C. H., Radovani, E., Nabeel-Shah, S., Sterne-Weiler, T., Wang, J., et al. (2017). Multilayered Control of Alternative Splicing Regulatory Networks by Transcription Factors. Mol. Cell 65, 539-553.e7. 10.1016/j.molcel.2017.01.011.
66. Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci. Publ. Protein Soc. 27, 14-25. 10.1002/pro.3235.
67. Pettersen, E. F., Goddard, T. D., Huang, C. C., Meng, E. C., Couch, G. S., Croll, T. I., Morris, J. H., and Ferrin, T. E. (2021). UCSF ChimeraX: Structure visualization for researchers, educators, and developers. Protein Sci. Publ. Protein Soc. 30, 70-82. 10.1002/pro.3943.
68. Demichev, V., Messner, C. B., Vernardis, S. I., Lilley, K. S., and Ralser, M. (2020). DIA-NN: Neural networks and interference correction enable deep proteome coverage in high throughput. Nat. Methods 17, 41-44. 10.1038/s41592-019-0638-x.
69. Nesvizhskii, A. I., Keller, A., Kolker, E., and Aebersold, R. (2003). A statistical model for identifying proteins by tandem mass spectrometry. Anal. Chem. 75, 4646-4658. 10.1021/ac0341261.
70. Hochberg, Y., and Benjamini, Y. (1990). More powerful procedures for multiple significance testing. Stat. Med. 9, 811-818. 10.1002/sim.4780090710.
71. Baltz, A. G., Munschauer, M., Schwanhäusser, B., Vasile, A., Murakawa, Y., Schueler, M., Youngs, N., Penfold-Brown, D., Drew, K., Milek, M., et al. (2012). The mRNA-Bound Proteome and Its Global Occupancy Profile on Protein-Coding Transcripts. Mol. Cell 46, 674-690. 10.1016/j.molcel.2012.05.021.
72. Castello, A., Fischer, B., Eichelbaum, K., Horos, R., Beckmann, B. M., Strein, C., Davey, N. E., Humphreys, D. T., Preiss, T., Steinmetz, L. M., et al. (2012). Insights into RNA Biology from an Atlas of Mammalian mRNA-Binding Proteins. Cell 149, 1393-1406. 10.1016/j.cell.2012.04.031.
73. Kwon, S. C., Yi, H., Eichelbaum, K., Fohr, S., Fischer, B., You, K. T., Castello, A., Krijgsveld, J., Hentze, M. W., and Kim, V. N. (2013). The RNA-binding protein repertoire of embryonic stem cells. Nat. Struct. Mol. Biol. 20, 1122-1130. 10.1038/nsmb.2638.
74. Bao, X., Guo, X., Yin, M., Tariq, M., Lai, Y., Kanwal, S., Zhou, J., Li, N., Lv, Y., Pulido-Quetglas, C., et al. (2018). Capturing the interactome of newly transcribed RNA. Nat. Methods 15, 213-220. 10.1038/nmeth.4595.
75. Huang, R., Han, M., Meng, L., and Chen, X. (2018). Transcriptome-wide discovery of coding and noncoding RNA-binding proteins. Proc. Natl. Acad. Sci. 115, E3879-E3887. 10.1073/pnas.1718406115.
76. Trendel, J., Schwarzl, T., Horos, R., Prakash, A., Bateman, A., Hentze, M. W., and Krijgsveld, J. (2019). The Human RNA-Binding Proteome and Its Dynamics during Translational Arrest. Cell 176, 391-403.e19. 10.1016/j.cell.2018.11.004.
77. Queiroz, R. M. L., Smith, T., Villanueva, E., Marti-Solano, M., Monti, M., Pizzinga, M., Mirea, D.-M., Ramakrishna, M., Harvey, R. F., Dezi, V., et al. (2019). Comprehensive identification of RNA-protein interactions in any organism using orthogonal organic phase separation (OOPS). Nat. Biotechnol. 37, 169-178. 10.1038/s41587-018-0001-2.
78. He, C., Bozler, J., Janssen, K. A., Wilusz, J. E., Garcia, B. A., Schorn, A. J., and Bonasio, R. (2021). TET2 chemically modifies tRNAs and regulates tRNA fragment levels. Nat. Struct. Mol. Biol. 28, 62-70. 10.1038/s41594-020-00526-w.
79. Blue, S. M., Yee, B. A., Pratt, G. A., Mueller, J. R., Park, S. S., Shishkin, A. A., Starner, A. C., Van Nostrand, E. L., and Yeo, G. W. (2022). Transcriptome-wide identification of RNA-binding protein binding sites using seCLIP-seq. Nat. Protoc. 17, 1223-1265. 10.1038/s41596-022-00680-z.
80. Martin, M. (2011). Cutadapt removes adapter sequences from high-throughput sequencing reads. EMBnet.journal 17, 10-12. 10.14806/ej.17.1.200.
81. Smith, T., Heger, A., and Sudbery, I. (2017). UMI-tools: modeling sequencing errors in Unique Molecular Identifiers to improve quantification accuracy. Genome Res. 27, 491-499. 10.1101/gr.209601.116.
82. Langmead, B., Wilks, C., Antonescu, V., and Charles, R. (2019). Scaling read aligners to hundreds of threads on general-purpose processors. Bioinformatics 35, 421-432. 10.1093/bioinformatics/bty648.
83. Langmead, B., and Salzberg, S. L. (2012). Fast gapped-read alignment with Bowtie 2. Nat. Methods 9, 357-359. 10.1038/nmeth.1923.
84. Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinforma. Oxf. Engl. 25, 2078-2079. 10.1093/bioinformatics/btp352.
85. Quinlan, A. R., and Hall, I. M. (2010). BEDTools: a flexible suite of utilities for comparing genomic features. Bioinformatics 26, 841-842. 10.1093/bioinformatics/btq033.
86. Zhang, Y., Liu, T., Meyer, C. A., Eeckhoute, J., Johnson, D. S., Bernstein, B. E., Nusbaum, C., Myers, R. M., Brown, M., Li, W., et al. (2008). Model-based Analysis of ChIP-Seq (MACS). Genome Biol. 9, R137. 10.1186/gb-2008-9-9-r137.
87. Fujiwara, T., O'Geen, H., Keles, S., Blahnik, K., Linnemann, A. K., Kang, Y.-A., Choi, K., Farnham, P. J., and Bresnick, E. H. (2009). Discovering Hematopoietic Mechanisms Through Genome-Wide Analysis of GATA Factor Chromatin Occupancy. Mol. Cell 36, 667-681. 10.1016/j.molcel.2009.11.001.
88. Dunham, I., Kundaje, A., Aldred, S. F., Collins, P. J., Davis, C. A., Doyle, F., Epstein, C. B., Frietze, S., Harrow, J., Kaul, R., et al. (2012). An integrated encyclopedia of DNA elements in the human genome. Nature 489, 57-74. 10.1038/naturel 1247.
89. Whyte, W. A., Orlando, D. A., Hnisz, D., Abraham, B. J., Lin, C. Y., Kagey, M. H., Rahl, P. B., Lee, T. I., and Young, R. A. (2013). Master Transcription Factors and Mediator Establish Super-Enhancers at Key Cell Identity Genes. Cell 153, 307-319. 10.1016/j.cell.2013.03.035.
90. Guo, Y. E., Manteiga, J. C., Henninger, J. E., Sabari, B. R., Dall'Agnese, A., Hannett, N. M., Spille, J.-H., Afeyan, L. K., Zamudio, A. V., Shrinivas, K., et al. (2019). Pol II phosphorylation regulates a switch between transcriptional and splicing condensates. Nature, 1-6. 10.1038/s41586-019-1464-0.
91. Sharma, D., Zagore, L. L., Brister, M. M., Ye, X., Crespo-Hernández, C. E., Licatalosi, D. D., and Jankowsky, E. (2021). The kinetic landscape of an RNA-binding protein in cells. Nature 591, 152-156. 10.1038/s41586-021-03222-x.
92. Mistry, J., Chuguransky, S., Williams, L., Qureshi, M., Salazar, G. A., Sonnhammer, E. L. L., Tosatto, S. C. E., Paladin, L., Raj, S., Richardson, L. J., et al. (2021). Pfam: The protein families database in 2021. Nucleic Acids Res. 49, D412-D419. 10.1093/nar/gkaa913.
93. Gerstberger, S., Hafner, M., and Tuschl, T. (2014). A census of human RNA-binding proteins. Nat. Rev. Genet. 15, 829-845. 10.1038/nrg3813.
94. Holehouse, A. S., Das, R. K., Ahad, J. N., Richardson, M. O. G., and Pappu, R. V. (2017). CIDER: Resources to Analyze Sequence-Ensemble Relationships of Intrinsically Disordered Proteins. Biophys. J. 112, 16-21. 10.1016/j.bpj.2016.11.3200.
95. Li, C. H., Coffey, E. L., Dall'Agnese, A., Hannett, N. M., Tang, X., Henninger, J. E., Platt, J. M., Oksuz, O., Zamudio, A. V., Afeyan, L. K., et al. (2020). MeCP2 links heterochromatin condensates and neurodevelopmental disease. Nature. 10.1038/s41586-020-2574-4.
96. Blum, M., Chang, H.-Y., Chuguransky, S., Grego, T., Kandasaamy, S., Mitchell, A., Nuka, G., Paysan-Lafosse, T., Qureshi, M., Raj, S., et al. (2021). The InterPro protein families and domains database: 20 years on. Nucleic Acids Res. 49, D344-D354. 10.1093/nar/gkaa977.
97. Bailey, T. L., Boden, M., Buske, F. A., Frith, M., Grant, C. E., Clementi, L., Ren, J., Li, W. W., and Noble, W. S. (2009). MEME Suite: tools for motif discovery and searching. Nucleic Acids Res. 37, W202-W208. 10.1093/nar/gkp335.
98. Emenecker, R. J., Griffith, D., and Holehouse, A. S. (2021). Metapredict: a fast, accurate, and easy-to-use predictor of consensus disorder and structure. Biophys. J. 120, 4312-4319. 10.1016/j.bpj.2021.08.039.
99. Ashkenazy, H., Abadi, S., Martz, E., Chay, O., Mayrose, I., Pupko, T., and Ben-Tal, N. (2016). ConSurf 2016: an improved methodology to estimate and visualize evolutionary conservation in macromolecules. Nucleic Acids Res. 44, W344-W350. 10.1093/nar/gkw408.
100. Bakan, A., Meireles, L. M., and Bahar, I. (2011). ProDy: Protein Dynamics Inferred from Theory and Experiments. Bioinformatics 27, 1575-1577. 10.1093/bioinformatics/btrl68.
101. Henikoff, S., Henikoff, J. G., Kaya-Okur, H. S., and Ahmad, K. (2020). Efficient chromatin accessibility mapping in situ by nucleosome-tethered tagmentation. eLife 9, e63274. 10.7554/eLife.63274.
102. Meers, M. P., Tenenbaum, D., and Henikoff, S. (2019). Peak calling by Sparse Enrichment Analysis for CUT&RUN chromatin profiling. Epigenetics Chromatin 12, 42. 10.1186/s13072-019-0287-4.
103. Serge, A., Bertaux, N., Rigneault, H., and Marguet, D. (2008). Dynamic multiple-target tracing to probe spatiotemporal cartography of cell membranes. Nat. Methods 5, 687-694. 10.1038/nmeth.1233.
104. Hansen, A. S., Woringer, M., Grimm, J. B., Lavis, L. D., Tjian, R., and Darzacq, X. (2018). Robust model-based analysis of single-particle tracking experiments with Spot-On. eLife 7, e33125. 10.7554/eLife.33125.
105. Banani, S. F., Afeyan, L. K., Hawken, S. W., Henninger, J. E., Dall'Agnese, A., Clark, V. E., Platt, J. M., Oksuz, O., Hannett, N. M., Sagi, I., et al. (2022). Genetic variation associated with condensate dysregulation in disease. Dev. Cell. 10.1016/j.devcel.2022.06.010.

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of modulating expression of a target gene, the method comprising:
a) providing an agent that modulates binding between a selected ribonucleic acid (RNA) transcribed from at least one regulatory element of a target gene and a transcription factor which binds to both the RNA and the at least one regulatory element, wherein the agent is selected to bind to an RNA having binding affinity for a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and
b) contacting the agent with a cell that exhibits aberrantly increased or decreased expression of the target gene or aberrantly increased or decreased activity of a gene product of the target gene.

2. The method of claim 1, further comprising identifying the RNA that binds the region of the transcription factor for the target gene.

3. The method of claim 2, wherein identifying the RNA that binds to the region of the transcription factor for the target gene comprises:
a) crosslinking the RNA to the transcription factor for the target gene by:
i) contacting the transcription factor with 4-thiouridine (4SU); and
ii) exposing the transcription factor to ultraviolet radiation, thereby generating an RNA-transcription factor complex;
b) immunoprecipitating the RNA-transcription factor complex;
c) lysing the RNA from the RNA-transcription factor complex; and
d) sequencing the RNA.

4. The method of claim 2, wherein identifying the RNA that binds to the region of the transcription factor for the target gene comprises: binding assays using libraries of oligonucleotides to form complexes of the RNA bound to the oligonucleotides, enriching the complexes of the RNA bound to the oligonucleotides by immunoprecipitation or filter binding, and amplifying (SELEX) or sequencing (RNA Bind-n-Seq) the bound RNA.

5. The method of claim 2, wherein identifying the RNA that binds to the region of the transcription factor for the target gene comprises: computational analysis of an overlap of genomic binding sites for the transcription factor and sequencing of RNA transcribed from the genomic binding site.

6. The method of claim 1, wherein the RNA is transcribed from a genomic locus within 1 kilobase of a genomic locus bound by the transcription factor.

7. The method of claim 1, wherein the RNA is transcribed from a genomic locus more than 1 kilobase of a genomic locus bound by the transcription factor.

8. The method of claim 1, wherein a first or last amino acid of the region of the transcription factor is within 10 amino acids of a DNA-binding domain of the transcription factor.

9. The method of claim 1, wherein binding between the oligonucleotide and the RNA causes a change in secondary structure of the RNA.

10. The method of claim 1, the RNA binds to the transcription factor with a Kd from 40 nM to 1200 nM.

11. The method of claim 1, wherein the RNA is seven to fifteen nucleotides.

12. The method of claim 1, wherein the RNA is eleven nucleotides.

13. The method of claim 1, wherein the RNA is at least seven nucleotides.

14. The method of claim 1, wherein the RNA is no more than fifteen nucleotides.

15. The method of claim 1, wherein at least 75% of amino acids of the region of the transcription factor are arginine or lysine.

16. The method of claim 1, wherein at least 80% of amino acids of the region of the transcription factor are arginine or lysine.

17. The method of claim 1, wherein at least 85% of amino acids of the region of the transcription factor are arginine or lysine.

18. The method of claim 1, wherein at least 90% of amino acids of the region of the transcription factor are arginine or lysine.

19. The method of claim 1, wherein the transcription factor comprises a DNA binding domain selected from the group consisting of a zinc finger, leucine zipper, helix-turn-helix, winged helix-turn-helix, helix-loop-helix, high mobility group (HMG) box, and OB-fold.

20. The method of claim 1, wherein the transcription factor is a human transcription factor.

21. A method of modulating expression of a target gene in a subject, the method comprising:
a) crosslinking a ribonucleic acid (RNA) to a transcription factor for the target gene by:
i) contacting the transcription factor with 4-thiouridine (4SU); and
ii) exposing the transcription factor to ultraviolet radiation, thereby generating an RNA-transcription factor complex;
b) immunoprecipitating the RNA-transcription factor complex;
c) lysing the RNA from the RNA-transcription factor complex;
d) sequencing the RNA; and
e) administering to the subject an oligonucleotide that is antisense to the RNA.

22. The method of claim 21, wherein the oligonucleotide binds a region of the transcription factor for the target gene, whereby binding between the oligonucleotide and the RNA inhibits binding between the RNA and the transcription factor, thereby modulating expression of the target gene.

23. The method of claim 21, wherein the region of the transcription factor is at least nine contiguous amino acids, at least one amino acid of the region is arginine, and a majority of amino acids of the region are arginine or lysine.

24. The method of claim 21, wherein the RNA is transcribed from a genomic locus within 1 kilobase of a genomic locus bound by the transcription factor.

25. The method of claim 21, wherein the RNA is transcribed from a genomic locus more than 1 kilobase of a genomic locus bound by the transcription factor.

26. The method of claim 21, wherein a first or last amino acid of the region of the transcription factor is within 10 amino acids of a DNA-binding domain of the transcription factor.

27. The method of claim 21, wherein binding between the oligonucleotide and the RNA causes a change in secondary structure of the RNA.

28. The method of claim 21, the RNA binds to the transcription factor with a Kd from 40 nM to 1200 nM.

29. The method of claim 21, wherein the RNA is seven to fifteen nucleotides.

30. The method of claim 21, wherein the RNA is eleven nucleotides.

31. The method of claim 21, wherein the RNA is at least seven nucleotides.

32. The method of claim 21, wherein the RNA is no more than fifteen nucleotides.

33. The method of claim 21, wherein at least 75% of amino acids of the region of the transcription factor are arginine or lysine.

34. The method of claim 21, wherein at least 80% of amino acids of the region of the transcription factor are arginine or lysine.

35. The method of claim 21, wherein at least 85% of amino acids of the region of the transcription factor are arginine or lysine.

36. The method of claim 21, wherein at least 90% of amino acids of the region of the transcription factor are arginine or lysine.

37. The method of claim 21, wherein the transcription factor comprises a DNA binding domain selected from the group consisting of a zinc finger, leucine zipper, helix-turn-helix, winged helix-turn-helix, helix-loop-helix, high mobility group (HMG) box, and OB-fold.

38. The method of claim 21, wherein the transcription factor is a human transcription factor.

39. A method of identifying transcription factors that bind to RNA, the method comprising:
a) crosslinking an RNA to the transcription factor by:
i) contacting the transcription factor with 4-thiouridine (4SU); and
ii) exposing the transcription factor to ultraviolet radiation, thereby generating an RNA-transcription factor complex; and
b) performing liquid chromatography with tandem mass spectrometry (LC-MS/MS) to identify transcription factors that bind to the RNA.

40. A method of modulating expression of a target gene in a subject, the method comprising:
administering to the subject an oligonucleotide that is antisense to a ribonucleic acid (RNA) that binds a region of a transcription factor for the target gene, whereby binding between the oligonucleotide and the RNA inhibits binding between the RNA and the transcription factor, thereby modulating expression of the target gene,
wherein the region of the transcription factor is at least nine contiguous amino acids, at least one amino acid of the region is arginine, and a majority of amino acids of the region are arginine or lysine.

41. A method of modulating expression of a target gene, the method comprising
a) providing an agent that modulates binding between a selected ribonucleic acid (RNA) transcribed from at least one regulatory element of a target gene and a transcription factor which binds to both the RNA and the at least one regulatory element, wherein the RNA is selected based on its ability to bind to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and
b) contacting the agent with a cell that exhibits aberrantly increased or decreased expression of the target gene or aberrantly increased or decreased activity of a gene product of the target gene.

42. A method of modulating expression of a target gene, the method comprising modulating binding between a ribonucleic acid (RNA) transcribed from at least one regulatory element of a target gene and a transcription factor which binds to both the RNA and the at least one regulatory element, wherein the RNA binds to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene.

43. A method of modulating expression of a target gene, the method comprising:
a) providing an agent that modulates binding between a selected ribonucleic acid (RNA) transcribed from at least one regulatory element of a target gene and a transcription factor which binds to both the RNA and the at least one regulatory element, wherein the selected RNA has been demonstrated to bind to a region of the transcription factor that is at least nine contiguous amino acids, at least one amino acid of the region is arginine, and a majority of amino acids of the region are arginine or lysine, and wherein modulating binding between the RNA and the transcription factor modulates expression of the target gene; and; and
b) contacting the agent with a cell that exhibits aberrantly increased or decreased expression of the target gene or aberrantly increased or decreased activity of a gene product of the target gene.
